(19) United States
(12) Patent Application Publication
(10) Pub. No.: US 2024/0287150 A1
(43) Pub. Date: Aug. 29, 2024

(54) METHODS AND COMPOSITIONS RELATING TO TISSUE-PROTECTIVE ERYTHROPOIETIN

(71) Applicant: PRESIDENT AND FELLOWS OF HARVARD COLLEGE, Cambridge, MA (US)

(72) Inventors: Jungmin LEE, Cambridge, MA (US); Jeffrey WAY, Cambridge, MA (US)

(73) Assignee: PRESIDENT AND FELLOWS OF HARVARD COLLEGE, Cambridge, MA (US)

(21) Appl. No.: 18/572,549

(22) PCT Filed: Jun. 24, 2022
(86) PCT No.: PCT/US2022/034846

§ 371 Date: Dec. 20, 2023

Related U.S. Application Data
(60) Provisional application No. 63/214,725, filed on Jun. 24, 2021.

(51) Int. Cl. C07K 14/505; A61K 38/00; C07K 16/28

(52) U.S. Cl. CPC C07K 14/505; C07K 16/2896; A61K 38/00; C07K 2317/569; C07K 2317/622; C07K 2319/00

(57) ABSTRACT

The technology described herein is directed to engineered polypeptides comprising an anti-GYPA antibody reagent and an EPO polypeptide, such as an EPO polypeptide that is engineered to be tissue-protective. Further provided herein are methods of treating a neurodegenerative disease or disorder, hypoxic tissue damage, traumatic brain injury, or stroke by administering said polypeptides.

### CROSS-REFERENCE TO RELATED APPLICATIONS

This application claims benefit under 35 U.S.C. § 119(e) of U.S. Provisional Application No. 63/214,725 filed Jun. 24, 2021, the contents of which are incorporated herein by reference in their entirety.

### GOVERNMENT SUPPORT

This invention was made with government support under HL119145 awarded by National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 24, 2022, is named 002806-190160WOPT_SL.txt and is 111,874 bytes in size.

### TECHNICAL FIELD

The technology described herein relates to engineered forms of erythropoietin, e.g., tissue-protective erythropoietin and methods of using such compositions.

### BACKGROUND

Erythropoietin (EPO) stimulates red blood cell (RBC) production in response to hypoxia. It inhibits apoptosis of late-stage erythroid precursors (e.g., Colony-Forming Unit-Erythroid (CFU-E), Burst-Forming Unit-Erythroid BFU-E) and promotes their proliferation and maturation into the fully committed erythroid lineage. Healthy human adult kidneys constitutively produce EPO at low levels, maintaining ˜1-5 pM of circulating EPO under normoxic conditions to sustain constant hemoglobin levels. In response to hypoxic stress or massive blood loss, EPO production is stimulated and the number of circulating erythrocytes increases, allowing for more efficient tissue oxygenation.

EPO, like other cytokines and hormones, is pleiotropic and performs several other biological functions in addition to hematopoiesis. Functional EPO receptors (EPORs) are expressed in many tissues other than erythroid precursors, such as endothelial cells, cardiomyocytes, and cells of the central nervous system. Deletion of EPORs in mouse embryos results not only in impaired erythropoiesis, but also in developmental defects in the heart, the vasculature, and the brain (see e.g., Ogunshola and Bogdanova, 2013, Methods Mol Biol, 982, 13-41, the contents of which are incorporated herein by reference in their entirety). Existence of functional EPORs in non-hematopoietic tissues indicates that EPO activates EPORs in different contexts to induce biological activities that are independent of erythropoiesis.

Non-hematopoietic functions of EPO include enhancement of blood clotting and tissue protection in response to hypoxia. These functions indicate that EPO mediates the body's response to hemorrhage, rather than simply being an RBC-producing hormone. When an animal is wounded, the immediate response by the body should be to stop bleeding, increase RBC production, promote tissue oxygenation, and ensure tissue survival until oxygen levels return to baseline. Pro-thrombotic effects have been observed as adverse side effects of EPO in the treatment of anemia. Chronic kidney failure patients receiving EPO exhibit higher incidences of strokes, hypertension, and death. Cancer patients treated with EPO had accelerated tumor growth and lower survival rate, possibly due to EPORs on cancer cells themselves, increased tumor angiogenesis, and deep vein thrombosis. EPO's tissue-protective effects in response to hypoxia have also been shown in animal models and are indicated in several clinical studies. Intravenous injections of high doses of EPO significantly reduced infarct size and serum markers of brain damage in acute ischemic stroke patients, and improved motor and cognitive function in multiple sclerosis patients. EPO treatment also resulted in a lower mortality rate and improved neurological recovery amongst traumatic brain injury (TBI) patients. The protective activity of EPO is general to all cellular insults tested so far, including hypoxia, TBI and neuronal excitotoxicity. See e.g., Drüeke et al., 2006, N Engl J Med, 355, 2071-2084; Singh et al., 2006, N Engl J Med, 355, 2085-2098; Pfeffer et al., 2009, N Engl J Med, 361, 2019-2032; Henke et al., 2003, Lancet, 362, 1255-1260; Okazaki et al., 2008, Neoplasia, 10, 932-939; Yasuda et al., 2003, Carcinogenesis, 24, 1021-1029; Ehrenreich et al., 2002, Mol Med, 8, 495-505; Ehrenreich et al., 2007, Brain, 130, 2577-2588; Aloizos et al., 2015, Turk Neurosurg, 25, 552-558; Fantacci et al., 2006, Proc Natl Acad Sci USA, 103, 17531-17536; Robinson et al., 2018, Front Neurol. 19, 9:451; Park et al., 2011, Neurotoxicology, 32, 879-887; the contents of each of which are incorporated herein by reference in their entireties.

Due to its erythropoietic and tissue-protective functions, EPO can be used as a therapeutic for various conditions that cause hypoxia, such as chronic obstructive pulmonary disease (COPD), right-side heart failure, and viral infection that requires use of a ventilator. However, two major challenges have limited the clinical use of EPO for tissue protection resulting from hypoxia. First, EPO has a pro-thrombotic effect that is observed at low doses, while the tissue-protective effect requires much higher doses. Thus, doses at which EPO might be effective for tissue protection are considered unsafe. Second, EPO (30.4 kDa) has a short plasma half-life of ˜8 hours after a single intravenous injection in humans (see e.g., Bunn, 2013, Cold Spring Harb Perspect Med, 3, a011619, the contents of which are incorporated herein by reference in their entirety). Its poor pharmacokinetic profile necessitates frequent dosing to maintain the high levels of EPO required for efficacy. There is thus great need for erythropoietic EPO that does not also substantially trigger thrombosis and which exhibits clinically practical pharmacokinetic characteristics.

EPO acts through two distinct receptor complexes (see e.g., FIG. 1A-1B). RBC production and clotting is mediated via EPOR homodimers, whereas the angiogenic and tissue-protective activities of EPO are thought to be regulated by heterodimers of EPOR and the co-receptor CD131 (also known as the receptor common beta subunit). See e.g., Hanazono et al., 1995, Biochem Biophys Res Commun, 208, 1060-1066; Brines et al., 2004, Proc Natl Acad Sci USA, 101, 14907-14912; Leist et al., 2004, Science, 305, 239-242; Bennis et al., 2012; the contents of each of which are incorporated herein by reference in their entireties. There is thus also great need for tissue-protective EPO that does not substantially interfere with CD131 binding. In particular, Taylor et al. (Protein Engineering, Design & Selection 23(4), 251-260, 2010), Burrill et al. PNAS 113:5245-5250 (2016), and Lee et al. (2020, ACS Synth Biol, 9, 191-197), the contents of each of which are incorporated herein by reference in their entireties, describe forms of engineered, targeted EPO in which EPO activity is directed to red blood cell precursors and away from cells that contribute to blood clotting; unfortunately, these designs are expected to not signal through EPO-R/CD131 heterodimeric receptor and therefore not be tissue-protective when cells are stressed by hypoxia. Therefore, there is a need in the art for proteins in which EPO activity is delivered to red blood cell precursors and tissues that may be stressed by hypoxia, while still reducing or eliminated the enhancement of blood clotting that is characteristic of natural EPO and drugs such as epoetin alpha and darbepoetin.

### SUMMARY

As described herein, the inventors have found that specific targeting elements, erythropoietin mutations, and specific linker sizes can be combined into a single protein to provide an engineered red blood cell-stimulating and tissue-protective erythropoietin which avoids the harmful side effects of existing EPO therapeutics. The compositions and methods described herein relate to forms of erythropoietin (“EPO”) that are targeted to both red blood cell precursors and to cells such as neurons that may be damaged by hypoxia, and away from EPO receptor-bearing cells on other cell types that may lead to side effects such as blood clotting. It is noted that these finding are particularly surprising as the structure of GYPA was not previously known well enough to predict how to achieve such spatial organization of the compositions described herein, and whether they could retain EPO activity when subject to the spatial limitations described herein. Furthermore, the EPO polypeptide is engineered to be tissue-protective by introducing at least one affinity-decreasing mutation in the weak face of EPO relative to wild-type EPO. Such mutations do not disrupt binding to the receptor CD131, allowing for a tissue protective effect, especially in neural tissue.

Accordingly, in one aspect described herein is a polypeptide comprising: (a) an engineered erythropoietin (EPO) comprising at least one affinity-decreasing mutation in the weak face of EPO relative to wild-type EPO; (b) an anti-glycophorin A (GYPA) antibody reagent that binds an epitope of SEQ ID NO: 89; and (c) a linker sequence separating the anti-GYPA antibody reagent and the engineered erythropoietin.

In some embodiments of any of the aspects, the weak face of EPO binds with a dissociation constant (K_{d}) of at least 1 μM to the EPO receptor (EPOR).

In some embodiments of any of the aspects, the at least one mutation is in Helix A (Ser9-Gly28 of SEQ ID NO: 1) and/or Helix C (Pro90-Leu112 of SEQ ID NO: 1) relative to wild-type EPO of SEQ ID NO: 1.

In some embodiments of any of the aspects, the at least one mutation is at an amino acid residue relative to SEQ ID NO: 1 selected from S104, R14, Y15, R103, and L108.

In some embodiments of any of the aspects, the at least one mutation is S104I, R14E, R14Q, R14N, Y15I, R103I, R103Q, R103K, or L108A.

In some embodiments of any of the aspects, the at least one mutation is at an amino acid residue relative to SEQ ID NO: 1 selected from R14, R103, and L108.

In some embodiments of any of the aspects, the at least one mutation is R14N, R103I, R103K, or L108A.

In some embodiments of any of the aspects, the at least one mutation is at an amino acid residue relative to SEQ ID NO: 1 selected from R103 and L108.

In some embodiments of any of the aspects, the at least one mutation is R103K or L108A.

In some embodiments of any of the aspects, the at least one mutation does not substantially affect binding to CD131.

In some embodiments of any of the aspects, the engineered erythropoietin does not comprise a mutation in a region of wild-type EPO that binds to CD131.

In some embodiments of any of the aspects, the engineered erythropoietin does not comprise a mutation in the strong face of EPO relative to wild-type EPO.

In some embodiments of any of the aspects, the strong face of EPO binds with a dissociation constant (K_{d}) of no more than 1 nM to EPOR.

In some embodiments of any of the aspects, the engineered erythropoietin does not comprise a mutation in Helix D (F138-C161 of SEQ ID NO: 1) or the AB loop (C29-E55 of SEQ ID NO: 1) relative to wild-type EPO of SEQ ID NO: 1.

In some embodiments of any of the aspects, the engineered erythropoietin does not comprise a mutation at an amino acid residue relative to SEQ ID NO: 1 selected from R150, K45, A30, H32, P87, W88, P90, R53, and E55.

In some embodiments of any of the aspects, the at least one mutation is not R150A, K45D, A30N, H32T, P87V, W88N, P90T, R53N, or E55T.

In some embodiments of any of the aspects, the anti-GYPA antibody reagent comprises one or more CDRs of IH4.

In some embodiments of any of the aspects, the anti-GYPA antibody reagent comprises the three CDRs of IH4.

In some embodiments of any of the aspects, the anti-GYPA antibody reagent comprises a VHH having the sequence of SEQ ID NO: 2 or 50.

In some embodiments of any of the aspects, the anti-GYPA antibody reagent comprises one or more CDRs of an antibody reagent selected from the group consisting of 10F7, 1C3, 2B-11, 2B-12, 2B-13, 2B-18, 2B-19, 2B-20, 2B-21, 2B-25, 2B-4, 2B-9, A63-B/C2, A88-A/F9, A88-D/C7, A88-E/H2, A96-D/A7, A96-E/F7, B14 (also known as BRIC 14), B89 (also known as BRIC 89), BRIC 116, BRIC 117, BRIC 119, BRIC 93, GPA 105, GPA 33, IH4, IH4v1, Mab 158, NaM10-2H12, NaM10-6G4, NaM16-IB10, NaM70-3C10, OSK4-1, R10, R7, and R18.

In some embodiments of any of the aspects, the anti-GYPA antibody reagent comprises one or more CDRs of an antibody reagent selected from R18, IH4, IH4v1, 10F7, and Table 13.

In some embodiments of any of the aspects, the anti-GYPA antibody reagent comprises the CDRs of an antibody reagent selected from R18, IH4, IH4v1, 10F7, and Table 13.

In some embodiments of any of the aspects, the anti-GYPA antibody reagent comprises the VH and VL sequences of an antibody reagent selected from R18, IH4, IH4v1, 10F7, and Table 13.

In some embodiments of any of the aspects, the anti-GYPA antibody reagent comprises an antibody reagent selected from R18, IH4, IH4v1, 10F7, and Table 13.

In some embodiments of any of the aspects, the antibody reagent is selected from 10F7, R18, IH4, IH4v1, 2B-21, 2B-25, 2B-9, 2B-20, 2B-19, NaM70-3C10, 2B-4, B14, B89, R7, and BRIC 93.

In some embodiments of any of the aspects, the antibody reagent is selected from 10F7, IH4, IH4v1, 2B-9, 2B-20, 2B-19, NaM70-3C10, 2B-4, B14, B89, R7, and BRIC 93.

In some embodiments of any of the aspects, the linker sequence is no more than 17 amino acids in length.

In some embodiments of any of the aspects, the linker sequence is 1, 2, 3, 4 or 5 amino acids in length.

In some embodiments of any of the aspects, the linker sequence is at least 5 amino acids in length.

In some embodiments of any of the aspects, the linker sequence is 5-35 amino acids in length.

In some embodiments of any of the aspects, the linker sequence is 5-7 amino acids in length.

In some embodiments of any of the aspects, the linker sequence is 7 or fewer amino acids in length.

In some embodiments of any of the aspects, the polypeptide comprises the amino acid sequence of SEQ ID NO: 148.

In some embodiments of any of the aspects, the polypeptide consists of the amino acid sequence of SEQ ID NO: 148.

In some embodiments of any of the aspects, the polypeptide comprises the amino acid sequence of SEQ ID NO: 148 and a detectable tag.

In some embodiments of any of the aspects, the detectable tag is selected from the group consisting of c-Myc, HA, VSV-G, HSV, FLAG, V5, HIS6, and biotin.

In some embodiments of any of the aspects, the polypeptide consists of the amino acid sequence of SEQ ID NO: 144.

In one aspect described herein is a nucleic acid encoding a polypeptide as described herein.

In one aspect described herein is a vector comprising a nucleic acid as described herein.

In one aspect described herein is a cell comprising a nucleic acid as described herein or a vector as described herein.

In one aspect described herein is a pharmaceutical composition comprising a polypeptide as described herein; a nucleic acid as described herein; a vector as described herein; or a cell as described herein.

In one aspect described herein is a method of increasing erythropoiesis comprising contacting a red blood cell with a polypeptide as described herein.

In one aspect described herein is a method of decreasing neurodegeneration comprising contacting a neuron with a polypeptide as described herein.

In some embodiments of any of the aspects, the neurodegeneration is caused by hypoxia, neuronal excitotoxicity, or traumatic brain injury.

In one aspect described herein is a method of treating a neurodegenerative disease or disorder, hypoxic tissue damage, traumatic brain injury, or stroke in a subject in need thereof, the method comprising administering an effective amount of a polypeptide as described herein to the subject.

In one aspect described herein is a method of treating altitude sickness or hypoxic tissue damage in a subject in need thereof, the method comprising administering an effective amount of a polypeptide as described herein to the subject.

In one aspect described herein is a method of enhancing physical performance in a subject in need thereof, the method comprising administering an effective amount of a polypeptide as described herein to the subject.

In some embodiments of any of the aspects, the polypeptide is at a concentration of at least 10⁻¹⁶ M.

In some embodiments of any of the aspects, the polypeptide is at a concentration of at most 10⁻⁸ M.

In one aspect described herein is a polypeptide as described herein, for use in treating a neurodegenerative disease or disorder, hypoxic tissue damage, traumatic brain injury, stroke, or altitude sickness in a subject in need thereof.

In one aspect described herein is a polypeptide as described herein, for use in enhancing physical performance in a subject in need thereof.

### BRIEF DESCRIPTION OF THE DRAWINGS

FIG. 1A-1P is a series of schematics showing exemplary EPO-H fusion proteins. FIG. 1A-1E show the protein interactions of natural EPO with homodimeric EPOR and heterodimeric EPOR-CD131. FIG. 1A shows that EPO binds asymmetrically to homodimeric EPOR via two distinct binding interfaces: the strong face (K_{D}=1 nM) and the weak face (K_{D}=1 μM). FIG. 1B shows that EPO can also bind to EPOR-CD131 receptors via its strong face. FIG. 1C shows the protein structure of EPO interacting with homodimeric EPOR (see e.g., Protein Data Bank (PDB) ID: 1EER). FIG. 1D shows a zoom-in of the strong binding interface. For receptor binding and activity, critical EPO residues are shown in black sticks (top: K45, bottom: R150). EPOR residues that are within 4 Å of these residues are shown in light grey. FIG. 1E shows a zoom-in of the weak binding interface. For receptor binding and activity, critical EPO residues are shown in medium grey sticks (top to bottom: R103, S104, L108, Y15, R14). EPOR residues that are within 4 Å of these residues are shown in light grey sticks. FIG. 1F shows an exemplary EPO-H fusion protein, comprising the IH4 nanobody, which targets GPA-expressing cells, attached to a mutant EPO by a five-amino acid linker. In this panel and subsequent panels of FIG. 1, IH4 is an exemplary anti-glycophorin A binding element and can be replaced by other anti-glycophorin A binding elements. For FIG. 1G-1J, L108A is shown as an exemplary weak-side mutant of EPO; non-limiting examples of further weak-side mutants are provided herein (see e.g., Tables 1-2). FIG. 1G shows that mutant EPO(L108A) has weakened affinity for homodimeric EPOR, and thus, has little effect on non-target cells that lack GPA. FIG. 1H shows that on erythropoietic target cells that express both GPA and EPOR, the binding of IH4 to GPA localizes the fusion protein to the target cell surface and allows activation of homodimeric EPOR. FIG. 1I shows that the L108A mutation in the EPO element does not disrupt EPO interaction with CD131. As a result, IH4-EPO(L108A) can induce tissue-protective activity via a heterodimeric EPOR-CD131 receptor complex. FIG. 1J shows that IH4-EPO(L108A) also binds to mature RBCs via GPA, thereby extending its plasma half-life. FIG. 1K shows an exemplary EPO-A fusion protein, comprising the IH4 nanobody, which targets GPA-expressing cells, attached to a mutant EPO by a linker, wherein the mutation is in the strong face of EPO. FIG. 1L-10 show the behavior of fusion protein that includes the anti-glycophorin A nanobody IH4 attached by a linker to an exemplary strong-side mutant of EPO; non-limiting examples of strong-side mutants are provided herein and in Taylor et al. (Protein Engineering, Design & Selection 23(4), 251-260, 2010). FIG. 1L shows that strong-face mutant EPO has weakened affinity for homodimeric EPOR, and thus, has little effect on non-target cells that lack GPA. FIG. 1M shows that on erythropoietic target cells that express both GPA and EPOR, the binding of IH4 to GPA localizes the fusion protein to the target cell surface and allows activation of homodimeric EPOR. FIG. 1N shows that the strong-face mutation in the EPO element DOES disrupt EPO interaction with EPO-R and thus with EPO-R/CD131 heterodimers. As a result, IH4-EPO(strong-side mutant) cannot induce tissue-protective activity via a heterodimeric EPOR-CD131 receptor complex. This is in contrast to FIG. 1I, wherein an otherwise identical IH4-EPO(weak-side mutant) fusion protein still interacts with EPO-R/CD131 heterodimers. FIG. 1O shows that IH4-EPO(L108A) also binds to mature RBCs via GPA, thereby extending its plasma half-life. FIG. 1P summarizes the contrasting properties of fusion proteins that comprise a glycophorin A binding element attached to an erythropoietin domain with either a weak face or strong face mutation.

FIG. 2A-2C is series of graphs and schematics showing that receptor activation by IH4-EPO(L108A or R103K) in TF-1 cells (an immortal cell line derived from human Erythroleukemia) follows a bell-shaped dose response curve. FIG. 2A is a series of line graphs; IH4-EPO(L108A or R103K) was tested for stimulation of proliferation of TF-1 cells, which express both EPOR and GPA. The fusion proteins showed extremely high potency, with EC₅₀ values at a low femtomolar range, and a drop in bioactivity at high concentrations. Data represent mean±S.E.M. of three replicates. Without wishing to be bound by theory, FIG. 2B-2C show schematics of proposed mechanisms for the bell-shaped dose response curve (see e.g., FIG. 2A). The fusion protein binds to GPA and one copy of EPOR via IH4 (grey oval) and the strong face of EPO, respectively. FIG. 2B shows that at low fusion protein concentrations, EPO has a brief interaction with the second copy of EPOR via the EPO weak face. This transient interaction activates EPOR signaling for cell proliferation, but does not last long enough to trigger receptor-mediated endocytosis. Thus, signaling does not terminate and a few signaling complexes per cell are sufficient to stimulate proliferation. FIG. 2C shows at high concentrations, fusion proteins saturate EPORs with a 1:1 stoichiometry via the strong-face interaction, resulting in a low number of complete signaling complexes composed of one ligand and two receptors.

FIG. 3A-3B is series of line graphs the erythropoietic activity of IH4-EPO(L108A) in vivo. Transgenic mice that express human GPA on their RBCs received a single intraperitoneal (i.p.) injection of saline, darbepoetin (i.e., a hyperglycosylated form of EPO), or IH4-EPO(L108A). Their reticulocyte and reticulated platelet levels were measured by flow cytometry on Days 0, 4 and 7 post-injection. FIG. 3A-3B show that while the untargeted EPO form, darbepoetin, stimulates the production of both reticulocytes and reticulated platelets (middle panels of FIG. 3A-3B), IH4-EPO(L108A) specifically stimulates RBC production and not platelet production in these mice (right panels of FIG. 3A-3B). Data represent mean±S.E.M of five mice per dose group.

FIG. 4A-4G is series of bar graphs and a table showing the ability of EPO variants to protect neuronal cells from CoCl₂-induced hypoxic damage in vitro. SH-SY5Y cells were co-treated with EPO and CoCl₂ for 24 hr and cell viability was measured. FIG. 4A-4B show that positive controls, EPO(WT) and EPO(S104I), protect neuronal cells from cell death in a dose-dependent manner, but FIG. 4C shows that a fusion protein containing a strong-face mutant, IH4-EPO(K45D), does not promote neuroprotection. FIG. 4D-4F show that EPO variants containing a weak-face mutation, e.g., EPO(L108A) and IH4-EPO(L108A or R103K), also show neuroprotective effect against CoCl₂-induced hypoxic damage. Data represent mean±S.E.M. of three replicates. FIG. 4G is a table showing a summary of tissue-protective activity of EPO variants. N indicates the number of repeat experiments, each containing two to four replicates. EC₅₀ values were estimated by the standard four-parameter non-linear fit. See e.g., Example 1 for more details.

FIG. 5A-5L is a series of line graphs showing that in vitro erythropoietic activities of EPO mutants in unfused and antibody-fused forms. Standard TF-1 cell proliferation assays were performed to measure the ability of EPO mutants to stimulate cell proliferation. The same mutants fused to an anti-GPA antibody fragment (IH4 nanobody or 10F7 scFv) via a five-amino acid linker were tested for GPA-dependent activation of EPOR. FIG. 5A shows wild-type EPO activity as a positive control.

FIG. 5B-5C shows that strong-face mutants, K45D and R150A, reduce the EPO activity but show enhanced activity upon fusion to IH4. FIG. 5D-5J show that many weak-face mutations at R14, Y15, S104, and R103 of EPO do not show any activity even when they are fused to an antibody element.

FIG. 5K-5L show that weak-face mutants, R103K and L108A, show slightly reduced and almost no activity by itself, respectively. Their fusion to IH4 exhibits inverted dose response curves, in which their activity is greatly enhanced at low concentrations but drops back to the baseline at high concentrations. Data represent mean±S.E.M. of three replicates.

FIG. 6A-6C is a series of graphs showing the in vivo erythropoietic activity of IH4-EPO(L108A). Transgenic mice that express human GPA on RBCs received a single i.p. injection of saline, darbepoetin, or IH4-EPO(L108A). Reticulocyte and reticulated platelet levels were measured by flow cytometry on Days 0, 4, and 7 post-injection. FIG. 6A-6B show that IH4-EPO(L108A) specifically stimulated RBC production and not platelet production. FIG. 6C shows that IH4-EPO(L108A) induced erythropoiesis in a dose-dependent manner, as shown on Day 4 post-injection. Data represent mean±S.E.M of five mice per dose group.

FIG. 7A-7F is a series of bar graphs showing the ability of EPO variants to protect neuronal cells from CoCl₂-induced hypoxic damage in vitro. SH-SY5Y cells were co-treated with EPO and 100 μM CoCl₂ for 24 hr and cell viability was measured. For each protein, at least two repeat experiments were performed. FIG. 7A-7B show that two positive controls, EPO(WT) and EPO(S104I), showed tissue-protective effect in a dose-dependent manner, although EPO(S104I) showed a weaker effect. FIG. 7C shows that a fusion protein containing a strong-face mutation, K45D, does not have tissue-protective activity. FIG. 7D-7E show that a fusion protein containing a weak-face mutation, R103K or L108A, protected neuronal cells from CoCl₂-induced cell death. FIG. 7F shows that EPO with the weak-face mutation L108A in the absence of fusion to the IH4 nanobody protected neuronal cells from CoCl₂-induced cell death. Note that the tissue-protective effects were reproducible. Data represent mean±S.E.M. of two to three replicates.

FIG. 8A-8F is a series of bar graphs showing that pre-exposure to EPO variants also protected neuronal cells from CoCl₂-induced hypoxic damage in vitro. SH-SY5Y cells were pre-treated with EPO 24 hr prior to adding CoCl₂. Cells were incubated for additional 24 hr after receiving 100 μM CoCl₂, and cell viability was measured. FIG. 8A-8B show that two positive controls, EPO(WT) and EPO(S104I), showed tissue-protective effect in a dose-dependent manner, although EPO(S104I) showed a weaker effect. FIG. 8C-8F show that EPO(R103K or L108A) in an unfused or antibody-fused form protected neuronal cells from CoCl₂-induced cell death. Note that the tissue-protective effects were reproducible. Data represent mean±S.E.M. of two to four replicates.

FIG. 9A-9B is a series of bar graphs showing the ability of EPO variants to protect SH-SY5Y cells from CoCl₂-induced hypoxic damage in vitro. SH-SY5Y cells were co-treated with EPO and CoCl₂ for 72 hr and cell viability was measured. Hypoxic damage was induced using 50 μM in FIG. 9A and 25 μM CoCl₂ in FIG. 9B. The positive control, EPO(WT), but not a strong-side mutant, EPO(K45D), showed tissue-protective activity. Note that the tissue-protective effects were reproducible. Data represent mean±S.E.M. of three replicates.

FIG. 10A-10B is a series of tables and schematics showing that EPO actions are mediated by different receptor complexes and can be modulated by implementing different mutations. FIG. 10A is a table showing that EPO mutants 1, 2, and 3 lack all or a subset of EPO activities. Their ability to promote RBC production can be rescued by fusing to an anti-GPA antibody fragment, depending on the strength of the mutation. FIG. 10B is a schematic showing the surface receptor binding of IH4-5-EPO(Mut) fusion protein on different cell types. Fusion IH4 to EPO increases serum half-life and reduces immunogenicity of the therapeutic protein. Different mutations in EPO can weaken interaction with only one or both of the receptor complexes. Particular bioactivities of EPO can be selectively included or excluded from therapeutic designs.

FIG. 11A-11B is a series of tables and schematics. FIG. 11A is a table showing a list of exemplary EPO mutations described herein. FIG. 11B is a schematic showing the structure of EPO in complex with EPO-R homodimer (see e.g., PDB ID: 1EER) and the EPO residues that are mutated herein. Note that these residues are located at both the strong and weak receptor-binding interfaces. Those at the strong and weak sides are shown in dark grey and light grey, respectively. EPO-R residues that are within 4 Å from these EPO residues are shown as sticks. EPO residues to be mutated make important contacts with EPO-R.

FIG. 12A-12B is a series of tables and graphs showing the erythropoietic activities of several EPO variants in vitro. FIG. 12A is a table summarizing the mutations at strong and weak sides of EPO and their in vitro erythropoietic activities measured by TF-1 cell proliferation assays. FIG. 12B is a series of line graphs; the typical TF-1 cell proliferation assay was performed to compare the stimulation of cell proliferation by darbepoetin (hyperglycosylated form of EPO) and two EPO mutants, EPO(R103K) and EPO(L108A), in unfused and fused forms. EPO(R103K) and EPO(L108A) fused to the IH4 nanobody via a 5 amino acid linker rescued or enhanced the activity of EPO mutants alone. Their targeted erythropoietic activity showed bell-shaped dose response curves with very high potency. Data represent mean±S.E.M. of three replicates. “N.A.” indicates no activity. “N.D.” indicates not determined.

FIG. 13 is a series of line graphs showing the erythropoietic activities of IH4-5-EPO(L108A) in vivo. IH4-5-EPO(L108A) specifically stimulated RBC production and not platelet production in transgenic mice that express human GPA on their RBCs. Erythropoietic response to IH4-5-EPO(L108A) in mice is dose-dependent. Data represent mean t S.E.M of five mice per dose group.

FIG. 14A-14I is a series of bar graphs showing the tissue-protective activities of several EPO variants in vitro. Viable cell counts were measured after treating cells with EPO variants and CoCl₂. FIG. 14A-14B show that wild-type EPO (EPO(WT)) and darbepoetin protected cells from cobalt-induced cell death. FIG. 14C shows that EPO(S104I), a positive control (see e.g., Gan et al., 2012, Stroke, 43, 3071-3077) also stimulated tissue protection in this assay. FIG. 14D-14E show that strong-side mutants, EPO(K45D) and EPO(R150A), did not protect cells from dying. FIG. 14F-14I show that weak-side mutants, EPO(R103K) and EPO(L108A), had tissue-protective effects in both unfused and fused forms. Data represent mean t S.E.M. of two or three replicates.

FIG. 15A-15C is a series of schematics showing the GM-CSF signaling complex. FIG. 15A shows the mechanism of assembly of GM-CSF signaling complex. GM-CSF initially forms a low affinity binary complex with GMRα and then forms a high affinity complex with CD131. The complete signaling complex is a dodecamer comprising four molecules of GM-CSF, GMRα, and CD131. FIG. 15B shows a side view of the GM-CSF-GMRα-CD131 dodecameric complex. FIG. 15C shows a top-down views of the GM-CSF-GMRα-CD131 dodecameric complex (see e.g., PDB ID: 4NKQ).

FIG. 16 is a table showing the three types of engineered EPO fusion proteins and their therapeutic applications.

FIG. 17 is a table showing structural alignment approaches to generate structural models of EPO-EPO—R-CD131 heterocomplexes.

FIG. 18 is a table showing EPO residues that are within 4 Å of CD131 domains 1 and 4 in structural alignment models. Those that make polar contacts with CD131 are underlined. Those that are mutated herein are shown in bold.

FIG. 19 is a table showing a summary of non-erythropoietic, tissue-protective EPO derivatives. The table includes the following sequences: UEELERALNSS (SEQ ID NO: 7, wherein U indicates pyroglutamate); GCAEHCSLNENITVPDTKV (SEQ ID NO: 8, corresponding to residues 28 to 46 of EPO, see e.g., SEQ ID NO: 1); SGLRSLITLLRA (SEQ ID NO: 9, corresponding to residues 100 to 111 of EPO, see e.g., SEQ ID NO: 1). See also, Leist et al., 2004, Science, 305, 239-242; Brines et al., 2008, PNAS 105(31), 10925-10930; Yuan et al., 2015, Neurotherapeutics 12(4):850-61; Gan et al. 2012, supra; Dmytriyeva et al., 2019, Neurobiol Aging 81:88-101; the contents of each of which are incorporated herein by reference in their entireties.

FIG. 20 is a series of line graphs showing the erythropoietic activities of different EPO mutants in unfused and fused forms. Typical TF-1 cell proliferation assays were performed to show the stimulation of cell proliferation by various EPO mutants. The same mutants fused to the IH4 nanobody via a 5 amino acid linker were tested for GPA-dependent activation of cell proliferation on the target cell surface. Data represent mean±S.E.M. of three replicates.

FIG. 21 is a bar graph showing that IH4-5-EPO(L108A) stimulated RBC production in transgenic mice that express human GPA on their RBCs. Erythropoietic response to IH4-5-EPO(L108A) in mice was dose-dependent. Data represent mean±S.E.M of five mice per dose group.

FIG. 22A-22B is a series of bar graphs showing optimization of experimental conditions for the in vitro neuroprotection assays. FIG. 22A shows the results of experiments performed to find toxic agents that yield 30-40% cell death. Six toxic agents were tested for their ability to induce cell death in the SH-SY5Y cell line. They were added to cells plated at high (5.0×10⁴ cells/well) or low (1.5×10⁴ cells/well) seeding densities. Each agent was tested at three concentrations (low, medium, and high) within the range reported previously by other groups. The effect of NMDA and glutamate may be complicated by the change in pH. STP=staurosporine; NMDA=N-methyl-d-aspartic acid; CoCl₂=cobalt chloride; H₂O₂=hydrogen peroxide. FIG. 22B is a bar graph showing SH-SY5Y cells seeded at high density challenged with 100 μM or 120 μM of CoCl₂ after 24 hr pre-treatment with varying concentrations of EPO(WT). Data represent mean±S.E.M. of two or three replicates.

FIG. 23A-23F is a series of schematics showing structural alignment models of EPO-EPO—R-CD131 heterocomplexes. FIG. 23A-23D show Models 1, 2, and 3, which show some steric clashes between EPO and CD131. FIG. 23E-23F show Model 4, which shows a slight steric clash between EPOs at the dodecameric interface. FIG. 23C and FIG. 23E show top-down views (i.e., looking down to the membrane) of the heterocomplex. FIG. 23A, FIG. 23B, FIG. 23D, and FIG. 23F show side views (i.e., perpendicular to the membrane) of a single receptor-ligand complex unit, showing EPO, EPO-R, CD131 D1, and CD131 D4. EPO residues that are within 4 Å of CD131 D1 and D4 are shown in light grey sticks. Steric clashes are indicated in circles.

FIG. 24A-24D is a series of schematics showing structural alignment models of EPO-EPO—R-CD131 heterocomplexes in which CD131 interfaces with the weak side of EPO. FIG. 24A-24B show Model 6, which shows severe steric clashes between EPOs as well as between EPO-R and CD131 at the dodecameric interface. FIG. 24C-24D show Model 7, which shows a steric clash between EPOs at the dodecameric interface. It also shows a slight steric clash between side chains of EPO and CD131. FIG. 24A and FIG. 24C show top-down views (i.e., looking down to the membrane) of the heterocomplex. FIG. 24B and FIG. 24D show side views (i.e., perpendicular to the membrane) of a single receptor-ligand complex unit, showing EPO, EPO-R, CD131 D1, and CD131 D4. EPO residues that are within 4 Å of CD131 D1 and D4 are shown in light grey sticks. Steric clashes are indicated in circles.

FIG. 25A-25D is a series of schematics showing structural alignment models of EPO-EPO—R-CD131 heterocomplexes in which CD131 interfaces with the strong side of EPO. FIG. 25A-25B show Model 8, which does not show any steric clash. FIG. 25C-25D show Model 10, which shows a slight steric clash between side chains of EPO and CD131. FIG. 25A and FIG. 25C show top-down views (i.e., looking down to the membrane) of the heterocomplex. FIG. 25B and FIG. 25D show side views (i.e., perpendicular to the membrane) of a single receptor-ligand complex unit, showing EPO, EPO-R, CD131 D1, and CD131 D4. EPO residues that are within 4 Å of CD131 D1 and D4 are shown in light grey sticks.

FIG. 26A-26H is a series of schematics showing amino acid residues that are in contact with a neighboring receptor or ligand in alignment models 1-4, 6-8, and 10. FIG. 26A, FIG. 26C, FIG. 26D, FIG. 26E, and FIG. 26F show Models 1, 3, 4, 6, and 7, which show contact areas spanning helices A and C of EPO. FIG. 26B and FIG. 26G show Models 2 and 8, which show contact areas spanning helices A and D as well as the AB loop of EPO. FIG. 26H shows Model 10, which shows a broad contact region spanning the N and C termini of EPO. EPO, CD131 D1, and CD131 D4 are shown as medium grey, light grey, and dark grey cartoons, respectively. Residues that are within 4 Å of nearby receptor/ligand are shown as sticks. Among these, EPO residues are shown in light grey, with those described herein highlighted in dark grey.

FIG. 27 is a schematic showing exemplary EPO mutants and EPO fusion proteins and their associated functions and applications.

FIG. 28 is a schematic showing exemplary strong side EPO mutant fusion proteins.

FIG. 29 is a schematic showing exemplary weak side EPO mutant fusion proteins.

FIG. 30A-30B are schematics showing protein sequence alignments of IH4 and other nanobodies. FIG. 30A shows the original IH4 sequence (IH4*; see e.g., SEQ ID NO: 50) was modified to match the consensus sequence. IH4* indicates the original protein sequence of the IH4 nanobody from the U.S. Pat. No. 9,879,090. Phe80 in the framework region 3 of IH4* is mutated to tyrosine (grey highlight), and a threonine residue is inserted between Gly117 and Gln118 in the framework region 4 of IH4* (grey highlight). The resulting sequence is shown as IH4 (bold; see e.g., SEQ ID NO: 2). Each dot indicates a position for a single amino acid residue. Numbers indicate amino acid positions every 10 residues. Complementarity-determining regions (CDRs) are shown with dots highlighted in gray and underlined sequences. Sequences between CDRs are framework regions. FIG. 30B shows PDB ID's and brief descriptions of six nanobodies used in sequence alignments (see e.g., SEQ ID NOs: 58-63, Table 10).

### DETAILED DESCRIPTION

As described herein, the inventors have found that specific targeting elements, erythropoietin mutations, and specific linker sizes can be combined into a single protein to provide an engineered red blood cell-stimulating and tissue-protective erythropoietin which avoids the harmful side effects of existing EPO therapeutics. The compositions and methods described herein relate to forms of erythropoietin (“EPO”) that are targeted to red blood cell precursors and also to cells that may be deleteriously affected by hypoxia, such as neurons, and away from EPO receptor-bearing cells on other cell types that may lead to side effects such as blood clotting. It is noted that these finding are particularly surprising as the structure of GYPA was not previously known well enough to predict how to achieve such spatial organization of the compositions described herein, and whether they could retain EPO activity when subject to the spatial limitations described herein. Furthermore, the EPO polypeptide is engineered to be tissue-protective by introducing at least one affinity-decreasing mutation in the weak face of EPO relative to wild-type EPO. Such mutations do not disrupt binding to the receptor CD131, allowing for a tissue protective effect, especially in neural tissue.

EPO has been considered to be an ‘anemia hormone’ whose primary purpose is to promote formation of red blood cells. EPO is produced primarily by the kidney. Kidney failure patients produce little or no EPO from the kidney, and have reduced levels of red blood cells as a result. (A small amount of EPO is also produced by the liver.) In fact, EPO is a pleiotropic hormone that signals in diverse cell types. EPO promotes the differentiation of RBC precursors, and recombinant EPO is used to treat anemia due to chronic kidney disease and myelosuppressive cancer chemotherapy; however, EPO also signals on megakaryocytes, capillary endothelial cells, and tumor cells. EPO action on these cells may promote the thrombosis and tumor progression, documented in clinical trials, that have led to “black box” warnings on EPO-based products.

Burrill et al. (PNAS 113:5245-5250 (2016)) constructed a targeted form of EPO, 10F7-linker-EPO(R150A), with the following characteristics:
1. The EPO molecule was mutated to weaken its affinity for EPO-R, using the mutation Arg150Ala, which reduces binding by about 12-fold;
2. The avidity for RBC precursors was rescued by tethering to a targeting element, an scFv form of the 10F7 antibody, which specifically binds the human RBC marker glycophorin A (huGYPA);
3. The linker between the 10F7 scFv and EPO was about 35 amino acids long, consisting primarily of glycine and serine.

Lee et al. (2020, ACS Synth Biol, 9, 191-197) constructed a targeted form of EPO, IH4-linker-EPO(R150A), with the following characteristics:
1. The EPO molecule was mutated to weaken its affinity for EPO-R, using the mutation Arg150Ala, which reduces binding by about 12-fold;
2. The avidity for RBC precursors was rescued by tethering to a targeting element, a nanobody form of the IH4 antibody, which specifically binds the human RBC marker glycophorin A (huGYPA);
3. The linker between the IH4 scFv and EPO was about 5 amino acids long, consisting primarily of glycine and serine.

Human Glycophorin A (huGYPA; see e.g., SEQ ID NO: 71) is a very abundant protein found on the surface of red blood cells (RBCs), with about 800,000 copies per cell. huGYPA is also present on late RBC precursors, with up to 50,000 copies per cell. The copy number of EPO receptor on late RBC precursors is about 100-1,000.

SEQ ID NO: 71, human GYPA (e.g., NCBI Ref Seq, NP_002090.4) MYGKIIFVLLLSEIVSISALSTTEVAMHTSTSSSVTKSYISSQTNDTHKRDTYAATPRAHEVSEI SVRTVYPPEEETGERVQLAHHFSEPEITLIIFGVMAGVIGTILLISYGIRRLIKKSPSDVKPLPSPD TDVPLSSVEIENPETSDQ. It should be noted that this sequence includes the signal sequence of GYPA (MYGKIIFVLLLSEIVSISA, SEQ ID NO: 140), and that the mature chain beginning at the sequence “LSTTE . . . , SEQ ID NO: 141” is used for numbering purposes herein, particularly in defining the positions of epitopes to which antibody elements bind within the sequence.

The concept of the “tissue-protective EPO” type of fusion protein is that the antibody element that binds to huGYPA first, and then the EPO element binds to its receptor and activates signal transduction through EPO-R homodimers on red blood cell precursors, and through EPO-R/CD131 heterodimeric receptors on cells that may be deleteriously affected by hypoxia. On red blood cell precursors, the EPO element is able to bind to its receptor (namely (EPO-R)₂ homodimers), in spite of the weakening mutation, because the prior binding of the antibody element to huGYPA places the EPO protein element in a very high local concentration around the cell surface. On cells that may be affected by hypoxia, the EPO element is able to bind to the EPO-R/CD131 heterodimeric receptor because mutations in the weak face of EPO generally do not affect binding to this receptor. However, on cells that promote blood clotting in response to EPO signaling (which is mediated by the (EPO-R)₂ homodimeric receptor), binding and signaling does not occur because of the weakening mutation and the absence of GYPA on these cells.

Described herein are optimal antibody elements and linkers for construction of an optimal form of tissue-protective EPO. This improved targeted EPO provides a surprising lack of negative side effects without compromising therapeutic efficacy. It is described herein that different anti-glycophorin antibody elements vary widely in their suitability for use in a tissue-protective EPO fusion protein. In particular, the antibody elements vary in their ability to be expressed, the strength of their binding to huGYPA, and their tendency to modulate an apparent inflammatory signal transduction pathway mediated by the interaction of huGYPA and a second RBC membrane protein, Band 3. The following antibody elements were used:

1. 10F7 (SEQ ID NO: 51)
>ANC33496.2 10F7-linker-EPO, partial
[synthetic construct]
Heavy:
QVKLQQSGAELVKPGASVKLSCKASGYTFNSYFMHWMKQRPVQGL
EWIGMIRPNGGTTDYNEKFKNKATLTVDKSSNTAYMQLNSLTSGD
SAVYYCARWEGSYYALDYWGQGTTVTVS
(SEQ ID NO: 64)
Linker:
SGGGGSGGGGSSGGGGSS (SEQ ID NO: 65)
Light:
DIELTQSPAIMSATLGEKVTMTCRASSNVKYMYWYQQKSGASPKL
WIYYTSNLASGVPGRFSGSGSGTSYSLTISSVEAEDAATYYCQQF
TSSPYTFGGGTKLEIK
(SEQ ID NO: 66)
2. 1C3 (SEQ ID NO: 52)
Heavy:
(SEQ ID NO: 67)
EVRLLESGGGPVQPGGSLKLSCAASGFDFSRYWMNWVRRAPGKGL
EWIGEINQQSSTINYSPPLKDKFIISRDNAKSTLYLQMNKVRSED
TALYYCARLSLTAAGFAYWGQGTLVTVS
Light: (SEQ ID NO: 68)
DIVMSQSPSSLAVSVGEKVSMSCKSSQSLFNSRTRKNYLTWYQQK
PGQSPKPLIYWASTRESGVPDRFTGSGSGTDFTLTISSVQAEDLA
DYYCKQSYNLRTFGGGTKLEIK
3. R18(see also SEQ ID NO: 53)
Heavy chain (SEQ ID NO: 69)
QVKLQQSGGGLVQPGGSLKLSCAAS GFTFSSYGMSWFRQTPDKR
LELVAIINSNGGTTYYPDSVKGRFTISRDNAKNTLYLQMSSLKSE
DTAMYYCARGGGRWLLDYWGQGTTVTVSS
Light chain (SEQ ID NO: 70)
DIELTQSPSSLAVSAGEKVTMSCKSSQSVLYSSNQKNYLAWYQQK
PGQSPKLLIYWASTRESGVPDRFTGSGSGTDFTLTISSVQAEDLA
VYYCHQYLSSSTFGGGTKLEIK
R18 ScFv (SEQ ID NO: 138)
QVKLQQSGGGLVQPGGSLKLSCAASGFTFSSYGMSWFRQTPDKRL
ELVAIINSNGGTTYYPDSVKGRFTISRDNAKNTLYLOMSSLKSED
TAMYYCARGGGRWLLDYWGQGTTVTVSSGGGGSGGGGSSGGGGSS
DIELTQSPSSLAVSAGEKVTMSCKSSQSVLYSSNQKNYLAWYQQK
PGQSPKLLIYWASTRESGVPDRFTGSGSGTDFTLTISSVQAEDLA
VYYCHQYLSSSTFGGGTKLEIK
R18 ScFv nucleic acid (SEQ ID NO: 139);
for SEQ ID NOs: 138-139, italicized text
indicates the variable region of the heavy
chain; bold text indicates a linker;
unformatted text indicates the variable
region of the light chain
CAGGTTAAACTCCAGCAAAGTGGTGGCGGGCTCGTACAACCAGGC
GGTTCCCTCAAGTTGTCCTGCGCCGCATCAGGGTTTACATTTAGC
TCTTATGGTATGTCTTGGTTTCGCCAGACGCCTGACAAGCGACTC
GAGCTGGTCGCTATCATCAATAGTAACGGAGGTACTACATATTAT
CCCGACAGTGTGAAGGGGCGATTTACCATTAGCCGGGACAACGCC
AAAAATACACTGTACCTCCAGATGTCAAGCTTGAAATCAGAAGAT
ACGGCCATGTACTATTGCGCTAGGGGGGGTGGAAGGTGGCTTCTG
GACTATTGGGGTCAGGGTACAACAGTGACAGTATCCTCCGGTGGA
GGTGGATCTGGTGGTGGAGGATCTTCAGGAGGTGGTGGATCTTCC
GACATAGAGCTTACACAATCTCCGTCATCACTGGCAGTCTCAGCC
GGGGAAAAAGTGACAATGTCATGCAAGTCAAGCCAGAGCGTTCTT
TATTCATCTAATCAGAAGAACTACCTGGCATGGTATCAGCAGAAG
CCGGGACAGTCCCCTAAGCTCCTCATCTACTGGGCAAGCACCAGG
GAATCCGGAGTGCCGGACAGGTTTACTGGGTCCGGTTCTGGGACG
GATTTTACGCTTACGATATCAAGTGTCCAAGCTGAGGACCTCGCA
GTATACTACTGTCACCAGTACCTGTCTTCTTCTACTTTTGGGGGT
GGAACGAAACTGGAAATAAAA
4. IH4
(SEQ ID NO: 50; also referred to as IH4*)
5. IH4v1 (SEQ ID NO: 2)
SEQ ID NO: 72-CDR1 of IH4 and IH4v1:
SGYTDSTYCVG
SEQ ID NO: 73-CDR2 of IH4 and IH4v1:
RINTISGRPWYADSVKG
SEQ ID NO: 74-CDR3 of IH4 and IH4v1:
TTANSRGFCSGGYNY

The data and insights of the invention indicate that the antibody elements IH4 and IH4v1 provide the best results in the tissue-protective EPO molecules described herein. A particular insight is that certain antibodies to glycophorin A can cause an inflammatory response by red blood cells, which includes stiffening of the RBC; reduction in membrane fluidity; release of reactive oxygen species such as hydrogen peroxide; and release of ATP, which is a mediator of inflammation and pain when it is outside of cells. Accordingly, the criteria for choosing a glycophorin-binding antibody element include its binding strength, the ability of the antibody element to be expressed from cultured mammalian cells, the epitope on glycophorin A that is bound, and the propensity of the antibody element, especially as a monomeric element, to induce glycophorin A-mediated inflammation as defined herein. With respect to these issues, the antibody elements have the following characteristics.

TABLE 11

| | | | | | | | | CDRs |
|---|---|---|---|---|---|---|---|---|
| | | | | | | | Sequence | (SEQ |
| Antibody | | Binding | | | | | (SEQ ID | ID |
| element | Format | strength | | Expression | Epitope | Inflammation | NO) | NO) |
| 10F7 | scFv | 100 | nM | Good | 27-38 | Strong | 51, 64-66 | |
| 1C3 | scFv | 30 | nM | Poor | ? | ? | 52, 65, 66 | |
| R18 | scFv, Fab | 400 | nM | Good | 49-52 | Weak/none | 53, 69, 70 | |
| IH4 | Nanobody | 30 | nM | OK | 52-55 | Weak/none | 50 | 72-74 |
| IH4v1 | Nanobody | 30 | nM | Good | 52-55 | Weak/none | 2 | 72-74 |

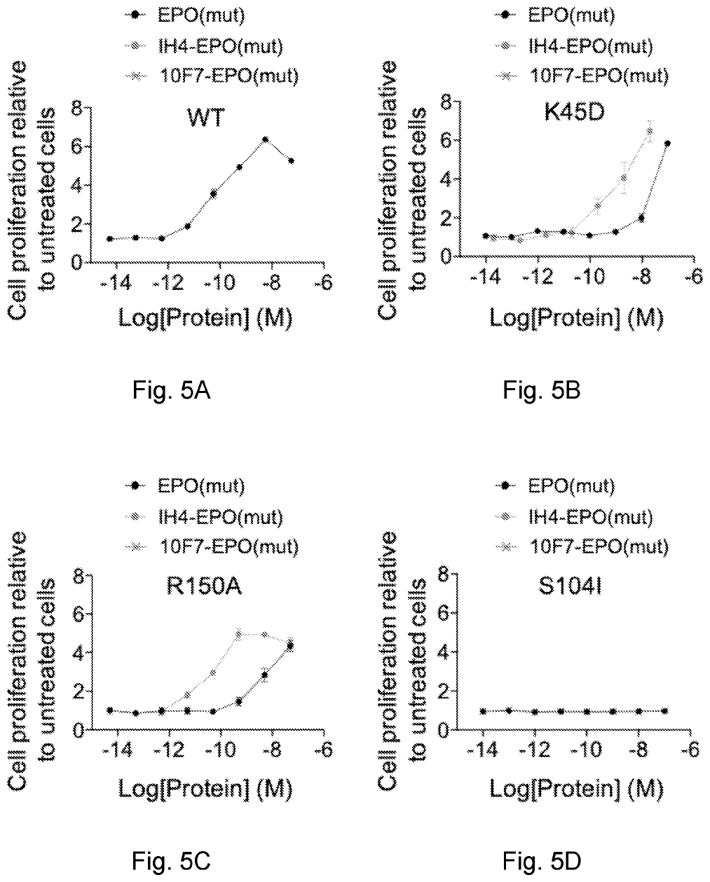

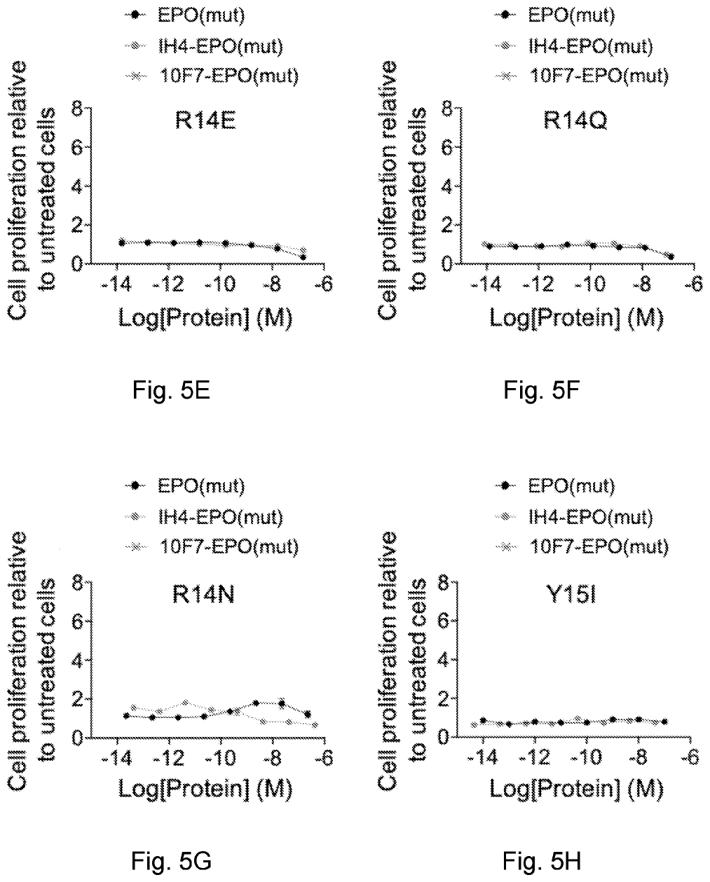

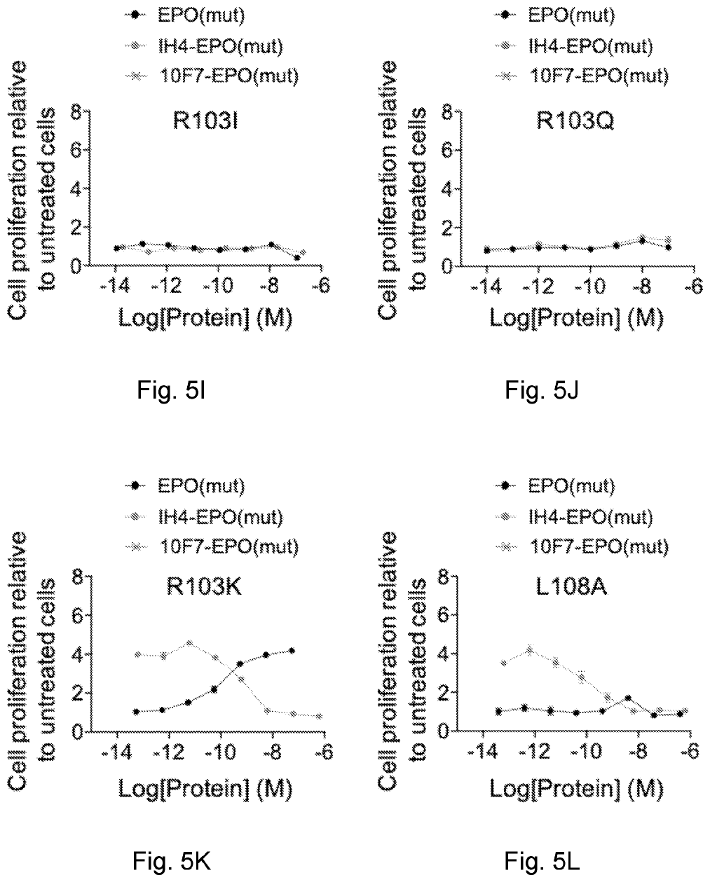

The position of the glycophorin A epitope to which an antibody element binds can have a significant effect on the usefulness of a given form of a tissue-protective EPO, especially when considered in combination with the length of the linker. Specifically, it is generally undesirable for the position of the binding epitope, in combination with a linker of a certain length, to allow the EPO element to extend beyond the N-terminus of the glycophorin A protein as situated on the red blood cell surface. FIGS. 5A-5B of International Patent Application WO 2020/132234, which corresponds to US Patent Publication 2022/0098261A1, illustrate this principle; the content of WO 2020/132234 and US Patent Publication 2022/0098261A1 is incorporated herein by reference in its entirety.

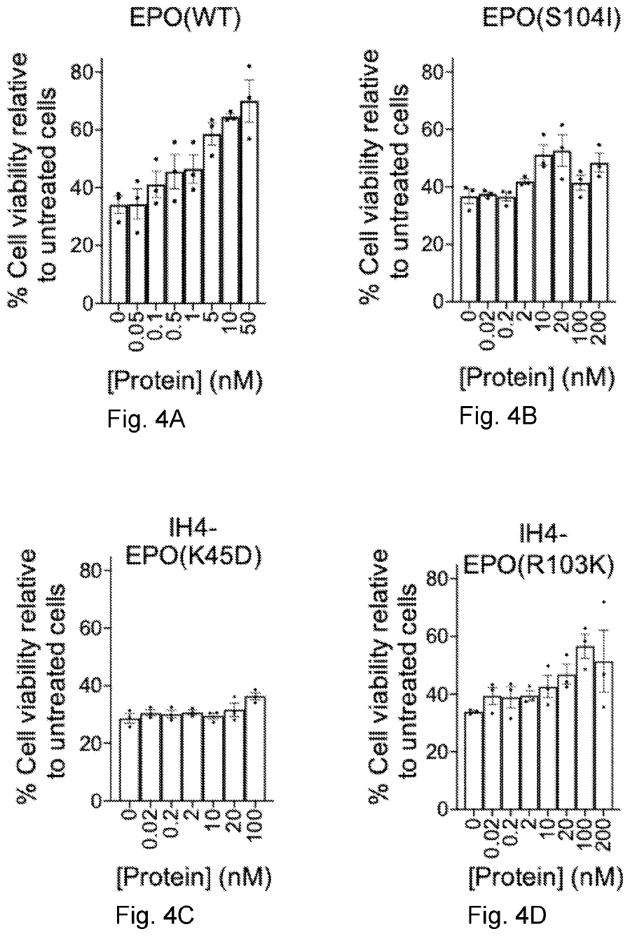

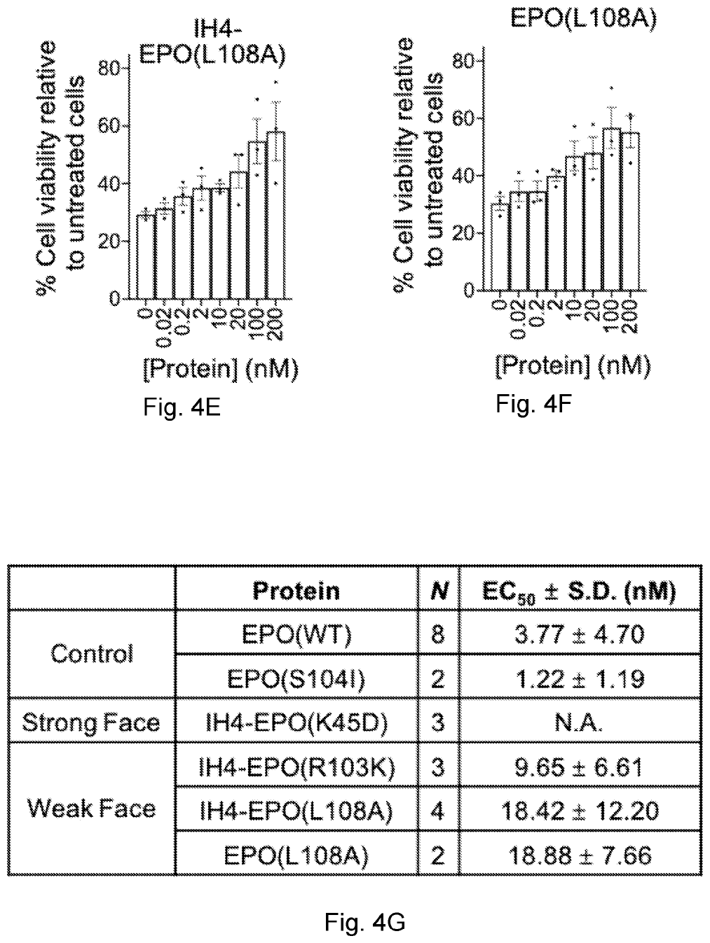

Without wishing to be bound by theory, it contemplated herein that extension of the EPO element beyond the membrane-distal N-terminus of glycophorin A may be deleterious because such EPO elements may interact with EPO receptors on vascular endothelial cells. The result would be that a red blood cell may stick to the wall of a capillary or a larger blood vessel. It is further contemplated herein that the extracellular domain of glycophorin A extends directly away from the cell membrane in a roughly linear manner, with the N-terminus being most distal and the Gly72 being most membrane proximal (see, e.g., FIG. 4 of International Patent Application WO 2020/132234).

The extracellular portion of glycophorin A is thought to be primarily an intrinsically disordered protein. The first 25 amino acids of the mature glycophorin A are highly and variably 0-glycosylated and there is an N-linked glycosylation site at position 26. The high level of glycosylation likely prevents this region from folding into a typical, compact protein structure. The amino acids from 27 to 69 have a high ratio of charged and hydrophilic amino acids compared to hydrophobic amino acids, and include several O-linked glycosylation sites. These characteristics are also consistent with the idea that much of this region is intrinsically disordered.

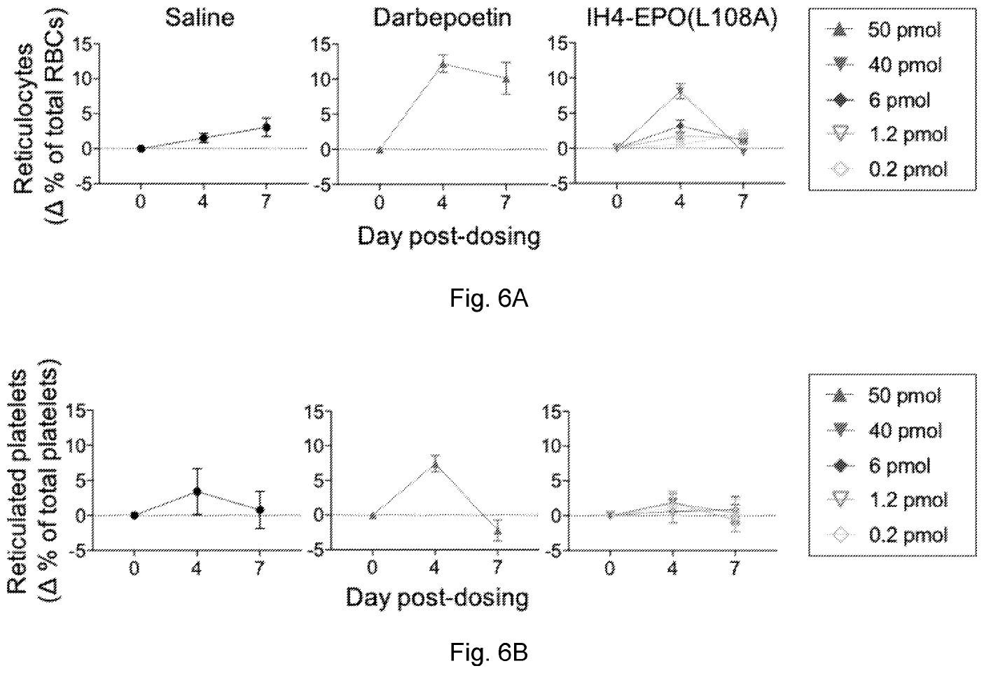

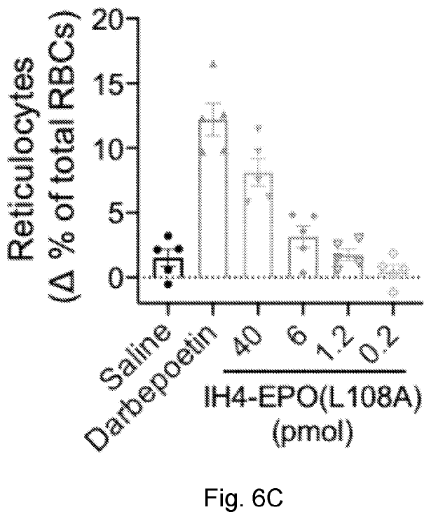

The density of glycophorin A on a red blood cell membrane is so high that this protein likely shields red blood cells from closely approaching each other or other cells. There are about 800,000 copies of glycophorin A on a red blood cell surface, and the surface area of a red blood cell is about 150 square microns, such that the average distance between glycophorin A monomers would be about 14 nanometers or 140 Angstroms. The distance of the glycophorin A N-terminus from the cell membrane may be as great as about 200 Angstroms. Moreover, the oligosaccharides at the N-terminus of glycophorin A are likely to extend laterally by 15-20 Angstroms from the peptide chain. FIGS. 6A-6B of International Patent Application WO 2020/132234 schematically present a top-down view of a red blood cell surface covered with glycophorin A.

SEQ ID NO: 89, Glycophorin A extracellular domain (the below sequence corresponds to residues 20-104 of SEQ ID NO: 71, full-length GYPA). O-linked glycosylation sites are shown in caps

```
                10 20 30 40 50 60 70
. . |. . |. . |. . |. . |. . |. . |. . |. . |. . |. . |. . |. . |. . |. . |
lSTTevamhTSTSssvTkSyiSsqTNdthkrdTyaaTprahevSeiSvrTvyppeeetgervqlahhfsepeitliifgvmagvi . . .
                                                    <-10F7 site> <IH4> <trans-membrane>
```

For an extended amino acid chain, such as a beta strand, the length is about 3 Angstroms per amino acid. This means that the IH4 binding site on glycophorin A may be about 60 Angstroms closer to the cell membrane than the 10F7 binding site. The distance from the antigen binding site of an scFv to the C-terminus of the VH domain (where the linker is attached) is about 32 Angstroms. Thus, if the scFv is angled away from the membrane, then this will increase the average distance of the EPO element from the red blood cell membrane. A fusion protein such as 10F7-(Gly+Ser)₃₉-EPO, when bound to the 10F7 site, likely allows the EPO element to extend beyond the N-terminus of glycophorin A. The binding site of 10F7 on glycophorin A is roughly 33 amino acids from the N-terminus, which is less than the length of the linker attaching 10F7 to EPO. Comparing the molecule IH4-(Gly+Ser)₅-EPO with 10F7-(Gly+Ser)₃₉-EPO, the IH4 epitope is about 20 amino acids farther from the glycophorin A N-terminus; and the linker in IH4-(Gly+Ser)₅-EPO is much shorter, which additionally minimizes the chance that the EPO element is ever membrane-distal to the glycophorin A N-terminus. For sequences of (Gly+Ser)₅, (Gly+Ser)₃₉, or other exemplary linkers, see e.g., SEQ ID NOs: 3, 5, 46-49, 54-57, 90-92, or 136.

Thus, for the IH4-(Gly+Ser)₅-EPO molecule, the EPO element will not be exposed membrane-distal to the N-terminus of glycophorin A, and will not cause side effects that might have resulted from EPO interacting with EPO receptors on the vascular lining while it is tethered to red blood cells. This specific example illustrates the general point that the form of tissue-protective EPO which has an antibody element that binds to a membrane-proximal region of glycophorin A, and has a short linker between the antibody element and the EPO element; is least likely to cause side effects, provided that such a molecule is still able to activate EPO receptors in a glycophorin-dependent manner.

Accordingly, in some embodiments of any of the aspects, a polypeptide described herein, e.g., a tissue-protective EPO satisfies the equation:

(Linker Length)+(Epitope center distance from GYPA's Glu72)<50

To precisely define these parameters, the linker length is defined as the number of amino acids before the Alanine in the EPO sequence Ala-Pro-Pro . . . and after the Lysine in the sequence . . . Lys-Leu-Glu-Ile-Lys (SEQ ID NO: 98) for a fusion to a VL region; after the Serine in the sequence . . . Gln-Val-Thr-Val-Ser (SEQ ID NO: 99) in a nanobody VH; or the second serine in the sequence Val-Thr-Val-Ser-Ser (SEQ ID NO: 10) for a VH region. The epitope center is defined as the central amino acid in a linear glycophorin epitope, if the epitope consists of an odd number of bases, or the center-right amino acid if the epitope has an even number of bases. Because glycophorin A is an intrinsically disordered protein, its antibody epitopes are linear peptides.

In some embodiments of any of the aspects, a polypeptide described herein, e.g., a tissue-protective EPO satisfies the equation:

(Linker Length)+(Epitope center distance from Glu72)<80,

In some embodiments of any of the aspects, a polypeptide described herein, e.g., a tissue-protective EPO satisfies one of the equations that define an “RBC Membrane Distance Metric”:

(Linker Length)+(Epitope center distance from Glu72)<60,

(Linker Length)+(Epitope center distance from Glu72)<40,

(Linker Length)+(Epitope center distance from Glu72)<30, or

(Linker Length)+(Epitope center distance from Glu72)<25.

By way of non-limiting illustration, the value of (Linker Length)+(Epitope center distance from Glu72) for 10F7-(Gly+Ser)₃₉-EPO is 78, while the value of (Linker Length)+(Epitope center distance from Glu72) for IH4-(Gly+Ser)₅-EPO is 23. Specifically, the epitope center of the 10F7 V regions is at about amino acid 33, so (72−33)+39=78. The epitope center of IH4 is at about amino acid 54, so (72−54)+5=23.

The net effect of the short linker in combination with an epitope that is close to the cell surface is to position the EPO element closer to the red blood cell surface than the N-terminus of glycophorin A. The density of glycophorin A on circulating red blood cells is so high that, for a tissue-protective EPO with an RBC Membrane Distance Metric of less than 25, the glycophorin A will prevent the EPO element from acting on EPO receptors on other cells.

Accordingly, in one aspect of any of the embodiments, described herein is polypeptide comprising a) an anti-GYPA antibody reagent that specifically binds the extracellular domain of GYPA C-terminal of residue 34 of GYPA and b) an engineered erythropoietin (e.g., comprising at least one affinity-decreasing mutation in the weak face of EPO relative to wild-type EPO). In one aspect of any of the embodiments, described herein is polypeptide comprising a) an anti-GYPA antibody reagent that specifically binds the extracellular domain of GYPA C-terminal of residue 34 of GYPA, b) an engineered erythropoietin (e.g., comprising at least one affinity-decreasing mutation in the weak face of EPO relative to wild-type EPO), and c) a linker separating the anti-GYPA antibody reagent and the erythropoietin. In one aspect of any of the embodiments, described herein is polypeptide comprising a) an anti-GYPA antibody reagent that specifically binds the extracellular domain of GYPA C-terminal of residue 34 of GYPA, b) an engineered erythropoietin (e.g., comprising at least one affinity-decreasing mutation in the weak face of EPO relative to wild-type EPO), and c) a polypeptide linker separating the anti-GYPA antibody reagent and the erythropoietin. In some embodiments of any of the aspects, the epitope of the anti-GYPA antibody reagent is entirely C-terminal of residue 34 of GYPA. Binding of the anti-GYPA antibody reagent less than about 100 A from the cell surface can restrict the activity of the tissue-protective EPO reagent to a single cell, avoiding inflammatory side effects. As depicted in FIG. 4 of International Patent Application WO 2020/132234, the extracellular domain located between residues 34 and 72 of GYPA is located within 100 A of the cell surface. Anti-GYPA antibodies which bind outside of this region of GYPA will not display the desired characteristics of the tissue-protective EPO reagents described herein (see, e.g., Table 11).

In some embodiments of any of the aspects, the polypeptide comprises an anti-GYPA antibody reagent that specifically binds the extracellular domain of GYPA between resides 34 and 72 of GYPA (e.g., SEQ ID NO: 71). Binding between two residues refers to the epitope of the antibody being located entirely between those residues (inclusive of those residues). An epitope located only partially within a specified range of residues is not considered to be located between those residues. In some embodiments of any of the aspects, the epitope of the anti-GYPA antibody reagent is located between resides 34 and 72 of GYPA (e.g., SEQ ID NO: 71). In some embodiments, it is desired that the anti-GYPA antibody reagent binds within 22-100 A of the cell surface, but binds to structures other than the alpha helices (depicted as a cylinder between residues 34 and 50 of GYPA in FIG. 4 of International Patent Application WO 2020/132234) of the extracellular domain of GYPA. Accordingly, in some embodiments of any of the aspects, the polypeptide comprises an anti-GYPA antibody reagent that specifically binds the extracellular domain of GYPA between resides 49 and 72 of GYPA (e.g., SEQ ID NO: 71). In some embodiments of any of the aspects, the epitope of the anti-GYPA antibody reagent is located between resides 49 and 72 of GYPA (e.g., SEQ ID NO: 71).

In some embodiments, it is desired that the anti-GYPA antibody reagent binds within 22-100 A of the cell surface, but binds to structures other than the alpha helix(ces) of the extracellular domain of GYPA. Accordingly, in some embodiments of any of the aspects, the polypeptide comprises an anti-GYPA antibody reagent that specifically binds the extracellular domain of GYPA between resides 49 and 58 of GYPA (e.g., SEQ ID NO: 71). In some embodiments of any of the aspects, the epitope of the anti-GYPA antibody reagent is located between resides 49 and 58 of GYPA (e.g., SEQ ID NO: 71).

In some embodiments of any of the aspects, the polypeptide comprises an anti-GYPA antibody reagent that specifically binds the extracellular domain of GYPA between resides 49 and 55 of GYPA (e.g., SEQ ID NO: 71). In some embodiments of any of the aspects, the epitope of the anti-GYPA antibody reagent is located between resides 49 and 55 of GYPA (e.g., SEQ ID NO: 71).

Antibody reagents are known and provided herein which bind to the specified epitopes/regions of GYPA. For instance, Table 11 describes the epitopes of R18, IH4, and IH4v1 which meet the specified epitope limitations. In some embodiments of any of the aspects, the polypeptide comprises an anti-GYPA antibody reagent comprising the CDRs of R18, IH4, or IH4v1. In some embodiments ofany of the aspects, the polypeptide comprises an an-GYPA antibody reagent comprising the VL and VH sequences of an antibody reagent selected from R 18, IH4, or IH4v1. In some embodiments ofany of the aspects, the polypeptide comprises an anti-GYPA antibody reagent comprising Re, IH4, or IH4v1.

Additional exemplary anti-GYPA antibody reagents known in the art, along with their binding specificity for GYPA (see e.g., SEQ ID NO: 71) are provided in Table 13.

TABLE 13
Note that the residues refer to the extracellular domain of GYPA,
see e.g., SEQ ID NO: 89 or residues 20-104 of SEQ ID NO: 71.
All references mentioned in this table are
incorporated by reference herein in their entireties.

| Antibody | Epitope | See, e.g., |
|---|---|---|
| 10F7 | Residues 34-38 (YAATP, SEQ | Chasis and Mohandas (1992) Blood 80, 1869- |
| | ID NO: 35) | 1879; Chasis et al. (1988) J. Cell Biol. 107, |
| | | 1351-1357; Catimel et al. (1993) J. Immunol. |
| | | Methods 165, 183-192 |
| IC3 | — | Catimel et al. (1993) J. Immunol. Methods 165, |
| | | 183-192; WO1994007921; WO1993024630 |
| R18 | Residues 49-52 (RTVY, SEQ | Gardner et al. (1989) Immunology 68, 283-289; |
| | ID NO: 36) | US patent 8900592 |
| IH4 or IH4v1 | Residues 52-55 (YPPE, SEQ ID | US patent 9879090; Lee et al., 2020, ACS |
| | NO: 37) | Synth Biol, 9, 191-197; Habib et al., 2013, |
| | | Anal Biochem, 438, 82-89 |
| BRIC 116 | Includes residues 35-36 | Gardner et al. Immunology 68:283-289 (1989) |
| R 10 | Includes residues 35-37 | Gardner et al. Immunology 68:283-289 (1989) |
| | | Anstee Eur. J. Immunol 12 (1982) |
| Mab 158 | Within residues 35-40 | Anstee et al. Journal of Immunogenetics |
| | (AATPRA (SEQ ID NO: 75)) | 17:301-308 (1990) |
| OSK4-1 | Residues 35-45 | Rasamoelisolo et al. Vox Sang 72:185-191 |
| | (AATPRAHEVSE (SEQ ID | (1997) |
| | NO: 76)) | Wasinowska et al. Mol Immunol 29:783-791 |
| | | (1992) |
| GPA 33 | Within residues 35-45 | Rasamoelisolo et al. Vox Sang 72:185-191 |
| | (AATPRAHEVSE (SEQ ID | (1997) |
| | NO: 76)) | |
| GPA 105 | Within residues 35-45 | Rasamoelisolo et al. Vox Sang 72:185-191 |
| | (AATPRAHEVSE (SEQ ID | (1997) |
| | NO: 76)) | |
| BRIC 117 | Includes residues 36-38 | Gardner et al. Immunology 68:283-289 (1989) |
| A88-A/F9 | Within residues 36-45 | Karsten et al. International |
| | (ATPRAHEVSE (SEQ ID NO: | Immunopharmacology 10: 1354-1360 (2010) |
| | 77)) | |
| A88-D/C7 | Within residues 36-45 | Karsten et al. International |
| | (ATPRAHEVSE (SEQ ID NO: | Immunopharmacology 10: 1354-1360 (2010) |
| | 77)) | |
| A88-E/H2 | Within residues 36-45 | Karsten et al. International |
| | (ATPRAHEVSE (SEQ ID NO: | Immunopharmacology 10: 1354-1360 (2010) |
| | 77)) | |
| A96-D/A7 | Within residues 36-45 | Karsten et al. International |
| | (ATPRAHEVSE (SEQ ID NO: | Immunopharmacology 10: 1354-1360 (2010) |
| | 77)) | |
| A96-E/F7 | Within residues 36-45 | Karsten et al. International |
| | (ATPRAHEVSE (SEQ ID NO: | Immunopharmacology 10: 1354-1360 (2010) |
| | 77)) | |
| BRIC 119 | Includes residues 37-39 | Gardner et al. Immunology 68:283-289 (1989) |
| 2B-18 | Residues 38-42 (PRAHE (SEQ | Wasinowska et al. TCB 1:73-75 (1997) |
| | ID NO 78)) | |
| 2B-12 | Residues 38-43 (PRAHEV | Wasinowska et al. TCB 1:73-75 (1997) |
| | (SEQ ID NO: 79)) | |
| 2B-13 | Residues 38-43 (PRAHEV | Wasinowska et al. TCB 1:73-75 (1997) |
| | (SEQ ID NO: 79)) | |
| 2B-11 | Residues 38-44 (PRAHEVS | Wasinowska et al. TCB 1:73-75 (1997) |
| | (SEQ ID NO: 80)) | |
| NaM10-2H12 | Residues 38-45 (PRAHEVSE | Rasamoelisolo et al. Vox Sang 72:185-191 |
| | (SEQ ID NO: 81)) | (1997) |
| NaM16-IB10 | Residues 38-45 (PRAHEVSE | Rasamoelisolo et al. Vox Sang 72:185-191 |
| | (SEQ ID NO : 81)) | (1997) |
| NaM10-6G4 | Residues 38-45 (PRAHEVSE | Rasamoelisolo et al. Vox Sang 72:185-191 |
| | (SEQ ID NO: 81)) | (1997) |
| A63-B/C2 | Within residues 46-55 | Karsten et al. International |
| | (ISVRTVYPPE (SEQ ID NO: | Immunopharmacology 10: 1354-1360 (2010) |
| | 82)) | |
| 2B-21 | Residues 49-56 (RTVYPPEE | Wasinowska et al. TCB 1:73-75 (1997) |
| | (SEQ ID NO: 83)) | |
| 2B-25 | Residues 49-56 (RTVYPPEE | Wasinowska et al. TCB 1:73-75 (1997) |
| | (SEQ ID NO: 83)) | |
| 2B-9 | Residues 52-56 (YPPEE (SEQ | Wasinowska et al. TCB 1:73-75 (1997) |
| | ID NO: 84)) | |
| 2B-20 | Residues 52-57 (YPPEEE (SEQ | Wasinowska et al. TCB 1:73-75 (1997) |
| | ID NO: 85)) | |
| 2B-19 | Residues 52-58 (YPPEEET | Wasinowska et al. TCB 1:73-75 (1997) |
| | (SEQ ID NO: 86)) | |
| NaM70- | Residues 53-57 (PPEEE (SEQ | Lisowska Adv Exp Med Biology 491:155-169 |
| 3C10 | ID NO: 87)) | (2001) |
| | | Rasamoelisolo et al. Hybridoma 17:283-288 |
| | | (1998) |
| 2B-4 | Residues 53-58 (PPEEET (SEQ | Wasinowska et al. TCB 1:73-75 (1997) |
| | ID NO: 88)) | |
| B14 (also | Between aa 56-67 | Ridgewell et al. Biochem J. 209:273-276 |
| known as | | (1983); |
| BRIC 14) | | Chasis et al. JBC 107:1351-1357 (1988); |
| | | Available as Cat. No. 9413 from the |
| | | International Blood Group Reference Lab, |
| | | Bristol UK |
| B89 (also | Includes residues 58-60 | Rasamoelisolo et al. Vox Sang 72:185-191 |
| known as | | (1997) |
| BRIC 89) | | Gardner et al. Immunology 68:283-289 (1989) |
| R7 | Includes residues 58-60 | Rasamoelisolo et al. Vox Sang 72:185-191 |
| | | (1997) |
| | | Anstee Eur. J. Immunol 12 (1982) |
| BRIC 93 | Includes residues 58-60 | Gardner et al. Immunology 68:283-289 (1989) |

In some embodiments of any of the aspects, the polypeptide comprises an anti-GYPA antibody reagent comprising the CDRs of an antibody reagent selected from Table 13. In some embodiments of any of the aspects, the polypeptide comprises an anti-GYPA antibody reagent comprising the VL and VH sequences of an antibody reagent selected from Table 13. In some embodiments of any of the aspects, the polypeptide comprises an anti-GYPA antibody reagent selected from Table 13.

In some embodiments of any of the aspects, the polypeptide comprises an anti-GYPA antibody reagent comprising the CDRs of an antibody reagent selected from Table 13, R18, 1H4, and IH4v1. In some embodiments of any of the aspects, the polypeptide comprises an anti-GYPA antibody reagent comprising the VL and VH sequences of an antibody reagent selected from Table 13, 10F7, R18, IH4, and IH4v1. In some embodiments of any of the aspects, the polypeptide comprises an anti-GYPA antibody reagent selected from Table 13, 10F7, R18, 1H4, and IH4v1.

In some embodiments of any of the aspects, the polypeptide comprises an anti-GYPA antibody reagent comprising the CDRs of an antibody reagent selected from 10F7, R18, IH4, IH4v1, 2B-21, 2B-25, 2B-9, 2B-20, 2B-19, NaM70-3C10, 2B-4, B14, B89, R7, and BRIC 93. In some embodiments of any of the aspects, the polypeptide comprises an anti-GYPA antibody reagent comprising the VL and VH sequences of an antibody reagent selected from 10F7, R18, IH4, IH4v1, 2B-21, 2B-25, 2B-9, 2B-20, 2B-19, NaM70-3C10, 2B-4, B14, B89, R7, and BRIC 93. In some embodiments of any of the aspects, the polypeptide comprises an anti-GYPA antibody reagent selected from 10F7, R18, IH4, IH4v1, 2B-21, 2B-25, 2B-9, 2B-20, 2B-19, NaM70-3C10, 2B-4, B14, B89, R7, and BRIC 93.

In some embodiments of any of the aspects, the polypeptide comprises an anti-GYPA antibody reagent comprising the CDRs of an antibody reagent selected from 10F7, IH4, IH4v1, 2B-9, 2B-20, 2B-19, NaM70-3C10, 2B-4, B14, B89, R7, and BRIC 93. In some embodiments of any of the aspects, the polypeptide comprises an anti-GYPA antibody reagent comprising the VL and VH sequences of an antibody reagent selected from 10F7, IH4, IH4v1, 2B-9, 2B-20, 2B-19, NaM70-3C10, 2B-4, B14, B89, R7, and BRIC 93. In some embodiments of any of the aspects, the polypeptide comprises an anti-GYPA antibody reagent selected from 10F7, IH4, IH4v1, 2B-9, 2B-20, 2B-19, NaM70-3C10, 2B-4, B14, B89, R7, and BRIC 93.

In some embodiments of any of the aspects, the polypeptide comprises an anti-GYPA antibody reagent comprising the CDRs of an antibody reagent selected from 10F7, 1C3, IH4, IH4v1, and R18. In some embodiments of any of the aspects, the polypeptide comprises an anti-GYPA antibody reagent comprising the VL and VH sequences of an antibody reagent selected from 10F7, 1C3, IH4, IH4v1, and R18. In some embodiments of any of the aspects, the polypeptide comprises an anti-GYPA antibody reagent selected from 10F7, 1C3, IH4, IH4v1, and R18.

In some embodiments of any of the aspects, a polypeptide comprising the CDRs of certain antibody reagent comprises six CDRs of the antibody reagent.

In one aspect of any of the embodiments, described herein is a polypeptide comprising a) an anti-GYPA antibody reagent that binds the epitope of SEQ ID NO: 25; b) an erythropoietin, and c) a linker sequence separating the anti-GYPA antibody reagent and the erythropoietin. In some embodiments of any of the aspects, the anti-GYPA antibody reagent comprises one or more CDRs of IH4 (or IH4v1). In some embodiments of any of the aspects, the anti-GYPA antibody reagent comprises the three CDRs of IH4 (or IH4v1), e.g., SEQ ID NOs: 72-74. In some embodiments of any of the aspects, the anti-GYPA antibody reagent comprises a VHH or nanobody having the sequence of SEQ ID NO: 2 or 50.

In some embodiments of any of the aspects, the anti-GYPA antibody reagent comprises one or more CDRs of R18. In some embodiments of any of the aspects, the anti-GYPA antibody reagent comprises the six CDRs of R18. In some embodiments of any of the aspects, the anti-GYPA antibody reagent comprises the VL and/or VH sequence of R18. In some embodiments of any of the aspects, the anti-GYPA antibody reagent comprises the sequences of SEQ ID NO: 53 or SEQ ID NOs: 69-70 or SEQ ID NO: 138.

In some embodiments of any of the aspects, the anti-GYPA antibody reagent comprises one or more CDRs of 10F7. In some embodiments of any of the aspects, the anti-GYPA antibody reagent comprises the six CDRs of 10F7. In some embodiments of any of the aspects, the anti-GYPA antibody reagent comprises the VL and/or VH sequence of 10F7. In some embodiments of any of the aspects, the anti-GYPA antibody reagent comprises the sequences of SEQ ID NO: 51 or SEQ ID NOs: 64-66.

In some embodiments of any of the aspects, the anti-GYPA antibody reagent comprises one or more CDRs of IC3. In some embodiments of any of the aspects, the anti-GYPA antibody reagent comprises the six CDRs of IC3. In some embodiments of any of the aspects, the anti-GYPA antibody reagent comprises the VL and/or VH sequence of IC3. In some embodiments of any of the aspects, the anti-GYPA antibody reagent comprises the sequences of SEQ ID NO: 52 or SEQ ID NOs: 67-68.

In some embodiments of any of the aspects, the anti-GYPA antibody reagent comprises the sequences of at least one of SEQ ID NOs: 2, 50-53, 64-70, 138, or an amino acid sequence that is at least 80%, at least 85%, at least 90%, at least 91%, at least 92%, at least 93%, at least 94%, at least 95%, at least 96%, at least 97%, at least 98%, or at least 99% identical to at least one of SEQ ID NOs: 2, 50-53, 64-70, 138 that maintains the same function (e.g., binding to GYPA). In some embodiments of any of the aspects, the anti-GYPA antibody reagent comprises the sequences of at least one of SEQ ID NOs: 2, 50-53, 64-70, 138, or an amino acid sequence that is at least 95% identical to at least one of SEQ ID NOs: 2, 50-53, 64-70, 138 that maintains the same function (e.g., binding to GYPA).

In some embodiments of any of the aspects, the anti-GYPA antibody reagent is encoded by a nucleic acid sequence comprising at least one of SEQ ID NOs: 4, 38-42, 139 or a nucleic acid sequence that is at least 80%, at least 85%, at least 90%, at least 91%, at least 92%, at least 93%, at least 94%, at least 95%, at least 96%, at least 97%, at least 98%, or at least 99% identical to at least one of SEQ ID NOs: 4, 38-42, 139 or a codon-optimized version thereof, wherein the encoded polypeptide maintains the same function (e.g., binding to GYPA). In some embodiments of any of the aspects, the anti-GYPA antibody reagent is encoded by a nucleic acid sequence comprising at least one of SEQ ID NOs: 4, 38-42, 139 or a nucleic acid sequence that is at least 95% identical to at least one of SEQ ID NOs: 4, 38-42, 139 or a codon-optimized version thereof, wherein the encoded polypeptide maintains the same function (e.g., binding to GYPA).

As used herein, “erythropoietin” or “EPO” refers to a cytokine produced primarily in the kidney and to a lesser extent the liver, and which stimulates erythropoiesis. EPO is a hormone that stimulates RBC production by binding to EPO-Rs on RBC precursors, and can cause a variety of other effects via EPO-Rs on other cell types, such as platelet activation and production, expression of tissue factor on endothelial cells, activation of the renin-angiotensin system, neuroprotection against hypoxia, and acceleration of tumor cell growth (Bunn Cold Spring Harb. Perspect. Med. (2013) 3). EPO plays multiple biological roles by binding to EPO receptors (EPO-R) on diverse cell types, including erythroid progenitors, macrophages, pro-megakaryocytes, cancer cells, and neurons (Jelkmann et al. Ann. Hematol. (2004) 83: 673-686; Bunn Cold Spring Harb. Perspect. Med. (2013) 3). Sequences for EPO nucleic acids and polypeptides are known for a number of species, including, e.g., human EPO (NCBI Gene ID: 2056) mRNA (e.g., NCBI Ref Seq: NM_00799.4) and polypeptides (e.g., NCBI Ref Seq: NP_00790.2 and SEQ ID NO: 1). A therapeutic goal of engineered proteins that include targeted EPO is to minimize the side effects of EPO by targeting the protein to red blood cell (“RBC”) precursors and away from other cell types. Recombinant EPO has been used for two decades to treat forms of anemia associated with end-stage renal failure, AIDS, chemotherapy, or hemoglobinopathies (Jelkmann et al. Ann. Hematol. (2004) 83: 673-686; Bunn Cold Spring Harb. Perspect. Med. (2013) 3) and the compositions described herein can be used in place of recombinant EPO for any therapeutic use for which recombinant EPO is utilized.

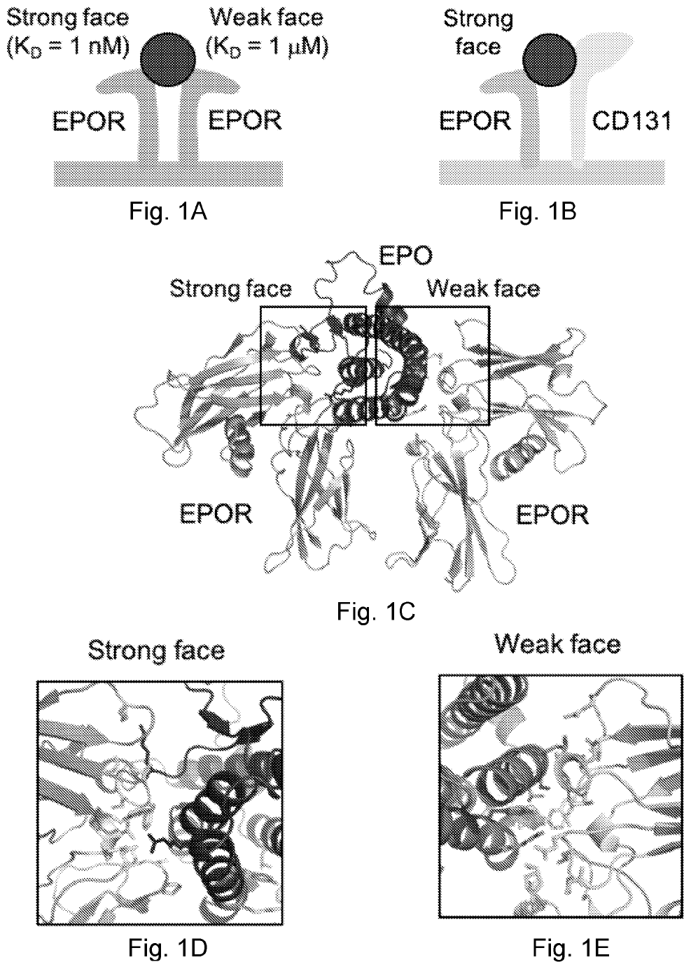

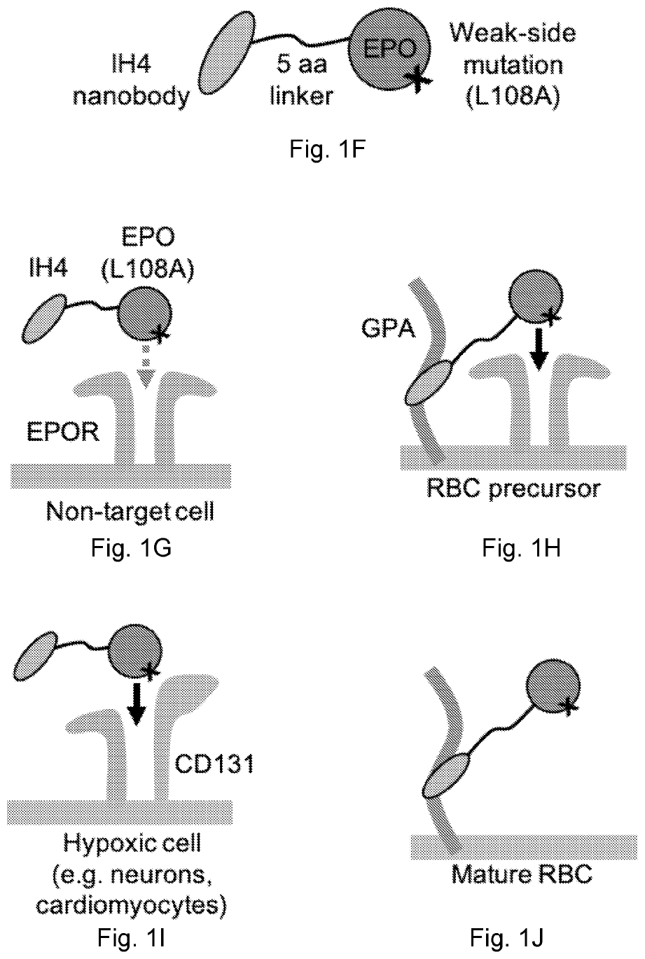

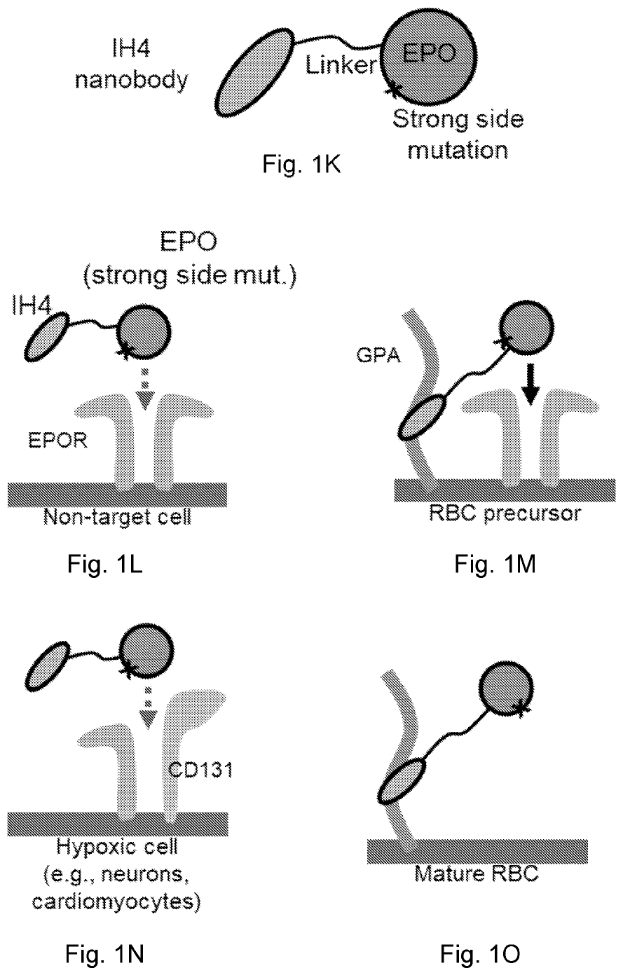

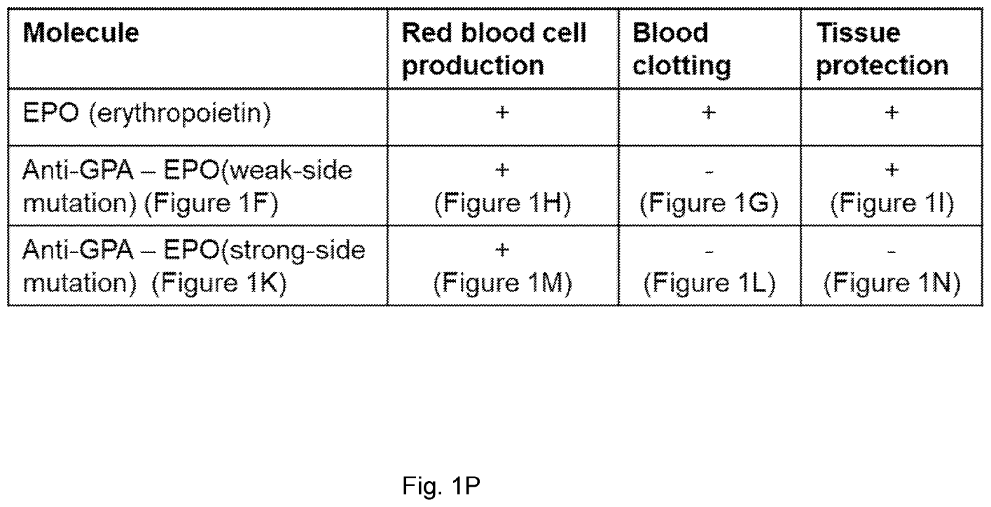

EPO monomers bind to EPOR homodimers through a strong interaction (K_{D}=1 nM) on one face involving residues such as N147 and R150 (referred to herein as the “strong face” or “strong side” of EPO) (see e.g., FIG. 1A, FIG. 1C and FIG. 1D), and through a weak interaction (K_{D}=1 μM) on another face involving residues such as S100, R103, S104 and L108 (referred to herein as the “weak face” or “weak side” of EPO) (see e.g., FIG. 1A, FIG. 1C and FIG. 1E). Described herein is engineered erythropoietin (EPO) comprising at least one affinity-decreasing mutation in the weak face of EPO relative to wild-type EPO. In some embodiments of any of the aspects, the engineered erythropoietin does not comprise a mutation in the strong face of EPO relative to wild-type EPO.

As used herein, the term “weak face” of EPO (also referred to here as “weak side,” “weak-binding interface,” “weak receptor-binding interface,” and the like) refers to the face of EPO that binds with a dissociation constant (K_{d}) of at least 1 μM to the EPO receptor (EPOR). In some embodiments of any of the aspects, the weak face of EPO comprises Helix A (Ser9-Gly28 of SEQ ID NO: 1) and/or Helix C (Pro90-Leu112 of SEQ ID NO: 1) relative to wild-type EPO of SEQ ID NO: 1.

As used herein, the term “strong face” of EPO (also referred to here as “strong side,” “strong-binding interface,” “strong receptor-binding interface,” and the like) refers to the face of EPO that binds with a dissociation constant (K_{d}) of no more than 1 nM to EPOR. In some embodiments of any of the aspects, the strong face of EPO comprises Helix D (F138-C161 of SEQ ID NO: 1) and/or the AB loop (C29-E55 of SEQ ID NO: 1) relative to wild-type EPO of SEQ ID NO: 1.

In some embodiments of any of the aspects, the engineered EPO comprises a wild-type strong face. In some embodiments of any of the aspects, the engineered EPO comprises a mutant weak face. In some embodiments of any of the aspects, the engineered EPO does not comprise a wild-type weak face. In some embodiments of any of the aspects, the engineered EPO does not comprises a mutant strong face.

Human EPO (GenBank: CAA26095.1)
SEQ ID NO: 1
APPRLICDSR VLERYLLEAK EAENITTGCA
EHCSLNENIT VPDTKVNFYA WKRMEVGQQA
VEVWQGLALL SEAVLRGQAL LVNSSQPWEP
LQLHVDKAVS GLRSLTTLLR ALGAQKEAIS
PPDAASAAPL RTITADTFRK LFRVYSNFLR
GKLKLYTGEA CRTGDR,

In SEQ ID NO: 1, bold letters indicate exemplary residues to be mutated (e.g., R14, R103, L108). Weak-side mutations are located in the following domains of EPO: Helix A (Ser9-Gly28 of SEQ ID NO: 1) and Helix C (Pro90-Leu112 of SEQ ID NO: 1), as indicated by the double-underlined regions of SEQ ID NO: 1. Strong-side mutations are located in Helix D (F138-C161 of SEQ ID NO: 1) and the AB loop (C29-E55 of SEQ ID NO: 1). These domains are based on the crystal structure of EPO (see e.g., PDB: lEER). In the lEER structure, chain A is EPO, and chains B and C are EPO-R. The weak face of EPO interacts with the chain C EPO-R. Amino acids in EPO with atoms that are within about 4 Angstroms of an amino acid in chain C are considered to be part of the weak face of EPO.

In some embodiments of any of the aspects, the erythropoietin can be a human erythropoietin. In some embodiments of any of the aspects, the erythropoietin can be an erythropoietin with reduced binding affinity for its receptor (e.g., EPOR) relative to a wild-type erythropoietin, e.g., an erythropoietin with reduced binding affinity to its receptor relative to the wild-type human erythropoietin of SEQ ID NO: 1. Reduced binding affinity can comprise a binding affinity which is reduced 2× or more relative to the reference binding affinity, e.g., reduced 2×, 3×, 4×, 5×, 10×, 12×, 15×, 20× or more. In some embodiments of any of the aspects, reduced binding affinity is a reduction of 10× or more relative to the reference binding affinity of the erythropoietin of SEQ ID NO: 1. In some embodiments of any of the aspects, reduced binding affinity is a reduction of 12× or more relative to the reference binding affinity of the erythropoietin of SEQ ID NO: 1.

In some embodiments of any of the aspects, the engineered erythropoietin comprises at least one mutation in Helix A (Ser9-Gly28 of SEQ ID NO: 1) and/or Helix C (Pro90-Leu112 of SEQ ID NO: 1) relative to wild-type EPO of SEQ ID NO: 1. In some embodiments of any of the aspects, the engineered erythropoietin comprises at least one mutation in Helix A (Ser9-Gly28 of SEQ ID NO: 1) relative to wild-type EPO of SEQ ID NO: 1. In some embodiments of any of the aspects, the engineered erythropoietin comprises at least one mutation in Helix C (Pro90-Leu112 of SEQ ID NO: 1) relative to wild-type EPO of SEQ ID NO: 1. In some embodiments of any of the aspects, the engineered erythropoietin comprises at least one mutation in Helix A (Ser9-Gly28 of SEQ ID NO: 1) and Helix C (Pro90-Leu112 of SEQ ID NO: 1) relative to wild-type EPO of SEQ ID NO: 1.

In some embodiments of any of the aspects, the at least one mutation in the engineered EPO is at an amino acid residue relative to SEQ ID NO: 1 selected from S104, R14, Y15, R103, and L08. In some embodiments of any of the aspects, the engineered erythropoietin comprises an erythropoietin having a sequence with at least one mutation at an amino acid residue of SEQ ID NO: 1 selected from S104, R14, Y15, R103, and L108. In some embodiments of any of the aspects, the engineered erythropoietin comprises an erythropoietin having a sequence with at least 90% identity to SEQ ID NO: 1 and having at least one mutation corresponding to an amino acid residue of SEQ ID NO: 1 selected from S104, R14, Y15, R103, and L108. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 selected from S104, R14, Y15, R103, and L108. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 selected from S104, R14, Y15, R103, and L108. In some embodiments of any of the aspects, the engineered erythropoietin comprises an erythropoietin having the sequence of SEQ ID NO: 1 except for one mutation corresponding to an amino acid residue of SEQ ID NO: 1 selected from S104, R14, Y15, R103, and L108.

In some embodiments of any of the aspects, the at least one mutation in the engineered EPO is S104I, R14E, R14Q, R14N, Y15I, R103I, R103Q, R103K, or L108A, relative to SEQ ID NO: 1. In some embodiments of any of the aspects, the engineered erythropoietin comprises an erythropoietin having a sequence with at least one mutation at an amino acid residue of SEQ ID NO: 1 wherein the at least one mutation is S104I, R14E, R14Q, R14N, Y15I, R103I, R103Q, R103K, or L08A. In some embodiments of any of the aspects, the engineered erythropoietin comprises an erythropoietin having a sequence with at least 90% identity to SEQ ID NO: 1 and having at least one mutation wherein the at least one mutation is S104I, R14E, R14Q, R14N, Y15I, R103I, R103Q, R103K, or L108A. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 wherein the at least one mutation is S104I, R14E, R14Q, R14N, Y15I, R103I, R103Q, R103K, or L108A. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 wherein the at least one mutation is S104I, R14E, R14Q, R14N, Y15I, R103I, R103Q, R103K, or L108A. In some embodiments of any of the aspects, the engineered erythropoietin comprises an erythropoietin having the sequence of SEQ ID NO: 1 except for one mutation corresponding to an amino acid residue of SEQ ID NO: 1 wherein the at least one mutation is S104I, R14E, R14Q, R14N, Y15I, R103I, R103Q, R103K, or L108A.

In some embodiments of any of the aspects, the at least one mutation in the engineered EPO is at an amino acid residue relative to SEQ ID NO: 1 selected from R14, R103, and L108. In some embodiments of any of the aspects, the engineered erythropoietin comprises an erythropoietin having a sequence with at least one mutation at an amino acid residue of SEQ ID NO: 1 selected from R14, R103, and L108. In some embodiments of any of the aspects, the engineered erythropoietin comprises an erythropoietin having a sequence with at least 90% identity to SEQ ID NO: 1 and having at least one mutation corresponding to an amino acid residue of SEQ ID NO: 1 selected from R14, R103, and L108. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 selected from R14, R103, and L108. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 selected from R14, R103, and L108. In some embodiments of any of the aspects, the engineered erythropoietin comprises an erythropoietin having the sequence of SEQ ID NO: 1 except for one mutation corresponding to an amino acid residue of SEQ ID NO: 1 selected from R14, R103, and L108.

In some embodiments of any of the aspects, the at least one mutation in the engineered EPO is R14N, R103I, R103K, or L108A, relative to SEQ ID NO: 1. In some embodiments of any of the aspects, the engineered erythropoietin comprises an erythropoietin having a sequence with at least one mutation at an amino acid residue of SEQ ID NO: 1 wherein the at least one mutation is R14N, R103I, R103K, or L108A. In some embodiments of any of the aspects, the engineered erythropoietin comprises an erythropoietin having a sequence with at least 90% identity to SEQ ID NO: 1 and having at least one mutation wherein the at least one mutation is R14N, R103L, R103K, or L108A. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 wherein the at least one mutation is R14N, R103I, R103K, or L108A. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 wherein the at least one mutation is R14N, R103I, R103K, or L108A. In some embodiments of any of the aspects, the engineered erythropoietin comprises an erythropoietin having the sequence of SEQ ID NO: 1 except for one mutation corresponding to an amino acid residue of SEQ ID NO: 1 wherein the at least one mutation is R14N, R103I, R103K, or L108A.

In some embodiments of any of the aspects, the at least one mutation in the engineered EPO is at an amino acid residue relative to SEQ ID NO: 1 selected from R103 and L108. In some embodiments of any of the aspects, the engineered erythropoietin comprises an erythropoietin having a sequence with at least one mutation at an amino acid residue of SEQ ID NO: 1 selected from R103 and L108. In some embodiments of any of the aspects, the engineered erythropoietin comprises an erythropoietin having a sequence with at least 90% identity to SEQ ID NO: 1 and having at least one mutation corresponding to an amino acid residue of SEQ ID NO: 1 selected from R103 and L108. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 selected from R103 and L108. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 selected from R103 and L108. In some embodiments of any of the aspects, the engineered erythropoietin comprises an erythropoietin having the sequence of SEQ ID NO: 1 except for one mutation corresponding to an amino acid residue of SEQ ID NO: 1 selected from R103 and L108.

In some embodiments of any of the aspects, the at least one mutation in the engineered EPO is R103K or L108A, relative to SEQ ID NO: 1. In some embodiments of any of the aspects, the engineered erythropoietin comprises an erythropoietin having a sequence with at least one mutation at an amino acid residue of SEQ ID NO: 1 wherein the at least one mutation is R103K or L108A. In some embodiments of any of the aspects, the engineered erythropoietin comprises an erythropoietin having a sequence with at least 90% identity to SEQ ID NO: 1 and having at least one mutation wherein the at least one mutation is R103K or L08A. In some embodiments of any of the aspects, the engineered erythropoietin comprises an erythropoietin having a sequence with at least 95% identity to SEQ ID NO: 1 and having at least one mutation corresponding to an amino acid residue of SEQ ID NO: 1 wherein the at least one mutation is R103K or L08A. In some embodiments of any of the aspects, the engineered erythropoietin comprises an erythropoietin having a sequence with at least 98% identity to SEQ ID NO: 1 and having at least one mutation corresponding to an amino acid residue of SEQ ID NO: 1 wherein the at least one mutation is R103K or L108A. In some embodiments of any of the aspects, the engineered erythropoietin comprises an erythropoietin having the sequence of SEQ ID NO: 1 except for one mutation corresponding to an amino acid residue of SEQ ID NO: 1 wherein the at least one mutation is R103K or L108A.

In some embodiments of any of the aspects, the at least one mutation in the weak face of the engineered erythropoietin does not affect binding of the engineered erythropoietin to CD131. CD131 (also referred to as CSF2RB or cytokine receptor common subunit beta) is a common subunit to the following type I cytokine receptors: GM-CSF receptor, IL-3 receptor, or IL-5 receptor. Tissue-protective (e.g., neuroprotective) signaling through EPOR-CD131 heterodimers is thought to involve EPO binding to EPOR through its strong face and an interaction through CD131 (see e.g., FIG. 1B).

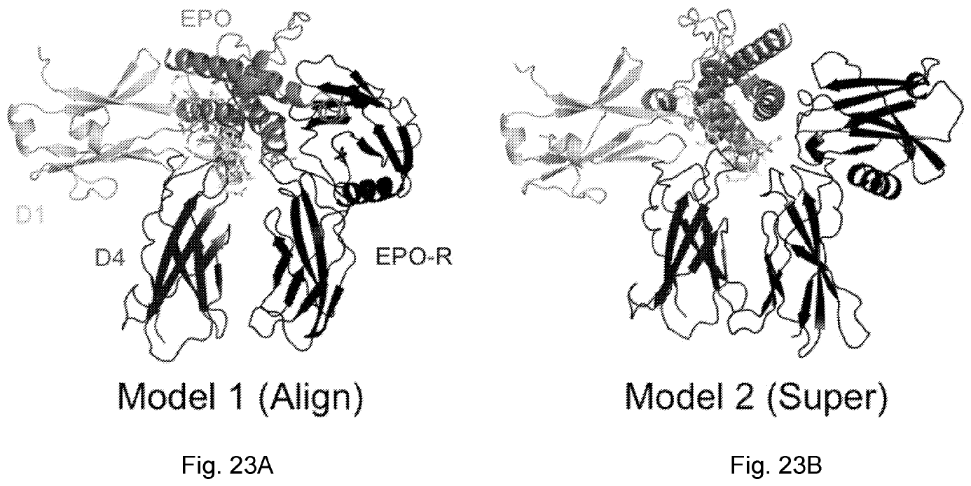

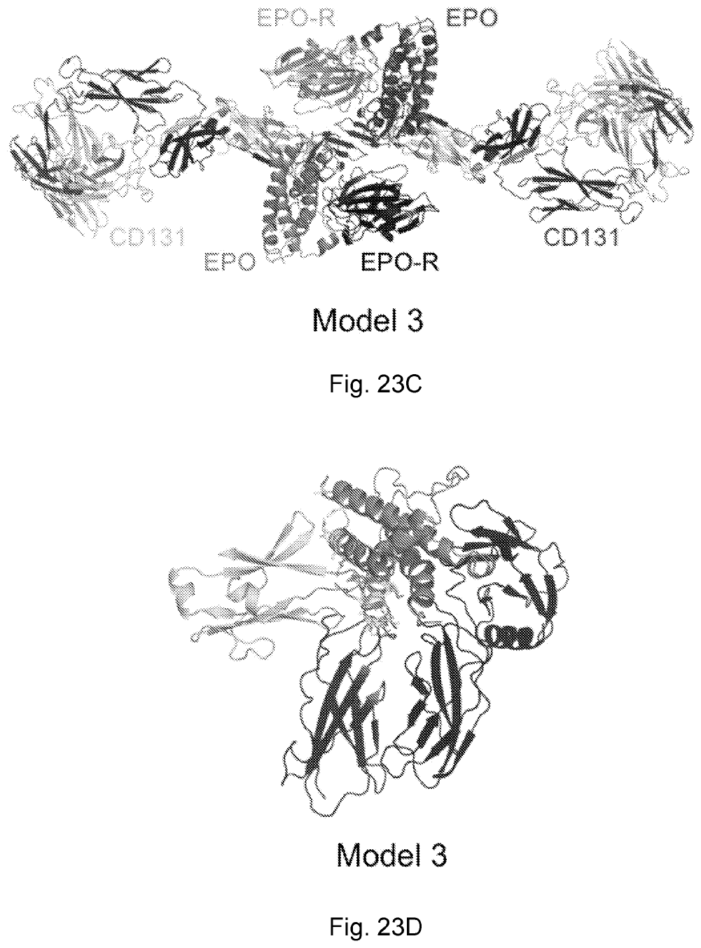

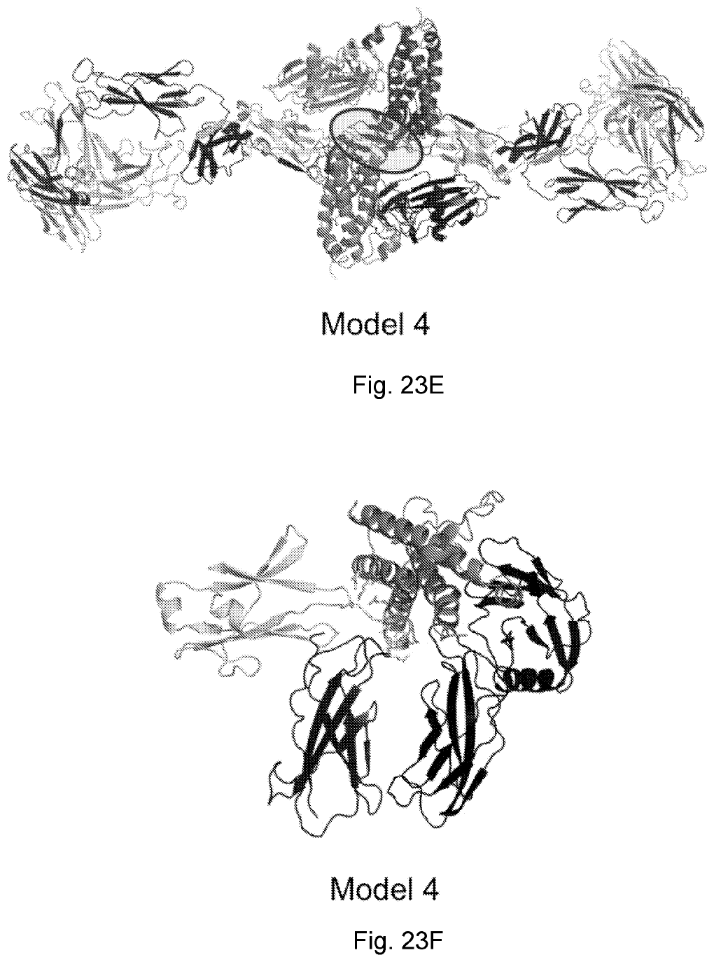

In some embodiments of any of the aspects, the at least one mutation in the weak face of the engineered erythropoietin does not substantially or significantly affect binding of the engineered erythropoietin to CD131. In some embodiments of any of the aspects, the at least one mutation in the weak face of the engineered erythropoietin decreases binding to CD131 by at most 0.1%, at most 0.5%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 25%, at most 30%, at most 40%, or at most 50% relative to the reference binding affinity between wild-type EPO (SEQ ID NO: 1) and CD131. In some embodiments of any of the aspects, the engineered erythropoietin does not comprise a mutation in a region of wild-type EPO that binds to CD131 (see e.g., FIG. 23-26 and Example 2).

In some embodiments of any of the aspects, the engineered erythropoietin does not comprise a mutation in the strong face of EPO relative to wild-type EPO. In some embodiments of any of the aspects, the engineered erythropoietin does not comprise a mutation in Helix D (F138-C161 of SEQ ID NO: 1) or the AB loop (C29-E55 of SEQ ID NO: 1) relative to wild-type EPO of SEQ ID NO: 1. In some embodiments of any of the aspects, the engineered erythropoietin does not comprise a mutation in Helix D (F138-C161 of SEQ ID NO: 1) relative to wild-type EPO of SEQ ID NO: 1. In some embodiments of any of the aspects, the engineered erythropoietin does not comprise a mutation in the AB loop (C29-E55 of SEQ ID NO: 1) relative to wild-type EPO of SEQ ID NO: 1.

In some embodiments of any of the aspects, the engineered erythropoietin does not comprise an erythropoietin having a sequence with at least one mutation at an amino acid residue of SEQ ID NO: 1 selected from R150, K45, A30, H32, P87, W88, P90, R53, and E55. In some embodiments of any of the aspects, the engineered erythropoietin does not comprise an erythropoietin having a sequence with at least 90% identity to SEQ ID NO: 1 and having at least one mutation corresponding to an amino acid residue of SEQ ID NO: 1 selected from R150, K45, A30, H32, P87, W88, P90, R53, and E55. In some embodiments of any of the aspects, the engineered erythropoietin does not comprise an erythropoietin having a sequence with at least 95% identity to SEQ ID NO: 1 and having at least one mutation corresponding to an amino acid residue of SEQ ID NO: 1 selected from R150, K45, A30, H32, P87, W88, P90, R53, and E55. In some embodiments of any of the aspects, the engineered erythropoietin does not comprise an erythropoietin having a sequence with at least 98% identity to SEQ ID NO: 1 and having at least one mutation corresponding to an amino acid residue of SEQ ID NO: 1 selected from R150, K45, A30, H32, P87, W88, P90, R53, and E55. In some embodiments of any of the aspects, the engineered erythropoietin does not comprise an erythropoietin having the sequence of SEQ ID NO: 1 except for one mutation corresponding to an amino acid residue of SEQ ID NO: 1 selected from R150, K45, A30, H32, P87, W88, P90, R53, and E55.

In some embodiments of any of the aspects, the engineered erythropoietin does not comprise an erythropoietin having a sequence with at least one mutation at an amino acid residue of SEQ ID NO: 1 wherein the at least one mutation is R150A, K45D, A30N, H32T, P87V, W88N, P90T, R53N, or E55T. In some embodiments of any of the aspects, the engineered erythropoietin does not comprise an erythropoietin having a sequence with at least 90% identity to SEQ ID NO: 1 and having at least one mutation wherein the at least one mutation is R150A, K45D, A30N, H32T, P87V, W88N, P90T, R53N, or E55T. In some embodiments of any of the aspects, the engineered erythropoietin does not comprise an erythropoietin having a sequence with at least 95% identity to SEQ ID NO: 1 and having at least one mutation corresponding to an amino acid residue of SEQ ID NO: 1 wherein the at least one mutation is R150A, K45D, A30N, H32T, P87V, W88N, P90T, R53N, or E55T. In some embodiments of any of the aspects, the engineered erythropoietin does not comprise an erythropoietin having a sequence with at least 98% identity to SEQ ID NO: 1 and having at least one mutation corresponding to an amino acid residue of SEQ ID NO: 1 wherein the at least one mutation is R150A, K45D, A30N, H32T, P87V, W88N, P90T, R53N, or E55T. In some embodiments of any of the aspects, the engineered erythropoietin does not comprise an erythropoietin having the sequence of SEQ ID NO: 1 except for one mutation corresponding to an amino acid residue of SEQ ID NO: 1 wherein the at least one mutation is R150A, K45D, A30N, H32T, P87V, W88N, P90T, R53N, or E55T.

In some embodiments of any of the aspects, the engineered erythropoietin comprises the sequence of one of SEQ ID NOs: 15-23 or an amino acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 15-23 that maintains the same function (e.g., binding to EPOR and/or CD131). In some embodiments of any of the aspects, the engineered erythropoietin comprises the sequence of one of SEQ ID NOs: 15-23 or an amino acid sequence that is at least 95% identical to one of SEQ ID NOs: 15-23 that maintains the same function (e.g., binding to EPOR and/or CD131).

In some embodiments of any of the aspects, the engineered erythropoietin is encoded by one of SEQ ID NOs: 26-34 or a nucleic acid sequence that is at least 80/a, at least 85%, at least 90%, at least 91%, at least 92%, at least 93%, at least 94%, at least 95%, at least 96%, at least 97%, at least 98%, or at least 99% identical to one of SEQ ID NOs: 26-34, or a codon-optimized version thereof, wherein the encoded polypeptide maintains the same function (e.g., binding to EPOR and/or CD131). In some embodiments of any of the aspects, the engineered erythropoietin is encoded by one of SEQ ID NOs: 26-34 or a nucleic acid sequence that is at least 95% identical to one of SEQ ID NOs: 26-34, or a codon-optimized version thereof, wherein the encoded polypeptide maintains the same function (e.g., binding to EPOR and/or CD131). In some embodiments of any of the aspects, the erythropoietin is a murine or primate erythropoietin.

The term “linker” refers to any means, entity or moiety used to join two or more entities. A peptide linker is typically used to connect the EPO element and the antibody element.

The attachment of the anti-GYPA antibody reagent and the EPO (or activity element) can be by means of linkers, chemical modification, peptide linkers, chemical linkers, covalent or non-covalent bonds, or protein fusion or by any means known to one skilled in the art. The joining can be permanent or reversible. In some embodiments of any of the aspects, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention. According to the present invention, the polypeptide or fragments, derivatives or variants thereof, can be linked to the first fusion partner via any suitable means, as known in the art, see for example U.S. Pat. Nos. 4,625,014, 5,057,301 and 5,514,363, which are incorporated herein in their entirety by reference. For example, the polypeptide e can be covalently conjugated to the anti-GYPA antibody reagent, either directly or through one or more linkers. In one embodiment, a polypeptide as disclosed herein is conjugated directly to the first fusion partner (e.g. anti-GYPA antibody reagent), and in an alternative embodiment, a polypeptide as disclosed herein can be conjugated to a first fusion partner (such as anti-GYPA antibody reagent) via a linker, e.g. a transport enhancing linker.

A large variety of methods for conjugation of two polypeptides are known in the art. Such methods are e.g. described by Hermanson (1996, Bioconjugate Techniques, Academic Press), in U.S. Pat. Nos. 6,180,084 and 6,264,914 which are incorporated herein in their entirety by reference and include e.g. methods used to link haptens to carrier proteins as routinely used in applied immunology. Suitable methods for conjugation of two polypeptides include e.g. carbodiimide conjugation (Bauminger and Wilchek, 1980, Meth. Enzymol. 70: 151-159) or as described by Nagy et al., Proc. Natd. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate two polypeptides as disclosed herein, for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multi-step protocols can offer a great control of conjugate size and the molar ratio of components.

A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling.

Linkers of 5, 7, 17, 18, 29, 35 and 39 amino acids can functionally attach EPO to the various antibody elements. When an scFv is used, typically there is a linker of at least 15 amino acids between the VH and VL elements. It is preferable to use 18 or more amino acids between the VH and VL elements, as these longer linkers minimize the dimer formation. Linkers of lengths from 0 (no linker) to about 100 amino acids may be used. In some embodiments of any of the aspects, the linkers comprise or consist primarily of glycine and serine, but may include any amino acid. In some embodiments of any of the aspects, the linkers consist of glycine and serine.

The most typical configuration for an scFv-EPO fusion protein is VH-linker-VL-linker-EPO. However, other configurations such as EPO-linker-VH-linker-VL, EPO-linker-VL-linker-VH, and VH-linker-VL-linker-EPO are also possible. These are roughly similar in their spatial geometries, because movement and rotation around the linker between the scFv element allows the EPO element to assume a large number of conformations.

In addition, configurations in which the EPO element is in between the VH and VL elements may be constructed. These configurations are possible because the N- and C-termini of the EPO element are spatially close together, so that the VH and VL can easily pair. Specific configurations include VH-linker-EPO-linker-VL and VL-linker-EPO-linker-VH. Configurations in which EPO is in between the VH and VL cause the EPO element to be constrained in the orientations that it can adopt, which can be useful in situations where the dis-allowed conformations may lead to undesired binding events and side effects.

In some embodiments of any of the aspects, the linker sequence is no more than 17 amino acids in length, e.g., the linker is 1, 2, 3, 4, 5, 6, 7, 8, 9, 10, 11, 12, 13, 14, 15, 16, or 17 amino acids in length. In some embodiments of any of the aspects, the linker sequence is at least 5 amino acids in length. In some embodiments of any of the aspects, the linker sequence is 5-35 amino acids in length. In some embodiments of any of the aspects, the linker sequence is 5-7 amino acids in length, e.g., the linker is 5, 6, or 7 amino acids in length. In some embodiments of any of the aspects, the linker sequence is at 7 or fewer amino acids in length, e.g., the linker is 1, 2, 3, 4, 5, 6, or 7 amino acids in length. Exemplary but non-limiting linker sequences are provided herein, e.g., at SEQ ID NOs: 3, 5, 46-49, 64-57, 89-92, 93-97, 100-134.

TABLE 13
Exemplary Linker Sequences

| SEQ ID | |
|---|---|
| NO | Sequence |
| 93 | (GGS)₁ |
| 94 | (GGS)₂ |
| 95 | (GGS)₃ |
| 96 | (GGS)₄ |
| 97 | (GGS)₅ |
| 100 | (GGS)₆ |
| 101 | (GGS)₇ |
| 102 | (GGS)₈ |
| 103 | (GGS)₉ |
| 104 | (GGS)₁₀ |
| 105 | (GGGS)₁ |
| 106 | (GGGS)₂ |
| 107 | (GGGS)₃ |
| 108 | (GGGS)₄ |
| 109 | (GGGS)₅ |
| 110 | (GGGS)₆ |
| 111 | (GGGS)₇ |
| 112 | (GGGS)₈ |
| 113 | (GGGS)₉ |
| 114 | (GGGS)₁₀ |
| 115 | (GGGGS)₁ |
| 116 | (GGGGS)₂ |
| 117 | (GGGGS)₃ |
| 118 | (GGGGS)₄ |
| 119 | (GGGGS)₅ |
| 120 | (GGGGS)₆ |
| 121 | (GGGGS)₇ |
| 122 | (GGGGS)₈ |
| 123 | (GGGGS)₉ |
| 124 | (GGGGS)₁₀ |
| 125 | (GGGGGS)₁ |
| 126 | (GGGGGS)₂ |
| 127 | (GGGGGS)₃ |
| 128 | (GGGGGS)₄ |
| 129 | (GGGGGS)₅ |
| 130 | (GGGGGS)₆ |
| 131 | (GGGGGS)₇ |
| 132 | (GGGGGS)₈ |
| 133 | (GGGGGS)₉ |
| 134 | (GGGGGS)₁₀ |

In one aspect of any of the embodiments, described herein is a polypeptide comprising an anti-GYPA antibody reagent, a linker of no more than 14 nm in length, and an activity element. In one aspect of any of the embodiments, described herein is a polypeptide comprising an anti-GYPA antibody reagent, a linker sequence of no more than 17 amino acids, and an activity element.

An activity element may be a receptor binding protein (or functional portion or domain thereof) that binds to one or more naturally-occurring receptors on a cell surface, thereby mediating signaling to the cell (e.g., via signal transduction). In some embodiments, the activity element is a portion of a cytokine or a hormone that is sufficient to bind to a receptor on the surface of the target cell and induce an activity in the cell. In some embodiments, the cytokine or hormone is a four-helix-bundle protein. In some embodiments, the activity element is a variant form (e.g., it is mutated) that has an intrinsic binding to its receptor that is weak as compared to the wild-type protein, to the point that binding of the chimeric protein to a cell is driven by the binding of the anti-GYPA antibody reagent to its receptor. In some embodiments, the activity element is a variant of a naturally occurring protein that activates cells by binding to one or more cell surface receptors. The variant is selected such that it has reduced or no cell activating properties in the absence of the anti-GYPA antibody reagent. In some embodiments, cell activation results in stimulation of red blood cell production.

As used herein, a “mutation” refers to a change in the nucleotide sequence encoding the activity element, relative to a wild-type form of the gene, and includes substitution, deletion, and insertion mutations. A change in the nucleotide sequence may or may not lead to a change in the amino acid sequence, the three-dimensional structure of the protein, and/or the activity of the protein, relative to the wild-type form of the protein. In some embodiments a mutation may be a naturally occurring variant of the gene. In some embodiments, the activity element comprises a mutation of at least 1, 2, 3, 4, 5, 6, 7, 8, 9, 10, or more amino acids relative to a wild type/non-mutated activity element. In some embodiments a mutation may be a single amino acid substitution, two or more amino acid substitutions, one or more deletions, one or more insertions, or any combination of two or more thereof, in the protein sequence of the activity element. It will be understood that the selection of a suitable mutation in an activity element for the creation of a chimeric molecule will depend on multiple factors and in some embodiments will need to be determined empirically for different proteins.

It should be appreciated that variant activity elements of the invention may have a reduced binding affinity for their receptor(s) without a loss (or without a significant loss) of signal function (e.g., they substantially or completely retain their ability to promote signal transduction when bound to their receptor(s) even though their affinity for the receptor(s) may be significantly reduced). It also should be appreciated that the reduced binding affinity of the variant activity element preferably does not result in a protein element that will not bind to its natural receptor(s), for example, due to steric hindrance or charge repulsion or other negative interaction between the variant activity element and its natural receptor(s), even when the anti-GYPA antibody reagent binds to a target molecule on the same cell (thereby increasing the local concentration of the variant activity element in the vicinity of its natural receptor(s)).

In some embodiments, appropriate levels of reduced binding affinity can be obtained by introducing one or more mutations in charged or hydrophilic amino acids (or amino acids thought to be pointing outward) that have the effect of shortening the side chain of the amino acid(s). According to aspects of the invention, the charged or hydrophilic side chains are likely to be pointing outward and not into the middle of the protein. Reducing the size of the amino acid side chain(s) removes a contact, but does not create steric hindrance that would completely block binding or signaling. In some embodiments, mutants may be created in an activity element, and those that reduce but do not abolish binding may be selected (e.g., using one or more binding and/or activity assays known to one of skill in the art) and used to construct a chimeric activator. In some embodiments, one or more mutation in an activity element may reduce binding of the activity element to one monomer of a receptor and not affect the binding of the activity element to one or more other monomers of the receptor.

A variant activity element may include one or more naturally occurring and/or engineered mutations that result in reduced binding to one or more (e.g., all) natural receptors that are bound by the wild-type activity element. For example, the binding affinity of the variant activity element for one or more of its receptors may be at least 2-fold lower and preferably at least 5-fold lower or 8-fold lower (e.g., at least 10-fold lower, about 10-50-fold lower, about 50-100-fold lower, about 100-150-fold lower, about 150-200-fold lower, or more than 200-fold lower) than the binding affinity of the wild-type activity element for its natural receptor(s). As a result, in some embodiments the activity element by itself (e.g., not part of a fusion or chimeric protein comprising an anti-GYPA antibody reagent) is significantly less active (e.g., substantially inactive) because it cannot bind or has reduced binding to its receptor(s). In some embodiments, the variant activity element stimulates less signaling to the cell as compared to a wild type activity element (e.g., an activity element that does not have the variation/mutation). In some embodiments, the variant activity element induces at least 2-fold, at least 3-fold, at least 4-fold, at least 5-fold, at least 10-fold, at least 20-fold, at least 50-fold, at least 100-fold or less signaling to the cell as compared to a wild type activity element. Accordingly, the polypeptides, e.g., fusion polypeptides of the invention are useful to avoid unwanted side effects caused by the activity element binding to its natural receptor on non-target cells. However, the activity element is active on target cells because the anti-GYPA antibody reagent provides the missing binding affinity required for activation.

As used herein, “binding affinity” refers to the apparent association constant or K_{A}. The K_{A} is the reciprocal of the dissociation constant (K_{D}). In some embodiments, the targeting element or the activity element described herein may have a binding affinity (K_{D}) of at least 10⁻⁵ M, 10⁻⁶ M, 10⁻⁷ M, 10⁻⁸ M, 10⁻⁹ M, 10⁻¹⁰ M, 10⁻¹¹ M or lower for the corresponding receptor or targeting molecule.

In some embodiments of any of the aspects, the activity element is erythropoietin. In some embodiments of any of the aspects, the activity element is an engineered erythropoietin (EPO) comprising at least one affinity-decreasing mutation in the weak face of EPO relative to wild-type EPO.

In addition, proteins of the invention may include one or more additional peptide elements (at the N-terminus, the C-terminus, or within the protein) for purification and/or detection (e.g., peptide tags), stability, function (e.g., secretion and or other function), etc., or any combination thereof.

In one aspect of any of the embodiments, described herein is a nucleic acid encoding a polypeptide described herein, e.g., a polypeptide comprising an anti-GYPA antibody reagent, an engineered erythropoietin comprising at least one affinity-decreasing mutation in the weak face of EPO relative to wild-type EPO, and optionally a linker. In one aspect of any of the embodiments, described herein is a vector comprising a nucleic acid encoding a polypeptide described herein, e.g., a polypeptide comprising an anti-GYPA antibody reagent, an engineered erythropoietin comprising at least one affinity-decreasing mutation in the weak face of EPO relative to wild-type EPO, and optionally a linker. A nucleic acid encoding a polypeptide can further comprise expression control elements, e.g., promoters, enhancers and the like operably linked to the sequence encoding the polypeptide.

In one aspect of any of the embodiments, described herein is a cell comprising 1) a nucleic acid encoding a polypeptide described herein, e.g., a polypeptide comprising an anti-GYPA antibody reagent, an engineered erythropoietin comprising at least one affinity-decreasing mutation in the weak face of EPO relative to wild-type EPO, and optionally a linker or 2) a vector comprising said nucleic acid. The cell can be a eukaryotic or prokaryotic cell, e.g., for expression of the polypeptide in a bacterial, yeast, or human cell. Suitable cells/cell lines for protein expression are well known in the art.

Proteins of the invention are often produced by expression of a DNA construct in a mammalian cell. Other cell types such as bacteria, yeast, and insect cells may also be used. An important consideration in production of the EPO fusion proteins of the invention is that the level of sialic acid modification on the N-linked oligosaccharides should be as high as possible. To maximize sialic acid modification, cell lines such as CHO (Chinese Hamster Ovary) cells or BHK (Baby Hamster Kidney) cells can be used. These cell lines are available from the American Type Culture Collection.

To express a protein of the invention, a DNA sequence encoding the protein sequence of interest is operably linked to elements that promote protein expression and secretion, such as enhancer(s), core promoter elements such as the TATA element, a Kozak sequence for optimal ribosome binding and translation, 3′ end elements such as a polyA addition sequence and transcription terminator, introns (optional), and a signal sequence to promote translocation into the endoplasmic reticulum and eventual secretion from the cell.

For purposes of DNA propagation and cell line construction, the DNA encoding the tissue-protective EPO protein is generally embedded in a plasmid that also includes a bacterial selectable marker such as ampicillin resistance, a bacterial origin of replication, and a selectable marker that can be used in mammalian cells, such as dihydrofolate reductase, hygromycin resistance, neomycin resistance, or zeocin resistance. SEQ ID NO: 135 is a typical expression cassette for a tissue-protective EPO protein as described herein.

SEQ ID NO: 135
CGATGTACGGGCCAGATATACGCGTTGACATTGATTATTGACTAGT
TATTAATAGTAATCAATTACGGGGTCATTAGTTCATAGCCCATAT
ATGGAGTTCCGCGTTACATAACTTACGGTAAATGGCCCGCCTGGC
TGACCGCCCAACGACCCCCGCCCATTGACGTCAATAATGACGTAT
GTTCCCATAGTAACGCCAATAGGGACTTTCCATTGACGTCAATGG
GTGGACTATTTACGGTAAACTGCCCACTTGGCAGTACATCAAGTG
TATCATATGCCAAGTACGCCCCCTATTGACGTCAATGACGGTAAA
TGGCCCGCCTGGCATTATGCCCAGTACATGACCTTATGGGACTTT
CCTACTTGGCAGTACATCTACGTATTAGTCATCGCTATTACCATG
GTGATGCGGTTTTGGCAGTACATCAATGGGCGTGGATAGCGGTTT
GACTCACGGGGATTTCCAAGTCTCCACCCCATTGACGTCAATGGG
AGTTTGTTTTGGCACCAAAATCAACGGGACTTTCCAAAATGTCGT
AACAACTCCGCCCCATTGACGCAAATGGGCGGTAGGCGTGTACGG
TGGGAGGTCTATATAAGCAGAGCTCTCTGGCTAACTAGAGAACCC
ACTGCTTACTGGCTTATCGAAATTAATACGACTCACTATAGGGAG
ACCCAAGCTGGCTAGCCACCATGGAGACAGACACACTCCTGCTAT
GGGTACTGCTGCTCTGGGTTCCAGGTTCCACTGGTCAGGTCCAAC
TGCAGGAGAGCGGCGGGGGGTCAGTTCAGGGGGGGGGAGTCTGCG
GTTGAGCTGCGTAGCTTCAGGCTACACTGACAGCACCTACTGCGT
GGGATGGTTTCGGCAGGCACCCGGCAAGGAACGAGAGGGCGTTGC
ACGGATCAACACTATCTCCGGTCGGCCTTGGTACGCAGATAGTGT
TAAGGGACGGTTTACTATTAGTCAGGATAACTCTAAGAATACCGT
CTACCTTCAGATGAATAGCCTGAAACCGGAAGACACGGCTATTTA
CTATTGCACCCTTACAACTGCCAACAGCAGAGGGTTTTGTTCTGG
GGGATATAACTACAAAGGACAGGGGACCCAAGTCACTGTCAGCTC
TGGTGGTGGTTCCGCTCCACCTAGATTGATTTGTGATTCCAGAGT
TTTGGAAAGATACTTGTTGGAAGCTAAGGAGGCTGAAAATATTAC
TACTGGTTGTGCTGAACATTGTTCTTTGAACGAGAATATTACTGT
TCCAGATACTAAGGTTAACTTTTACGCTTGGAAGAGAATGGAAGT
TGGTCAGCAAGCTGTTGAAGTTTGGCAAGGTTTGGCTTTGTTGTC
TGAAGCTGTTTTGAGAGGTCAAGCTTTGTTGGTTAATTCTTCTCA
ACCATGGGAACCATTGCAATTGCATGTTGATAAGGCTGTTTCTGG
TTTGAGATCTTTGACTACCGCGTTGAGAGCTTTGGGTGCTCAAAA
GGAAGCTATTTCTCCTCCAGATGCTGCTTCTGCCGCTCCATTGAG
AACTATTACTGCTGATACTTTTAGAAAGTTGTTTAGAGTTTACTC
TAACTTCTTGAGAGGTAAGTTGAAGTTGTACACTGGTGAAGCTTG
TAGAACTGGTGATCGGGGGCCCGAACAAAAACTCATCTCAGAAGA
GGATCTGAATAGCGCCGTCGACCATCATCATCATCATCATTGAGT
TTAAACCCGCTGATCAGCCTCGACTGTGCCTTCTAGTTGCCAGCC
ATCTGTTGTTTGCCCCTCCCCCGTGCCTTCCTTGACCCTGGAAGG
TGCCACTCCCACTGTCCTTTCCTAATAAAATGAGGAAATTGCATC
GCATTGTCTGAGTAGGTGTCATTCTATTCTGGGGGGTGGGGGGGG
CAGGACAGCAAGGGGGAGGATTGGGAAGACAATAGCAGGCATGCT
GGGGA

In this sequence, the distinct segments consist of a CMV promoter element; a 7 promoter element; a Kozak sequence (gcc(G/A)ccATGG (SEQ ID NO: 137), indicated by bold italicized text in SEQ ID NO: 135 above) and upstream sequences compatible with efficient translation; a murine IgK leader sequence to promote secretion from the cell; a sequence encoding a mature fusion protein consisting of the IH4v1 nanobody (IH4v1 indicated by bold text in SEQ ID NO: 135 above; see e.g., SEQ ID NO: 38) and a 5 amino acid linker (linker indicated by italicized text in SEQ ID NO: 135 above; see e.g., SEQ ID NO: 5) and EPO L108A (engineered EPO indicated by double-underlined text and the L108A mutation indicated by bolded double-underlined text in SEQ ID NO: 135 above; see e.g., SEQ ID NO: 34); a sequence encoding a cMyc tag, a 6× His tag and a stop codon; and a BGH polyadenylation sequence.

The protein coding sequence embedded within SEQ ID NO: 135 is:

(SEQ ID NO: 142)
ATGGAGACAGACACACTCCTGCTATGGGTACTGCTGCTCTGGGTT
CCAGGTTCCACTGGTCAGGTCCAACTGCAGGAGAGCGGCGGGGGG
TCAGTTCAGGCGGGGGGGAGTCTGCGGTTGAGCTGCGTAGCTTCA
GGCTACACTGACAGCACCTACTGCGTGGGATGGTTTCGGCAGGCA
CCCGGCAAGGAACGAGAGGGCGTTGCACGGATCAACACTATCTCC
GGTCGGCCTTGGTACGCAGATAGTGTTAAGGGACGGTTTACTATT
AGTCAGGATAACTCTAAGAATACCGTCTACCTTCAGATGAATAGC
CTGAAACCGGAAGACACGGCTATTTACTATTGCACCCTTACAACT
GCCAACAGCAGAGGGTTTTGTTCTGGGGGATATAACTACAAAGGA
CAGGGGACCCAAGTCACTGTCAGCTCTGGTGGTGGTTCCGCTCCA
CCTAGATTGATTTGTGATTCCAGAGTTTTGGAAAGATACTTGTTG
GAAGCTAAGGAGGCTGAAAATATTACTACTGGTTGTGCTGAACAT
TGTTCTTTGAACGAGAATATTACTGTTCCAGATACTAAGGTTAAC
TTTTACGCTTGGAAGAGAATGGAAGTTGGTCAGCAAGCTGTTGAA
GTTTGGCAAGGTTTGGCTTTGTTGTCTGAAGCTGTTTTGAGAGGT
CAAGCTTTGTTGGTTAATTCTTCTCAACCATGGGAACCATTGCAA
TTGCATGTTGATAAGGCTGTTTCTGGTTTGAGATCTTTGACTACC
GCGTTGAGAGCTTTGGGTGCTCAAAAGGAAGCTATTTCTCCTCCA
GATGCTGCTTCTGCCGCTCCATTGAGAACTATTACTGCTGATACT
TTTAGAAAGTTGTTTAGAGTTTACTCTAACTTCTTGAGAGGTAAG
TTGAAGTTGTACACTGGTGAAGCTTGTAGAACTGGTGATCGGGGG
CCCGAACAAAAACTCATCTCAGAAGAGGATCTGAATAGCGCCGTC
GACCATCATCATCATCATCAT

The protein sequence that is encoded by the above DNA sequence is METDTLLLWVLLLWVPGSTGQVQLQESGGGSVQAGGSLRLSCVASGYTDSTYCVGWFRQA PGKEREGVARINTISGRPWYADSVKGRFTISQDNSKNTVYLQMNSLKPEDTAIYYCTLTTANS RGFCSGGYNYKGQGTQVTVSSGGGSAPPRLICDSRVLERYLLEAKEAENITTGCAEHCSLNE NITVPDTKVNFYAWKRMEVGQQAVEVWQGLALLSEAVLRGQALLVNSSQPWEPLQLHVDK AVSGLRSLTTALRALGAQKEAISPPDAASAAPLRTITADTFRKLFRVYSNFLRGKLKLYTGEA CRTGDRGPEQKLISEEDLNSAVDHHHHHH (SEQ ID NO: 143). This is the primary, unprocessed protein product that is produced from the ribosome by translation, after transcription of the expression cassette of SEQ ID NO: 135.

Because of the presence of the signal sequence at the N-terminus of this protein, the final secreted product has the sequence:

(SEQ ID NO: 144)
QVQLQESGGGSVQAGGSLRLSCVASGYTDSTYCVGWFRQAPGKER
EGVARINTISGRPWYADSVKGRFTISQDNSKNTVYLQMNSLKPED
TAIYYCTLTTANSRGFCSGGYNYKGQGTQVTVSSGGGSAPPRLIC
DSRVLERYLLEAKEAENITTGCAEHCSLNENITVPDTKVNFYAWK
RMEVGQQAVEVWQGLALLSEAVLRGQALLVNSSQPWEPLQLHVDK
AVSGLRSLTTALRALGAQKEAISPPDAASAAPLRTITADTFRKLF
RVYSNFLRGKLKLYTGEACRTGDRGPEQKLISEEDLNSAVDHHHH
HH.

This SEQ ID NO: 144 protein includes a cMyc tag and a His6 tag, which can be useful in initial pilot scale expression and purification studies, and are acceptable in non-human animal testing. In some embodiments of any of the aspects, the polypeptides described herein can be labeled (e.g., on the N-terminus or C-terminus) with a detectable tag, such as c-Myc, HA, VSV-G, HSV, FLAG, V5, HIS6, or biotin, which can assist in purification or detection. However, it is contemplated herein that a human-grade pharmaceutical protein should not contain such detectable tags.

Therefore, an exemplary expression cassette for production of a human-grade protein appropriate for clinical trials and commercial markets is:

(SEQ ID NO: 145)
CGATGTACGGGCCAGATATACGCGTTGACATTGATTATTGACTAG
TTATTAATAGTAATCAATTACGGGGTCATTAGTTCATAGCCCATA
TATGGAGTTCCGCGTTACATAACTTACGGTAAATGGCCCGCCTGG
CTGACCGCCCAACGACCCCCGCCCATTGACGTCAATAATGACGTA
TGTTCCCATAGTAACGCCAATAGGGACTTTCCATTGACGTCAATG
GGTGGACTATTTACGGTAAACTGCCCACTTGGCAGTACATCAAGT
GTATCATATGCCAAGTACGCCCCCTATTGACGTCAATGACGGTAA
ATGGCCCGCCTGGCATTATGCCCAGTACATGACCTTATGGGACTT
TCCTACTTGGCAGTACATCTACGTATTAGTCATCGCTATTACCAT
GGTGATGCGGTTTTGGCAGTACATCAATGGGCGTGGATAGCGGTT
TGACTCACGGGGATTTCCAAGTCTCCACCCCATTGACGTCAATGG
GAGTTTGTTTTGGCACCAAAATCAACGGGACTTTCCAAAATGTCG
TAACAACTCCGCCCCATTGACGCAAATGGGCGGTAGGCGTGTACG
GTGGGAGGTCTATATAAGCAGAGCTCTCTGGCTAACTAGAGAACC
CACTGCTTACTGGCTTATCGAAATTAATACGACTCACTATAGGGA
GACCCAAGCTGGCTAGCCACCATGGAGACAGACACACTCCTGCTA
TGGGTACTGCTGCTCTGGGTTCCAGGTTCCACTGGTCAGGTCCAA
CTGCAGGAGAGCGGCGGGGGGTCAGTTCAGGCGGGGGGGAGTCTG
CGGTTGAGCTGCGTAGCTTCAGGCTACACTGACAGCACCTACTGC
GTGGGATGGTTTCGGCAGGCACCCGGCAAGGAACGAGAGGGCGTT
GCACGGATCAACACTATCTCCGGTCGGCCTTGGTACGCAGATAGT
GTTAAGGGACGGTTTACTATTAGTCAGGATAACTCTAAGAATACC
GTCTACCTTCAGATGAATAGCCTGAAACCGGAAGACACGGCTATT
TACTATTGCACCCTTACAACTGCCAACAGCAGAGGGTTTTGTTCT
GGGGGATATAACTACAAAGGACAGGGGACCCAAGTCACTGTCAGC
TCTGGTGGTGGTTCCGCTCCACCTAGATTGATTTGTGATTCCAGA
GTTTTGGAAAGATACTTGTTGGAAGCTAAGGAGGCTGAAAATATT
ACTACTGGTTGTGCTGAACATTGTTCTTTGAACGAGAATATTACT
GTTCCAGATACTAAGGTTAACTTTTACGCTTGGAAGAGAATGGAA
GTTGGTCAGCAAGCTGTTGAAGTTTGGCAAGGTTTGGCTTTGTTG
TCTGAAGCTGTTTTGAGAGGTCAAGCTTTGTTGGTTAATTCTTCT
CAACCATGGGAACCATTGCAATTGCATGTTGATAAGGCTGTTTCT
GGTTTGAGATCTTTGACTACCGCGTTGAGAGCTTTGGGTGCTCAA
AAGGAAGCTATTTCTCCTCCAGATGCTGCTTCTGCCGCTCCATTG
AGAACTATTACTGCTGATACTTTTAGAAAGTTGTTTAGAGTTTAC
TCTAACTTCTTGAGAGGTAAGTTGAAGTTGTACACTGGTGAAGCT
TGTAGAACTGGTGATCGGTGAGTTTAAACCCGCTGATCAGCCTCG
ACTGTGCCTTCTAGTTGCCAGCCATCTGTTGTTTGCCCCTCCCCC
GTGCCTTCCTTGACCCTGGAAGGTGCCACTCCCACTGTCCTTTCC
TAATAAAATGAGGAAATTGCATCGCATTGTCTGAGTAGGTGTCAT
TCTATTCTGGGGGGGGGGTGGGGCAGGACAGCAAGGGGGAGGATT
GGGAAGACAATAGCAGGCATGCTGGGGA.

The protein coding sequence embedded within SEQ ID NO: 145 above is:

(SEQ ID NO: 146)
ATGGAGACAGACACACTCCTGCTATGGGTACTGCTGCTCTGGGTT
CCAGGTTCCACTGGTCAGGTCCAACTGCAGGAGAGCGGCGGGGGG
TCAGTTCAGGCGGGGGGGAGTCTGCGGTTGAGCTGCGTAGCTTCA
GGCTACACTGACAGCACCTACTGCGTGGGATGGTTTCGGCAGGCA
CCCGGCAAGGAACGAGAGGGCGTTGCACGGATCAACACTATCTCC
GGTCGGCCTTGGTACGCAGATAGTGTTAAGGGACGGTTTACTATT
AGTCAGGATAACTCTAAGAATACCGTCTACCTTCAGATGAATAGC
CTGAAACCGGAAGACACGGCTATTTACTATTGCACCCTTACAACT
GCCAACAGCAGAGGGTTTTGTTCTGGGGGATATAACTACAAAGGA
CAGGGGACCCAAGTCACTGTCAGCTCTGGTGGTGGTTCCGCTCCA
CCTAGATTGATTTGTGATTCCAGAGTTTTGGAAAGATACTTGTTG
GAAGCTAAGGAGGCTGAAAATATTACTACTGGTTGTGCTGAACAT
TGTTCTTTGAACGAGAATATTACTGTTCCAGATACTAAGGTTAAC
TTTTACGCTTGGAAGAGAATGGAAGTTGGTCAGCAAGCTGTTGAA
GTTTGGCAAGGTTTGGCTTTGTTGTCTGAAGCTGTTTTGAGAGGT
CAAGCTTTGTTGGTTAATTCTTCTCAACCATGGGAACCATTGCAA
TTGCATGTTGATAAGGCTGTTTCTGGTTTGAGATCTTTGACTACC
GCGTTGAGAGCTTTGGGTGCTCAAAAGGAAGCTATTTCTCCTCCA
GATGCTGCTTCTGCCGCTCCATTGAGAACTATTACTGCTGATACT
TTTAGAAAGTTGTTTAGAGTTTACTCTAACTTCTTGAGAGGTAAG
TTGAAGTTGTACACTGGTGAAGCTTGTAGAACTGGTGATCGG

The protein sequence that is encoded by the above DNA sequence is METDTLLLWVLLLWVPGSTGQVQLQESGGGSVQAGGSLRLSCVASGYTDSTYCVGWFRQA PGKEREGVARINTISGRPWYADSVKGRFTISQDNSKNTVYLQMNSLKPEDTAIYYCTLTTANS RGFCSGGYNYKGQGTQVTVSSGGGSAPPRLICDSRVLERYLLEAKEAENITTGCAEHCSLNE NITVPDTKVNFYAWKRMEVGQQAVEVWQGLALLSEAVLRGQALLVNSSQPWEPLQLHVDK AVSGLRSLTTALRALGAQKEAISPPDAASAAPLRTITADTFRKLFRVYSNFLRGKLKLYTGEA CRTGDR (SEQ ID NO: 147). This is the primary, unprocessed protein product that is produced from the ribosome by translation, after transcription of the above expression cassette in SEQ ID NO: 145.

Because of the presence of the signal sequence at the N-terminus of the protein above, the final secreted product has the sequence

(SEQ ID NO: 148)
QVQLQESGGGSVQAGGSLRLSCVASGYTDSTYCVGWFRQAPGKER
EGVARINTISGRPWYADSVKGRFTISQDNSKNTVYLQMNSLKPED
TAIYYCTLTTANSRGFCSGGYNYKGQGTQVTVSSGGGSAPPRLIC
DSRVLERYLLEAKEAENITTGCAEHCSLNENITVPDTKVNFYAWK
RMEVGQQAVEVWQGLALLSEAVLRGQALLVNSSQPWEPLQLHVDK
AVSGLRSLTTALRALGAQKEAISPPDAASAAPLRTITADTFRKLF
RVYSNFLRGKLKLYTGEACRTGDR.

SEQ ID NO: 148 is a specific exemplary human-grade sequence of a red blood-cell stimulating, tissue protective, non-thrombotic protein.

In some embodiments of any of the aspects, the engineered erythropoietin fusion protein comprises the sequence of one of SEQ ID NOs: 143, 144, 147, 148, or an amino acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 143, 144, 147, or 148 that maintains the same function (e.g., binding to EPOR and/or CD131). In some embodiments of any of the aspects, the engineered erythropoietin fusion protein comprises the sequence of one of SEQ ID NOs: 143, 144, 147, 148, or an amino acid sequence that is at least 95% identical to one of SEQ ID NOs: 143, 144, 147, or 148 that maintains the same function (e.g., binding to EPOR and/or CD131).

In some embodiments of any of the aspects, the engineered erythropoietin fusion protein is encoded by one of SEQ ID NOs: 142, 146, 149, or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 142, 146, 149, or a codon-optimized version thereof, wherein the encoded polypeptide maintains the same function (e.g., binding to EPOR and/or CD131). In some embodiments of any of the aspects, the engineered erythropoietin fusion protein is encoded by one of SEQ ID NOs: 142, 146, 149, or a nucleic acid sequence that is at least 95% identical to one of SEQ ID NOs: 142, 146, 149, or a codon-optimized version thereof, wherein the encoded polypeptide maintains the same function (e.g., binding to EPOR and/or CD131). In some embodiments of any of the aspects, the erythropoietin is a murine or primate erythropoietin.

In some embodiments of any of the aspects, the engineered erythropoietin fusion protein is encoded an expression cassette comprising one of SEQ ID NOs: 135, 145 or a nucleic acid sequence that is at least 80%, at least 85%, at least 90%, at least 91%, at least 92%, at least 93%, at least 94%, at least 95%, at least 96%, at least 97%, at least 98%, or at least 99% identical to one of SEQ ID NOs: 135, 145, or a codon-optimized version thereof, wherein the encoded polypeptide maintains the same function (e.g., binding to EPOR and/or CD131). In some embodiments of any of the aspects, the engineered erythropoietin fusion protein is encoded an expression cassette comprising one of SEQ ID NOs: 135, 145 or a nucleic acid sequence that is at least 95% identical to one of SEQ ID NOs: 135, 145, or a codon-optimized version thereof, wherein the encoded polypeptide maintains the same function (e.g., binding to EPOR and/or CD131).

To produce protein for initial testing of candidate forms of tissue-protective EPO, mammalian cells such as HEK293F (Human Embryo Kidney) cells are transiently transfected with an expression plasmid comprising a sequence such as SEQ ID: 135 or SEQ ID: 145. Example 11 of International Patent Application WO 2020/132234 demonstrates a typical transient transfection. The resulting protein can be tested for expression level, aggregation and in vitro activity in cell-based assays. However, tissue-protective EPO proteins expressed in HEK293F cells generally have reduced levels of sialic acid compared to natural EPO and to the desired product that should be used in vivo. One effect of less of sialic acid is to reduce the negative charge on the protein. Because the binding of EPO to EPO receptor is partly driven by the relative positive charges on EPO and negative charges on the receptor, reducing the number of negatively charged sialic acid residues, and thus increasing the binding of the EPO element for its receptor relative to and EPO protein that has been produced from CHO or BHK cells. This increased receptor binding has the in vivo effect of increasing EPO receptor-mediated endocytosis and degradation, thus decreasing the plasma half-life of EPO-based molecules and paradoxically decreasing the in vivo activity.

Proteins of the invention may also be produced from CHO cells by transient or stable transfection. The amount of material produced from CHO cells upon transient transfection is generally less than from a HEK293 cell transfection, but the sialic acid content is higher.

A useful type of CHO cells is the CHO-DG44 line, which is DHFR deficient. According to the invention, a plasmid encoding a tissue-protective EPO and also encoding a DHFR gene is inserted into the CHO-DG44 cell line by selection for growth in the absence of hypoxanthine and thymidine. The expression of a gene of interest can be further improved by growth in the presence of methotrexate. Yeo et al. (Biotechnology Journal 12:1700175 (2017)) describes detailed procedures for such cell line construction and is incorporated by reference herein.

Tissue-protective EPO proteins of the invention are purified by standard procedures. Example 11 of International Patent Application WO 2020/132234 illustrates specific methods. For example, the tissue-protective EPOs are purified by nickel-IDA or cobalt affinity methods (see, e.g., “His60 Ni Superflow Resin & Gravity Columns User Manual” ClonTech Laboratories, Inc. PT5017-1 (031716) followed by size exclusion chromatography using endotoxin-free reagents. Goldwasser et al. (Proc. Nat. Acad. Sci. USA Vol. 68, No. 4, pp. 697-698, April 1971) also describes purification methods for erythropoietin itself; these may be readily adapted for purification of tissue-protective EPO. Each of the foregoing references is incorporated by reference herein in its entirety.

Merely by way of non-limiting example, to formulate the tissue-protective EPO proteins of the invention, the following buffers and excipients may be used: phosphate-buffered saline, human serum albumin, arginine, mannitol, sorbitol, citrate, Tween such as Tween80, and so on. Formulation of protein drugs is well-known in the art of pharmaceutical development.

The activity and pharmacological efficacy of the polypeptides described herein (e.g., tissue-protective EPO proteins) can be measured by any of the assays described herein. By way of example, purified tissue-protective EPO, crude cell supernatants, and material from intermediate stages in purification may be tested by methods including, but not limited to, liquid-chromatography/mass-spectrometry, dynamic light scattering, analytical size exclusion chromatography, SDS-PAGE, isoelectric focusing, lectin binding, sandwich ELISA in which one moiety is captured and the other is detected, binding to EPO receptor, and binding to glycophorin A. These methods are used to determine purity and extent of contamination with host cell protein, extent of aggregation, chemical degradation of amino acid side chains, proteolysis, extent of glycosylation and particularly the level of sialic modification, and correct folding of, e.g., the EPO moiety and of the IH4 antibody element. These methods are generally well known in the state of the art for pharmaceutical industry protein characterization. Glycophorin A binding as assayed with red blood cells, is described in Example 6 of International Patent Application WO 2020/132234.

Assays for contamination by microbial material include testing for endotoxin by standard Limulus amebocyte lysate (LAL) methods (e.g., those available from Charles River (Wilmington, MA), e.g., on the world wide web at criver.com/products-services/qc-microbial-solutions/endotoxin-testing/lal-reagents-accessories, and culturing for live microbes by standard microbiological techniques.

The erythropoietic activity of an EPO element in a fusion protein can be assayed using cells that express the EPO receptor. The cells may optionally also express glycophorin A. For example, the cell lines TF-1 and UT-7 express both EPO receptor and glycophorin A. Taylor et al. (PEDS 23(4), 251-260, 2010) and Burrill et al. (PNAS 113:5245-5250 (2016)) describe the use of these cell lines to assay various forms of tissue-protective EPO, and are incorporated by reference herein. Other cell lines such as MCF-7 express EPO receptor but not glycophorin A. These cell lines may be used to specifically measure EPO activity independently of membrane-localizing effects of the antibody element and glycophorin A.

Typically, such cell-based assays are performed as follows. Cells are distributed into microtiter plates such as a 96-well plate. They are allowed to adhere and begin to grow. Well before the cells become confluent, the medium is changed to a somewhat poor medium, and various concentrations of tissue-protective EPO protein are added to the wells. The extent of cell growth is measured about 3 days later by standard methods.

Red blood cells may be used to assay glycophorin A binding. Typically, a tissue-protective EPO protein is added to a preparation of red blood cells; a labeled secondary antibody that recognizes either a peptide tag, the EPO element, or the targeting element of the tissue-protective EPO is added for detection; cells are washed; and then the cells are analyzed by flow cytometry. Use of a tertiary labeled antibody is sometimes necessary. Example 6 of International Patent Application WO 2020/132234 illustrates details of such methods. Assays based on binding to red blood cells provide several types of information. For example, batch-to-batch variation may be tested and bad batches of drug product can be identified. Additionally, during a research phase, binding to red blood cells can be used to measure antibody accessibility of drug, which correlates with the potential of the EPO element of a bound drug to contact other cells. FIG. 5A of International Patent Application WO 2020/132234 illustrates this principle.

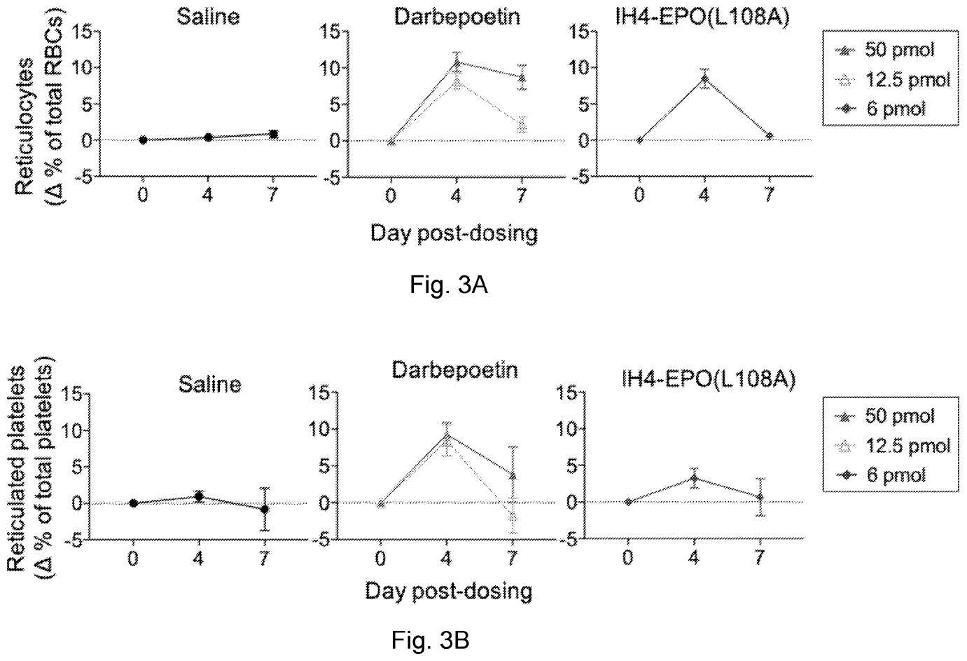

The polypeptides described herein can also be tested in vivo, e.g., using animal models to demonstrate activity and safety. Typically, an animal is injected intravenously, and the bleeding time and/or levels of reticulocytes and reticulated platelets can be measured, e.g., as described in FIG. 3A-3B or FIG. 6A-6C herein, or Example 7 of International Patent Application WO 2020/132234.

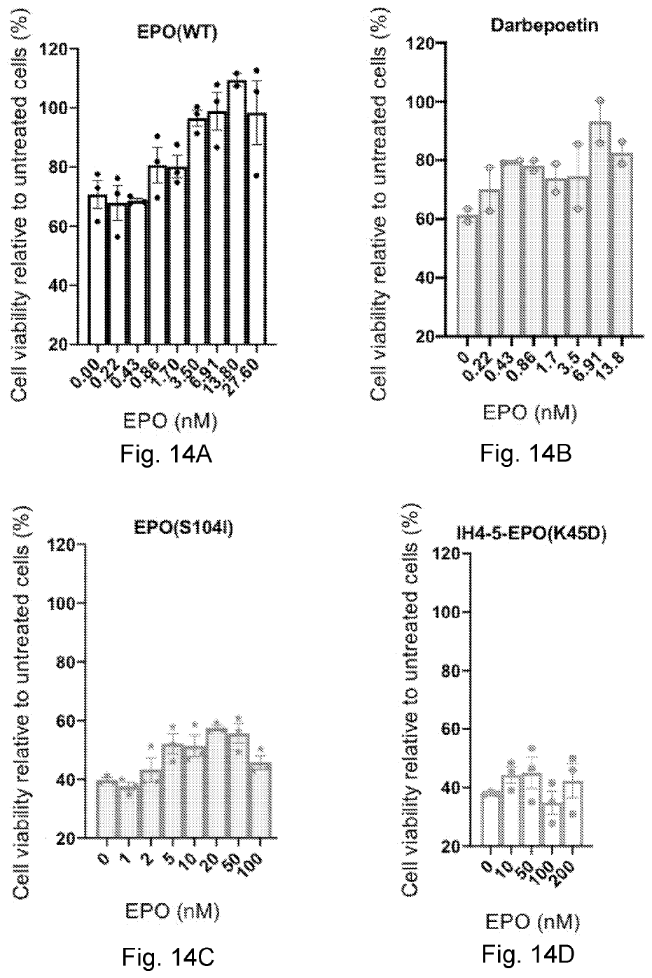

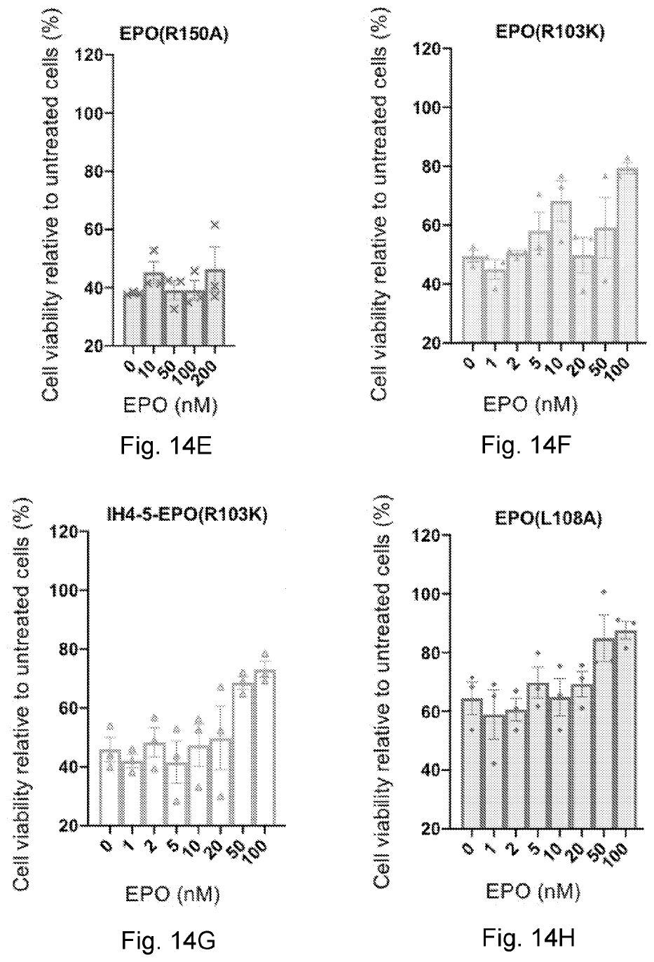

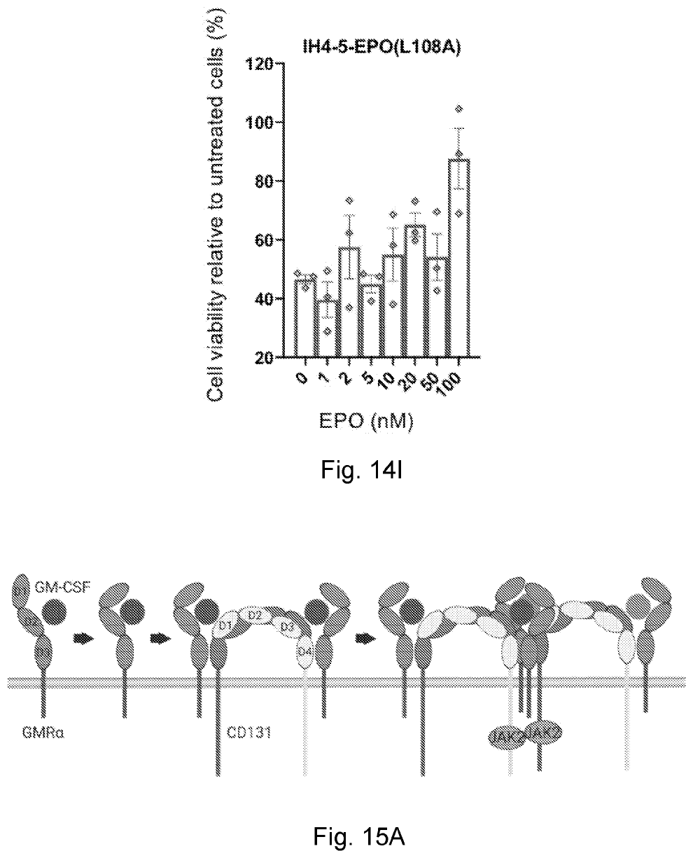

It is important to consider the species specificity of binding for both the EPO element and the glycophorin-binding element when choosing an animal test system. Among mammals, human EPO generally acts on the EPO receptors of other animals, since this system is highly conserved. In contrast, the extracellular portion of glycophorin A is not highly conserved. For example, the mouse and human glycophorin A extracellular domains are not recognizably related, while the transmembrane and intracellular domains can easily be aligned. Some animals do not encode a functional glycophorin A. Even within primates, the sequences of glycophorin A show significant variation. The alignment depicted in FIG. 14 of International Patent Application WO 2020/132234 provides a comparison of the glycophorin A sequences of humans, chimpanzees, orangutans, gorillas, bonobo chimpanzees, rhesus monkeys, cynomolgus monkeys (aka the crab-eating macaque), and the black snub-nosed monkey (Rhinopithecus bieti). Also indicated are O-linked and N-linked glycosylation sites (all in the extracellular domain, underlined), the epitopes for the anti-glycophorin antibodies R10, 10F7, and IH4, and the transmembrane and intracellular domains. Inspection of the glycophorin sequences indicates that the epitopes for these various antibodies (all of which were selected for binding to human glycophorin A) are altered in some non-human primates, and thus that these primates would not be appropriate for testing forms of tissue-protective EPO that use the corresponding antibody. For example, the gorilla has the mutations Asp27Lys, Thr28Lys, and Thr37Pro in the region of the 10F7 epitope, and thus gorillas glycophorin may not be bound by glycophorin A based on this analysis. Similarly, the IH4 epitope appears to be Tyr52, Pro53, Pro54, Glu55, Glu56 (Habib et al.), which is altered in the orangutan, and Rhinopithecus; binding to the gorilla sequence could also be disrupted due to the Glu57Tyr change that might lead to steric hindrance. Thus, orangutan, Rhinopithecus and gorilla would not be the first choice for primate test systems, while chimps, rhesus monkeys and cynomolgus monkeys would likely be better primate test systems because the glycophorin A on their red blood cells should be bound by IH4.

It is also useful to perform animal testing in a rodent model system. The glycophorin A sequences from non-primate mammals is generally so divergent from the human sequence that antibodies to human glycophorin A are not expected to bind at all. Auffray et al. (Blood 97:2972-2979 (2001)) developed a transgenic mouse that expresses human glycophorin A. The breeding of this mouse strain is described by Auffray et al and by Burrill et al., which are incorporated by reference herein in their entireties. In brief, the human glycophorin A transgene causes no deleterious phenotype in the mice when heterozygous, but is homozygous lethal. During breeding, the presence of the transgene may be monitored by PCR testing, or by staining of blood samples with a labeled antibody directed against human glycophorin A, followed by flow cytometry. From a cross of a transgene/+male with a wild-type female, about half of the offspring will carry the transgene.

The testing of forms of tissue-protective EPO to demonstrate stimulation of red blood cell production and lack of platelet production is described by Burrill et al., and in FIG. 3A-3B or FIG. 6A-6C herein, or Example 6 and 7 of International Patent Application WO 2020/132234.

Long-term toxicity testing may also be performed in transgenic mice or in primates. For example, glycophorin A-transgenic mice are injected with a tissue-protective EPO such as IH4-(5aa)-EPO(L108A or R103K) twice per week for eight weeks. Blood is withdrawn periodically and the plasma fraction is tested for the presence of anti-drug antibodies. In addition, the hematocrit, platelet, reticulocyte and reticulated platelet counts are determined. Antibodies to the drug may develop. Such antibodies may be, for example, in the form of weak, non-neutralizing antibodies that do not inhibit the function of the drug, and in fact have the effect of extending the plasma half-life of the drug. Alternatively, drug-specific neutralizing antibodies that block drug activity may form. These can be detected by ELISA in the plasma of the treated mice. Such antibodies will have the in vivo effect of preventing drug action, so that red blood cell levels and reticulocyte levels will not be elevated during long-term treatment. The worst-case scenario is the formation of anti-drug antibodies that cross-react with the endogenous EPO of the animal. In such a case, the animal may become anemic and in some cases may become aplastic (complete failure to produce red blood cells), which is generally fatal if untreated.

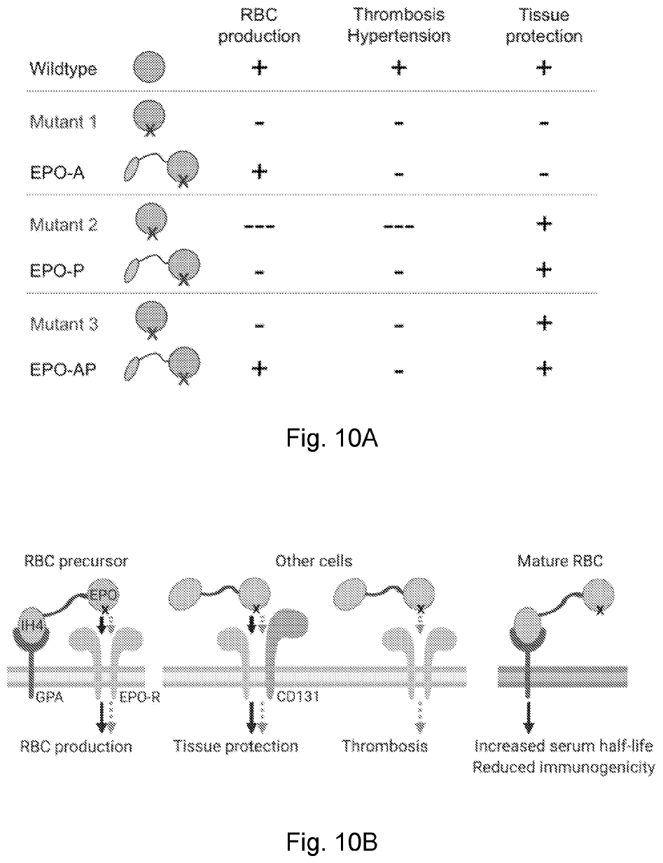

Another set of toxicological animal tests relate to blood clotting. Erythropoietin itself enhances blood clotting and markers relating to blood clotting. In human clinical trials there is evidence that patients treated with erythropoiesis-stimulating agents (ESAs) such as epoetin alpha, epoetin beta, or darbepoetin show an increased frequency of stroke, cardiac arrest, and deep vein thrombosis. These observations are the motivation for the present invention. In addition, Kirkeby et al. (Thromb Haemost 99:720-728 (2008))) showed that erythropoietin treatment of rats had the following effects: (1) shortening the bleeding time as measured in a tail transection assay; (2) increasing plasma P-selectin; (3) increasing platelet sensitivity to thrombin receptor agonist peptides; (4) increasing mean platelet volume; (5) increasing levels of P2Y₁, P2Y₁₂, MEK1/2, and GSK3beta; and other effects. Treatment of glycophorin A-transgenic mice with erythropoietin or darbepoetin shortens the bleeding time by about 30% (see e.g., FIGS. 10A, 10B of International Patent Application WO 2020/132234). Different forms of tissue-protective EPO can have different effects in the tail transection/bleeding time assay, which is summarized below.

TABLE 12

| | Mouse genotype | Shortening |
|---|---|---|
| | (transgenic mice | bleeding |
| Treatment | express human GPA) | time? |
| Saline | Wild-type (non-transgenic) | No (baseline- |
| | | defining |
| | | treatment) |
| Saline | Wild-type (transgenic) | No (baseline- |
| | | defining |
| | | treatment) |
| Erythropoietin | Wild-type (non-transgenic) | Yes |
| Darbepoetin | Wild-type (non-transgenic) | Yes |
| Erythropoietin | Wild-type (transgenic) | Yes |
| Darbepoetin | Wild-type (transgenic) | Yes |
| 10F7-(long linker, | Wild-type (transgenic) | Yes |
| e.g., 35AA)-EPO(weak | | |
| side mutant, e.g., | | |
| L108A or R103K) | | |
| 10F7-(long linker, | Wild-type (non-transgenic) | No |
| e.g., 35AA)-EPO(weak | | |
| side mutant, e.g., | | |
| L108A or R103K) | | |
| IH4v1-(long linker, | Wild-type (transgenic) | Yes |
| e.g., 35AA)-EPO(weak | | |
| side mutant, e.g., | | |
| L108A or R103K) | | |
| IH4v1-(short linker, | Wild-type (transgenic) | No |
| e.g., 5AA)-EPO(weak | | |
| side mutant, e.g., | | |
| L108A or R103K) | | |

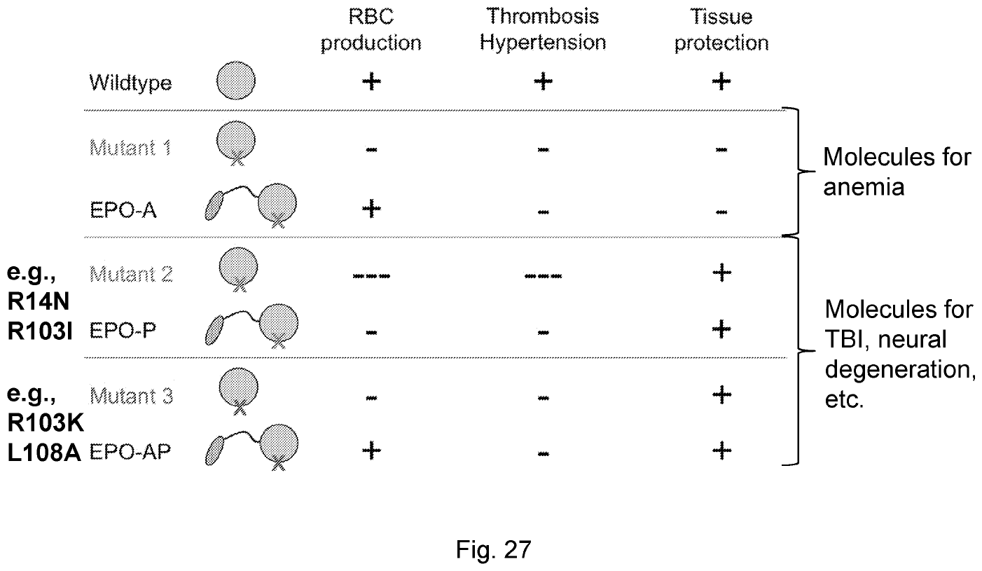

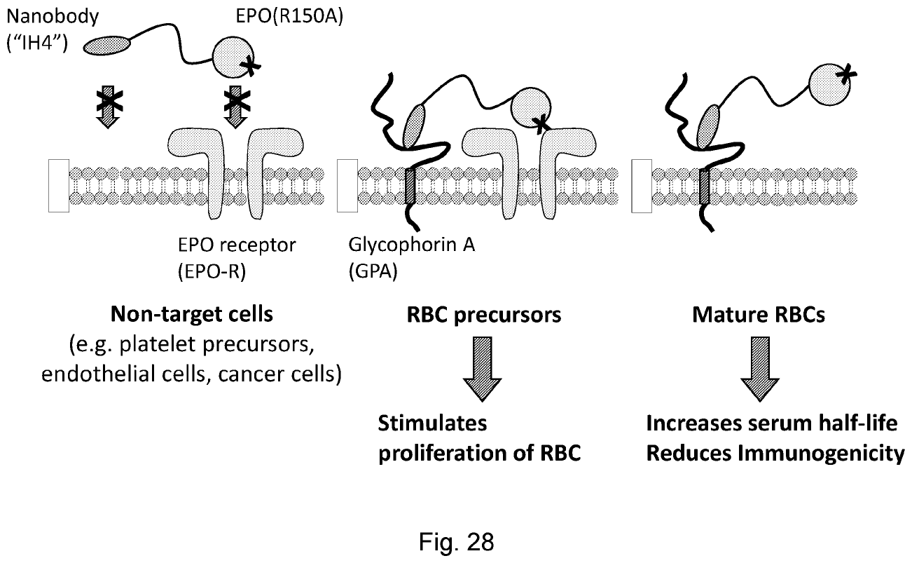

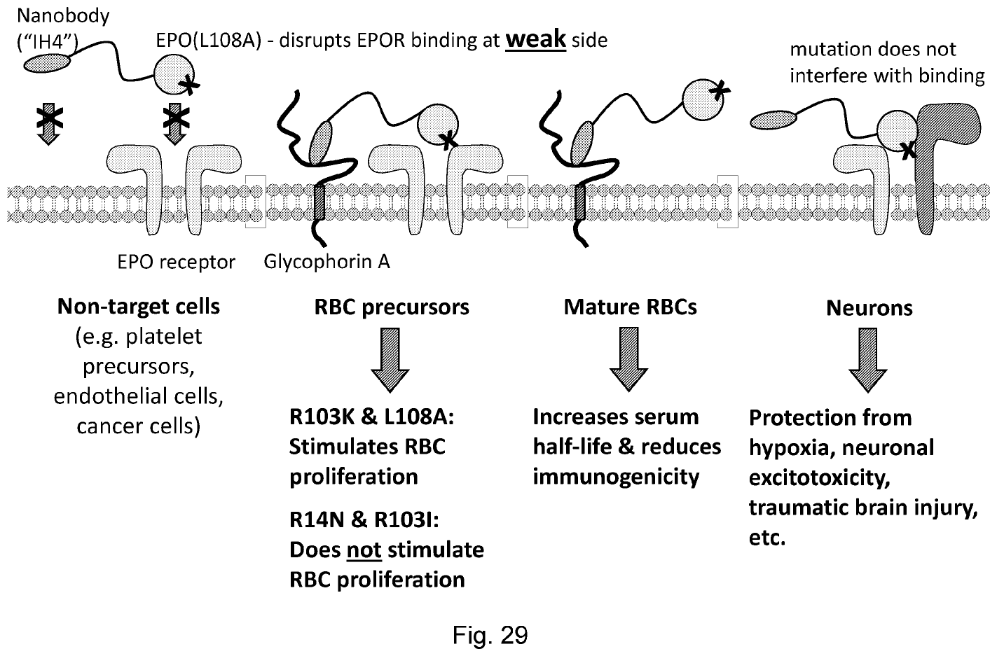

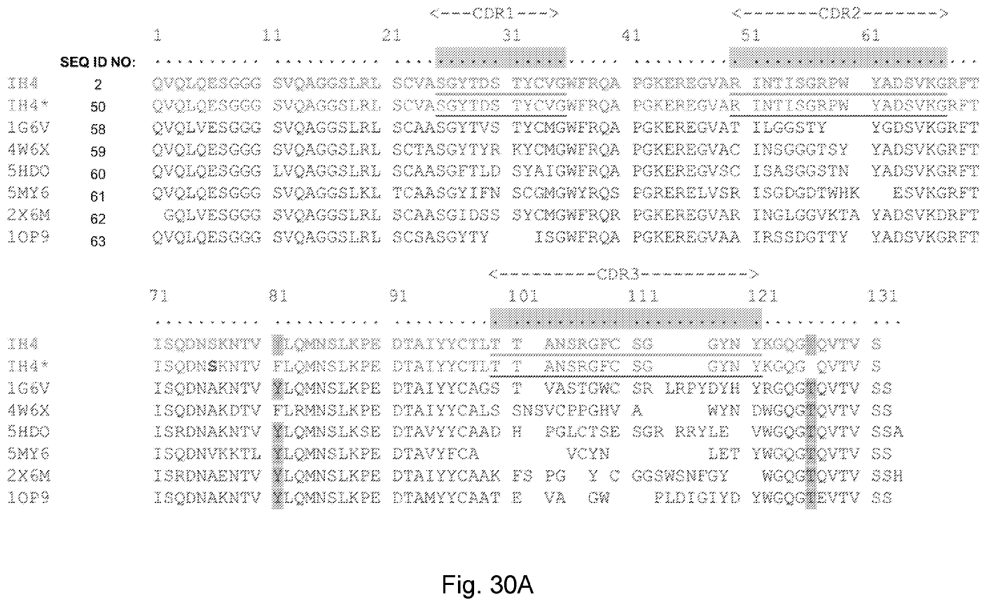

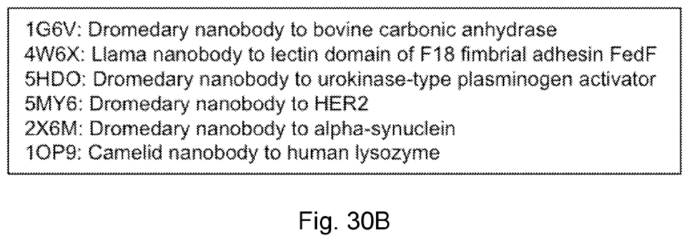

Without wishing to be bound by theory, it is contemplated that weak-side EPO mutants show similar effects as the R150A mutant in bleeding time experiments; i.e., weak-side mutants do not induce shortened bleeding time (see e.g., last row of Table 12, with IH4v1-5AA-EPO(L108A or R103K); see also FIG. 27), indicating reduced pro-thrombotic side effects of the EPO element. This is based on the fact that the weak-side mutations greatly reduce EPO interaction with homodimeric EPOR, which mediates the thrombotic effect of EPO.

The tail transection assay of bleeding time can be performed on mice as follows. On day 0, mice are injected with a test protein (or vehicle). When testing an unknown protein, it is important to also include mice that are injected with saline or PBS vehicle as a negative control, and EPO or darbepoetin as a positive control. Typically, 10 mice per dose group are used. It is important that the experiment is performed in a blind manner. For example, one experimenter performs the injections of proteins into the mice, maintains the key, performs the injection of anesthetic, and then hands the mice in a random order to a second, blinded experimenter. The second experimenter performs the tail transfections and measures the bleeding time. On day 1, the tail transection and bleeding time is measured. In principle, the tail transection could be performed on day 2, 3, 4 or later. However, the advantage of performing the measurement 1 day after treatment is that after only 24 hours, the level of circulating red blood cells will not have changed, so effects on blood clotting are due to direct effects on some element of the clotting system, and not due to changes in blood viscosity.

Tail transection can be performed as follows. The mice are first anaesthetized using anesthetic ketamine and xylazine. These anesthetics are chosen because they are thought to not affect blood clotting. Acepromazine is not used because it has the effect of reducing clotting. Mice are weighed, and mice are then injected with 120-160 mg ketamine/kg and 10-16 mg xylazine/kg of body weight. For older and heavier mice, sometimes an additional injection of about 25% of the first injection was required. After a mouse became unresponsive to a stimulus such as significant pressure to a hind foot, the mouse is placed on a heated pad on a platform over a water bath. The water bath is maintained at 37° C. 50-ml blue-cap tubes (SARSTEDT) are filled with 50 mLs of a solution of 0.85 to 0.9% NaCl that has been equilibrated to 37° C. in a separate water bath. The animal is placed on a Chux pad or equivalent.

A position on the tail that is 3 millimeters from the tip, not counting hair, is marked with a felt-tip pen using calipers. (The tail is also inspected for signs of bruising that may be due to fighting, and data from such a mouse is discarded if the transected tail does not bleed at all. The decision to discard the data must be made in a blinded manner). The tail is transected with a flat razor blade using a section of the blade that has not been used previously. Within 1 second, the transected tail is placed in a tube with pre-warmed saline, and then the body of the mouse is placed on the heated pad above the water bath. At the moment that the tail is transected by the blinded experimenter, the non-blinded experimenter starts a timer. The body of the mouse is then positioned on the heated pad so that only the tip of the tail—about 0.5 to 2 mm—is in the saline and the rest is in the air. When observing the bleeding tail, the tube should be rotated so that the white stripe is behind the tail, providing contrast, and the room should be well-lit. The rack holding the 50-ml tube should be white or yellow to provide contrast. The bleeding time is recorded by noting when bleeding stops, and then observing the submerged tail for up to one minute. If bleeding re-starts within this minute, the first recorded time is not counted. Bleeding may stop and re-start several times. If the tail is still bleeding when 10 minutes have elapsed, the time is recorded as 10 minutes.

The median and mean bleeding times are calculated for each treatment group. Calculating the median has the advantage that extreme events, such as 10 minute timepoints, do not disproportionately contribute to the calculation.

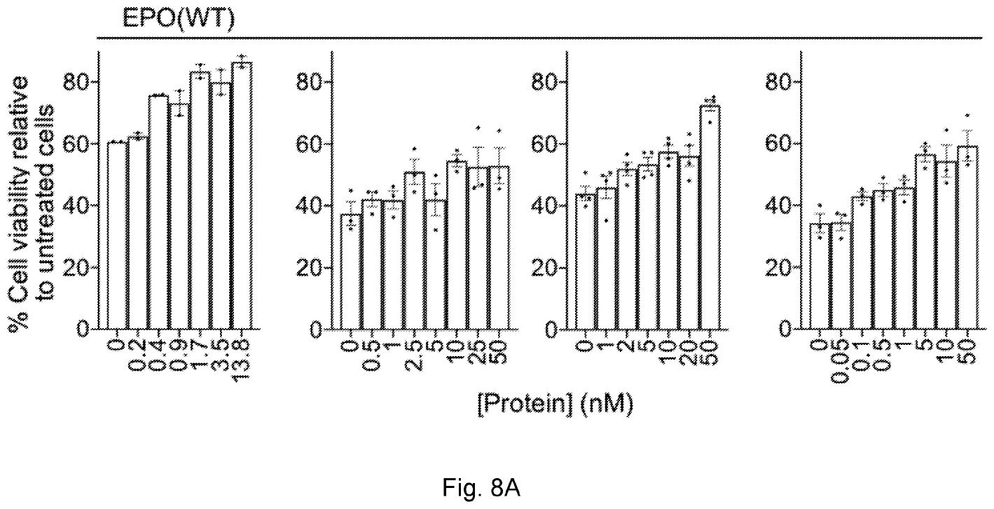

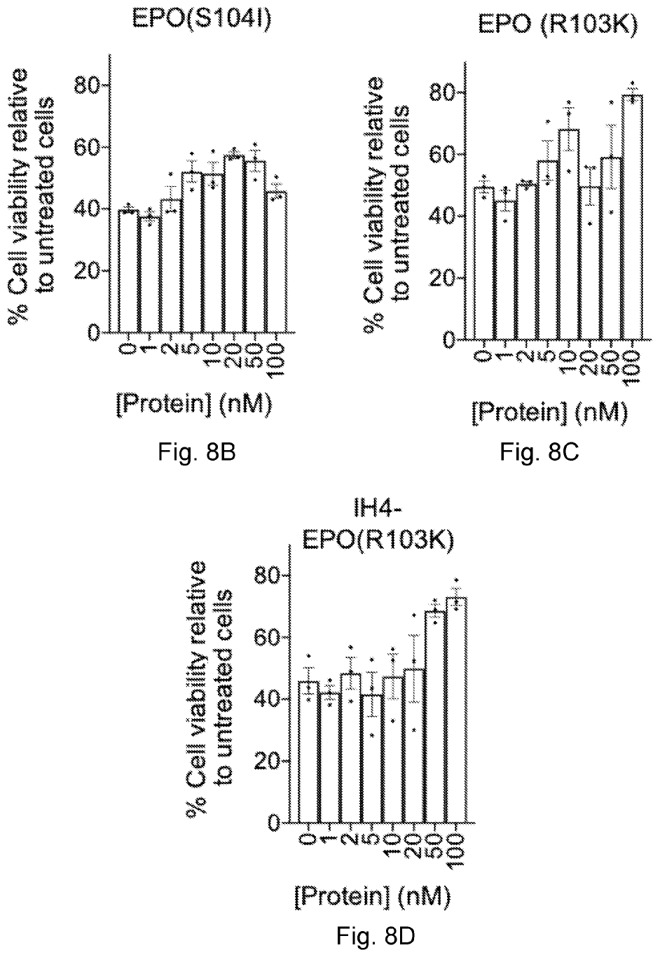

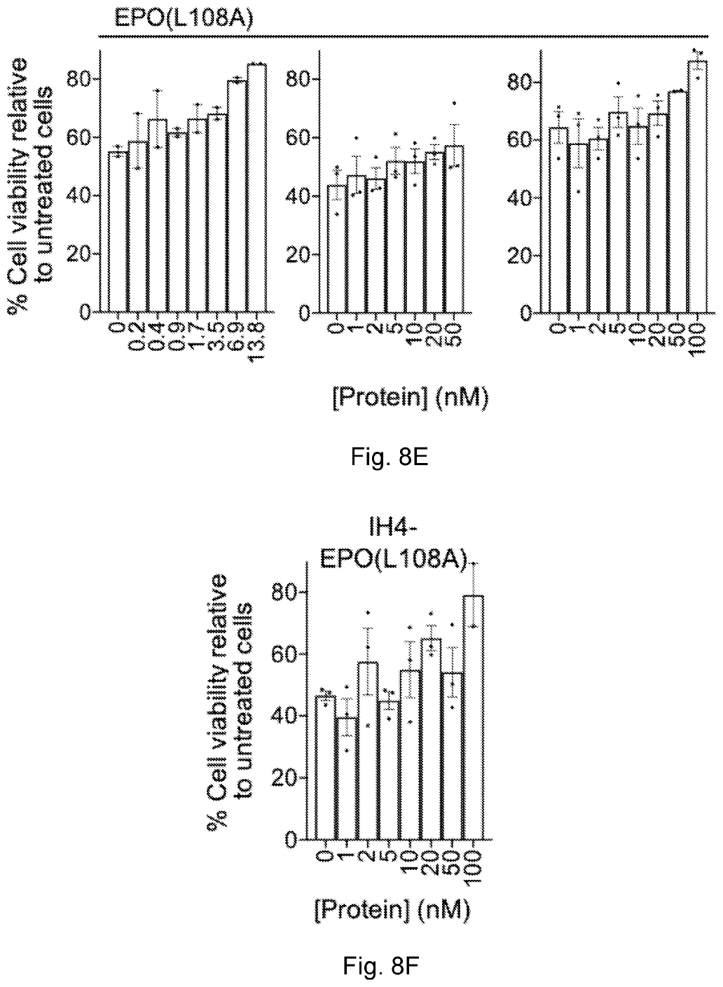

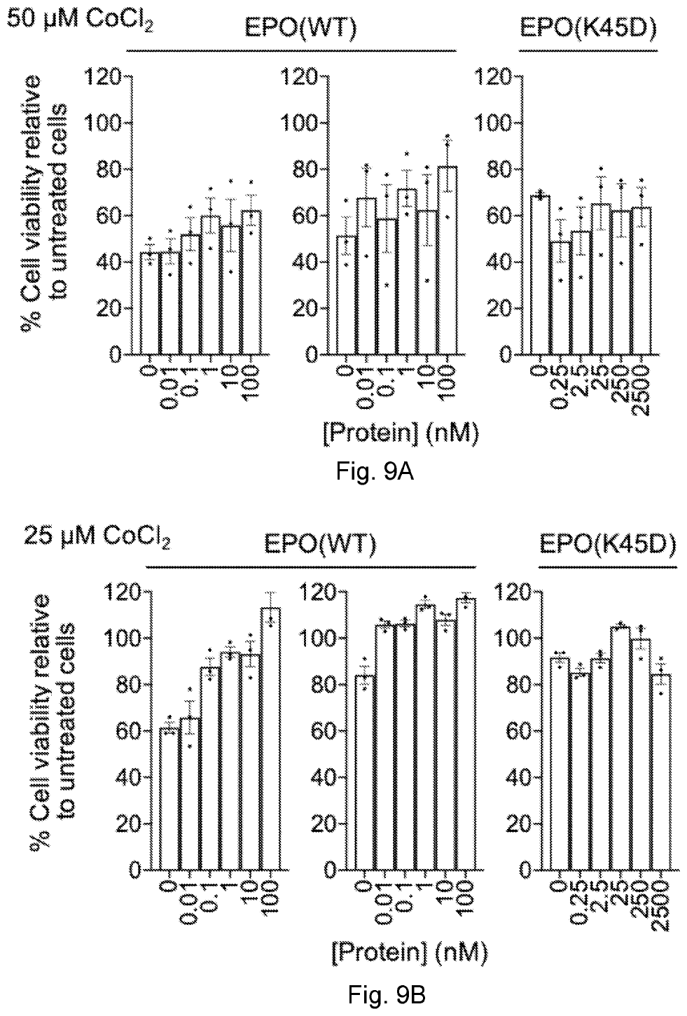

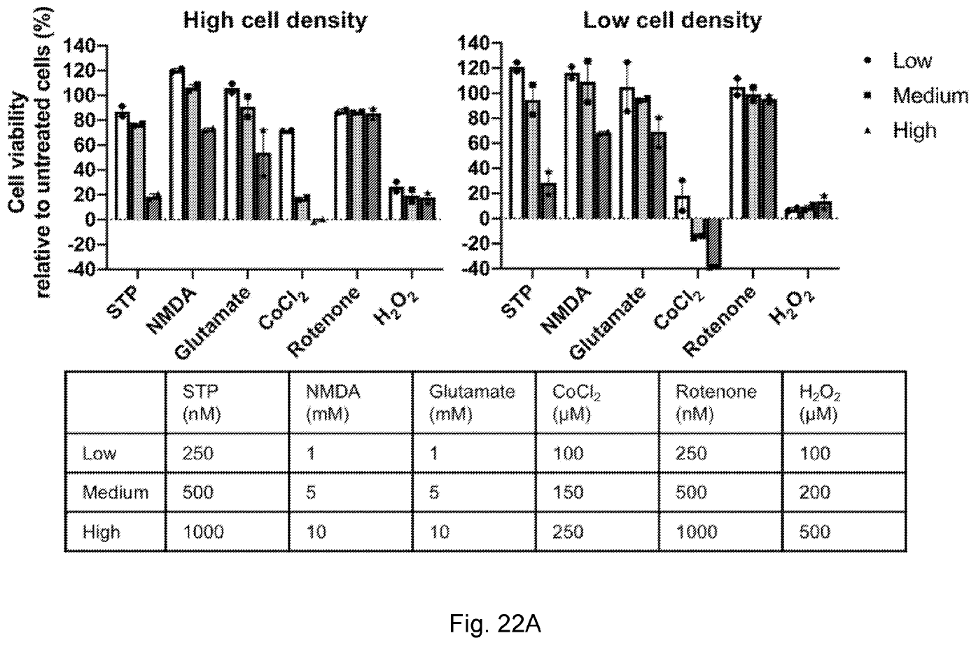

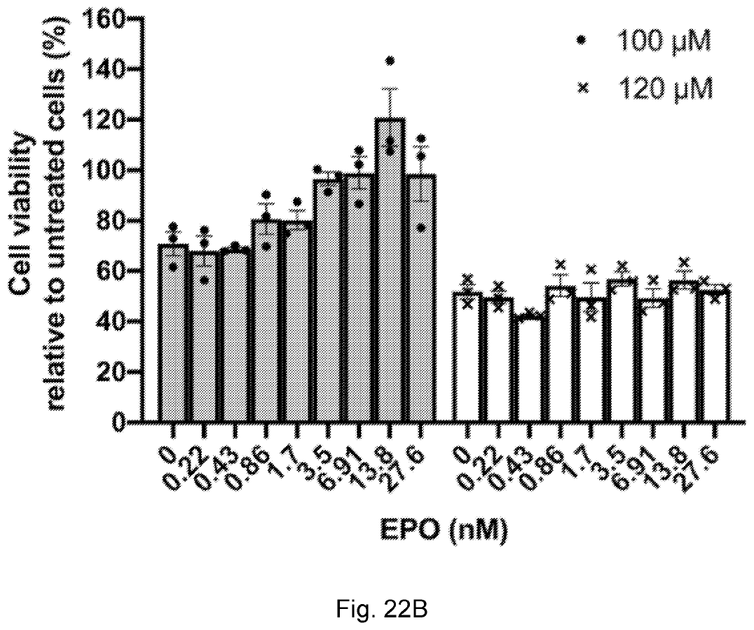

Another set of tests relate to tissue protection. The testing of forms of tissue-protective EPO to demonstrate tissue protection, e.g., reduction of neurodegeneration, is described in e.g., FIG. 4A-4G, 7A-7F, FIG. 8A-8F, FIG. 9A-9B, or FIG. 22A-22B herein. In some embodiments, a neuronal cell line is used, e.g., the SH-SY5Y cell line; SH-SY5Y is a thrice-subcloned cell line derived from the SK—N—SH neuroblastoma cell line. In some embodiments, the neuronal cell line is contacted with a toxic agent, non-limiting examples of which include staurosporine (STP); N-methyl-d-aspartic acid (NMDA); cobalt chloride (CoCl₂); hydrogen peroxide (H₂O₂); glutamate; or rotenone. In some embodiments, the ability of the tissue-protective EPO to protect neural tissue is determined by comparing cell viability of cells treated with both the tissue-protective EPO and a toxic agent, to cells treated with only the toxic agent and not the tissue-protective EPO. It is contemplated that in vivo tests can be used to demonstrate tissue protection, e.g., animal models of a neurodegenerative disease or disorder, hypoxic tissue damage, traumatic brain injury, or stroke.

As described in the Examples herein, mice dosed with tissue-protective EPO exhibited elevated RBC levels, with only minimal platelet effects; see e.g., FIG. 3A-3B or FIG. 6A-6C. This in vivo selectivity can depend on the identity of the GYPA epitope being targeted and the linker sequence length. In a therapeutic context, the compositions and methods described herein permit higher restorative doses of EPO without platelet-mediated side effects, and also can improve drug pharmacokinetics. These results demonstrate how rational drug design can improve in vivo specificity, with potential application to diverse protein therapeutics.

In one aspect of any of the embodiments, described herein is a method of increasing erythropoiesis comprising contacting a red blood cell with a polypeptide described herein, e.g., a tissue-protective EPO protein. The red blood cell can be in vitro or in vivo. In one aspect of any of the embodiments, the methods described herein relate to increasing erythropoiesis in a subject in need thereof by administering to the subject a polypeptide described herein, e.g., a tissue-protective EPO protein. In some embodiments of any of the aspects, the erythropoiesis is increased by at least 1/a, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to a neuron that is not contacted with a tissue-protective EPO protein as described herein or a subject that is not administered a tissue-protective EPO protein as described herein.

In one aspect of any of the embodiments, described herein is a method of decreasing neurodegeneration comprising contacting a neuron with a polypeptide described herein, e.g., a tissue-protective EPO protein. The neuron can be in vitro or in vivo. In one aspect of any of the embodiments, the methods described herein relate to decreasing neurodegeneration in a subject in need thereof by administering to the subject a polypeptide described herein, e.g., a tissue-protective EPO protein.

In some embodiments of any of the aspects, the neurodegeneration is caused by hypoxia, neuronal excitotoxicity, or traumatic brain injury. In excitotoxicity, neurons suffer damage or death when the levels of otherwise necessary and safe neurotransmitters such as glutamate, α-amino-3-hydroxy-5-methyl-4-isoxazolepropionic acid, or N-methyl-D-aspartic acid become pathologically high resulting in excessive stimulation of receptors. Excitotoxicity can be involved in cancers, spinal cord injury, stroke, traumatic brain injury, hearing loss (e.g., through noise overexposure or ototoxicity), and in neurodegenerative diseases of the central nervous system such as multiple sclerosis, Alzheimer's disease, amyotrophic lateral sclerosis (ALS), Parkinson's disease, Huntington's disease, alcoholism, alcohol withdrawal, hyperammonemia, or over-rapid benzodiazepine withdrawal. Other common conditions that cause excessive glutamate concentrations around neurons are hypoglycemia.

In some embodiments of any of the aspects, the neurodegeneration is decreased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 9000/, or more, or at least 10×, 20×, 30×, 40×, 50×, 60×, 70×, 80×, 90×, 100×, 500×, 1,000×, or more higher compared to a neuron that is not contacted with a tissue-protective EPO protein as described herein or a subject that is not administered a tissue-protective EPO protein as described herein.

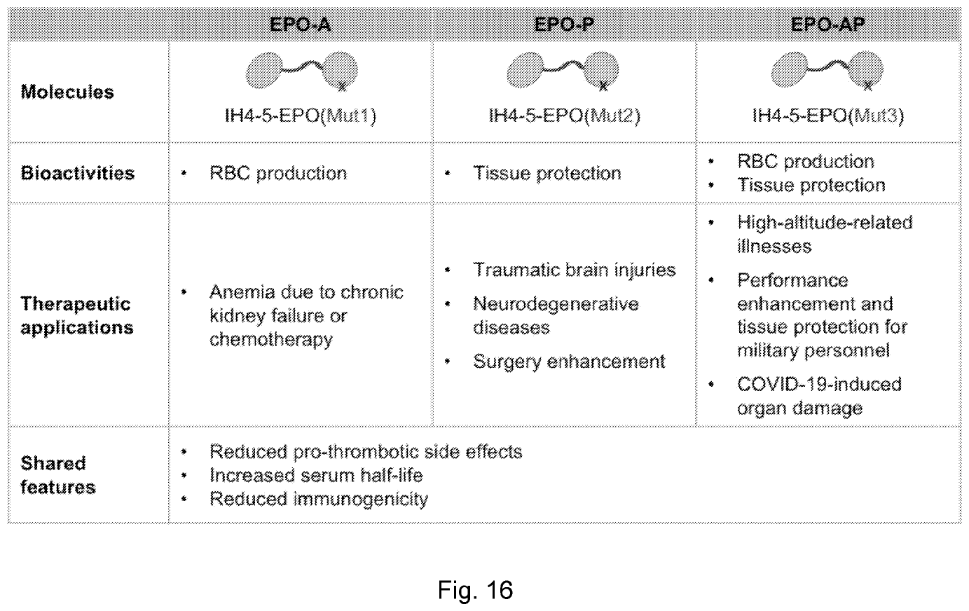

In one aspect of any of the embodiments, the methods described herein relate to treating a subject having or diagnosed as having anemia with a polypeptide described herein, e.g., a tissue-protective EPO protein. Exemplary engineered EPOs that can be used to treat anemia are designated herein as EPO-A (see e.g., FIG. 16, FIG. 27). Subjects having anemia can be identified by a physician using current methods of diagnosing anemia. Symptoms and/or complications of anemia which characterize these conditions and aid in diagnosis are well known in the art and include but are not limited to, fatigue, weakness, pale or yellowish skin, irregular heartbeat, shortness of breath, dizziness, chest pain, cold extremities, and headache. Tests that may aid in a diagnosis of, e.g. anemia include, but are not limited to, a blood count and phenotypic analysis of red blood cells. A family history of anemia, or exposure to risk factors for anemia can also aid in determining if a subject is likely to have anemia or in making a diagnosis of anemia. In some embodiments of any of the aspects, a subject with anemia or in need of erythropoiesis can be a subject having or diagnosed as having chronic renal failure or altitude sickness or who has received chemotherapy.

In one aspect of any of the embodiments, the methods described herein relate to treating a subject having or diagnosed as having need of tissue-protection with a tissue-protective EPO protein as described herein. Exemplary engineered EPOs that can be used for tissue-protection are designated herein as EPO-P (see e.g., FIG. 16, FIG. 27); non-limiting examples of EPO-P include engineered EPO comprising the R14N or R103I mutations. Subjects in need of tissue-protection include those having or diagnosed as having neurodegenerative diseases or disorders, any conditions that accompany short-term hypoxic damage or other pro-apoptotic damage, such as traumatic brain injury (TBI), strokes, or surgeries. Non-limiting examples of neurodegenerative diseases or disorders include Alzheimer's disease, Parkinson's disease, Huntington's disease, amyotrophic lateral sclerosis (ALS), frontotemporal dementia, chronic traumatic encephalopathy (CTE), multiple sclerosis, epilepsy, and neuroinflammation, among others. The molecular mechanisms of neuroinflammation and neurodegenerative diseases are further described, e.g., in Amor, S., et al. (2010), “Inflammation in neurodegenerative diseases.” Immunology, 129: 154-169; Barnham, K., Masters, C. & Bush, A. “Neurodegenerative diseases and oxidative stress.” Nat Rev Drug Discov 3, 205-214 (2004); the contents of each of which are incorporated herein by reference in their entireties.

Subjects having a neurodegenerative disease or disorder, hypoxic tissue damage, traumatic brain injury, or stroke can be identified by a physician using current methods of diagnosing a neurodegenerative disease or disorder, hypoxic tissue damage, traumatic brain injury, or stroke. Symptoms and/or complications of a neurodegenerative disease or disorder, hypoxic tissue damage, traumatic brain injury, or stroke which characterize these conditions and aid in diagnosis are well known in the art and include but are not limited to memory loss, forgetfulness, apathy, anxiety, agitation, a loss of inhibition, or mood changes.

Symptoms and/or complications of hypoxic tissue damage which characterize these conditions and aid in diagnosis are well known in the art and include but are not limited to bluish discoloration of skin, lips, and oral cavity; decreased level of consciousness; cough, fast heart rate; difficulty breathing; slow heart rate; palpitations; sweating; temporary memory loss; reduced ability to move the body; difficulty paying attention; difficulty making sound decisions; seizure; coma; brain death; necroptotic cell death; or apoptotic cell death.

Symptoms and/or complications of a traumatic brain injury which characterize these conditions and aid in diagnosis are well known in the art and include but are not limited to headache; nausea or vomiting; fatigue or drowsiness; problems with speech; dizziness or loss of balance; sensory problems, such as blurred vision, ringing in the ears, a bad taste in the mouth or changes in the ability to smell; sensitivity to light or sound; loss of consciousness for a few seconds to a few minutes; no loss of consciousness, but a state of being dazed, confused or disoriented; memory or concentration problems; mood changes or mood swings; feeling depressed or anxious; difficulty sleeping; sleeping more than usual; loss of consciousness from several minutes to hours; persistent headache or headache that worsens; repeated vomiting or nausea; convulsions or seizures; dilation of one or both pupils of the eyes; clear fluids draining from the nose or ears; inability to awaken from sleep; weakness or numbness in fingers and toes; loss of coordination; profound confusion; agitation, combativeness or other unusual behavior; slurred speech; coma and other disorders of consciousness; change in eating or nursing habits; unusual or easy irritability; persistent crying and inability to be consoled; change in ability to pay attention; change in sleep habits; seizures; sad or depressed mood; drowsiness; or loss of interest in favorite toys or activities.

Symptoms and/or complications of a stroke which characterize these conditions and aid in diagnosis are well known in the art and include but are not limited to trouble speaking and understanding what others are saying; paralysis or numbness of the face, arm or leg; problems seeing in one or both eyes; headache; or trouble walking.

Tests that may aid in a diagnosis of, e.g. a neurodegenerative disease or disorder, hypoxic tissue damage, traumatic brain injury, or stroke include, but are not limited to, imaging (e.g., of the brain by a CT scan, PET scan, MRI, or the like), genetic testing for associated disease markers, cognitive testing (e.g., the clock-drawing test for neurodegenerative diseases or disorders), behavioral testing, physical stamina testing, etc. A family history of a neurodegenerative disease or disorder, hypoxic tissue damage, traumatic brain injury, or stroke, or exposure to risk factors for a neurodegenerative disease or disorder, hypoxic tissue damage, traumatic brain injury, or stroke can also aid in determining if a subject is likely to have a neurodegenerative disease or disorder, hypoxic tissue damage, traumatic brain injury, or stroke or in making a diagnosis of a neurodegenerative disease or disorder, hypoxic tissue damage, traumatic brain injury, or stroke.

In one aspect of any of the embodiments, the methods described herein relate to treating a subject having or diagnosed as having anemia and in need of tissue-protection with a tissue-protective EPO protein as described herein. Exemplary engineered EPOs that can be used for both anemia treatment (i.e., erythropoiesis) and tissue-protection are designated herein as EPO-AP (see e.g., FIG. 16, FIG. 27); non-limiting examples of EPO-AP include engineered EPO comprising the R103K or L08A mutations. Subjects in need of both anemia treatment and tissue-protection include those having or diagnosed as having high altitude-related illnesses, or tissue damage caused by hypoxia, e.g., in COVID-19 patients.

Subjects having altitude sickness or hypoxic tissue damage can be identified by a physician using current methods of diagnosing altitude sickness or hypoxic tissue damage. Symptoms and/or complications of altitude sickness which characterize these conditions and aid in diagnosis are well known in the art and include but are not limited to headache; nausea; dizziness; tiredness; loss of appetite; shortness of breath; fluid build-up in the lungs or brain. Tests that may aid in a diagnosis of, e.g. altitude sickness include, but are not limited to, a chest X-ray to check for fluid in the chest or a brain MRI or CT scan to check for fluid in the brain.

Symptoms and/or complications of hypoxic tissue damage which characterize these conditions and aid in diagnosis are well known in the art and include but are not limited to bluish discoloration of skin, lips, and oral cavity; decreased level of consciousness; cough, fast heart rate; difficulty breathing; slow heart rate; palpitations; sweating; temporary memory loss; reduced ability to move the body; difficulty paying attention; difficulty making sound decisions; seizure; coma; brain death; necroptotic cell death; or apoptotic cell death. Tests that may aid in a diagnosis of, e.g. hypoxic tissue damage include, but are not limited to, physical examination or by using oxygen monitors (pulse oximeters), determining, the oxygen level in a blood gas sample and may include pulmonary function tests. A family history of altitude sickness or hypoxic tissue damage, or exposure to risk factors for altitude sickness or hypoxic tissue damage can also aid in determining if a subject is likely to have altitude sickness or hypoxic tissue damage or in making a diagnosis of altitude sickness or hypoxic tissue damage.

In one aspect of any of the embodiments, the methods described herein relate to enhancing physical performance in a subject in need thereof with a tissue-protective EPO protein as described herein. Exemplary engineered EPOs that can be used for enhancing physical performance include those that can be used for both anemia treatment (i.e., erythropoiesis) and tissue-protection, designated herein as EPO-AP (see e.g., FIG. 16, FIG. 27); non-limiting examples of EPO-AP include engineered EPO comprising the R103K or L108A mutations. Subjects in need of enhanced physical performance include subjects (e.g., military personnel, athletes) acclimating to high-altitude regions, subjects (e.g., military personnel, athletes) seeking safe ways to enhance physical performance (e.g., run time, run distance, or other markers of enhanced physical performance), or subjects (e.g., military personnel) needing general protection from physical threats (e.g., chemical warfare). The compositions and methods described herein can be administered to a subject having or diagnosed as having, e.g., anemia, a neurodegenerative disease or disorder, hypoxic tissue damage, traumatic brain injury, stroke, or altitude sickness. In some embodiments, the methods described herein comprise administering an effective amount of compositions described herein, e.g. a polypeptide as described herein to a subject in order to alleviate a symptom of anemia, a neurodegenerative disease or disorder, hypoxic tissue damage, traumatic brain injury, stroke, or altitude sickness. As used herein, “alleviating a symptom” is ameliorating any condition or symptom associated with the disease or condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of the therapeutic composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay described herein. The dosage can be determined by a physician and adjusted, as necessary, to suit observed effects of the treatment.

In some embodiments, the technology described herein relates to a pharmaceutical composition comprising a polypeptide as described herein, e.g., a tissue-protective EPO protein, and optionally a pharmaceutically acceptable carrier. In some embodiments, the active ingredients of the pharmaceutical composition comprise a polypeptide as described herein, e.g., a tissue-protective EPO protein as described herein. In some embodiments, the active ingredients of the pharmaceutical composition consist essentially of a polypeptide as described herein, e.g., a tissue-protective EPO protein as described herein. In some embodiments, the active ingredients of the pharmaceutical composition consist of a polypeptide as described herein, e.g., a tissue-protective EPO protein as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a polypeptide as described herein, e.g., a tissue-protective EPO protein as described herein.

In some embodiments, the pharmaceutical composition comprising a polypeptide as described herein, e.g., a tissue-protective EPO protein as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a polypeptide as described herein, e.g. a tissue-protective EPO protein as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, a polypeptide as described herein, e.g. a tissue-protective EPO protein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 Bi; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, a polypeptide as described herein, e.g., a tissue-protective EPO protein described herein is administered as a monotherapy, e.g., another treatment for anemia, a neurodegenerative disease or disorder, hypoxic tissue damage, traumatic brain injury, stroke, or altitude sickness, or to increase erythropoiesis, neuroprotection, or physical performance is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include iron supplements, transfusions, and RBC transfusions. Additional non-limiting examples of a second agent and/or treatment can include oxygen therapy, anti-seizure drugs, coma-inducing drugs, diuretics, surgery, an anticoagulant, statin, antihypertensive drug, or ACE inhibitor. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising a polypeptide as described herein, e.g., a tissue-protective EPO protein as described herein can be administered to a patient once. In certain embodiments, an effective dose of a composition comprising a polypeptide as described herein, e.g., a tissue-protective EPO protein can be administered to a patient repeatedly. For systemic administration, subjects can be administered a therapeutic amount of a composition comprising a polypeptide as described herein, e.g., a tissue-protective EPO protein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, the tissue-protective EPO is at a concentration of at least 10⁻¹⁶ M. In some embodiments of any of the aspects, the tissue-protective EPO is at a concentration of at most 10⁻⁸ M. In some embodiments of any of the aspects, the tissue-protective EPO is at a concentration of at least 10⁻¹⁶ M, at least 10⁻¹⁵ M, at least 10⁻⁴ M, at least 10⁻⁴ M, at least 10⁻¹² M, at least 10⁻¹¹ M, at least 10⁻¹⁰ M, or at least 10⁻⁹ M. In some embodiments of any of the aspects, the tissue-protective EPO is at a concentration of at least 6 pmol/200 uL (30 nM). In some embodiments of any of the aspects, the tissue-protective EPO is at a concentration of at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 250 nM, at least 300 nM, at least 350 nM, at least 400 nM, at least 450 nM, or at least 500 nM, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a polypeptide as described herein, e.g., a tissue-protective EPO protein can be administered over a period of time, such as over a 5 minute, 10 minute, 15 minute, 20 minute, or 25 minute period.

The dosage ranges for the administration of a polypeptide as described herein, e.g., a tissue-protective EPO protein, according to the methods described herein depend upon, for example, the form of the polypeptide, its potency, and the extent to which symptoms, markers, or indicators of a condition described herein are desired to be reduced, for example the percentage reduction desired for anemia symptoms or the extent to which, for example, red blood cell numbers are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a polypeptide as described herein, e.g. a tissue-protective EPO protein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in in vitro or animal models of a condition described herein, e.g., according to the various assays described in detail elsewhere herein.

By way of non-limiting example, a human patient may be treated with a protein of the invention as follows. A patient with chronic kidney disease who is being treated by dialysis may be given a tissue-protective EPO of the invention by iv administration, post-dialysis, using the same iv line that has been established for the dialysis. For an adult human patient, a typical initial dose of a tissue-protective EPO protein is about 400 micrograms, administered once per two weeks. For patients that respond with a rapid rise in hemoglobin (such as more than 1 g/dL in any 2-week period), the dose of tissue-protective EPO should be reduced by 25% or more as needed to reduce rapid responses. For patients who do not respond adequately—for example if the hemoglobin has not increased by more than 1 g/dL after 4 weeks of therapy—the dose should be increased by 25%. The response to tissue-protective EPO varies from patient to patient and hemoglobin levels should be monitored.

In some embodiments of any of the aspects, the starting doses of a polypeptide as described herein, e.g. a tissue-protective EPO protein is about 1 microgram to 100 milligrams, 10 micrograms to 10 milligrams, 50 micrograms to 3.2 milligrams, 100 micrograms to 1.6 milligrams, 200 micrograms to 800 micrograms, or about 400 micrograms. The tissue-protective EPO of the invention is typically administered by iv infusion, or subcutaneous, intramuscular or intradermal injections, either once per week, once per two weeks, or once per month. These are typical doses for adult patients. Depending on the weight of the patient in kilograms, the invention provides starting doses of about 0.2 micrograms/kg to 200 micrograms/kg, 0.75 micrograms/kg to 50 micrograms/kg, 1.5 micrograms/kg to 24 micrograms/kg, 3 micrograms/kg to 12 micrograms/kg, or about 6 micrograms/kg.

As tissue-protective EPO proteins can be erythropoiesis-stimulating agents (ESAs), general considerations about when to treat, methods of administration and so on are the same as for other ESAs such as Epogen™ or Procrit™, and are described in the package inserts that accompany these drugs. The package insert information may also be obtained, for example, on the world wide web at pi.amgen.com/˜/media/amgen/repositorysites/pi-amgen-com/epogen/epogen_pi_hcp_english.pdf. In addition, for self-injecting pre-dialysis patients administering a tissue-protective EPO protein at home, the methods for safe self-injection of Epogen available on the world wide web at pi.amgen.com/-/media/amgen/repositorysites/pi-amgen-com/epogen/epogen_piu_pt_english.pdf also apply. It should be noted that the black-box warnings in package inserts for other ESAs such as Epogen, Procrit, and Aranesp™ do not apply to the tissue-protective EPO molecules of the invention, particularly those using the IH4 V domain, a linker of 7 amino acids or less, and a mutation in the EPO element comprising at least one affinity-decreasing mutation in the weak face of EPO relative to wild-type EPO, e.g., corresponding to R103K or L108A.

By way of non-limiting example, a 70-kg adult human patient with chronic kidney disease and uncontrolled hypertension can be treated as follows. The patient has a hematocrit of less than 25, corresponding to a hemoglobin level of less than about 8 gm/dL, before treatment is initiated. The patient is given between 10 micrograms and 10 milligrams, but preferably about 400 micrograms, of a tissue-protective EPO protein as described herein. The protein is administered after a dialysis procedure, using the same iv line that was set up for dialysis; thus, the administration is intravenous. After 2 weeks, the patient's hemoglobin level is measured and found to be 8.5 gm/dL. This is considered to be an acceptable rate of increase, so at this time the patient is given another dose of the same protein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of anemia, chronic kidney disease, a neurodegenerative disease or disorder, hypoxic tissue damage, traumatic brain injury, stroke, or altitude sickness. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Tr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Other boundaries defining CDRs overlapping with the Kabat CDRs have been described by Padlan (FASEB J. 9:133-139 (1995)) and MacCallum (J Mol Biol 262(5):732-45 (1996)) and Chothia (J. Mol. Biol. 196:901-917 (1987) and Nature 342:877-883 (1989)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat defined CDRs. The CDR's identified herein, e.g., SEQ ID NOs 72-74 are identified by the Kabat system (see, e.g. FIGS. 4 and 5 of International Patent Application WO 2020/132234).

The term “antigen-binding portion” of an antibody refers to one or more portions of an antibody as described herein, said portions) still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a dAb portion consisting of a VH domain or of VH, CH1, CH2, DH3, or VH, CH2, CH3 (dAbs, or single domain antibodies, comprising only V_{L} domains have also been shown to specifically bind to target epitopes). Although the two domains of the Fv portion, namely VL and VH, are encoded by separate genes, they may further be linked to one another using a synthetic linker, e.g., a poly-G4S amino acid sequence (‘G4S’ disclosed in SEQ ID NOs: 65, 115-124), and recombinant methods, making it possible to prepare them as a single protein chain in which the VL and VH regions combine in order to form monovalent molecules (known as single chain Fv (ScFv)). The term “antigen-binding portion” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody, antibody reagent, or antigen-binding portion thereof as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cysteine residue, a marker peptide and a C-terminal polyhistidinyl, e.g., hexahistidinyl tag (‘hexahistidinyl tag’) in order to produce bivalent and biotinylated scFv molecules.

In some embodiments, the antibody, antibody reagent, or antigen-binding portion thereof described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin). The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody, or antigen-binding portion thereof as described herein. Such functional activities include binding to cancer cells and/or anti-cancer activity. Additionally, a polypeptide having functional activity means the polypeptide exhibits activity similar, but not necessarily identical to, an activity of a reference antibody, antibody reagent, or antigen-binding portion thereof as described herein, including mature forms, as measured in a particular assay, such as, for example, a biological assay, with or without dose dependency. In the case where dose dependency does exist, it need not be identical to that of the reference antibody, antibody reagent, or antigen-binding portion thereof but rather substantially similar to the dose-dependence in a given activity as compared to the reference antibody, antibody reagent, or antigen-binding portion thereof as described herein (i.e., the candidate polypeptide will exhibit greater activity, or not more than about 25-fold less, about 10-fold less, or about 3-fold less activity relative to the antibodies, antibody reagents, and/or antigen-binding portions described herein).

In some embodiments, the antibody reagents (e.g., antibodies) described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

In some embodiments, the antibody, antibody reagent, and/or antigen-binding portion thereof is an isolated polypeptide. In some embodiments, the antibody, antibody reagent, and/or antigen-binding portion thereof is a purified polypeptide. In some embodiments, the antibody, antibody reagent, and/or antigen-binding portion thereof is an engineered polypeptide.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama, and Lama vicugna) have been characterized with respect to size, structural complexity and antigenicity for human subjects. Certain IgG antibodies from this family of mammals as found in nature lack light chains, and are thus structurally distinct from the typical four chain quatemary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from ABLYNX, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

“Glycophorin A” or “GYPA” (sometimes referred to as GPA or CD235a in the art) is a sialoglycoprotein found on erythrocyte membranes which carries antigenic determinants for the MN and Ss blood groups. Sequences for GYPA in a number of species are known in the art, e.g., human GYPA (NCBI Gene ID: 2993) mRNA (NM_001308187.1; NM_001308190.1; NM_002099.8) and protein (NP_001295116.1; NP_001295119.1; NP_002090.4) sequences; see e.g., SEQ ID NOs: 71, 89.

In some embodiments, a nucleic acid encoding a polypeptide as described herein (e.g. a tissue-protective EPO polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. anemia, a neurodegenerative disease or disorder, hypoxic tissue damage, traumatic brain injury, stroke, or altitude sickness. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A polypeptide comprising:
   a) an engineered erythropoietin (EPO) comprising at least one affinity-decreasing mutation in the weak face of EPO relative to wild-type EPO;
   b) an anti-glycophorin A (GYPA) antibody reagent that binds an epitope of SEQ ID NO: 89; and
   c) a linker sequence separating the anti-GYPA antibody reagent and the engineered erythropoietin.
2. The polypeptide of paragraph 1, wherein the weak face of EPO binds with a dissociation constant (K_{d}) of at least 1 μM to the EPO receptor (EPOR).
3. The polypeptide of paragraph 1 or 2, wherein the at least one mutation is in Helix A (Ser9-Gly28 of SEQ ID NO: 1) and/or Helix C (Pro90-Leu112 of SEQ ID NO: 1) relative to wild-type EPO of SEQ ID NO: 1.
4. The polypeptide any one of paragraphs 1-3, wherein the at least one mutation is at an amino acid residue relative to SEQ ID NO: 1 selected from S104, R14, Y15, R103, and L108.
5. The polypeptide of paragraph 4, wherein the at least one mutation is S104I, R14E, R14Q, R14N, Y15I, R103I, R103Q, R103K, or L108A.
6. The polypeptide of any one of paragraphs 1-5, wherein the at least one mutation is at an amino acid residue relative to SEQ ID NO: 1 selected from R14, R103, and L108.
7. The polypeptide of paragraph 6, wherein the at least one mutation is R14N, R103I, R103K, or L108A.
8. The polypeptide of any one of paragraphs 1-7, wherein the at least one mutation is at an amino acid residue relative to SEQ ID NO: 1 selected from R103 and L108.
9. The polypeptide of paragraph 8, wherein the at least one mutation is R103K or L108A.
10. The polypeptide of any one of paragraphs 1-9, wherein the at least one mutation does not substantially affect binding to CD131.
11. The polypeptide of any one of paragraphs 1-10, wherein the engineered erythropoietin does not comprise a mutation in a region of wild-type EPO that binds to CD131.
12. The polypeptide of any one of paragraphs 1-11, wherein the engineered erythropoietin does not comprise a mutation in the strong face of EPO relative to wild-type EPO.
13. The polypeptide of paragraph 12, wherein the strong face of EPO binds with a dissociation constant (K_{d}) of no more than 1 nM to EPOR.
14. The polypeptide of any one of paragraphs 1-13, wherein the engineered erythropoietin does not comprise a mutation in Helix D (F138-C161 of SEQ ID NO: 1) or the AB loop (C29-E55 of SEQ ID NO: 1) relative to wild-type EPO of SEQ ID NO: 1.
15. The polypeptide of any one of paragraphs 1-14, wherein the engineered erythropoietin does not comprise a mutation at an amino acid residue relative to SEQ ID NO: 1 selected from R150, K45, A30, H32, P87, W88, P90, R53, and E55.
16. The polypeptide of paragraph 15, wherein the at least one mutation is not R150A, K45D, A30N, H32T, P87V, W88N, P90T, R53N, or E55T.
17. The polypeptide of any one of paragraphs 1-16, wherein the anti-GYPA antibody reagent comprises one or more CDRs of IH4.
18. The polypeptide of any one of paragraphs 1-17, wherein the anti-GYPA antibody reagent comprises the three CDRs of IH4.
19. The polypeptide of any one of paragraphs 1-18, wherein the anti-GYPA antibody reagent comprises a VHH having the sequence of SEQ ID NO: 2 or 50.
20. The polypeptide of paragraph 1, wherein the anti-GYPA antibody reagent comprises one or more CDRs of an antibody reagent selected from the group consisting of 10F7, 1C3, 2B-11, 2B-12, 2B-13, 2B-18, 2B-19, 2B-20, 2B-21, 2B-25, 2B-4, 2B-9, A63-B/C2, A88-A/F9, A88-D/C7, A88-E/H2, A96-D/A7, A96-E/F7, B14 (also known as BRIC 14), B89 (also known as BRIC 89), BRIC 116, BRIC 117, BRIC 119, BRIC 93, GPA 105, GPA 33, IH4, IH4v1, Mab 158, NaM10-2H12, NaM10-6G4, NaM16-IB10, NaM70-3C10, OSK4-1, R 10, R7, and R18.
21. The polypeptide of paragraph 1, wherein the anti-GYPA antibody reagent comprises one or more CDRs of an antibody reagent selected from R18, IH4, IH4v1, 10F7, and Table 13.
22. The polypeptide of paragraph 1, wherein the anti-GYPA antibody reagent comprises the CDRs of an antibody reagent selected from R18, IH4, IH4v1, 10F7, and Table 13.
23. The polypeptide of paragraph 1, wherein the anti-GYPA antibody reagent comprises the VH and VL sequences of an antibody reagent selected from R18, IH4, IH4v1, 10F7, and Table 13.
24. The polypeptide of paragraph 1, wherein the anti-GYPA antibody reagent comprises an antibody reagent selected from R18, IH4, IH4v1, 10F7, and Table 13.
25. The polypeptide of any of paragraphs 20-24, wherein the antibody reagent is selected from 10F7, R18, IH4, IH4v1, 2B-21, 2B-25, 2B-9, 2B-20, 2B-19, NaM70-3C10, 2B-4, B14, B89, R7, and BRIC 93.
26. The polypeptide of any of paragraphs 20-24, wherein the antibody reagent is selected from 10F7, IH4, IH4v1, 2B-9, 2B-20, 2B-19, NaM70-3C10, 2B-4, B14, B89, R7, and BRIC 93.
27. The polypeptide of any of paragraphs 1-26, wherein the linker sequence is no more than 17 amino acids in length.
28. The polypeptide of any of paragraphs 1-27, wherein the linker sequence is 1, 2, 3, 4 or 5 amino acids in length.
29. The polypeptide of any of paragraphs 1-28, wherein the linker sequence is at least 5 amino acids in length.
30. The polypeptide of any of paragraphs 1-29, wherein the linker sequence is 5-35 amino acids in length.
31. The polypeptide of any of paragraphs 1-30, wherein the linker sequence is 5-7 amino acids in length.
32. The polypeptide of any of paragraphs 1-31, wherein the linker sequence is 7 or fewer amino acids in length.
33. The polypeptide of any of paragraphs 1-31, wherein the polypeptide comprises the amino acid sequence of SEQ ID NO: 148.
34. The polypeptide of any of paragraphs 1-31, wherein the polypeptide consists of the amino acid sequence of SEQ ID NO: 148.
35. The polypeptide of any of paragraphs 1-31, wherein the polypeptide comprises the amino acid sequence of SEQ ID NO: 148 and a detectable tag.
36. The polypeptide of paragraph 35, wherein the detectable tag is selected from the group consisting of c-Myc, HA, VSV-G, HSV, FLAG, V5, HIS6, and biotin.
37. The polypeptide of any of paragraphs 1-36, wherein the polypeptide consists of the amino acid sequence of SEQ ID NO: 144.
38. A nucleic acid encoding the polypeptide of any one of paragraphs 1-37.
39. A vector comprising the nucleic acid of paragraph 38.
40. A cell comprising the nucleic acid of paragraph 38 or the vector of paragraph 39.
41. A pharmaceutical composition comprising the polypeptide of any one of paragraphs 1-37; the nucleic acid of paragraph 38; the vector of paragraph 43; or the cell of paragraph 39.
42. A method of increasing erythropoiesis comprising contacting a red blood cell with a polypeptide of any of paragraphs 1-37.
43. A method of decreasing neurodegeneration comprising contacting a neuron with a polypeptide of any of paragraphs 1-37.
44. The method of paragraph 43, wherein the neurodegeneration is caused by hypoxia, neuronal excitotoxicity, or traumatic brain injury.
45. A method of treating a neurodegenerative disease or disorder, hypoxic tissue damage, traumatic brain injury, or stroke in a subject in need thereof, the method comprising administering an effective amount of a polypeptide of any one of paragraphs 1-37 to the subject.
46. A method of treating altitude sickness or hypoxic tissue damage in a subject in need thereof, the method comprising administering an effective amount of a polypeptide of any one of paragraphs 1-37 to the subject.
47. A method of enhancing physical performance in a subject in need thereof, the method comprising administering an effective amount of a polypeptide of any one of paragraphs 1-37 to the subject.
48. The method of any one of paragraphs 42-47, wherein the polypeptide is at a concentration of at least 10⁻¹⁶ M.
49. The method any one of paragraphs 42-48, wherein the polypeptide is at a concentration of at most 10⁻⁸ M.
50. A polypeptide of any of paragraphs 1-37, for use in treating a neurodegenerative disease or disorder, hypoxic tissue damage, traumatic brain injury, stroke, or altitude sickness in a subject in need thereof.
51. A polypeptide of any of paragraphs 1-37, for use in enhancing physical performance in a subject in need thereof.

Some embodiments of the technology described herein can be defined according to any of the following numbered paragraphs:
1. A polypeptide comprising:
   a) an engineered erythropoietin (EPO) comprising at least one affinity-decreasing mutation in the weak face of EPO relative to wild-type EPO;
   b) an anti-glycophorin A (GYPA) antibody reagent that binds an epitope of SEQ ID NO: 89; and
   c) a linker sequence separating the anti-GYPA antibody reagent and the engineered erythropoietin.
2. The polypeptide of paragraph 1, wherein the weak face of EPO binds with a dissociation constant (K_{d}) of at least 1 μM to the EPO receptor (EPOR).
3. The polypeptide of paragraph 1 or 2, wherein the at least one mutation is in Helix A (Ser9-Gly28 of SEQ ID NO: 1) and/or Helix C (Pro90-Leu112 of SEQ ID NO: 1) relative to wild-type EPO of SEQ ID NO: 1.
4. The polypeptide any one of paragraphs 1-3, wherein the at least one mutation is at an amino acid residue relative to SEQ ID NO: 1 selected from S104, R14, Y15, R103, and L108.
5. The polypeptide of paragraph 4, wherein the at least one mutation is S104I, R14E, R14Q, R14N, Y15I, R103I, R103Q, R103K, or L108A.
6. The polypeptide of any one of paragraphs 1-5, wherein the at least one mutation is at an amino acid residue relative to SEQ ID NO: 1 selected from R14, R103, and L108.
7. The polypeptide of paragraph 6, wherein the at least one mutation is R14N, R103I, R103K, or L108A.
8. The polypeptide of any one of paragraphs 1-7, wherein the at least one mutation is at an amino acid residue relative to SEQ ID NO: 1 selected from R103 and S108.
9. The polypeptide of paragraph 8, wherein the at least one mutation is R103K or L108A.
10. The polypeptide of any one of paragraphs 1-9, wherein the at least one mutation does not substantially affect binding to CD131.
11. The polypeptide of any one of paragraphs 1-10, wherein the engineered erythropoietin does not comprise a mutation in a region of wild-type EPO that binds to CD131.
12. The polypeptide of any one of paragraphs 1-11, wherein the engineered erythropoietin does not comprise a mutation in the strong face of EPO relative to wild-type EPO.
13. The polypeptide of paragraph 12, wherein the strong face of EPO binds with a dissociation constant (K_{d}) of no more than 1 nM to EPOR.
14. The polypeptide of any one of paragraphs 1-13, wherein the engineered erythropoietin does not comprise a mutation in Helix D (F138-C161 of SEQ ID NO: 1) or the AB loop (C29-E55 of SEQ ID NO: 1) relative to wild-type EPO of SEQ ID NO: 1.
15. The polypeptide of any one of paragraphs 1-14, wherein the engineered erythropoietin does not comprise a mutation at an amino acid residue relative to SEQ ID NO: 1 selected from R150, K45, A30, H32, P87, W88, P90, R53, and E55.
16. The polypeptide of paragraph 15, wherein the at least one mutation is not R150A, K45D, A30N, H32T, P87V, W88N, P90T, R53N, or E55T.
17. The polypeptide of any one of paragraphs 1-16, wherein the anti-GYPA antibody reagent comprises one or more CDRs of IH4.
18. The polypeptide of any one of paragraphs 1-17, wherein the anti-GYPA antibody reagent comprises the three CDRs of IH4.
19. The polypeptide of any one of paragraphs 1-18, wherein the anti-GYPA antibody reagent comprises a VHH having the sequence of SEQ ID NO: 2 or 50.
20. The polypeptide of paragraph 1, wherein the anti-GYPA antibody reagent comprises one or more CDRs of an antibody reagent selected from the group consisting of 10F7, 1C3, 2B-11, 2B-12, 2B-13, 2B-18, 2B-19, 2B-20, 2B-21, 2B-25, 2B-4, 2B-9, A63-B/C2, A88-A/F9, A88-D/C7, A88-E/H2, A96-D/A7, A96-E/F7, B14 (also known as BRIC 14), B89 (also known as BRIC 89), BRIC 116, BRIC 117, BRIC 119, BRIC 93, GPA 105, GPA 33, IH4, IH4v1, Mab 158, NaM10-2H12, NaM10-6G4, NaM16-IB10, NaM70-3C10, OSK4-1, R 10, R7, and R18.
21. The polypeptide of paragraph 1, wherein the anti-GYPA antibody reagent comprises one or more CDRs of an antibody reagent selected from R18, IH4, IH4v1, 10F7, and Table 13.
22. The polypeptide of paragraph 1, wherein the anti-GYPA antibody reagent comprises the CDRs of an antibody reagent selected from R18, IH4, IH4v1, 10F7, and Table 13.
23. The polypeptide of paragraph 1, wherein the anti-GYPA antibody reagent comprises the VH and VL sequences of an antibody reagent selected from R18, IH4, IH4v1, 10F7, and Table 13.
24. The polypeptide of paragraph 1, wherein the anti-GYPA antibody reagent comprises an antibody reagent selected from R18, IH4, IH4v1, 10F7, and Table 13.
25. The polypeptide of any of paragraphs 20-24, wherein the antibody reagent is selected from 10F7, R18, IH4, IH4v1, 2B-21, 2B-25, 2B-9, 2B-20, 2B-19, NaM70-3C10, 2B-4, B14, B89, R7, and BRIC 93.
26. The polypeptide of any of paragraphs 20-24, wherein the antibody reagent is selected from 10F7, IH4, IH4v1, 2B-9, 2B-20, 2B-19, NaM70-3C10, 2B-4, B14, B89, R7, and BRIC 93.
27. The polypeptide of any of paragraphs 1-26, wherein the linker sequence is no more than 17 amino acids in length.
28. The polypeptide of any of paragraphs 1-27, wherein the linker sequence is at least 5 amino acids in length.
29. The polypeptide of any of paragraphs 1-28, wherein the linker sequence is 5-35 amino acids in length.
30. The polypeptide of any of paragraphs 1-29, wherein the linker sequence is 5-7 amino acids in length.
31. The polypeptide of any of paragraphs 1-30, wherein the linker sequence is 7 or fewer amino acids in length.
32. A nucleic acid encoding the polypeptide of any one of paragraphs 1-31.
33. A vector comprising the nucleic acid of paragraph 32.
34. A cell comprising the nucleic acid of paragraph 32 or the vector of paragraph 33.
35. A pharmaceutical composition comprising the polypeptide of any one of paragraphs 1-31; the nucleic acid of paragraph 32; the vector of paragraph 33; or the cell of paragraph 34.
36. A method of increasing erythropoiesis comprising contacting a red blood cell with a polypeptide of any of paragraphs 1-31.
37. A method of decreasing neurodegeneration comprising contacting a neuron with a polypeptide of any of paragraphs 1-31.
38. The method of paragraph 37, wherein the neurodegeneration is caused by hypoxia, neuronal excitotoxicity, or traumatic brain injury.
39. A method of treating a neurodegenerative disease or disorder, hypoxic tissue damage, traumatic brain injury, or stroke in a subject in need thereof, the method comprising administering an effective amount of a polypeptide of any one of paragraphs 1-31 to the subject.
40. A method of treating altitude sickness or hypoxic tissue damage in a subject in need thereof, the method comprising administering an effective amount of a polypeptide of any one of paragraphs 1-31 to the subject.
41. A method of enhancing physical performance in a subject in need thereof, the method comprising administering an effective amount of a polypeptide of any one of paragraphs 1-31 to the subject.
42. The method of any one of paragraphs 36-41, wherein the polypeptide is at a concentration of at least 10⁻¹⁶ M.
43. The method any one of paragraphs 36-42, wherein the polypeptide is at a concentration of at most 10⁻⁸ M.
44. A polypeptide of any of paragraphs 1-31, for use in treating a neurodegenerative disease or disorder, hypoxic tissue damage, traumatic brain injury, stroke, or altitude sickness in a subject in need thereof.
45. A polypeptide of any of paragraphs 1-31, for use in enhancing physical performance in a subject in need thereof.

The technology described herein is further illustrated by the following examples which in no way should be construed as being further limiting.

### EXAMPLES

### Example 1: Rational Engineering of an Erythropoietin Fusion Protein to Treat Hypoxia

Erythropoietin enhances oxygen delivery and reduces hypoxia-induced cell death, but its pro-thrombotic activity is problematic for use of erythropoietin in treating hypoxia. Described herein is a fusion protein that stimulates red blood cell production and neuroprotection without triggering platelet production, a marker for thrombosis. An exemplary fusion protein comprises an anti-glycophorin A nanobody and an erythropoietin mutant (L108A). The mutation reduces activation of erythropoietin receptor homodimers that induce erythropoiesis and thrombosis, but maintains the tissue-protective signaling. The binding of the nanobody element to glycophorin A rescues homodimeric erythropoietin receptor activation on red blood cell precursors. In a cell proliferation assay, the fusion protein is active at 10⁻¹⁴ M, allowing an estimate of the number of receptor-ligand complexes needed for signaling. This fusion protein stimulated erythroid cell proliferation in vitro and in mice, and shows neuroprotective activity in vitro. The erythropoietin fusion protein can be used for treating hypoxia.

Erythropoietin (EPO) stimulates red blood cell (RBC) production in response to hypoxia. It inhibits apoptosis of late-stage erythroid precursors (e.g., Colony-Forming Unit-Erythroid (CFU-E), Burst-Forming Unit-Erythroid BFU-E) and promotes their proliferation and maturation into the fully committed erythroid lineage. Healthy human adult kidneys constitutively produce EPO at low levels, maintaining ˜1-5 pM of circulating EPO under normoxic conditions to sustain constant hemoglobin levels. In response to hypoxic stress or massive blood loss, EPO production is stimulated and the number of circulating erythrocytes increases, allowing for more efficient tissue oxygenation.

EPO, like other cytokines and hormones, is pleiotropic and performs several other biological functions in addition to hematopoiesis. Functional EPO receptors (EPORs) are expressed in many tissues other than erythroid precursors, such as endothelial cells, cardiomyocytes, and cells of the central nervous system. Deletion of EPORs in mouse embryos results not only in impaired erythropoiesis, but also in developmental defects in the heart, the vasculature, and the brain (see e.g., Ogunshola and Bogdanova, 2013, Methods Mol Biol, 982, 13-41, the contents of which are incorporated herein by reference in their entirety). Existence of functional EPORs in non-hematopoietic tissues indicates that EPO activates EPORs in different contexts to induce biological activities that are independent of erythropoiesis.

Non-hematopoietic functions of EPO include enhancement of blood clotting and tissue protection in response to hypoxia. These functions indicate that EPO mediates the body's response to hemorrhage, rather than simply being an RBC-producing hormone. When an animal is wounded, the immediate response by the body should be to stop bleeding, increase RBC production, promote tissue oxygenation and ensure tissue survival until oxygen levels return to baseline. Pro-thrombotic effects have been observed as adverse side effects of EPO in the treatment of anemia. Chronic kidney failure patients receiving EPO exhibit higher incidences of strokes, hypertension and death. Cancer patients treated with EPO had accelerated tumor growth and lower survival rate, possibly due to EPORs on cancer cells themselves, increased tumor angiogenesis, and deep vein thrombosis. EPO's tissue-protective effects in response to hypoxia have also been shown in animal models and are indicated in several clinical studies. Intravenous injections of high doses of EPO significantly reduced infarct size and serum markers of brain damage in acute ischemic stroke patients, and improved motor and cognitive function in multiple sclerosis patients. EPO treatment also resulted in a lower mortality rate and improved neurological recovery amongst traumatic brain injury (TBI) patients. The protective activity of EPO is general to all cellular insults tested so far, including hypoxia, TBI and neuronal excitotoxicity. See e.g., Drüeke et al., 2006, N Engl J Med, 355, 2071-2084; Singh et al., 2006, N Engl J Med, 355, 2085-2098; Pfeffer et al., 2009, N Engl J Med, 361, 2019-2032; Henke et al., 2003, Lancet, 362, 1255-1260; Okazaki et al., 2008, Neoplasia, 10, 932-939; Yasuda et al., 2003, Carcinogenesis, 24, 1021-1029; Ehrenreich et al., 2002, Mol Med, 8, 495-505; Ehrenreich et al., 2007, Brain, 130, 2577-2588; Aloizos et al., 2015, Turk Neurosurg, 25, 552-558; Fantacci et al., 2006, Proc Natl Acad Sci USA, 103, 17531-17536; Robinson et al., 2018, Front Neurol. 19, 9:451; Park et al., 2011, Neurotoxicology, 32, 879-887; the contents of each of which are incorporated herein by reference in their entireties.

Due to its erythropoietic and tissue-protective functions, EPO can be used as a therapeutic for various conditions that cause hypoxia, such as chronic obstructive pulmonary disease (COPD), right-side heart failure and viral infection that requires use of a ventilator. However, two major challenges have limited the clinical use of EPO for tissue protection resulting from hypoxia. First, EPO has a pro-thrombotic effect that is observed at low doses, while the tissue-protective effect requires much higher doses. Thus, doses at which EPO might be effective for tissue protection are considered unsafe. Second, EPO (30.4 kDa) has a short plasma half-life of ˜8 hours after a single intravenous injection in humans (see e.g., Bunn, 2013, Cold Spring Harb Perspect Med, 3, a011619, the contents of which are incorporated herein by reference in their entirety). Its poor pharmacokinetic profile necessitates frequent dosing to maintain the high levels of EPO required for efficacy.

EPO acts through two distinct receptor complexes (see e.g., FIG. 1A-1B). RBC production and clotting is mediated via EPOR homodimers, whereas the angiogenic and tissue-protective activities of EPO are thought to be regulated by heterodimers of EPOR and the co-receptor CD131 (also known as the receptor common beta subunit). EPO monomers bind to EPOR homodimers through a strong interaction (K_{D}=1 nM) on one face involving residues such as N147 and R150 (referred to herein as the “strong face” or “strong side” of EPO) (see e.g., FIG. 1A, FIG. 1C and FIG. 1D), and through a weak interaction (K_{D}=1 μM) on another face involving residues such as S100, R103, S104 and L108 (referred to herein as the “weak face” or “weak side” of EPO) (see e.g., FIG. 1A, FIG. 1C and FIG. 1E). Without wishing to be bound by theory, tissue-protective signaling through EPOR-CD131 heterodimers is thought to involve EPO binding to EPOR through its strong face and an interaction through CD131 (see e.g., FIG. 1B). This configuration is indicated by the fact that while weak-face mutations (e.g., S100E and R103E) disrupt EPOR homodimer signaling and RBC production, there is essentially no effect on neuroprotective signaling. Without wishing to be bound by theory, it is hypothesized that proteins comprising mutations on the weak face of EPO mediate neuroprotection—i.e., the mutations do not disrupt a possible interaction with CD131. Thus, the weak face of EPO can be manipulated for protein engineering purposes and still maintain its tissue-protective function. See e.g., Gan et al. (2012) Stroke, 43, 3071-3077; Hanazono et al., 1995, Biochem Biophys Res Commun, 208, 1060-1066; Brines et al., 2004, Proc Natl Acad Sci USA, 101, 14907-14912; Leist et al., 2004, Science, 305, 239-242; Bennis et al., 2012, J Thromb Haemost, 10, 1914-1928; Elliott et al., 1997, Blood, 89, 493-502; Syed et al., 1998, Nature, 395, 511-516; the contents of each of which are incorporated herein by reference in their entireties.

Described herein are ‘chimeric activator’ proteins in which a mutated EPO with lower receptor affinity is fused to an antibody element that binds to glycophorin A (GPA). A weakened form of EPO with a mutation in the strong face (e.g., R150A) that is also fused to an anti-GPA antibody element can specifically activate production of RBCs and not platelets. Such an anti-GPA/EPO(strong-side mutant) fusion protein can specifically activate RBC formation without stimulation of blood clotting, provided that the fusion protein cannot mediate adhesion of cells bearing GPA (e.g., RBCs) and other cells bearing EPORs. There is also a correlation between stimulation of platelet production and stimulation of thrombosis, indicating that enhancement of platelet formation can be used as a surrogate marker for EPO-induced thrombosis. The mutations described above (e.g., R150A) affected the strong face of EPO and those fusion proteins are therefore expected to affect formation of both EPOR homodimers and EPOR-CD131 heterodimers. These engineered molecules stimulate RBC formation without activating thrombosis or tissue-protective activity. Described herein is a molecule that is able to stimulate both RBC production and tissue protection without stimulating platelet formation, a surrogate marker of the pro-thrombotic side effect of EPO. See e.g., International Patent Applications WO 2020/132234, WO/2017015141; Taylor et al., 2010, Protein Eng Des Sel, 23, 251-260; Burrill et al., 2016, Proc Natl Acad Sci USA, 113, 5245-5250; Lee et al., 2020, ACS Synth Biol, 9, 191-197; the contents of each of which are incorporated herein by reference in their entireties.

### Results

### Rational Design of EPO Fusion Proteins to Address Hypoxia

This work aims to improve the pharmacokinetics and therapeutic window of EPO, so to harness both its erythropoietic and tissue-protective effects while avoiding thrombosis. To achieve this goal, described herein are EPO fusion proteins (EPO-H; H for hypoxia) based on the concept of a ‘chimeric activator’.

An exemplary EPO-H comprises the nanobody element IH4, which binds to the target antigen GPA, and a mutated version of EPO, fused via a flexible five-amino acid linker (see e.g., FIG. 1F). A mutation in the EPO element can weaken its affinity to homodimeric EPOR, thereby avoiding undesired pro-thrombotic effects triggered by homodimeric EPOR signaling on non-target cells (see e.g., FIG. 1G). The desired erythropoietic activity is rescued by targeted EPO activity on RBC precursor cells directed by the binding of the antibody element, IH4, to the target antigen, GPA (see e.g., FIG. 1H). This way, EPO activates homodimeric EPORs only on RBC precursors, mitigating the unwanted thrombotic side effects via non-target cells.

At the same time, it is important to ensure that the same mutation in the EPO element does not disrupt EPO binding to heterodimers of EPOR and the co-receptor CD131 when the tissue-protective activity is desired (see e.g., FIG. 1I). Several EPO mutants were designed based on mutagenesis studies. The predicted behaviors of these EPO mutants, either alone or when fused to an anti-GPA antibody element, are outlined in Table 1. As part of the design strategy, “leaky mutations” are used that reduce but do not abolish binding; in practice these are mutations in which an amino acid with a long side chain is replaced by one with a shorter side chain, so that no steric hindrance results and binding is possible. As controls, “tight mutations” were also used in which a side chain is lengthened or the charge of a side chain is reversed, so that a binding activity would be completely lost due to the specific mutation. Because the binding mode of EPO to EPOR-CD131 is not elucidated, unlike that of EPO to homodimeric EPOR, several single point mutations were made in both of the known EPOR contact regions (strong and weak faces, each with K_{D} of 1 nM and 1 μM, respectively). Two residues on the strong face, K45 and R150, and five residues on the weak face, R14, Y15, R103, S104 and L108, were mutated and tested for targeted erythropoietic and tissue-protective activities (see e.g., Table 2 and FIG. 5A-5L).

EPO-H also has enhanced pharmacokinetics. Fusing mutated EPO to the IH4 nanobody not only increases the size of the molecule to avoid renal clearance, but it also directs the fusion protein to mature RBCs in circulation, further extending serum half-life (see e.g., FIG. 1J). See e.g., Burrill et al., 2016, supra; Kontos and Hubbell, Molecular pharmaceutics 7(6), 2141-2147, 2010; Kontos et al., PNAS 110(1), E60-E68, 2013; the contents of each of which are incorporated herein by reference in their entireties. Through these strategies, EPO-H fusion proteins were constructed that allow administration of high doses required for tissue protection but avoid thrombosis, thereby achieving prolonged activity in the body and reduced dosing frequency.

### Erythropoietic Activity of EPO Variants In Vitro

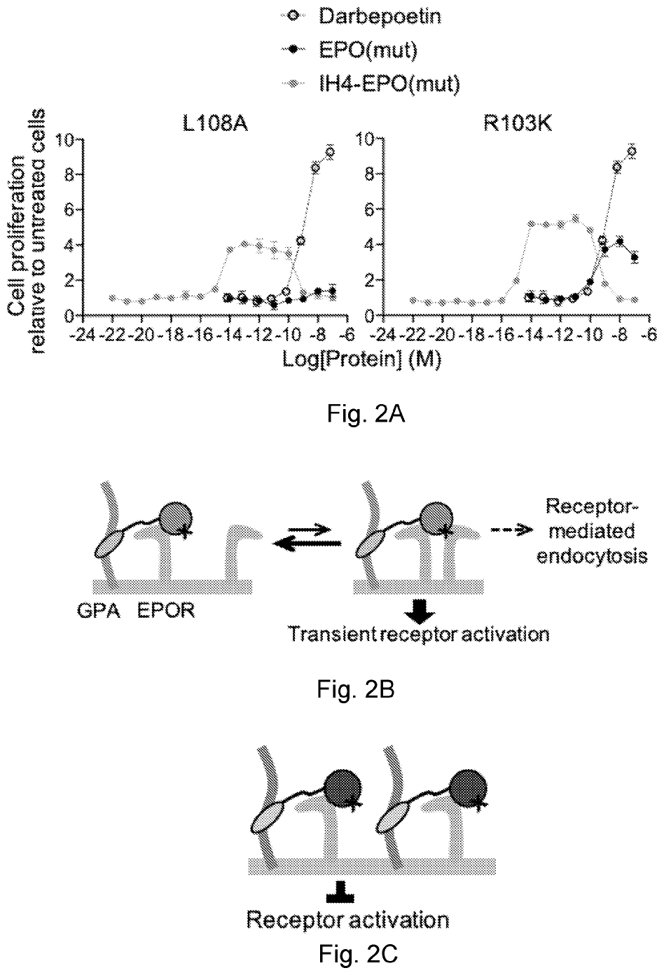

The ability of different EPO mutants to promote RBC production was tested in vitro via TF-1 cell proliferation assays. TF-1 is an immature erythroid cell line that expresses both EPOR and GPA (1620±140 and 3860±780 molecules per cell, respectively), and requires EPO, GM-CSF, or IL-3 for growth. TF-1 cells were starved of cytokines overnight and then exposed to EPO variants for 72 hr. Their proliferation was measured by a standard tetrazolium-based assay. Wild-type EPO (epoetin alfa) and hyperglycosylated EPO (darbepoetin) exhibited EC₅₀ values of ˜0.1 nM and ˜1 nM, respectively. EPO mutations on the strong binding face reduced activity of unfused EPO by ˜120- to 3400-fold relative to epoetin alfa. When these mutants were fused to IH4, their activities were rescued by ˜180-fold to 340-fold relative to unfused mutants, showing comparable activity to epoetin alfa and darbepoetin (see e.g., Table 1 and FIG. 5A-5L). The unfused EPOs with mutations on the weak face showed no activity at concentrations ranging from 10⁻¹⁴ to 10⁻⁷ M, except for EPO(R103K), which had a slightly lower EC₅₀ value compared to epoetin alfa and approximately two-fold lower efficacy (Eₘₐₓ) (see e.g., Table 2 and FIG. 5A-5L). When EPO(R103K) was fused to IH4, the fusion protein exhibited significantly enhanced activity, with its EC₅₀ value in a low femtomolar range. Among the weak-face mutants that completely lacked erythropoietic activity, only IH4-EPO(L108A) exhibited targeted erythropoietic activity, while the others remained inactive even after fusion. Similar to IH4-EPO(R103K), IH4-EPO(L108A) also had an EC₅₀ of ˜1 fM-10 fM (see e.g., Table 2 and FIG. 2A).

The dose-response curve of weak-face mutants fused to IH4 showed two unusual features. First, when EPO(L108A or R103K) was fused to IH4, the potency of the fusion protein was enhanced by four to five orders of magnitude relative to wild-type EPO and other EPO fusion proteins. The EC₅₀ is ˜1 fM-10 fM (see e.g., FIG. 2A). Secondly, the dose-response curve of IH4-EPO(L108A or R103K) was bell-shaped, with stimulation falling off at ˜1 nM, whereas fusion proteins containing strong-face mutants (K45D and R150A) showed standard sigmoidal dose-response curves (see e.g., FIG. 2A and FIG. 5A-5L). Without wishing to be bound by theory, it is hypothesized that these features result from distinct receptor binding properties of weak-face mutants. These mutations further reduce EPO-EPOR interaction at the weak face, resulting in an extremely rapid off-rate. At low concentrations of the fusion protein, the binding of EPOR to EPO's weak face, needed for the formation of a complete signaling complex, may be so transient that the fusion protein activates EPORs for cell proliferation but cannot stay long enough to be endocytosed. This has the net effect of increasing the frequency of EPOR activation with a limited amount of the fusion protein (see e.g., FIG. 2B). At high concentrations, the fusion protein saturates EPORs in a non-signaling, monomeric form via the strong side, and blocks receptor activation (see e.g., FIG. 2C).

### Targeted Erythropoietic Activity of EPO-H in Mice

One of the fusion proteins, IH4-EPO(L108A), was tested for targeted erythropoietic activity in transgenic mice expressing human GPA. IH4-EPO(L108A) was chosen because EPO(L108A) by itself showed essentially no homodimeric EPOR activation in TF-1 cell proliferation assay, indicating that potential pro-thrombotic side effects would be greatly reduced. Mice received a single intraperitoneal (i.p.) injection of saline, darbepoetin (50 pmol=2 μg; 12.5 pmol=0.5 μg) or IH4-EPO(L108A) (6 pmol=0.3 μg). Target cell specificity and drug efficacy were measured by staining for reticulocytes and reticulated platelets in blood samples on Days 0, 4 and 7 post-injection. Reticulocyte and reticulated platelet levels remained at baseline (Day 0) throughout the experiment in the saline-treated mice, but increased significantly in mice treated with darbepoetin, a control for the untargeted form of EPO. Mice treated with IH4-EPO(L108A) had elevated reticulocyte counts (8.47%) that were comparable to those in mice treated with 12.5 pmol of darbepoetin (8.17%) on Day 4 (see e.g., FIG. 3A), but did not have significantly increased reticulated platelet counts (see e.g., FIG. 3B). When various doses were tested (40 pmol=2 μg; 6 pmol=0.3 μg; 1.2 pmol=0.06 μg; 0.2 pmol=0.01 μg), IH4-EPO(L108A) induced reticulocyte responses in a dose-dependent manner: 40 pmol and 6 pmol resulted in 8.13% and 3.16% increases in reticulocyte counts on Day 4 relative to Day 0, respectively, while lower doses (1.2 pmol and 0.2 pmol) did not have significant effects (see e.g., FIG. 6A-6B).

### Tissue-Protective Activity of EPO Variants In Vitro

EPO mutants that displayed targeted erythropoietic activity were further evaluated to confirm their expected tissue-protective effects in cell-based assays. The ability of a fusion protein to protect cells was measured in vitro by estimating the number of surviving cells after treatment with EPO and a cobalt chloride (CoCl₂), which induces a hypoxia response via hypoxia-inducible factor-alpha (HIF-1α) due to its inhibition of prolyl hydroxylase. SH-SY5Y, a neuroblastoma cell line that expresses both EPOR and CD131, was co-treated with engineered EPO variants and 100 μM of CoCl₂. Viable cells were measured 24 hr later by standard tetrazolium dye-based assays. The optimal cell density and concentration of CoCl₂ were chosen to cause ˜30-40% cell viability in the absence of EPO.

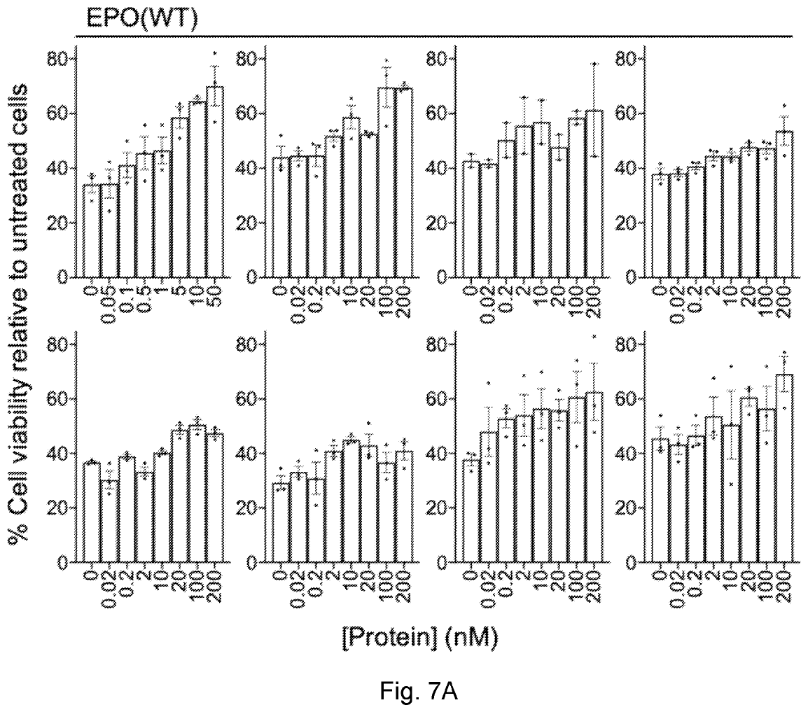

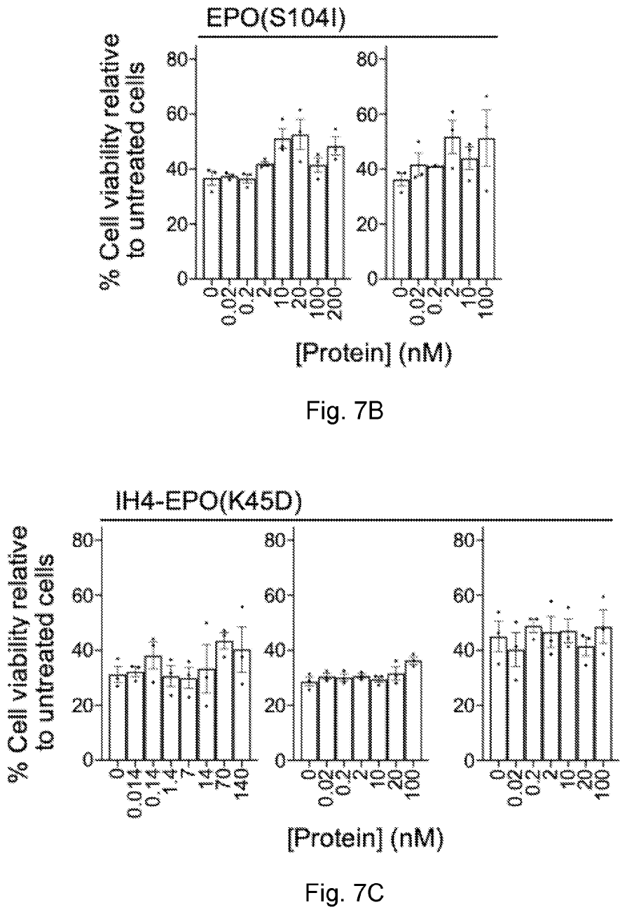

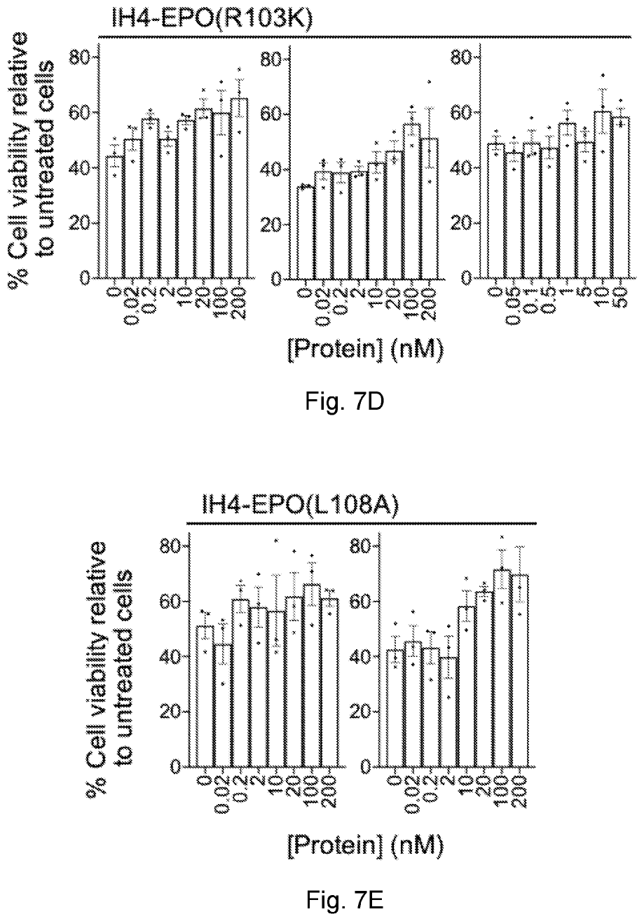

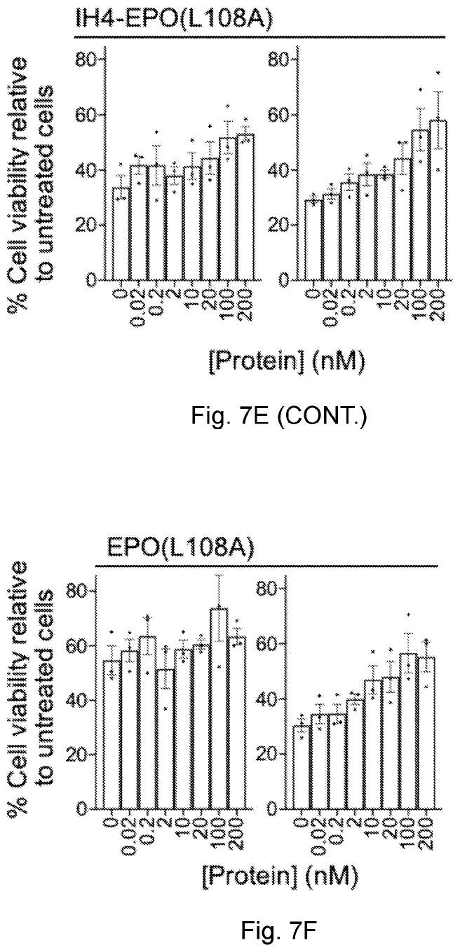

The control proteins, wild-type EPO (EPO(WT)) and EPO(S104I), protected neuroblastoma cells from CoCl₂ insult, although EPO(S104I) had a much weaker effect than the wild-type (see e.g., FIG. 4A, FIG. 4B and FIG. 7A-7B). This is consistent with results that EPO(WT) and EPO(S104I) protected primary neurons from N-methyl-D-aspartic acid (NMDA)-induced excitotoxicity; see e.g., Gan et al., 2012, supra. While a fusion protein containing a strong-face mutant, IH4-EPO(K45D), did not protect cells from CoCl₂-induced cell death (see e.g., FIG. 4C and FIG. 7C), fusion proteins containing a weak-face mutant, IH4-EPO(R103K) and IH4-EPO(L108A), exhibited neuroprotective effects (see e.g., FIG. 4D, FIG. 4E and, FIG. 7D-7E). Similarly, a weak-face mutant, EPO(L108A), also showed neuroprotective effect against CoCl₂-induced hypoxic damage in the absence of fusion to the IH4 nanobody (see e.g., FIG. 4F and FIG. 7F). Four-parameter fits provided rough estimates for the potency of each variant. The EC₅₀ values of EPO(WT) and EPO(S104I) were ˜1 nM-5 nM. The EC₅₀ values of IH4-EPO(R103K), IH4-EPO(L108A) and EPO(L108A) were estimated to be ˜10 nM-20 nM (see e.g., FIG. 4G).

These EPO variants showed reproducible effects when they were repeated several times and even when they were tested under different experimental conditions (see e.g., FIG. 7-9). When SH-SY5Y cells were pre-exposed to EPO variants 24 hr before receiving 100 μM of CoCl₂, EPO(L108A or R103K) in both unfused and fused forms protected cells from hypoxia-induced cell death (see e.g., FIG. 8A-8F).

### Discussion

Described herein are EPO fusion proteins that can provide both erythropoietic and tissue-protective effects without causing thrombotic side effects. This molecule is designed to prevent or treat hypoxia-mediated damage in patients suffering from illnesses, such as chronic obstructive pulmonary disease (COPD) and right-side heart failure, to prevent altitude sickness in military personnel acclimating to high altitude regions and possibly to enhance physical performance. It may also alleviate organ damage caused by hypoxia in patients at risk of requiring a ventilator, e.g., COVID-19 (coronavirus disease of 2019) patients.

However, achieving a safe and tissue-protective dose of EPO is a challenge: the maximum allowed dose in patients with chronic kidney disease is limited by its pro-thrombotic effects, but the dose of EPO required for tissue-protective effects is at least as high or higher than for erythropoiesis. If doses are limited to non-thrombotic “safe” levels, then EPO is likely to fail in clinical trials for tissue protection because such doses are below what is effective for tissue protection and not necessarily because the drug itself is not effective. The chimeric activator design described herein addressed two major challenges in using EPO activity for the treatment of hypoxia—retaining both the erythropoietic activity and tissue-protective functions of EPO while reducing or eliminating its pro-thrombotic activity.

Other EPO derivatives lack the desired features for treatment of hypoxia. Darbepoetin simply extends the plasma half-life but has the same activities as EPO itself. Carbamylated EPO and weak-face EPO mutants (such as EPO(S104I) retain neuroprotective activity but completely lack erythropoietic and pro-thrombotic activity. Targeted EPO molecules with strong-face mutations retain erythropoietic activity and are not pro-thrombotic, but are predicted to lack tissue-protective activity because they contain a mutation in the surface of EPO that strongly binds to EPOR; this surface is predicted to be critical for binding to EPOR-CD131 heterodimers that mediate tissue protection. See e.g., Egrie and Browne, 2001, Br J Cancer, 84 Suppl 1, 3-10; Egrie et al., 2003, Exp Hematol, 31, 290-299; Leist et al., 2004, supra; Gan et al., 2012, supra; Burrill et al., 2016, supra; Lee et al., 2020, supra; the contents of each of which are incorporated herein by reference in their entireties.

The EPO derivatives described herein therefore combine two features: (1) a mutation on the surface of EPO that interacts weakly with EPOR, since such mutant EPOs retain tissue-protective activity, and (2) an antibody-based GPA-binding element that rescues activity on RBC precursors. The EPO mutation is in the weak face instead of the strong face with respect to interaction with EPOR. Many mutations in the weak face (e.g., R14E, R14Q, R14N, Y151, R103I and R103Q) abolished erythropoietic activity in cell-based proliferation assays, and the mutation R103K caused only a slight reduction in this assay. In contrast, EPO containing the mutation L108A completely lacked in vitro erythropoietic activity, but this activity was rescued when the mutant protein was fused to the GPA-targeting nanobody IH4 (see e.g., Table 2, FIG. 2A and FIG. 5L). In addition, this fusion protein retained erythropoietic activity in vivo (see e.g., FIG. 3A-3B and FIG. 6A-6B), with a potency similar to other targeted EPOs and EPO itself. Finally, the IH4-EPO(L108A) fusion protein still showed neuroprotective activity in vitro in an assay in which neuroblastoma cells were treated with CoCl₂, which induces a hypoxia response (see e.g., FIG. 4A-4G, FIG. 7A-7F and FIG. 8A-8F). Thus, the fusion proteins described herein, e.g., IH4-EPO(L108A), can be used for the treatment of hypoxia.

In in vitro testing for erythropoietic activity, the IH4-EPO(L108A) and IH4-EPO(R103K) fusion proteins showed two unusual properties: (1) extreme potency, with activity detectable at ˜1 fM ˜10 fM concentrations, and (2) loss of activity at ˜1 nM concentration (see e.g., FIG. 2A). These observations were made based on TF-1 erythroleukemia cell proliferation assays, in which cells were stimulated by wild-type EPO and engineered proteins. These effects are likely not relevant in vivo, since IH4-EPO(L108A) stimulates erythropoiesis at doses similar to EPO itself and does not show signs of extreme potency or auto-inhibition (see e.g., FIG. 3A-3B and FIG. 6A-6C).

Without wishing to be bound by theory, two mechanisms could, in combination, explain the extremely high potency of IH4-EPO(L108A or R103K). First, the attachment of the fusion protein to GPA could prevent receptor-mediated endocytosis and degradation of the signaling protein. In differentiating erythroleukemic cells, GPA is attached to a stable actin cytoskeleton that may preclude internalization. In non-differentiating erythroleukemic cells, GPA is internalized by a clathrin-mediated pathway but at a much slower rate compared to other membrane proteins. Therefore, binding to GPA may interfere with internalization and degradation of the fusion protein. However, simple attachment of an EPO fusion protein to GPA does not profoundly enhance its potency, since highly potent activity is not observed with other anti-GPA/EPO fusion proteins. Second, the fusion protein may form a highly stable complex with GPA and one copy of EPOR via the strongly interacting side of EPO, but interaction with the second EPOR to form a complete signaling complex may be weak and dissociate rapidly due to a mutation. The interaction may last long enough to phosphorylate a subset of the tyrosine residues important in signal transduction into the nucleus but may not be long enough to phosphorylate residues involved in signaling to the clathrin system for receptor-mediated endocytosis (see e.g., FIG. 2B). In these assays, stimulation of proliferation is observed with as few as six to sixty molecules of fusion protein per cell, suggesting that this is the minimum number of molecules needed to promote erythropoietic signaling (see below for a quantitative explanation).

The loss of activity by IH4-EPO(L108A or R103K) at >1 nM concentrations can be explained by receptor saturation that has been observed in other systems that require more than two receptors for signaling. Similar to EPO, human growth hormone (hGH) asymmetrically binds to two hGH receptors to trigger signaling. Wild-type hGH inhibits signaling at >2 μM, and mutation of the weak-binding face of hGH further reduces the IC₅₀ value to ˜100 nM. It has been demonstrated that the antagonistic behaviors resulted from the disruption of receptor dimerization, using divalent monoclonal antibodies for hGH receptors. See e.g., Fuh et al., 1992, Science, 256, 1677-1680; the content of which is incorporated herein by reference in its entirety. Similarly, the fusion protein described herein containing a weak-face EPO mutation may saturate monomeric EPOR in a 1:1 stoichiometry and block the formation of a complete signaling complex consisting of homodimeric EPOR, resulting in auto-inhibition of EPO signaling (see e.g., FIG. 2C).

It is important to note that an enhanced potency of IH4-EPO(L108A) was observed in the cell based assay, in the in vivo erythropoiesis experiments the potency of this molecule was similar to that of darbepoetin and other EPO fusion proteins. In the in vitro assay, there may be essentially no removal of the fusion protein, while in vivo the normal clearance mechanisms would likely still operate, such as pinocytosis and degradation by Kupffer cells and/or binding to EPORs on non-erythroid cells and removal by non-signaling receptor-mediated endocytosis.

Taken together, these results indicate that IH4-EPO(L108A) can be used for treatment of hypoxia. The fusion protein is expected to enhance oxygen delivery and prevent hypoxia-induced cell death, without causing thrombosis. This work demonstrates that the engineering strategies allow for selective utilization of beneficial EPO activities and inhibition of undesired effects. More broadly, it further solidifies the value of the “chimeric activator” approach in designing targeted protein therapeutics.

### Methods

### Cell Culture

FREESTYLE 293-F and FREESTYLE CHO—S cell lines were obtained from INVITROGEN (Carlsbad, CA) and cultured in FREESTYLE 293 Expression Medium and complete FREESTYLE CHO Expression Medium (INVITROGEN), respectively. Human erythroleukemia TF-1 and human neuroblastoma SH-SY5Y were obtained by ATCC (Manassas, VA). TF-1 was cultured in RPMI-1640 with 10% FBS, 100 U/mL penicillin, 100 U/mL streptomycin, and 2 ng/mL recombinant human granulocyte macrophage colony-stimulating factor (GM-CSF; PEPROTECH) unless specified otherwise. SH-SY5Y was cultured in 1:1 DMEM/F-12 with 10% FBS. 293-F and CHO—S were cultured at 37° C. in 8% CO₂ with shaking at 2.35×g. TF-1 and SH-SY5Y were cultured at 37° C. in 5% Co₂.

### DNA Constructs

The DNA sequence for EPO wild-type was from GENBANK (accession no. KX026660, see e.g., SEQ ID NO: 1). EPO mutant sequences were constructed by introducing a codon change into the wild-type sequence. The DNA sequence for the IH4 nanobody was derived by reverse translating and codon optimizing (INTEGRATED DNA TECHNOLOGIES) the protein sequence adapted from U.S. Pat. No. 9,879,090. It was modified to include a point mutation (Phe80Tyr) in the framework region 3 to reflect the consensus of the germline sequences, and an additional amino acid (Thr118) in the framework region 4, as the reported sequence had a typographical error. See e.g., SEQ ID NOs: 2, 4, 38-39, 50 for individual sequences.

### Protein Expression and Purification

Transient expression was performed in 293-F and CHO—S cells using pSecTag2A or pOptiVEC plasmids according to the supplier's protocol. 4-6 days after transfection, protein expression was assayed by Western blotting cell supernatant using anti-6×His-HRP antibody (ABCAM). Proteins from transient transfection were purified as follows. Supernatant was concentrated to 5-8 mL using a 10 kDa cut-off MACROSEP ADVANCE centrifugal device (PALL). Concentrated protein was bound to 0.5-1 mL of His60 nickel or HisTalon cobalt resin (TAKARA BIO) for 0.5-1 hr at 4° C. while rotating in a 10-mL PIERCE disposable column (THERMO SCIENTIFIC), and was washed and eluted using His60 or HisTalon Buffer Set (TAKARA BIO) according to the supplier's protocol. Cell supernatant and each purification fraction were analyzed by SDS-PAGE followed by Coomassie Blue staining. Eluted proteins were combined, desalted into endotoxin-free PBS (TEKNOVA: 137 mM NaCl, 1.4 mM KH₂PO₄, 4.3 mM Na₂HPO₄, and 2.7 mM KCl, pH 7.4) using Econo-Pac 10DG columns (BIO-RAD), and concentrated to <1 mL using MACROSEP ADVANCE centrifugal device.

For in vivo experiments, contaminating proteins were further removed by anion-exchange chromatography (AIEX) on HiPrep DEAE FF 16/10, followed by size exclusion chromatography (SEC) on SUPERDEX 200 10/300 GL columns (CYTIVA), using AKTA FPLC system (CYTIVA). For AIEX, 1 M Tris-HCl, pH 8.0 was used as the starting buffer and a linear gradient up to 1 M NaCl was used for elution. For SEC, endotoxin-free PBS was used as the running buffer. Desired protein fractions were combined and concentrated to <1 mL using MACROSEP ADVANCE centrifugal device. Proteins were stored at 4° C. throughout the described process, ultimately stored as aliquots at −80° C., and thawed once before use. Only endotoxin-free reagents were used.

### TF-1Cell Proliferation Assays

TF-1 cells were seeded in a 96-well plate at 9.0×10³ cells per well in 90 μL of RPMI-1640 with serum and antibiotics (no GM-CSF). The purified proteins were serially diluted by 10-fold (10⁻⁷ to 10⁻¹¹ or 10⁻²¹ M) or 100-fold (10⁻⁷ to 10⁻²¹ M) and added to the cells. Cells were incubated at 37° C. in 5% CO₂ for 72 hr. Cell proliferation was determined by CellTiter 96® AQUEOUS ONE SOLUTION CELL PROLIFERATION ASSAY (PROMEGA) or adding 10 μL of WST-1 reagent (ROCHE). 2-4 hr after adding the reagent, absorbance at 490 nm (and background absorbance at 650 nm when using WST-1) was read on a BIOTEK SYNERGY NEO HTS microplate reader. Reported data represent mean f SEM of three replicates.

### Measuring Mouse Reticulocytes and Reticulated Platelets

Human GPA-transgenic FVB mice were used. This strain underwent embryo re-derivation. The homozygous human GPA transgene is embryonic-lethal but heterozygotes are phenotypically normal, so a breeding colony was maintained with screening for human GPA at each generation. Transgene expression was measured as described before (see e.g., Burrill et al., 2016, supra).

Five mice per dose group received a single intraperitoneal (i.p.) injection with saline, darbepoetin or EPO fusion protein in a 200 μL volume (diluted in saline or PBS) on Day 0.1 μL-5 μL of whole blood was collected by tail-nick in EDTA-coated tubes on Days 0, 4 and 7 post-injection. Blood was analyzed immediately after collection by flow cytometry as described before (see e.g., Burrill et al., 2016, supra). Briefly, thiazole orange (SIGMA-ALDRICH) was used to stain residual RNA in reticulocytes and reticulated platelets, and anti-CD41-PE antibody (BD PHARMINGEN) was used to stain total platelets. A stock solution (1 mg/mL) of thiazole orange was prepared in 100% methanol and was diluted 1:5,000 in PBS to make a 2× working solution. Anti-CD41-PE antibody was diluted 1:500 in either the 2× working solution of thiazole orange for stained samples or PBS for gating thiazole orange-negative population. Whole blood was diluted 1:1,000 in PBS. Equal volumes (e.g., 100 μL) of 2× working solution of anti-CD41-PE antibody with or without thiazole orange and diluted whole blood were mixed in a 96-well U-bottom plate and incubated for 30 min in the dark at 23° C. The fluorescence was measured on a LSRFORTESSA SORP flow cytometer equipped with an optional HTS sampler (BD BIOSCIENCES) using the following filter configuration: PE excitation, 561/50 mW; emission filter, BP 582/15; YFP excitation, 488/100 mW; emission filter, BP 540/25.

### Tissue Protection Assay

SH-SY5Y cells were seeded in a 96-well plate at 4.8×10⁴ cells per well in 80 μL of 1:1 DMEM/F-12 with 10% FBS, and allowed to adhere overnight at 37° C. in 5% CO₂. In co-treatment experiments, cells received varying concentrations of purified proteins (0.02 nM to 200 nM) and 100 μM of cobalt chloride (CoCl₂), a hypoxia mimicking agent, and were incubated at 37° C. in 5% CO₂ for 24 hr. In pre-treatment experiments (see e.g., FIG. 8A-8F), cells were treated with purified proteins 24 hr before receiving CoCl₂ and were incubated at 37° C. in 5% CO₂ for additional 24 hr after adding CoCl₂. Cell viability was measured by CELLTITER 96® AQ_{UEOUS} ONE SOLUTION CELL PROLIFERATION ASSAY (PROMEGA). 2-4 hr after adding the reagent, absorbance at 490 nm was read on a BIOTEK SYNERGY NEO HTS microplate reader. In experiments shown in FIG. 9A-9B, SH-SY5Y cells were seeded in a 96-well plate at 1.2×10⁴ cells per well in 80 μL. On the next day, cells were co-treated with varying concentrations of purified proteins (0.02 nM to 200 nM) and 25 μM of CoCl₂ or 50 μM of CoCl₂, and incubated at 37° C. in 5% CO₂ for 72 hr. Cell viability was measured by adding 10 μL of WST-1 reagent (ROCHE). 4 hr after adding the reagent, absorbance at 490 nm and 650 nm (background) was read on a BIOTEK SYNERGY NEO HTS microplate reader. Reported data represent mean±S.E.M of two to four replicates.

### Quantitative Explanation of Extreme Potency of IH4-EPO(L108A or R103K)

Without wishing to be bound by theory, it is hypothesized that the extremely potent signaling of IH4-EPO(L108A or R103K) (see e.g., Table 2 and FIG. 2A) can be explained by a lack of receptor-mediated endocytosis, such that signaling is not terminated after receptor activation (see e.g., FIG. 2B). Some receptor tyrosine kinase systems involve a rapid phosphorylation event(s) that initiates signaling that results in transcriptional modulation, followed by slower phosphorylation events that lead to receptor-mediated endocytosis and degradation of the receptor and/or ligand. According to this hypothesis, mutation of the weak face of EPO could further reduce the stability of the EPO-(EPOR)₂ complex, such that this complex is rapidly forming but also dissociates so rapidly that endocytosis-stimulating phosphorylation does not occur.

Despite the extreme potency of IH4-EPO(L108A) in vitro, this fusion protein does not have enhanced potency in vivo, and behaves similarly to our other targeted EPOs with respect to RBC production and lack of platelet production (see e.g., FIG. 3A-3B). In a culture dish, receptor-mediated endocytosis by a single cell type is the only mechanism to terminate signaling, but in vivo the fusion protein may disappear through renal clearance, bulk fluid pinocytosis, and by binding to EPOR on non-hematopoietic cells, followed by non-signaling endocytosis and protein degradation that is part of normal membrane turnover. Thus, IH4-EPO(L108A) is expected to have a clearance rate and potency that would be compatible with its use as a treatment for hypoxia and related disorders.

The following calculations provide quantitative explanations for this hypothesis. Kinetics parameters relevant to these calculations can be found in Table 3 below.

### 1. Minimum Number of Fusion Protein Molecules Needed for Signaling

The extreme potency of IH4-EPO(L108A or R103K) allows an estimate of about 6 to 60 fusion protein molecules per cell are required for EPO-induced signaling in TF-1 cells. At the EC₅₀ of ˜1 fM-10 fM for stimulation of TF-1 cell proliferation, there are about 6 to 60 molecules of the fusion protein per cell at the start of the assay. Specifically, there are about 9,000 cells and 600,000 fusion protein molecules per 100 μL in a well at the start of the proliferation assay. This provides an estimate for the minimum number of receptor-ligand complexes required to trigger EPO-induced signaling in TF-1 cells.

2. Slow Dissociation of a Fusion Protein from GPA and EPOR

When IH4-EPO(L108A or R103K) binds to the surface of a TF-1 cell, binding is stabilized through simultaneous interaction with GPA (K_{D}=33 nM) and EPOR via the strong-binding face of EPO (K_{D}=0.1 nM-1 nM). In this state, the local concentration of the bound fusion protein can be estimated by the number of receptor-bound fusion protein molecules and effective volume occupied by these molecules around the cell surface. The effective volume is approximated as 1 μm³ by multiplying the cell surface area (1000 μm²) by the distance from the cell surface in which the fusion protein is trapped (about 1 nm), so one molecule has a concentration of about 1.66×10⁻⁹ M. This means that the local concentration of GPA on the surface of a TF-1 cell is about (1.6×10⁻⁹ M)×(3860 GPA molecules)=6.2×10⁻⁶ M. Given the K_{D} value of IH4 to GPA is 33 nM, about 99.5% of GPA molecules will be also bound by fusion proteins via IH4, when they are bound to EPOR via the strong face of the EPO element. Due to this avidity effect, the effective dissociation half-time may be increased by ˜200-fold. Based on these calculations, the effective dissociation half-time of the fusion protein from both EPOR and GPA is estimated to be ˜100 hr, which is longer than the length of the experiment (72 hr). Non-signaling membrane proteins are normally endocytosed more slowly and recycling more efficiently, such that they have a metabolic turnover in the range of 12 hours or more. Moreover, it is possible that GPA is anchored to the cytoskeleton in a way that slows or prevents this process even further. Thus, in proliferation assay wells with EPO in concentrations 1 fM to 100 fM, EPO is in molar excess relative to EPO receptors, essentially all of the EPO is bound to at least one receptor, and turnover of EPO is likely to be slow enough that much of it survives the 72-hour incubation of the assay.

### 3. Rapid Association and Dissociation Between a Second EPOR and the GPA-EPOR-Fusion Protein Complex

In the configuration where the fusion protein is bound to GPA and an EPOR, binding to a second EPOR would occur rapidly because the EPO element is positioned at the correct height from the membrane and in the correct orientation for such binding, and the binding would rely predominantly on the two-dimensional diffusion within the membrane. The on-rate of a fusion protein already bound to GPA and an EPOR for a second EPOR is assumed to be high because the effective molarity of the cell-bound fusion protein is high, and because the EPO element is rotationally constrained to place its weak EPOR-binding face in the correct orientation relative to the second EPOR.

However, the mutation (e.g. L-A) on the weak face of EPO likely allows for rapid dissociation of this second EPOR. The interaction with EPOR of wild-type EPO through its weak face is estimated to have a K_{D} of about 2 μM (for the soluble interaction). Assuming that the diffusion-limited kₒₙ EPO to EPOR via the weak face is the same as for the strong-face interaction (kₒₙ=8.3×10⁶ M⁻¹ s⁻¹), the dissociation rate constant (k_{off}) would be about 18 s⁻¹, such that the complex dissociates in <0.1 second in the absence of other interactions, such as the EPOR Box1-Box2/JAK2 FERM dimerization.

### Tables

TABLE 1
Properties of Wild-Type EPO and Engineered EPO-H Variants
Table 1 depicts predicted properties of wild type EPO and engineered EPO-H variants.
Predicted properties: RBC production, thrombosis, tissue protection, and expected half-life.

| | | | RBC | | Tissue | Expected |
|---|---|---|---|---|---|---|
| | Mutation | Protein | production | Thrombosis | protection | Half-life |
| | Wild type | EPO | + | + | + | Short |
| Strong- | R150A | EPO(R150A) | − | − | − | Short |
| face | (Leaky) | IH4- | + | − | − | Extended |
| mutation | | EPO(R150A) | | | | |
| | K45D | EPO(K45D) | − | − | − | Short |
| | (Tight) | IH4- | − | − | − | Extended |
| | | EPO(K45D) | | | | |
| Weak- | L108A | EPO(L108A) | − | − | + | Short |
| face | (Leaky) | IH4- | + | − | + | Extended |
| mutation | | EPO(L108A) | | | | |
| | S104I | EPO(S104I) | − | − | + | Short |
| | (Tight) | IH4- | − | − | + | Extended |
| | | EPO(S104I) | | | | |

“+” = increase, “−” = decrease or no effect.

Table 2 shows the in vitro stimulation of TF-1 cell proliferation by EPO mutants and their fusion to IH4. N indicates the number of repeat experiments, each containing three replicates. N.D.=Not determined. N.A.=Not active.

TABLE 2
In vitro stimulation of TF-1 cell proliferation

| | | EPO | | | IH4-EPO | | |
|---|---|---|---|---|---|---|---|
| | | | Log(EC₅₀) (M) ± | EC₅₀ relative | | Log(EC₅₀) (M) ± | EC₅₀ relative |
| | Protein | N | S.D. | to epoetin alfa | N | S.D. | to epoetin alfa |
| Control | Epoetin alfa | 21 | −10.22 ± 0.32 | 1 | 0 | N.D. | N.D. |
| | (Epogen ®) | | | | | | |
| | Darbepoetin | 10 | −9.06 ± 0.27 | 14.20 | 0 | N.D. | N.D. |
| | (Aranesp ®) | | | | | | |
| Strong | K45D | 4 | −6.68 ± 0.65 | 3425.96 | 2 | −9.21 ± 0.49 | 10.04 |
| Face | R150A | 8 | −8.14 ± 0.06 | 119.32 | 9 | −10.41 ± 0.29 | 0.64 |
| Weak | S104I | 2 | N.A. | N.A. | 2 | N.D. | N.D. |
| Face | R14E | 1 | N.A. | N.A. | 3 | N.A. | N.A. |
| | R14Q | 1 | N.A. | N.A. | 2 | N.A. | N.A. |
| | R14N | 1 | N.A. | N.A. | 2 | N.A. | N.A. |
| | Y15I | 2 | N.A. | N.A. | 2 | N.A. | N.A. |
| | R103I | 1 | N.A. | N.A. | 3 | N.A. | N.A. |
| | R103Q | 2 | N.A. | N.A. | 3 | N.A. | N.A. |
| | R103K | 6 | −9.93 ± 0.40 | 1.94 | 8 | −13.16 ± 1.74 | 1.14 × 10⁻³ |
| | L108A | 5 | N.A. | N.A. | 5 | −13.74 ± 1.54 | 2.99 × 10⁻⁴ |

Table 3 shows relevant binding parameters. “*” indicates that for comparison, the internalization rate constant (kᵢₙₜ) of EPO(WI) is ˜1.0×10⁻³ s⁻¹, giving an internalization half-time of ˜11.5 min for receptor-mediated endocytosis. “**” indicates that the K_{D} of the weak-face EPO L108A mutant protein to EPOR via the weak side interaction is estimated to be about 10-fold weaker than for a wild-type weak-face interaction. This is based on cell-based assay results and typical effects of a leucine-to-alanine mutation that removes a protein-protein interaction contact without otherwise affecting protein structure. “#” indicates that the diffusion-limited kₒₙ of a weak-face EPO mutant to EPOR is assumed to Wythe same as the strong-side interaction. “##” indicates that the diffusion-limited kₒₙ of a weak-face EPO mutant to EPOR is assumed to stay the same as the strong-side interaction.

TABLE 3
Relevant Binding Parameters

| Receptor | Ligand | K_{D} (M) | kₒₙ (M⁻¹s⁻¹) | k_{off} (s⁻¹) | T_{1/2}* | |
|---|---|---|---|---|---|---|
| GPA | IH4 | 3.37 × 10⁻⁸ | 5.73 × 10⁵ | 1.9 × 10⁻² | 0.6 | min |
| Soluble EPOR | EPO(WT) | 2.0 × 10⁻¹⁰ | — | — | — | |
| | | (Strong face) | | | | |
| | | 2.1 × 10⁻⁶ | — | — | — | |
| | | (Weak face) | | | | |
| EPOR on cells | EPO(WT) | 6.0 × 10⁻¹¹ | 8.33 × 10⁶ | 5.0 × 10⁻⁴ | 23 | min |
| EPOR-Fc | EPO(WT) | 5.4 × 10⁻⁹ | 3.9 × 10⁴ | 2.1 × 10⁻⁴ | 55 | min |
| Soluble EPOR | EPO(WT) | 2.1 × 10⁻⁶ | ~10⁶ | ~2.1 × 10⁻⁰ | 0.5 | sec |
| | | (Weak face) | | | | |
| EPOR on cells | EPO(WT) | 6.0 × 10⁻⁷ | ~8.33 × 10⁶ | ~5 × 10⁰ | 0.2 | sec |
| | | (Weak face) | | | | |
| EPOR-Fc | EPO(WT) | 5.4 × 10⁻⁵ | ~3.9 × 10⁴ | ~2.1 × 10⁰ | 0.5 | sec |
| EPOR (via weak | Weak-face | **6.0 × 10⁻⁶ | ^{#}8.33 × 10⁶ | ~5 × 10¹ | 0.02 | s |
| face) | EPO mutant | (Weak face) | | | | |
| EPOR (via weak | Weak-face | **5.4 × 10⁻⁴ | ^{##}3.9 × 10⁴ | ~2.1 × 10¹ | 0.05 | s |
| face) | EPO mutant | (Weak face) | | | | |

TABLE 4
DNA and Protein Sequences. For exemplary full-length fusion
proteins see SEQ ID NOs: 11-12, 144, or 148.
Exemplary full-length fusion proteins include the collective
sequence, from N to C terminus order, of SEQ ID NO: 2,
SEQ ID NO: 3, and one of SEQ ID NOs: 1 or 13-23.
Exemplary nucleic acids encoding full-length fusion proteins
include SEQ ID NO: 142, SEQ ID NO: 146, SEQ ID NO: 149, or
the collective sequence, from 5′ to 3′ order, of SEQ ID NO:
4, SEQ ID NO: 5, and one of SEQ ID NOs: 6 or 24-34.

| | Protein sequence | DNA sequence |
|---|---|---|
| IH4 | QVQLQESGGGSVQ | CAGGTCCAACTGCAAGAGAGCGGCGGGGGGTCAGTTCAGGC |
| | AGGSLRLSCVASG | GGGGGGGAGTCTGCGGTTGAGCTGCGTAGCTTCAGGCTACAC |
| | YTDSTYCVGWFRQ | TGACAGCACCTACTGCGTGGGATGGTTTCGGCAGGCACCCGG |
| | APGKEREGVARINT | CAAGGAACGAGAGGGCGTTGCACGGATCAACACTATCTCCG |
| | ISGRPWYADSVKG | GTCGGCCTTGGTACGCAGATAGTGTTAAGGGACGGTTTACTA |
| | RFTISQDNSKNTVY | TTAGTCAGGATAACTCTAAGAATACCGTCTACCTTCAGATGA |
| | LQMNSLKPEDTAIY | ATAGCCTGAAACCGGAAGACACGGCTATTTACTATTGCACCC |
| | YCTLTTANSRGFCS | TTACAACTGCCAACAGCAGAGGGTTTTGTTCTGGGGGATATA |
| | GGYNYKGQGTQVT | ACTACAAAGGACAGGGGACCCAAGTCACTGTCAGC (SEQ ID |
| | VS (SEQ ID NO: 2) | NO: 4) |
| 5 AA linker | SGGGS (SEQ ID | TCTGGTGGTGGTTCC (SEQ ID NO: 5) |
| | NO: 3) | |
| EPO(WT) | APPRLICDSRVLER | GCCCCACCTAGATTGATTTGTGATTCCAGAGTTTTGGAAAGA |
| | YLLEAKEAENITTG | TACTTGTTGGAAGCTAAGGAGGCTGAAAATATTACTACTGGT |
| | CAEHCSLNENITVP | TGTGCTGAACATTGTTCTTTGAACGAGAATATTACTGTTCCAG |
| | DTKVNFYAWKRM | ATACTAAGGTTAACTTTTACGCTTGGAAGAGAATGGAAGTTG |
| | EVGQQAVEVWQG | GTCAGCAAGCTGTTGAAGTTTGGCAAGGTTTGGCTTTGTTGTC |
| | LALLSEAVLRGQAL | TGAAGCTGTTTTGAGAGGTCAAGCTTTGTTGGTTAATTCTTCT |
| | LVNSSQPWEPLQLH | CAACCATGGGAACCATTGCAATTGCATGTTGATAAGGCTGTT |
| | VDKAVSGLRSLTTL | TCTGGTTTGAGATCTTTGACTACCTTGTTGAGAGCTTTGGGTG |
| | LRALGAQKEAISPP | CTCAAAAGGAAGCTATTTCTCCTCCAGATGCTGCTTCTGCCGC |
| | DAASAAPLRTITAD | TCCATTGAGAACTATTACTGCTGATACTTTTAGAAAGTTGTTT |
| | TFRKLFRVYSNFLR | AGAGTTTACTCTAACTTCTTGAGAGGTAAGTTGAAGTTGTAC |
| | GKLKLYTGEACRT | ACTGGTGAAGCTTGTAGAACTGGTGATCGG (SEQ ID NO: 6; |
| | GDR (SEQ ID NO: 1) | see also SEQ ID NO: 43) |
| IH4-5 AA | QVQLQESGGGSVQ | CAGGTCCAACTGCAAGAGAGCGGCGGGGGGTCAGTTCAGGC |
| linker- | AGGSLRLSCVASG | GGGGGGGAGTCTGCGGTTGAGCTGCGTAGCTTCAGGCTACAC |
| EPO(WT) | YTDSTYCVGWFRQ | TGACAGCACCTACTGCGTGGGATGGTTTCGGCAGGCACCCGG |
| | APGKEREGVARINT | CAAGGAACGAGAGGGCGTTGCACGGATCAACACTATCTCCG |
| | ISGRPWYADSVKG | GTCGGCCTTGGTACGCAGATAGTGTTAAGGGACGGTTTACTA |
| | RFTISQDNSKNTVY | TTAGTCAGGATAACTCTAAGAATACCGTCTACCTTCAGATGA |
| | LQMNSLKPEDTAIY | ATAGCCTGAAACCGGAAGACACGGCTATTTACTATTGCACCC |
| | YCTLTTANSRGFCS | TTACAACTGCCAACAGCAGAGGGTTTTGTTCTGGGGGATATA |
| | GGYNYKGQGTQVT | ACTACAAAGGACAGGGGACCCAAGTCACTGTCAGC |
| | VS SGGGS | TCTGGTGGTGGTTCC |
| | APPRLICDSRVLER | GCCCCACCTAGATTGATTTGTGATTCCAGAGTTTTGGAAAGA |
| | YLLEAKEAENITTG | TACTTGTTGGAAGCTAAGGAGGCTGAAAATATTACTACTGGT |
| | CAEHCSLNENITVP | TGTGCTGAACATTGTTCTTTGAACGAGAATATTACTGTTCCAG |
| | DTKVNFYAWKRM | ATACTAAGGTTAACTTTTACGCTTGGAAGAGAATGGAAGTTG |
| | EVGQQAVEVWQG | GTCAGCAAGCTGTTGAAGTTTGGCAAGGTTTGGCTTTGTTGTC |
| | LALLSEAVLRGQAL | TGAAGCTGTTTTGAGAGGTCAAGCTTTGTTGGTTAATTCTTCT |
| | LVNSSQPWEPLQLH | CAACCATGGGAACCATTGCAATTGCATGTTGATAAGGCTGTT |
| | VDKAVSGLRSLTTL | TCTGGTTTGAGATCTTTGACTACCTTGTTGAGAGCTTTGGGTG |
| | LRALGAQKEAISPP | CTCAAAAGGAAGCTATTTCTCCTCCAGATGCTGCTTCTGCCGC |
| | DAASAAPLRTITAD | TCCATTGAGAACTATTACTGCTGATACTTTTAGAAAGTTGTTT |
| | TFRKLFRVYSNFLR | AGAGTTTACTCTAACTTCTTGAGAGGTAAGTTGAAGTTGTAC |
| | GKLKLYTGEACRT | ACTGGTGAAGCTTGTAGAACTGGTGATCGG (SEQ ID NO: 12) |
| | GDR (SEQ ID NO: | |
| | 11) | |
| IH4-(5AA)- | QVQLQESGGGSVQ | CAGGTCCAACTGCAAGAGAGCGGCGGGGGGTCAGTTCAGGC |
| EPO | AGGSLRLSCVASG | GGGGGGGAGTCTGCGGTTGAGCTGCGTAGCTTCAGGCTACAC |
| (L108A) | YTDSTYCVGWFRQ | TGACAGCACCTACTGCGTGGGATGGTTTCGGCAGGCACCCGG |
| | APGKEREGVARINT | CAAGGAACGAGAGGGCGTTGCACGGATCAACACTATCTCCG |
| | ISGRPWYADSVKG | GTCGGCCTTGGTACGCAGATAGTGTTAAGGGACGGTTTACTA |
| | RFTISQDNSKNTVY | TTAGTCAGGATAACTCTAAGAATACCGTCTACCTTCAGATGA |
| | LQMNSLKPEDTAIY | ATAGCCTGAAACCGGAAGACACGGCTATTTACTATTGCACCC |
| | YCTLTTANSRGFCS | TTACAACTGCCAACAGCAGAGGGTTTTGTTCTGGGGGATATA |
| | GGYNYKGQGTQVT | ACTACAAAGGACAGGGGACCCAAGTCACTGTCAGC |
| | VS SGGGS | TCTGGTGGTGGTTCC |
| | APPRLICDSRVLER | GCCCCACCTAGATTGATTTGTGATTCCAGAGTTTTGGAAAGA |
| | YLLEAKEAENITTG | TACTTGTTGGAAGCTAAGGAGGCTGAAAATATTACTACTGGT |
| | CAEHCSLNENITVP | TGTGCTGAACATTGTTCTTTGAACGAGAATATTACTGTTCCAG |
| | DTKVNFYAWKRM | ATACTAAGGTTAACTTTTACGCTTGGAAGAGAATGGAAGTTG |
| | EVGQQAVEVWQG | GTCAGCAAGCTGTTGAAGTTTGGCAAGGTTTGGCTTTGTTGTC |
| | LALLSEAVLRGQAL | TGAAGCTGTTTTGAGAGGTCAAGCTTTGTTGGTTAATTCTTCT |
| | LVNSSQPWEPLQLH | CAACCATGGGAACCATTGCAATTGCATGTTGATAAGGCTGTT |
| | VDKAVSGLRSLTTa | TCTGGTTTGAGATCTTTGACTACCgcaTTGAGAGCTTTGGGTGC |
| | LRALGAQKEAISPP | TCAAAAGGAAGCTATTTCTCCTCCAGATGCTGCTTCTGCCGCT |
| | DAASAAPLRTITAD | CCATTGAGAACTATTACTGCTGATACTTTTAGAAAGTTGTTTA |
| | TFRKLFRVYSNFLR | GAGTTTACTCTAACTTCTTGAGAGGTAAGTTGAAGTTGTACA |
| | GKLKLYTGEACRT | CTGGTGAAGCTTGTAGAACTGGTGATCGG (SEQ ID NO: 149) |
| | GDR (SEQ ID NO: | |
| | 148) | |

TABLE 5
EPO Mutations (e.g., compare to WT EPO, SEQ ID NOs: 1, 6, or 43)

| Relative to WT | | SEQ ID NO for | | SEQ ID NO for |
|---|---|---|---|---|
| EPO | Amino acid | mutant EPO | Exemplary codon | mutant EPO |
| (SEQ ID NO: 1) | change | protein | change | DNA |
| K45D (strong) | Lys → Asp | 13 | AAG → GAT | 24 or 45 |
| R150A (strong) | Arg → Ala | 14 | AGA → GCC | 25 or 44 |
| R14E (weak) | Arg → Glu | 15 | AGA → GAA | 26 |
| R14Q (weak) | Arg → Gln | 16 | AGA → CAA | 27 |
| R14N (weak) | Arg → Asn | 17 | AGA → AAC | 28 |
| Y15I (weak) | Tyr → Ile | 18 | TAC → ATC | 29 |
| R103I (weak) | Arg → Ile | 19 | AGA → ATC | 30 |
| R103Q (weak) | Arg → Gln | 20 | AGA → CAG | 31 |
| R103K (weak) | Arg → Lys | 21 | AGA → AAA | 32 |
| S104I (weak) | Ser → Ile | 22 | TCT → ATC | 33 |
| L108A (weak) | Leu → Ala | 23 | TTG → GCG | 34 |

TABLE 6
A list of weak-side mutants; the analysis described herein
focused on four mutants: R14N, R103K, R103I, L108A.

| | In vitro TF-1 proliferation | In vitro |
|---|---|---|
| EPO mutation | (RBC production) | neuroprotection |
| R14E | No | Not tested |
| R14Q | No | Not tested |
| R14N* | No | Yes |
| Y15I | No | Yes |
| R103K* | GPA-dependent | Yes |
| R103Q | No | No |
| R103I* | No | Yes |
| S104I | No (Fusion not tested) | Yes |
| L108A* | GPA-dependent | Yes |

TABLE 7
Summary of epitopes and binding kinetics of anti-GPA antibody
fragments and EPO (e.g., wild type and R150A mutant).

| Protein | kₒₙ (M⁻¹s⁻¹) | K_{off} (s⁻¹) | K_{D} (nM) | Antibody form | GPA epitope |
|---|---|---|---|---|---|
| 10F7 | — | — | 95 | Fab | ₃₄YAATP₃₈ (SEQ ID NO: 35) |
| 1C3 | — | — | 230 | Fab | — |
| | — | — | 62 | scFv | — |
| R18 | — | — | 52 | IgG | ₄₉RTVY₅₂ (SEQ ID NO: 36) |
| | — | — | 400 | Fab | |
| IH4 | 5.73 × 10⁵ | — | 33.72 | Nanobody (VHH) | ₅₂YPPE₅₅ (SEQ ID NO: 37) |
| EPO(WT) | 3.9 × 10⁴ | 2.1 × 10⁻⁴ | 5.4 | N/A | N/A |
| EPO(R150A) | 4.2 × 10⁴ | 3.4 × 10⁻³ | 81 | N/A | N/A |

TABLE 8
A list of DNA sequences encoding exemplary anti-GPA antibody fragments,
linkers, and EPO.

| Name | Sequence | SEQ ID NO |
|---|---|---|
| IH4 | CAGGTCCAACTGCAGGAGAGCGGGGGGGGTCAGTTCAGGCGGGG | 38; italicized text |
| nanobody | GGGAGTCTGCGGTTGAGCTGCGTAGCTTCAGGCTACACTGACAGCA | indicates the |
| | CCTACTGCGTGGGATGGTTTCGGCAGGCACCCGGCAAGGAACGAGA | variable region of |
| | GGGCGTTGCACGGATCAACACTATCTCCGGTCGGCCTTGGTACGCA | the heavy chain; |
| | GATAGTGTTAAGGGACGGTTTACTATTAGTCAGGATAACTCTAAGAAT | bold double- |
| | ACCGTC [symbol] CTTCAGATGAATAGCCTGAAACCGGAAGACACGGCTAT | underlined text |
| | TTACTATTGCACCCTTACAACTGCCAACAGCAGAGGGTTTTGTTCTGG | indicates a |
| | GGGATATAACTACAAAGGACAGGGG [symbol] CAAGTCACTGTCAGC | Phe80Tyr point |
| | | mutation and an |
| | | additional amino |
| | | acid Thr118 |
| IH4* | CAGGTCCAACTGCAAGAGAGCGGAGGAGGGTCTGTTCAAGCTGGCG | 39; italicized text |
| nanobody | GTTCCCTCCGGCTTTCTTGCGTGGCGTCAGGCTATACTGACAGCACA | indicates the |
| | TACTGCGTGGGCTGGTTCAGGCAGGCCCCTGGAAAGGAGCGCGAG | variable region of |
| | GGCGTAGCCCGCATAAATACTATATCTGGCAGACCGTGGTACGCTGA | the heavy chain; |
| | CAGCGTGAAGGGACGGTTTACAATCAGTCAAGATAACTCTAAAAACA | bold double- |
| | CCGTG [symbol] CTTCAAATGAATTCTTTGAAACCCGAAGATACTGCCATCT | underlined text |
| | ATTATTGCACACTTACGACCGCGAACTCACGCGGTTTTTGTAGCGGA | indicates Phe80 |
| | GGATATAACTATAAAGGGCAAGGGCAGGTAACTGTATCC | |
| 10F7 scFv | CAAGTTAAGTTGCAACAATCTGGTGCTGAATTGGTTAAGCCAGGTGC | 40; see e.g., |
| | TTCTGTTAAGTTGTCTTGTAAGGCTTCTGGTTACACCTTCAACTCTTAC | GenBank |
| | TTTATGCATTGGATGAAGCAAAGACCAGTTCAAGGTTTGGAATGGATT | accession |
| | GGTATGATTAGACCAAACGGTGGTACTACCGATTACAACGAGAAGTT | no. |
| | TAAGAACAAGGCTACTTTGACTGTTGATAAGTCCTCTAACACTGCTTA | KX026660-3; |
| | CATGCAATTGAACTCTTTGACTTCTGGTGATTCTGCTGTTTACTACTGT | italicized text |
| | GCTAGATGGGAAGGTTCTTACTACGCTTTGGATTACTGGGGTCAAGG | indicates the |
| | TACCACTGTTACTGTTTCTTCCGGTGGAGGTGGATCTGGTGGTG | variable region of |
| | GAGGATCTTCAGGAGGTGGTGGATCTTCCGATATTGAGTTGA | the heavy chain; |
| | CTCAATCTCCAGCTATTATGTCTGCTACCTTGGGTGAGAAGGTTA | bold text indicates |
| | CTATGACTTGTAGAGCTTCATCTAACGTTAAGTACATGTACTGGT | a linker; |
| | ACCAACAGAAGTCTGGTGCTTCTCCAAAGTTGTGGATTTACTAC | unformatted text |
| | ACTTCTAACTTGGCTTCTGGTGTTCCAGGTAGATTTTCTGGTTCA | indicates the |
| | GGTTCTGGTACTTCCTACTCTTTGACTATTTCCTCTGTTGAAGCT | variable region of |
| | GAAGATGCTGCTACTTACTACTGTCAACAATTCACTTCTTCCCCA | the light chain |
| | TACACTTTTGGAGGAGGTACTAAGTTGGAAATCAAG | |
| 1C3 scFv | GAAGTCCGTCTGCTGGAAAGCGGGGGTGGTCCTGTGCAGCCTGGTG | 41; italicized text |
| | GGTCCCTGAAACTGTCCTGTGCCGCAAGCGGGTTCGATTTTTCCAGA | indicates the |
| | TACTGGATGAACTGGGTGAGGAGGGCTCCAGGCAAGGGCCTGGAGT | variable region of |
| | GGATCGGCGAGATCAACCAGCAGTCCAGCACCATCAATTACTCTCCC | the heavy chain; |
| | CCTCTGAAGGACAAGTTCATCATCAGCCGCGATAACGCTAAGTCTAC | bold text indicates |
| | ACTGTATCTGCAGATGAATAAGGTGAGAAGCGAGGACACCGCCCTGT | a linker; |
| | ACTATTGCGCTCGCCTGTCTCTGACAGCCGCTGGCTTTGCCTATTGG | unformatted text |
| | GGCCAGGGCACCCTGGTGACAGTGTCTGCTGGAGGAGGCTCTTC | indicates the |
| | CGGAGGATCCGGCAGCTCTGGCGGCTCCAGCTCTGGCGGCG | variable region of |
| | ATATCGTGATGAGCCAGTCTCCCTCCAGCCTGGCCGTGTCCGTG | the light chain |
| | GGAGAGAAGGTGTCCATGAGCTGTAAGTCTTCCCAGTCTCTGTT | |
| | CAACTCCAGAACCCGCAAGAATTACCTGACATGGTATCAGCAGA | |
| | AGCCTGGCCAGAGCCCCAAGCCTCTGATCTACTGGGCCAGCACC | |
| | AGAGAGTCTGGAGTGCCAGACCGCTTCACCGGCTCTGGATCCGG | |
| | CACAGACTTCACCCTGACAATCAGCTCTGTGCAGGCCGAGGACC | |
| | TGGCTGATTACTATTGCAAGCAGTCCTATAATCTGAGGACCTTTG | |
| | GCGGCGGCACAAAGCTGGAGATCAAG | |
| R18 scFv | CAGGTTAAACTCCAGCAAAGTGGTGGCGGGCTCGTACAACCAGGCG | 42; italicized text |
| | GTTCCCTCAAGTTGTCCTGCGCCGCATCAGGGTTTACATTTAGCTCTT | indicates the |
| | ATGGTATGTCTTGGTTTCGCCAGACGCCTGACAAGCGACTCGAGCTG | variable region of |
| | GTCGCTATCATCAATAGTAACGGAGGTACTACATATTATCCCGACAGT | the heavy chain; |
| | GTGAAGGGGCGATTTACCATTAGCCGGGACAACGCCAAAAATACACT | bold text indicates |
| | GTACCTCCAGATGTCAAGCTTGAAATCAGAAGATACGGCCATGTACT | a linker; |
| | ATTGCGCTAGGGGGGGTGGAAGGTGGCTTCTGGACTATTATGGTCA | unformatted text |
| | GGGTACAACAGTGACAGTATCCTCCGGTGGAGGTGGATCTGGTG | indicates the |
| | GTGGAGGATCTTCAGGAGGTGGTGGATCTTCCGACATAGAGC | variable region of |
| | TTACACAATCTCCGTCATCACTGGCAGTCTCAGCCGGGGAAAAA | the light chain |
| | GTGACAATGTCATGCAAGTCAAGCCAGAGCGTTCTTTATTCATC | |
| | TAATCAGAAGAACTACCTGGCATGGTATCAGCAGAAGCCGGGA | |
| | CAGTCCCCTAAGCTCCTCATCTACTGGGCAAGCACCAGGGAATC | |
| | CGGAGTGCCGGACAGGTTTACTGGGTCCGGTTCTGGGACGGATT | |
| | TTACGCTTACGATATCAAGTGTCCAAGCTGAGGACCTCGCAGTA | |
| | TACTACTGTCACCAGTACCTGTCTTCTTCTACTTTTGGGGGTGGA | |
| | ACGAAACTGGAAATAAAA | |
| EPO (WT) | GCTCCACCTAGATTGATTTGTGATTCCAGAGTTTTGGAAAGATA | 43 (see e.g., |
| | CTTGTTGGAAGCTAAGGAGGCTGAAAATATTACTACTGGTTGTG | GenBank |
| | CTGAACATTGTTCTTTGAACGAGAATATTACTGTTCCAGATACTA | (accession |
| | AGGTTAACTTTTACGCTTGGAAGAGAATGGAAGTTGGTCAGCAA | no. |
| | GCTGTTGAAGTTTGGCAAGGTTTGGCTTTGTTGTCTGAAGCTGTT | KX026660-3) |
| | TTGAGAGGTCAAGCTTTGTTGGTTAATTCTTCTCAACCATGGGAA | |
| | CCATTGCAATTGCATGTTGATAAGGCTGTTTCTGGTTTGAGATCT | |
| | TTGACTACCTTGTTGAGAGCTTTGGGTGCTCAAAAGGAAGCTAT | |
| | TTCTCCTCCAGATGCTGCTTCTGCCGCTCCATTGAGAACTATTAC | |
| | TGCTGATACTTTTAGAAAGTTGTTTAGAGTTTACTCTAACTTCTT | |
| | GAGAGGTAAGTTGAAGTTGTACACTGGTGAAGCTTGTAGAACTG | |
| | GTGATCGG | |
| EPO | GCTCCACCTAGATTGATTTGTGATTCCAGAGTTTTGGAAAGATA | 44 (see e.g., |
| (R150A) | CTTGTTGGAAGCTAAGGAGGCTGAAAATATTACTACTGGTTGTG | GenBank |
| | CTGAACATTGTTCTTTGAACGAGAATATTACTGTTCCAGATACTA | (accession no. |
| | AGGTTAACTTTTACGCTTGGAAGAGAATGGAAGTTGGTCAGCAA | KX026660-3); |
| | GCTGTTGAAGTTTGGCAAGGTTTGGCTTTGTTGTCTGAAGCTGTT | bolded double- |
| | TTGAGAGGTCAAGCTTTGTTGGTTAATTCTTCTCAACCATGGGAA | underlined text |
| | CCATTGCAATTGCATGTTGATAAGGCTGTTTCTGGTTTGAGATCT | indicates the |
| | TTGACTACCTTGTTGAGAGCTTTGGGTGCTCAAAAGGAAGCTAT | R150A mutation |
| | TTCTCCTCCAGATGCTGCTTCTGCCGCTCCATTGAGAACTATTAC | |
| | TGCTGATACTTTTAGAAAGTTGTTTAGAGTTTACTCTAACTTCTT | |
| | G [symbol] GGTAAGTTGAAGTTGTACACTGGTGAAGCTTGTAGAACTG | |
| | GTGATCGG | |
| EPO | GCTCCACCTAGATTGATTTGTGATTCCAGAGTTTTGGAAAGATA | 45 GenBank |
| (K45D) | CTTGTTGGAAGCTAAGGAGGCTGAAAATATTACTACTGGTTGTG | (accession |
| | CTGAACATTGTTCTTTGAACGAGAATATTACTGTTCCAGATACT [symbol] | no. |
| | [symbol] GTTAACTTTTACGCTTGGAAGAGAATGGAAGTTGGTCAGCAA | KX026660-3); |
| | GCTGTTGAAGTTTGGCAAGGTTTGGCTTTGTTGTCTGAAGCTGTT | bolded double- |
| | TTGAGAGGTCAAGCTTTGTTGGTTAATTCTTCTCAACCATGGGAA | underlined text |
| | CCATTGCAATTGCATGTTGATAAGGCTGTTTCTGGTTTGAGATCT | indicates the |
| | TTGACTACCTTGTTGAGAGCTTTGGGTGCTCAAAAGGAAGCTAT | K45D mutation |
| | TTCTCCTCCAGATGCTGCTTCTGCCGCTCCATTGAGAACTATTAC | |
| | TGCTGATACTTTTAGAAAGTTGTTTAGAGTTTACTCTAACTTCTT | |
| | GAGAGGTAAGTTGAAGTTGTACACTGGTGAAGCTTGTAGAACTG | |
| | GTGATCGG | |
| 5AA linker | TCTGGTGGTGGTTCC | 5 |
| (Gly + Ser)₅ | | |
| 7AA linker | GGAGGATCTGGTGGTGGTTCC | 46 |
| 17AA linker | GGAGGATCCGGTGGTGGAGGATCATCTGGTGGAGGATCTGGTG | 47 |
| | GTGGTTCC | |
| 29AA linker | GGAGGAAGTTCCGGTGGTGGATCTTCTTCTGGAGGTGGAGGATC | 48 |
| | CGGTGGTGGAGGATCATCTGGTGGAGGATCTGGTGGTGGTTCC | |
| 35AA linker | GGTGGAGGTGGTTCCGGAGGAGGAAGTTCCGGTGGTGGATCTTC | 49 |
| | TTCTGGAGGTGGAGGATCCGGTGGTGGAGGATCATCTGGTGGAG | |
| | GATCTGGTGGTGGTTCC | |
| 18AA linker | GGTGGAGGTGGATCTGGTGGTGGAGGATCTTCAGGAGGTGGTG | 136 |
| | GATCTTCC | |
| 39AA linker | GGTGGAAGTAGTGGTGGAGGTGGTTCCGGAGGAGGAAGTTCCG | 90 |
| (Gly + Ser)₃₉ | GTGGTGGATCTTCTTCTGGAGGTGGAGGATCCGGTGGTGGAGGA | |
| | TCATCTGGTGGAGGATCTGGTGGTGGTTCC | |

TABLE 9
A list of protein sequences for exemplary anti-GPA antibody fragments,
linkers, and EPO.

| Name | Sequence | SEQ ID NO |
|---|---|---|
| IH4 | QVQLQESGGGSVQAGGSLRLSCVASGYTDSTYCVGW | 2; italicized text indicates the |
| nanobody | FRQAPGKEREGVARINTISGRPWYADSVKGRFTISQD | variable region of the heavy chain; |
| (also | NSKNTV [symbol] LQMNSLKPEDTAIYYCTLTTANSRGFCSGG | bold double-underlined text indicates |
| referred to | YNYKGQG [symbol] QVTVS | a Phe80Tyr point mutation and an |
| as IH4v1) | | additional amino acid Thr118 |
| IH4 | QVQLQESGGGSVQAGGSLRLSCVASGYTDSTYCVGW | 50; italicized text indicates the |
| nanobody | FRQAPGKEREGVARINTISGRPWYADSVKGRFTISQD | variable region of the heavy chain; |
| | NSKNTV [symbol] LQMNSLKPEDTAIYYCTLTTANSRGFCSGG | bold double-underlined text indicates |
| | YNYKGQGQVTVS | Phe80 |
| 10F7 scFv | QVKLQQSGAELVKPGASVKLSCKASGYTFNSYFMHW | 51; italicized text indicates the |
| | MKQRPVQGLEWIGMIRPNGGTTDYNEKFKNKATLT | variable region of the heavy chain |
| | VDKSSNTAYMQLNSLTSGDSAVYYCARWEGSYYALDY | (see e.g., SEQ ID NO: 64); bold text |
| | WGQGTTVTVSSGGGGSGGGGSSGGGGSSDIELTQ | indicates a linker (see e.g., SEQ ID |
| | SPAIMSATLGEKVTMTCRASSNVKYMYWYQQKS | NO: 65); unformatted text indicates |
| | GASPKLWIYYTSNLASGVPGRFSGSGSGTSYSLTI | the variable region of the light chain |
| | SSVEAEDAATYYCQQFTSSPYTFGGGTKLEIK | (see e.g., SEQ ID NO: 66) |
| 1C3 scFv | EVRLLESGGGPVQPGGSLKLSCAASGFDFSRYWMN | 52; italicized text indicates the |
| | WVRRAPGKGLEWIGEINQQSSTINYSPPLKDKFIISRD | variable region of the heavy chain |
| | NAKSTLYLQMNKVRSEDTALYYCARLSLTAAGFAYW | (see e.g., SEQ ID NO: 67); bold text |
| | GQGTLVTVSAGGGSSGGSGSSGGSSSGGDIVMSQ | indicates a linker; unformatted text |
| | SPSSLAVSVGEKVSMSCKSSQSLFNSRTRKNYLT | indicates the variable region of the |
| | WYQQKPGQSPKPLIYWASTRESGVPDRFTGSGSG | light chain (see e.g., SEQ ID NO: |
| | TDFTLTISSVQAEDLADYYCKQSYNLRTFGGGTK | 68) |
| | LEIK | |
| R18 scFv | QVKLQQSGGGLVQPGGSLKLSCAASGFTFSSYGMS | 53; italicized text indicates the |
| | WFRQTPDKRLELVAIINSNGGTTYYPDSVKGRFTISR | variable region of the heavy chain |
| | DNAKNTLYLQMSSLKSEDTAMYYCARGGGRWLLDY | (see e.g., SEQ ID NO: 69); bold text |
| | YGQGTTVTVSSGGGGSGGGGSSGGGGSSDIELTQ | indicates a linker; unformatted text |
| | SPSSLAVSAGEKVTMSCKSSQSVLYSSNQKNYLA | indicates the variable region of the |
| | WYQQKPGQSPKLLIYWASTRESGVPDRFTGSGSG | light chain (see e.g., SEQ ID NO: |
| | TDFTLTISSVQAEDLAVYYCHQYLSSSTFGGGTKL | 70). See also SEQ ID NO: 138. |
| | EIK | |
| EPO (WT) | APPRLICDSRVLERYLLEAKEAENITTGCAEHCSL | 1 |
| | NENITVPDTKVNFYAWKRMEVGQQAVEVWQGL | |
| | ALLSEAVLRGQALLVNSSQPWEPLQLHVDKAVS | |
| | GLRSLTTLLRALGAQKEAISPPDAASAAPLRTITA | |
| | DTFRKLFRVYSNFLRGKLKLYTGEACRTGDR | |
| EPO | APPRLICDSRVLERYLLEAKEAENITTGCAEHCSL | 14; bolded double-underlined text |
| (R150A) | NENITVPDTKVNFYAWKRMEVGQQAVEVWQGL | indicates the R150A mutation |
| | ALLSEAVLRGQALLVNSSQPWEPLQLHVDKAVS | |
| | GLRSLTTLLRALGAQKEAISPPDAASAAPLRTITA | |
| | DTFRKLFRVYSNFL [symbol] GKLKLYTGEACRTGDR | |
| EPO | APPRLICDSRVLERYLLEAKEAENITTGCAEHCSL | 13; bolded double-underlined text |
| (K45D) | NENITVPDT [symbol] VNFYAWKRMEVGQQAVEVWQGL | indicates the K45D mutation |
| | ALLSEAVLRGQALLVNSSQPWEPLQLHVDKAVS | |
| | GLRSLTTLLRALGAQKEAISPPDAASAAPLRTITA | |
| | DTFRKLFRVYSNFLRGKLKLYTGEACRTGDR | |
| 5AA linker | SGGGS | 3 |
| (Gly+Ser)₅ | | |
| 7AA linker | GGSGGGS | 54 |
| 17AA linker | GGSGGGGSSGGGSGGGS | 55 |
| 29AA linker | GGSSGGGSSSGGGGSGGGGSSGGGSGGGS | 56 |
| 35AA linker | GGGGSGGGSSGGGSSSGGGGSGGGGSSGGGSGG | 57 |
| | GS | |
| 18AA linker | GGGGSGGGGSSGGGGSS | 91 |
| 39AA linker | GGSSGGGGSGGGSSGGGSSSGGGGSGGGGSSGG | 92 |
| (Gly+Ser)₃₉ | GSGGGS | |

TABLE 10
Nanobody Amino Acid Sequences from FIG. 30A

| PDB ID | Description | Sequence | SEQ ID NO |
|---|---|---|---|
| 1G6V | Dromedary nanobody | QVQLVESGGGSVQAGGSLRLSCAASGYTVSTYCM | 58 |
| | to bovine carbonic | GWFRQAPGKEREGVATILGGSTYYGDSVKGRFTIS | |
| | anhydrase | QDNAKNTVYLQMNSLKPEDTAIYYCAGSTVASTG | |
| | | WCSRLRPYDYHYRGQGTQVTVSS | |
| 4W6X | Llama nanobody to | QVQLQESGGGSVQAGGSLRLSCTASGYTYRKYCM | 59 |
| | lectin domain of F18 | GWFRQAPGKEREGVACINSGGGTSYYADSVKGRF | |
| | fimbrial adhesin FedF | TISQDNAKDTVFLRMNSLKPEDTAIYYCALSSNS | |
| | | VCPPGHVAWYNDWGQGTQVTVSS | |
| 5HDO | Dromedary nanobody | QVQLQESGGGLVQAGGSLRLSCAASGFTLDSYAIG | 60 |
| | to urokinase-type | WFRQAPGKEREGVSCISASGGSTNYADSVKGRFTI | |
| | plasminogen activator | SRDNAKNTVYLQMNSLKSEDTAVYYCAADHPGLCT | |
| | | SESGRRRYLEVWGQGTQVTVSSA | |
| 5MY6 | Dromedary nanobody | QVQLQESGGGSVQAGGSLKLTCAASGYIFNSCGMG | 61 |
| | to HER2 | WYRQSPGRERELVSRISGDGDTWHKESVKGRFTI | |
| | | SQDNVKKTLYLQMNSLKPEDTAVYFCAVCYNLETY | |
| | | WGQGTQVTVSS | |
| 2X6M | Dromedary nanobody | GQLVESGGGSVQAGGSLRLSCAASGIDSSSYCMGW | 62 |
| | to alpha-synuclein | FRQRPGKEREGVARINGLGGVKTAYADSVKDRFTIS | |
| | | RDNAENTVYLQMNSLKPEDTAIYYCAAKFSPGYCG | |
| | | GSWSNFGYWGQGTQVTVSSH | |
| 1OP9 | Camelid nanobody to | QVQLQESGGGSVQAGGSLRLSCSASGYTYISGWFR | 63 |
| | human lysozyme | QAPGKEREGVAAIRSSDGTTYYADSVKGRFTISQD | |
| | | NAKNTVYLQMNSLKPEDTAMYYCAATEVAGWPLDI | |
| | | GIYDYWGQGTEVTVSS | |

See e.g., International Patent Applications WO01994007921, WO01993024630; U.S. Pat. Nos. 9,879,090, 8,900,592; Habib et al., 2013, Anal Biochem, 438, 82-89; Philo et al., 1996, Biochemistry, 35, 1681-1691; Gross & Lodish, 2006, J Biol Chem, 281, 2024-2032; Burrill et al., 2016, supra; Elliott et al., 1997, supra; Piehler et al., 2000, J Biol Chem, 275, 40425-40433; Chasis and Mohandas (1992) Blood 80, 1869-1879; Chasis et al. (1988) J. Cell Biol. 107, 1351-1357; Catimel et al. (1993) J. Immunol. Methods 165, 183-192; Gardner et al. (1989) Immunology 68, 283-289; Habib et al., 2013, supra; Burrill et al., 2016, supra; Lee et al., 2020, supra; the contents of each of which are incorporated herein by reference in their entireties.

### Example 2: Therapeutic Applications and Structural Analyses of Tissue-Protective Erythropoietin

Erythropoietin (EPO) not only stimulates red blood cell (RBC) proliferation and maturation but also promotes blood coagulation, angiogenesis, and general protection from cell damage and death. The tissue-protective feature of EPO is thought to be mediated by an alternative receptor complex composed of an EPO receptor (EPO-R) and CD131, and has the potential to provide therapeutic benefits to a broad spectrum of diseases, in which the general property of tissue protection can be helpful. Described herein is the structure-function relationships of EPO in both hematopoietic and tissue-protective contexts in order to selectively exploit a subset of functions that are desired for a particular medical condition. EPO variants were constructed that exert one or both of RBC-proliferative and tissue-protective effects but not blood coagulation. A handful of EPO mutants were screened for their erythropoietic and tissue-protective activity in vitro in unfused form as well as in Targeted EPO form, as a non-limiting example characterized by an EPO mutant fused to an anti-GPA antibody fragment via a 5 amino acid linker (IH4-5-EPO(Mut)). Forms of Targeted EPO containing EPO mutations at the strong receptor-binding site, such as K45D and R150A, stimulated erythroid cell proliferation but did not protect neuronal cells from cell death. Hence, these molecules are suitable for the treatment for anemia. Forms of Targeted EPO containing EPO mutations at the weak receptor-binding site, such as R103K and L108A, induced both erythroid cell proliferation and tissue protection, and therefore, are useful for the treatment of high-altitude-related illnesses, high-altitude acclimatization, various military applications, and organ damage due to hypoxia from COVID-19, especially in patients on ventilators. Surprisingly, forms of Targeted EPO with the mutations R103K or L108A have an EC50 in the low femtomolar range, about 100,000-fold lower than other forms of Targeted EPO. Described herein are possible mechanisms for this profound enhancement. Structural alignment models of the EPO-EPO—R-CD131 heterocomplex were also built based on the structural homology of EPO to granulocyte-macrophage colony-stimulating factor (GM-CSF). These models, combined with the cell-based assay data, indicate potential binding modes of the EPO-EPO—R-CD131 heterocomplex as well as EPO residues that are important for tissue protection. This work demonstrates that rational mutagenesis of a pleiotropic protein permits both the selective extraction of desired functions and silencing of undesired and potentially adverse effects.

### INTRODUCTION

### Pleiotropic Effects of Erythropoietin

Erythropoietin (EPO) is regarded as a “red blood cell (RBC)-producing” hormone, for its ability to stimulate RBC production in response to hypoxia, but this is not a complete description of EPO's activities. EPO also inhibits apoptosis of late-stage erythroid precursor cells, such as BFU-E and CFU-E, and promotes their proliferation and maturation into the fully committed erythroid lineage. The kidneys of a healthy human adult constitutively produce EPO at a very low level, maintaining about 1-5 pM of circulating EPO under normoxic conditions to keep hemoglobin levels constant. Under hypoxic stress or after massive blood loss, the body responds by increasing the production of EPO, which in turn, heightens the number of erythrocytes in circulation and allows for more efficient tissue oxygenation. EPO stimulates RBC production by activating homodimeric EPO receptors (EPO-Rs), which are abundantly expressed in late erythroid precursor cells but then gradually disappear as the cells mature and get released into the bloodstream as reticulocytes and mature RBCs.

Accumulating information indicates that EPO, like other cytokines and hormones, is pleiotropic and performs several other biological functions in addition to hematopoiesis. Expression of functional EPO-Rs has been confirmed in many tissues other than erythroid precursors, such as endothelial cells, cardiomyocytes, and cells of the central nervous system, including the brain. Deletion of EPO-Rs in mouse embryos resulted not only in impaired erythropoiesis, but also in developmental defects in the heart, the vasculature, and the brain. The existence of functional EPO-Rs in non-hematopoietic tissues indicates that EPO can activate EPO-Rs in different contexts to induce biological activities that are independent of erythropoiesis.

Non-hematopoietic functions of EPO that have been elucidated so far include aiding blood clotting, vascular growth, and tissue protection. These functions reshape the conception of EPO, indicating that it was cleverly designed by nature as a “wound-healing” hormone, rather than a mere “RBC-producing” hormone. When an animal is wounded, the immediate response by the body should be to stop bleeding, to make more RBCs, to increase tissue oxygenation, and to ensure the survival of tissues until oxygen is supplied. EPO has displayed such non-hematopoietic effects in several experimental and clinical studies. For instance, recombinant human EPO, used for the treatment of anemia in patients with chronic kidney diseases and those undergoing chemotherapy, induced side effects that were pro-thrombotic, hypertensive, tumor angiogenic, and tumor proliferative. EPO promotes the expression and secretion of factors involved in blood clotting and hypertension by activating endothelial EPO-Rs, and that it enhances tumor growth by providing anti-apoptotic and proliferative signals via EPO-Rs on tumor cells and endothelial cells in the nearby vasculature. EPO has also shown tissue-protective effects. It reduced neuronal cell damage and improved motor function in animal models of traumatic brain injury (TBI) or stroke. In human clinical trials, intravenous injections of high doses of EPO significantly reduced infarct size and serum markers of brain damage in acute ischemic stroke patients. EPO treatment also resulted in a lowered mortality rate and improved neurological recovery amongst TBI patients. Based on these observations, it was proposed that EPO should be considered a hormone that mediates the body's response to hemorrhage, rather than hypoxia. This view underlies the interpretation of data in this Example. See e.g., Drüeke et al., 2006, supra; Singh et al., 2006, supra; Pfeffer et al., 2009, supra; Henke et al., 2003, supra; Vaziri and Zhou, Nephrology Dialysis Transplantation, 24, 1082-1088, 2009; Brines et al., 2004, supra; Robinson et al., 2018, supra; Cherian et al., Journal of Pharmacology and Experimental Therapeutics 322(2), 2007: 789-794; Yu et al., Molecular medicine reports, 8(5) (2013): 1315-1322; Ehrenreich et al., 2002, supra; Aloizos et al., 2015, supra; Burrill et al., 2016, supra; Lee et al. ACS Synthetic Biology, 9(2), 191-197, 2020; the contents of each of which are incorporated herein by reference in their entireties.

Due to its hematopoietic and tissue-protective functions, EPO holds can be used as a therapeutic for various applications. EPO's hematopoietic activity has been utilized to correct anemia in renal failure and cancer patients. Experimental studies and clinical trials have been carried out to harness the tissue-protective effects of EPO for several different neurological indications. However, the pleiotropic nature of EPO also complicates its therapeutic translation. Depending on the diseases being targeted, only the desired functions should be exploited independent of other unnecessary effects to ensure drug safety; while some functions may be beneficial, others may be useless or even deleterious in the treatment of a particular disease. Such separation of functions can be implemented by targeting EPO to the specific cell types that are involved in activating desired effects, by silencing undesired functions, or by choosing a dose at which only the desired functions are effective. Mechanistic and structural understanding of tissue-protective action of EPO is still lacking, and therefore, limits the capability to custom-design drugs that perform only the desired functions.

### Role of EPO—R-CD131 Heterocomplex in Tissue Protection by EPO

Pleiotropic effects of a cytokine can arise by several mechanisms, such as acting on different cell types or cells at different stages of development, inducing signaling for differing durations of time, and binding to different receptor complexes. EPO's tissue-protective effects can be mediated by a receptor complex that is different from the canonical EPO-R homodimer. In erythroid precursors, where EPO-Rs are most abundantly expressed (about 1000 copies per cell in CFU-E), EPO binds to homodimeric EPO-Rs and induces the dimerization and autophosphorylation of JAK2, which then phosphorylates tyrosine residues at the cytoplasmic tail of EPO-Rs and activates downstream signaling pathways, most notably the JAK2-STAT5 pathway. As a result, anti-apoptotic genes are turned on and prevent erythrocyte precursors from dying so that these cells are able to proliferate and fully commit to the erythroid lineage. On the other hand, cytokine receptor common beta subunit (βcR), also known as CD131, in complex with EPO-R, has been implicated in tissue-protective EPO signaling. CD131 is a signaling subunit for the receptors of granulocyte-macrophage colony-stimulating factor (GM-CSF), interleukin-3 (IL-3), and IL-5. Although CD131 alone cannot bind these cytokines, it is essential for the high affinity binding of ligands to the receptor complexes composed of CD131 and cytokine-specific alpha subunits, as well as for ligand-induced signal transduction. Expression of both EPO-R and CD131 has been verified in various tissues in the central and peripheral nervous systems, retina, heart, kidney, muscle, and endothelium. See e.g., Brines et al., 2004, supra; Leist et al., 2004, supra; Bunn, 2013, supra; Ghezzi and Brines, Cell Death & Differentiation, 11(1) (2004): S37-S44; Ogunshola and Bogdanova, 2013, supra; Hanazono et al., 1995, supra; the contents of each of which are incorporated herein by reference in their entireties.

Engagement of CD131 in EPO signaling has been shown at a functional level in vitro and in vivo. EPO induced phosphorylation of CD131 in the UT-7 cell line within 1-30 minutes after stimulation, indicating that CD131 directly responds to EPO binding and triggers downstream signaling. The addition of neutralizing antibodies for EPO-R or CD131 ablated the anti-apoptotic effect of a tissue-protective variant of EPO that lacks erythropoietic activity (carbamylated EPO; CEPO) in a neuronal cell line. The same molecule, CEPO, restored motor function after spinal compression in wild-type mice but not in CD131 knockout mice. These results indicate that EPO can signal via EPO—R-CD131 to induce tissue-protective effects in vitro and in vivo. See e.g., Hanazono et al., 1995, supra; Brines et al., 2004, supra; Chamorro et al., Biochimica et Biophysica Acta (BBA)-Molecular Cell Research, 1833(8) (2013): 1960-1968; the contents of each of which are incorporated herein by reference in their entireties.

Although data supports tissue protection by EPO, EPO-R, and CD131, many of them do not address how they interact to form a signaling complex for tissue protection at a biochemical, biophysical, or structural level. One biophysical study claims that EPO does not induce any interaction between EPO-R and CD131, rejecting previous hypotheses and experimental results in support of CD131 as an alternative receptor for EPO; see e.g., Shing et al., Scientific reports, 8(12457) (2018): 1-16, the content of which is incorporated herein by reference in its entirety. However, this study used the soluble, extracellular domains of EPO-R and CD131, and relative concentrations of interacting partners were not reasonable enough to come to a conclusion. Such a lack of information about the protein structures and binding kinetics of EPO, EPO-R and CD131 limits understanding of receptor-ligand interaction, which is crucial for the rational design of protein therapeutics.

### Tissue-Protective EPO

Recombinant human EPO can improve neurological outcomes in patients or animal models with brain damage caused by TBI, stroke, and multiple sclerosis. In rats with mild TBI, administration of EPO led to a significant reduction in lesion size. In patients who had severe closed brain injuries from road traffic crashes, those who received EPO treatment showed a lower mortality rate and better neurological function. In multiple sclerosis patients, EPO treatment improved motor and cognitive function. In stroke patients, EPO treatment reduced infarct size and serum S100b levels, and improved stroke scale scores. High doses of EPO were administered to patients or animals within 6 hours after the injury or onset of symptoms for 3-7 days. When patients were given insufficient doses or a single injection of EPO, the treatment did not have significant protective effects on brain damage. However, using high doses of EPO on stroke patients increased the incidences of adverse events, including deaths. See e.g., Ehrenreich et al., 2002, supra; Ehrenreich et al., 2007, supra; Aloizos et al., 2015, supra; Gaddam and Robertson, Tissue-Protective Cytokines, 141-162, Springer, 2013; Nirula et al., Critical care research and practice, vol. 2010, 2010; Ehrenreich et al. Stroke 40(12) e647-e656, 2009; the contents of each of which are incorporated herein by reference in their entireties.

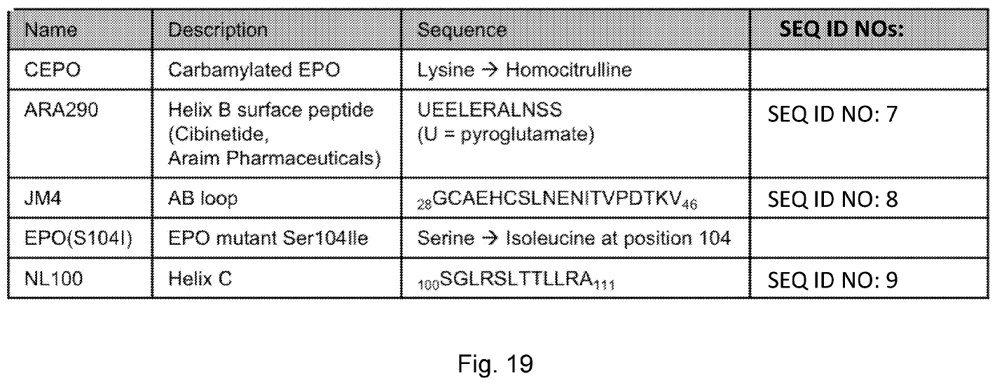

In an effort to develop safer EPO drugs for treating neurological conditions, EPO derivatives have been engineered that provide tissue protection without its other effects, including erythropoiesis (see e.g., FIG. 19). For example, carbamylated EPO (“CEPO”), in which all the lysine residues are modified with a carbamoyl group (−CONH₂) and converted to homocitrulline, completely loses erythropoietic activity in vitro and in vivo due to extensively altered receptor contacts. Despite such changes, this molecule retained tissue-protective effects in vitro (defined by preventing apoptosis of cultured neurons subjected to hypoxic stress), and was able to protect neuronal cells from stroke, spinal cord compression, and diabetic neuropathy in rat models. The observations that such a broad chemical modification did not affect the tissue-protective effects of EPO indicate that a region of EPO that did not contain any lysine residues may include a potential recognition site for the tissue-protective EPO receptor complex. Helix B and the AB loop of EPO do not have lysine residues, and small peptide fragments from these two regions maintained tissue-protective effects despite loss of erythropoietic effects. An 11-mer peptide fragment was derived from helix B by combining the amino acids that are at the aqueous face of helix B, mimicking its solvent-exposed surface structure. This peptide, named ARA290, was also shown to be tissue-protective but not erythropoietic, and has undergone clinical trials for several neurological conditions. Site-directed mutagenesis of EPO revealed mutants that disrupt erythropoietic activity but not tissue-protective activity. Several mutants in helix A and C, such as S104I, exhibited significantly weakened or completely abolished erythropoietic activity, and showed comparable tissue-protective effects to wild-type EPO. Thus, the receptor-interacting epitopes are different for erythropoietic and tissue-protective effects, and the two effects can be separated by distorting or removing certain parts of EPO. See e.g., Leist et al., 2004, supra; Brines et al., 2008, supra; Campana et al., International journal of molecular medicine, 1(1), 235-276, 1998; Erbayraktar et al., Molecular medicine, 15, 235-241, 2009; Ahmet et al, Molecular medicine, 17, 194-200, 2011; Swartjes et al.; Anesthesiology: The Journal of the American Society of Anesthesiologists, 115(5), 1084-1092, 2011; van Rijt et al., Transplant International, 27(3), 241-248, 2014; Gan et al., 2012, supra; the contents of each of which are incorporated herein by reference in their entireties.

### Limitations in Therapeutic Translation of Tissue-Protective EPO

EPO and its derivatives can be effective therapeutics for various neurological indications and TBI, but their poor pharmacokinetic properties and high dose requirements have led to clinical failures. EPO is about 30.4 kDa with a short serum half-life of about 8 hours after a single intravenous injection in humans. Aforementioned EPO derivatives are similar in size to or smaller than wild-type EPO, and thus, have serum half-lives of 8 hours or shorter. The peptide ARA290, for instance, is about 1.3 kDa and has a serum half-life of about 20 minutes after a single subcutaneous injection and about 2 minutes after a single intravenous injection in healthy humans. Such short half-lives necessitate frequent injections or high doses of drugs. Weaker receptor interactions and the need to cross the blood-brain barrier (BBB) also increase the dose requirement. EPO binds to homodimeric EPO-R with high affinity (K_{D}=0.1-1 nM) while it binds to the tissue-protective receptor complex with about 100-fold lower affinity. This indicates that higher doses of EPO are needed for tissue-protection than for RBC production. Additionally, in the context of tissue protection of the central nervous system, such as the brain, the amount of EPO available for target tissues is restricted by its efficiency crossing the BBB. Although EPO and its derivatives are much larger than the cut-off (400-600 Da) for BBB permeability by passive lipid-mediated transport, EPO is still able to pass through the BBB. Mice that received a single intravenous injection of EPO had significantly elevated EPO levels in the brain, and stroke patients who received an intravenous infusion of EPO had 60-100 times higher levels of EPO in the cerebrospinal fluid than untreated patients. It has been shown in rodents that only a tiny fraction of systemically injected EPO gets into the brain by either active or passive pathway. EPO crosses the BBB in the absence of any neural insult, potentially through EPO-R-mediated transcytosis on the capillary endothelial cells at the brain-periphery interface. About 0.05-0.1% of intravenously injected EPO per gram of brain weight crossed the BBB in mice at a similar rate to albumin, indicating that EPO might cross the BBB via extracellular pathways mediated by diffusion through a leaky BBB. Therefore, clinically relevant doses of EPO must be much higher to deliver to target tissues in the central nervous system. However, the amount of EPO that is allowed in clinical trials is limited by previous studies that were focused on anemia in renal or cancer patients. These studies showed that high doses of EPO increased the chances of thrombovascular incidences in chronic renal failure patients, and consequently, the maximum allowed dose of EPO in clinical settings has been restricted to a non-thrombotic “safe” level that was likely below the effective dose for tissue protection. See e.g., Bunn, 2013, supra; Ehrenreich et al., 2002, supra; Drüeke et al., 2006, supra; Singh et al., 2006, supra; Pfeffer et al., 2009, supra; Philo et al. Biochemistry 35(5), 1681-1691, 1996; Elliott et al. Annals of hematology, 93(2), 181-192, 2014; Masuda et al. Journal of Biological Chemistry 268(15), 11208-11216, 1993; Schuler et al. The FASEB Journal, 26(9), 3884-3890, 2012; Brines et al., PNAS 97(19), 10526-10531, 2000; Banks et al. European journal of pharmacology, 505(1-3), 93-101, 2004; the contents of each of which are incorporated herein by reference in their entireties.

Described herein are EPO-based protein therapeutics rationally designed for various applications in which the general property of tissue protection is useful. The strength of different EPO activities was modulated by incorporating various EPO mutants into a chimeric activator design. Such EPO mutants in the chimeric activator design improve pharmacokinetics and therapeutic windows, allowing the administration of clinically relevant doses that are not limited by the known adverse effects of EPO drugs. Furthermore, these engineering strategies permit customized designs of various flavors of EPO therapeutics that protect neuronal cells from damage caused by various conditions, such as TBI, high-altitude-related illnesses, and neurodegenerative disorders.

### Results and Discussion

### Rationale for the Design of Tissue-Protective Forms of EPO

The goal of this Example was to custom-design EPO therapeutics to accommodate different medical conditions by modulating EPO's erythropoietic and tissue-protective functions. The general strategy for designing such molecules was based on the concept of a “chimeric activator.” In short, a chimeric activator is a fusion protein composed of an antibody element that is targeted to specific cell types expressing antigens, a flexible peptide linker, and an activity element (cytokine or hormone) with a mutation that weakens its affinity for receptors. This way, the activity element acts only on target cells, mitigating unwanted effects via non-target cells; see e.g., Burrill et al., 2016, supra; Taylor et al., 2010, supra; Lee et al. 2020, supra; the contents of each of which are incorporated herein by reference in their entireties. The fine-tuning between the erythropoietic and tissue-protective effects of EPO was achieved by using different mutant proteins with different binding affinities for each of the responsible receptor complexes. By modulating the type and strength of mutations, three types of EPO fusion proteins were designed: EPO-A, EPO-P, and EPO-AP (“A” indicates use in anemia; “P” indicates protection for tissue; “AP” indicates uses for both anemia and tissue-protection, which activities are particularly useful for treating hypoxia, such that EPO-AP and EPO-H refer to the same set of molecules), each of which has a different subset of EPO functions (see e.g., FIG. 10A-10B and FIG. 16). EPO-A only has an erythropoietic effect, and is designed as a replacement for current ESAs that carry lethal thrombotic side effects. It can be used for safer treatment of anemia in chronic kidney disease patients and chemotherapy patients. EPO-P only has a tissue-protective effect, and is useful for treating neurodegenerative disorders as well as any conditions that accompany short-term hypoxic damage or other pro-apoptotic damage, such as TBI, strokes, and surgeries. EPO-AP has both erythropoietic and tissue-protective effects, and is beneficial for patients suffering from high altitude-related illnesses as well as military personnel acclimating to high-altitude regions, seeking safe ways to enhance physical performance, and needing general protection from potential threats, such as chemical warfare. EPO-AP can also alleviate organ damages caused by hypoxia, e.g., in COVID-19 patients (see e.g., FIG. 16).

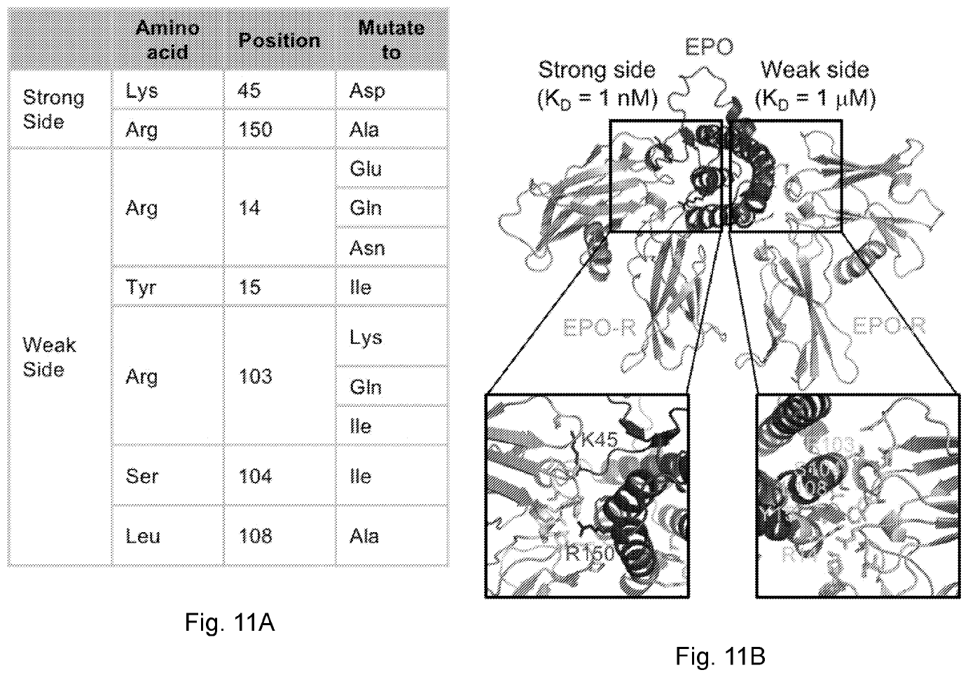

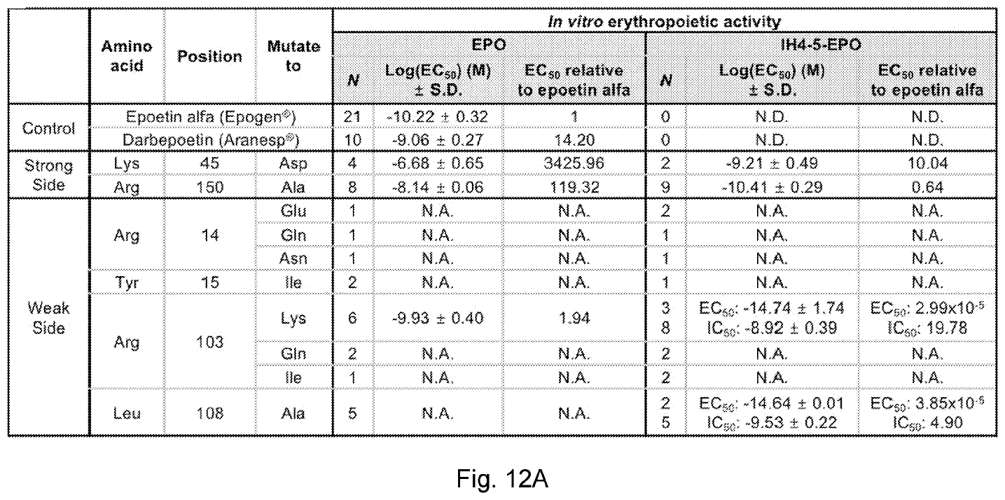

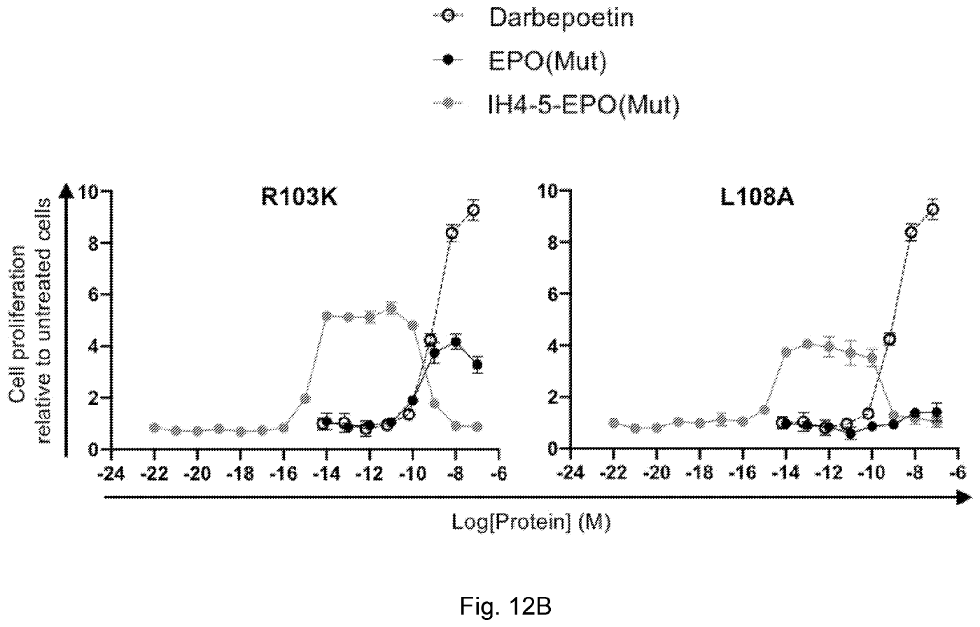

All of these molecules share common features that improve drug safety and pharmacokinetics. Fusing mutated EPO to the anti-glycophorin A (GPA) nanobody element, IH4, via a short 5 amino acid linker not only increases the size of the molecule to above the renal filtration cut-off but also directs the fusion proteins to mature RBCs in circulation, extending serum half-life and reducing immunogenicity of the drug by inducing tolerance. EPO mutations in all three molecules generally weaken its affinity to homodimeric EPO-R to avoid undesired pro-thrombotic effects, triggered by homodimeric EPO-R signaling on non-target cells. The extent to which each of these mutations disrupts homodimeric EPO-R interaction is based on whether or not erythropoietic activity is desired. At the same time, it is important to ensure that the same mutation does not disrupt EPO binding to the EPO—R-CD131 complex when the tissue-protective activity of EPO is desired (see e.g., FIG. 10A-10B). Several EPO mutants were designed based on EPO mutagenesis studies. Because the binding mode of EPO to EPO—R-CD131 is not elucidated, unlike that of EPO to homodimeric EPO-R, single point mutations were made in both of the known EPO-R contact regions (i.e., strong and weak interaction sides) (see e.g., FIG. 11A-11B). These EPO mutants were expressed and purified in an unfused form (EPO(Mut)) and an antibody-fused form (IH4-5-EPO(Mut)), and were tested for erythropoietic and tissue-protective activities. See e.g., Burrill et al., 2016, supra; Kontos and Hubbell, 2010, supra; Kontos et al., 2013, supra; Gan et al., 2012, supra; Elliott et al., 1997, supra; the contents of each of which are incorporated herein by reference in their entireties.

### Erythropoietic Activity of EPO Variants

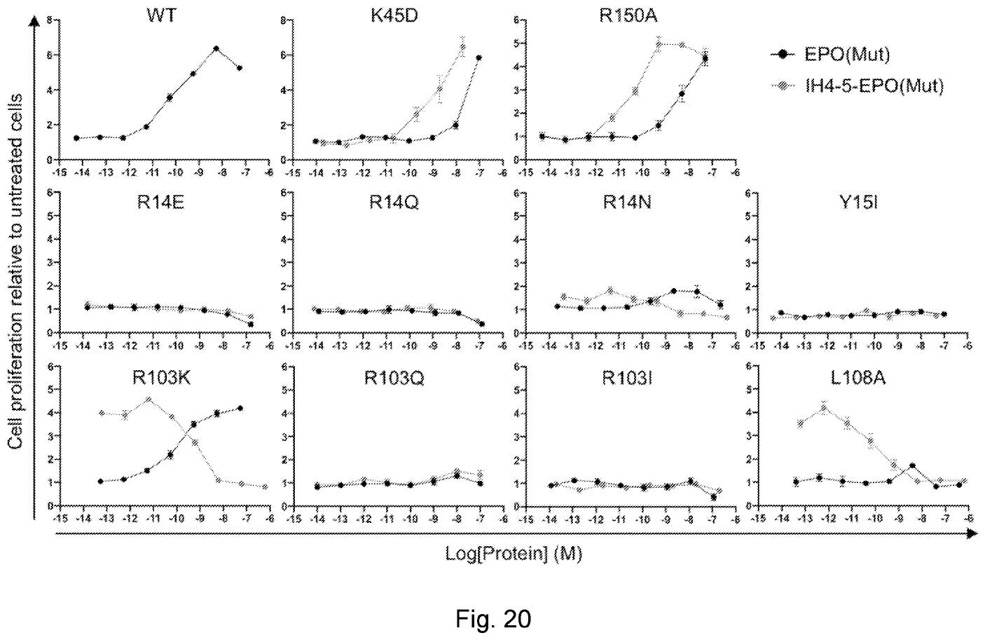

The ability of different EPO mutants to promote RBC production was tested in vitro via TF-1 cell proliferation assays. The TF-1 cell line is of immature erythroid origin, expresses both EPO-R and GPA (1620±140 and 3860±780 molecules per cell, respectively), and requires EPO, GM-CSF, or IL-3 for growth. TF-1 cells that were starved of cytokines overnight were incubated with different EPO variants for 72 hr, and then their proliferation was measured by standard tetrazolium-based assay. Wild-type EPO (epoetin alfa, Epogen®, AMGEN) and the hyperglycosylated form of EPO (darbepoetin alfa, Aranesp®, AMGEN) exhibited EC50 values of about 0.1 nM and 1 nM, respectively. EPO mutations on the strong side reduced the activity of unfused EPO by about 120-fold to 3400-fold relative to epoetin alfa. When these mutants were fused to the IH4 nanobody, their activities were rescued by about 180- to 340-fold relative to unfused mutants, showing comparable activity to epoetin alfa and darbepoetin (see e.g., FIG. 12A). All unfused EPOs with mutations on the weak side did not show any activity at concentrations ranging from 10⁻¹⁴ to 10⁻⁷ M, except for EPO(R103K) (see e.g., FIG. 12A-12B and FIG. 20). EPO(R103K) had a comparable EC50 value to epoetin alfa but about two-fold lower efficacy (E.).

The dose-response curve of weak-side mutant EPOs fused to IH4 had three unusual features. First, when EPO(R103K) was fused to IH4, the potency of the fusion protein was enhanced by five orders of magnitude relative to the unfused mutant and to other EPO fusion proteins. The EC50 was about 1 fM-5 fM (see e.g., FIG. 12A-12B). Among the weak-side mutants that lack erythropoietic activity, only EPO(L108A) exhibited targeted erythropoietic activity upon fusion to IH4 and also had an EC50 of about 1 fM-5 fM, while the others remained inactive even after fusion (see e.g., FIG. 12A-12B and FIG. 20). Both of these mutations have the effect of reducing the size of a side chain and thus removing a contact with the receptor, but do not create steric hindrance for binding. Secondly, the maximal stimulation of TF-1 cell proliferation by IH4-5-EPO(R103K or L108A) was about ⅓ to ½ of the stimulation seen with wild-type EPO and other EPO fusion proteins (see e.g., FIG. 12B). Lastly, the dose-response curve of IH4-5-EPO(R103K or L108A) was bell-shaped, with stimulation falling off at about 0.1 nM-0.5 nM, whereas fusion proteins containing strong-side mutants (K45D and R150A) showed standard sigmoidal dose-response curves (see e.g., FIG. 12B and FIG. 20). Without wishing to be bound by theory, hypotheses to explain each of these results are proposed as follows.

First, two possible mechanisms could explain the extremely high potency of IH4-5-EPO(R103K or L108A).

1. The attachment of the fusion protein to GPA can prevent receptor-mediated endocytosis and degradation of the signaling protein. GPA is attached to a stable actin cytoskeleton that may preclude internalization.

2. The fusion protein may form a highly stable complex with GPA and one copy of EPO-R via the strongly interacting side of EPO, but the interaction with the second EPO-R to form a complete signaling complex may be very weak and dissociate rapidly due to a mutation. The interaction may last long enough to phosphorylate a subset of the tyrosine residues important in signal transduction into the nucleus but may not be long enough to phosphorylate those involved in signaling to the clathrin system for receptor-mediated endocytosis.

The former model is contradicted by the fact that highly potent activity was not observed with other targeted EPO fusion proteins. However, the underlying concept of this model may explain how such a small amount of fusion protein can trigger cell proliferation without being cleared by non-receptor-mediated endocytosis that routinely occurs as part of the membrane turnover. Additionally, the ability of IH4-5-EPO(R103K or L108A) to stimulate cell proliferation at very low concentrations indicates that only a small number of complete receptor-ligand complexes are required and sufficient to induce proliferative signaling. In this experiment, each well of a 96-well plate initially contained 9,000 cells and about 6.022×104 copies of fusion proteins (when 1 fM was given), thereby resulting in a molar ratio of 1:1.67 between the number of cells and that of fusion proteins per well. Assuming that cells and proteins are evenly distributed, only 1 to 2 copies of fusion proteins on average would bind to receptors on each cell at a given time and stimulate cell proliferation. This extraordinary feature of IH4-5-EPO(R103K or L108A) may be possible because complete dissociation of the fusion protein from GPA and EPO-R occurs at a very low rate. IH4-5-EPO(R103K or L108A) practically has a bivalent targeting element consisting of IH4 and the strong side of EPO. Having two target sites increases the net affinity (“avidity effect”) and reduces the overall dissociation rate of the fusion protein from its targets. When one part of the fusion protein, IH4 for example, dissociates from its target site, GPA, the other part of the fusion protein, EPO in this case, may remain bound to its target site, EPO-R, and force IH4 to stay in proximity such that re-binding can occur rapidly.

Secondly, the failure to form a long-lasting stable signaling complex could explain the low maximal stimulation of cell proliferation. The fusion protein may form a tight complex with GPA and one copy of EPO-R but its binding to the second copy of EPO-R is very weak (K_{D}>1 μM). Rapid dissociation of the second EPO-R may interfere with full activation of EPO-R, thereby explaining the activity profile resembling that of a typical partial agonist, with less than half of the maximal efficacy observed with wild-type EPO and other fusion proteins.

Lastly, the bell-shaped dose-response curve of IH4-5-EPO(R103K or L108A) can be explained by receptor saturation that has been observed in other systems that require more than two receptors for signaling. The fusion protein may saturate monomeric EPO-R in a 1:1 stoichiometry and block the formation of a complete signaling complex consisting of homodimeric EPO-R. Calculations based on the dissociation constant (K_{D}) of EPO for EPO-R on the strong side and the concentrations of the applied fusion protein and EPO-R show that the addition of 0.1 nM of fusion protein would result in about 9.1% of EPO-R being bound by the fusion protein via the strong interaction side. Similarly, adding 1 nM and 10 nM would lead to about 50.0% and 90.8% of EPO-R being occupied by the fusion protein, respectively. This is consistent with the experimental data showing that the activity of IH4-5-EPO(R103K or L108A) begins to fall off at 0.1 nM-0.5 nM and is completely lost at >5-10 nM (see e.g., FIG. 12B).

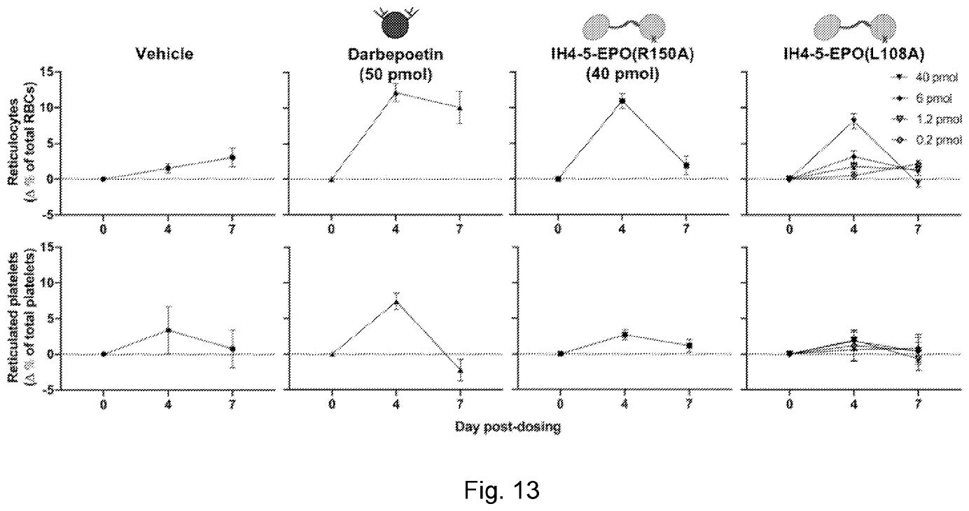

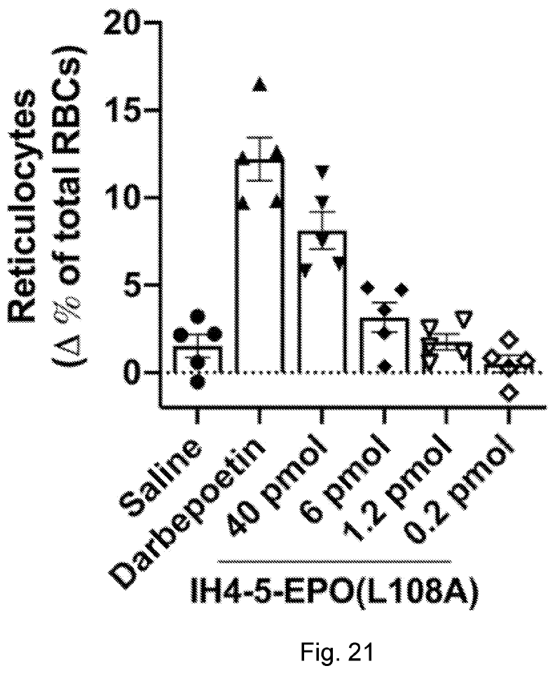

These results indicate that IH4-5-EPO fusion proteins composed of the K45D, R150A, R103K, or L108A mutants induce erythroid cell proliferation in vitro, and therefore, can be used as EPO-A or EPO-AP. IH4-5-EPO(R150A) had been tested extensively for its targeted erythropoietic activity as well as reduced systemic pro-coagulative effects in mice. Here, one of the weak-side mutant fusion proteins, IH4-5-EPO(L108A), was further tested for its targeted erythropoietic activity in transgenic mice expressing human GPA. Mice received a single intraperitoneal (i.p.) injection of saline, darbepoetin (50 pmol=1.8 μg), IH4-5-EPO(R150A) (40 pmol=2 μg), or various doses of IH4-5-EPO(L108A) (40 pmol=2 μg; 6 pmol=0.3 μg; 1.2 pmol=0.06 μg; 0.2 pmol=0.01 μg). Target cell specificity and drug efficacy were measured by staining for reticulocytes and reticulated platelets in blood samples on Days 0, 4, and 7. Both the reticulocyte and reticulated platelet levels remained at baseline (Day 0) throughout the experiment in the saline-treated mice, but increased significantly in mice treated with darbepoetin, a control for the non-targeted form of EPO. Mice treated with IH4-5-EPO(R150A) had significantly elevated reticulocyte levels (10.9%) that were comparable to those in the darbepoetin-treated mice (12.2%) on Day 4, but did not have increased reticulated platelet counts. IH4-5-EPO(L108A) behaved similarly to IH4-5-EPO(R150A), in that it elevated reticulocyte levels but not reticulated platelet levels. IH4-5-EPO(L108A) induced reticulocyte responses in a dose-dependent manner. 40 pmol and 6 pmol induced 8.13% and 3.16% increases in reticulocyte counts on Day 4 relative to Day 0, respectively, while lower doses (1.2 pmol and 0.2 pmol) did not have significant effects (see e.g., FIG. 13 and FIG. 21). A wider range of concentrations can be tested to see how the in vitro bell-shaped dose-response curves translate in animal studies.

### Tissue-Protective Activity of EPO Variants

EPO mutants were further tested for their tissue-protective effects in cell-based assays. Those that showed targeted erythropoietic activity were evaluated for their potentials for either EPO-A or EPO-AP. Those that lacked erythropoietic activity were considered for EPO-P. The ability of a fusion protein to protect cells from a toxic damage was measured in vitro by estimating the number of surviving cells after treatment with EPO and a toxic agent. In more detail, SH-SY5Y, a neuroblastoma cell line that expresses both EPO-R and CD131, was pre-treated with EPO or an EPO variant for 24 hr, and then received 100 μM of cobalt chloride (CoCl2), which mimics hypoxia by driving cellular responses initiated by hypoxia-inducible factor-alpha (HIF-1a). 24 hr later, the number of viable cells were measured by standard tetrazolium dye-based assays. The optimal cell density and concentration of CoCl2 were determined experimentally to cause about 40% cell death (see e.g., FIG. 22).

Results showed that wild-type EPO (EPO(WT)) and EPO(S104I) protected neuroblastoma cells from CoCl2 insult. This is consistent with the results that EPO(WT) and EPO(S104I) protected primary neurons from NMDA-induced excitotoxicity (see e.g., FIG. 14A, FIG. 14C; Gan et al., 2012, supra). Darbepoetin, which is hyperglycosylated EPO, provided some level of tissue protection (see e.g., FIG. 14B). This indicates that the additional oligosaccharides did not completely disrupt the receptor-ligand interaction essential for tissue protection. Among the EPO mutants that stimulated TF-1 cell proliferation upon fusion to IH4, two weak-side mutants, EPO(R103K) and EPO(L108A), exhibited tissue-protective effects in both unfused and fused forms (see e.g., FIG. 14F-14I). In contrast, two strong-side mutants, EPO(K45D) and EPO(R150A), did not protect cells from CoCl2-induced cell death (see e.g., FIG. 14D-14E). Four-parameter fits provided rough estimates for the potency of each mutant. The EC50 values of EPO(WT) and EPO(S104I) were about 2 nM, and darbepoetin about 290 nM (ambiguous fit). The EC50 values of EPO(R103K) and EPO(L108A) were predicted to be around 30 nM.

Since the K45D and R150A mutants do not have significant tissue-protective activities, they can be used for EPO-A. IH4-5-EPO(R103K) and IH4-5-EPO(L108A) have both erythropoietic and tissue-protective activities, and therefore, hold can be used for EPO-AP. Due to the complete lack of homodimeric EPO-R activation by its unfused counterpart, IH4-5-EPO(L108A) was chosen for EPO-AP, and can be investigated in an animal model of TBI to corroborate its tissue-protective effects in vivo. The EPO mutants that completely lost erythropoietic activity in both unfused and fused forms, such as EPO(R14E/Q/N), EPO(Y15I), and EPO(R103Q/I), can be tested for their tissue-protective activities to determine their use for EPO-P.

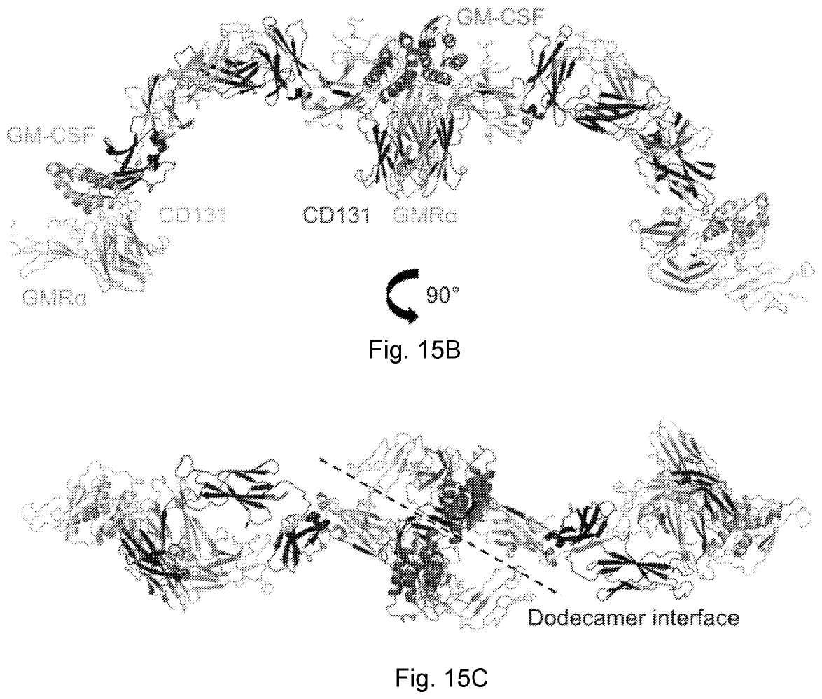

EPO interacts with homodimeric EPO-R via two asymmetric binding interfaces. One side of EPO binds to EPO-R with strong affinity (K_{D}≈1 nM) while the other side binds with much weaker affinity (K_{D}≈1 μM) (see e.g., FIG. 11B). On the other hand, there is not enough structural and kinetics evidence to determine the binding mode of EPO in complex with EPO-R and CD131. In this section, the structure of the EPO-EPO—R-CD131 heterocomplex was modeled based on the known structure of a signaling complex of another type 1 cytokine, GM-CSF, that requires CD131 for receptor activation and signaling and has a similar three-dimensional structure to EPO. The structure of GM-CSF in complex with its receptor alpha subunit, GMRα, and CD131 has been extensively studied, and a set of crystal structures (see e.g., PDB ID: 2GYS, 3CXE, 4NKQ, 4RS1) collectively provides information about the GM-CSF signaling complex. GM-CSF first forms a binary complex with a ligand-specific, low affinity GM-CSF receptor alpha subunit (GMRα) with a K_{D} of about 2 nM-10 nM. Then CD131, which lacks affinity for GM-CSF alone, binds to the binary complex and forms a high affinity ternary complex with a K_{D} of about 50 pM-100 pM. During the transition to the ternary state, GM-CSF adopts a new conformation by tilting, such that the appropriate side chains are exposed to CD131 in the right orientations for optimal interactions. Because CD131 exists as an antiparallel interlocked homodimer, each CD131 dimer forms two sets of ternary complexes, generating a hexamer. This hexamer then assembles into a complete dodecameric signaling complex, such that the cytoplasmic tails of two CD131 is are close enough to induce JAK2 dimerization and phosphorylation. In this structure, a single GM-CSF interacts with GMRα and the domains 1 (D1) and 4 (D4) of each of the two CD131s in a homodimer, and this minimal structure resembles that of EPO-(EPO-R)₂ (see e.g., FIG. 15A-15C). See e.g., Philo et al., 1996, supra; Murphy et al. Journal of Biological Chemistry 278(12), 10572-10577, 2003; Broughton et al. Structure 24(8), 1271-1281, 2016; Stomski et al. Molecular and cellular biology 16(6), 3035-3046, 1996; the contents of each of which are incorporated herein by reference in their entireties.

Without wishing to be bound by theory, four possible ways that EPO could form a functional signaling complex with EPO-R and CD131 were extrapolated from the known structures of the EPO-(EPO-R)₂ complex and the GM-CSF-GMRα-CD131 complex.

1. EPO may bind to EPO-R via the strong side and to CD131 via the weak side. This is kinetically similar to the mechanism in which GM-CSF binds to GMRα and CD131. The ligand binds to the ligand-specific receptor subunit first, and then the binding of CD131 locks the complex into a high-affinity signaling conformation.

2. EPO may bind to EPO-R via the weak side and to CD131 via the strong side. This scenario is kinetically more challenging because CD131 has to compete with EPO-R for the strong side of EPO. However, this hypothesis could be true if EPO behaves differently from other type 1 cytokines and exhibits greater affinity for CD131 than for EPO-R on the strong side, or if the binding of CD131 to the strong side of EPO is driven by the relative abundance of CD131 on the cell surface. Alternatively, EPO-R and CD131 might exist as a pre-formed receptor complex so that CD131 can bind to the strong side of EPO preemptively.

3. EPO may bind to EPO-R via the strong side and to CD131 via regions of EPO other than the strong and weak sides, such as the AB loop and the N- and C-termini. It has been shown that peptide fragments derived from helix B and the AB loop of EPO retained tissue-protective effects but not erythropoietic effects, demonstrating that these regions are sufficient to activate receptors for tissue protection; see e.g., Brines et al., 2008, supra; Campana et al., supra; the contents of each of which are incorporated herein by reference in their entireties.

4. CD131 may not directly interact with EPO but may stabilize a receptor conformation that is amenable to tissue-protective signaling.

To learn which configurations may be structurally plausible, 10 structural alignment models of EPO-EPO—R-CD131 were created by aligning the structure of EPO-(EPO-R)₂ (see e.g., PDB ID: lEER) to that of GM-CSF-GMRα-CD131 (see e.g., PDB ID: 4NKQ) in PyMOL. In most of these models, side chains of EPO and CD131 slightly overlapped possibly due to the lack of an energy minimization step. Models that showed steric clashes at the dodecameric interface were not excluded from further analyses, as structural models of the IL-5 signaling complex showed that structures equivalent to dodecameric complexes of GM-CSF or IL-3 may not be the only possible signaling unit for type 1 cytokines. Alignment of (IL-5)₂-IL-5Ra to the GM-CSF-GMRα-CD131 complex revealed that the formation of such a high-order complex may be sterically impossible for some type 1 cytokines; see e.g., Kusano et al., Protein Science 21(6), 850-864, 2012; the content of which is incorporated herein by reference in its entirety.

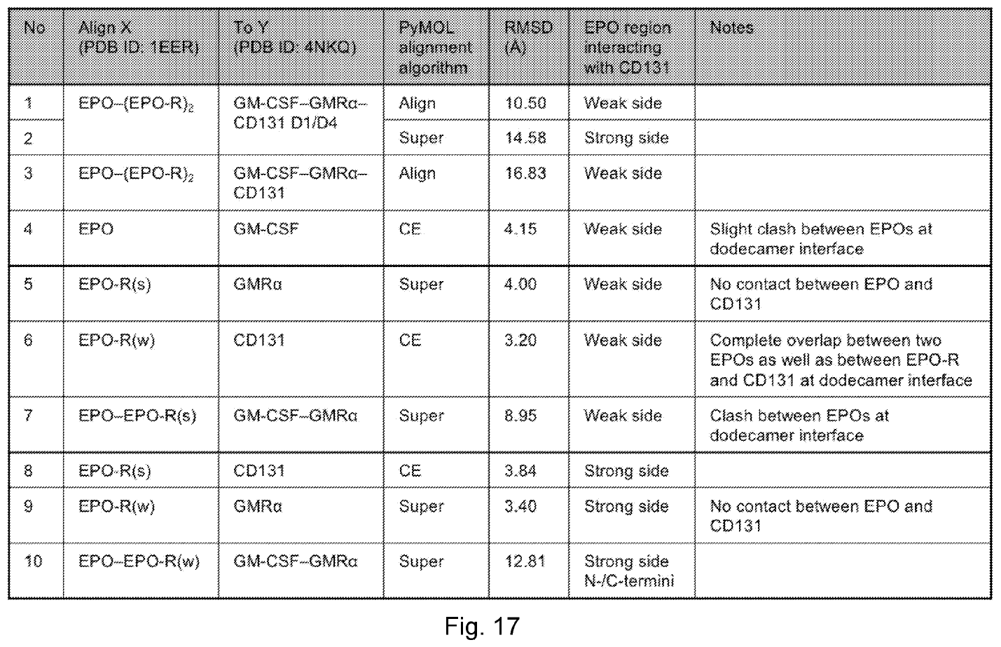

The first two models were generated by aligning EPO-(EPO-R)₂ to GM-CSF in complex with GMRα as well as D1 and D4 of CD131, as these two structures exhibit high structural similarity. Interestingly, in this process, the use of two different algorithms resulted in two distinct modes of formation of the EPO-EPO—R-CD131 heterocomplex (models 1 and 2). CD131 interfaced with the weak and strong sides of EPO in models 1 and 2, respectively (see e.g., FIG. 17 and FIG. 23A-23B). When the complete dodecameric complex of GM-CSF-GMRα-CD131 was used instead, CD131 was positioned at the weak side of EPO (model 3) (see e.g., FIG. 17 and FIG. 23C-23D). The alignment of EPO to GM-CSF (model 4) also resulted in the binding of CD131 on the weak side of EPO (see e.g., FIG. 17 and FIG. 23E-23F). The algorithm used for generating models 1 and 3 (Align) takes into account the sequence identity between proteins that are being aligned whereas the algorithms used for models 2 and 4 (Super and CE, respectively) do not. This indicates that the weak side of EPO has higher sequential and structural similarity to the part of GM-CSF that binds CD131 compared to the strong side of EPO but that the structures of the whole receptor-ligand complexes resemble better when CD131 faces the strong side of EPO.

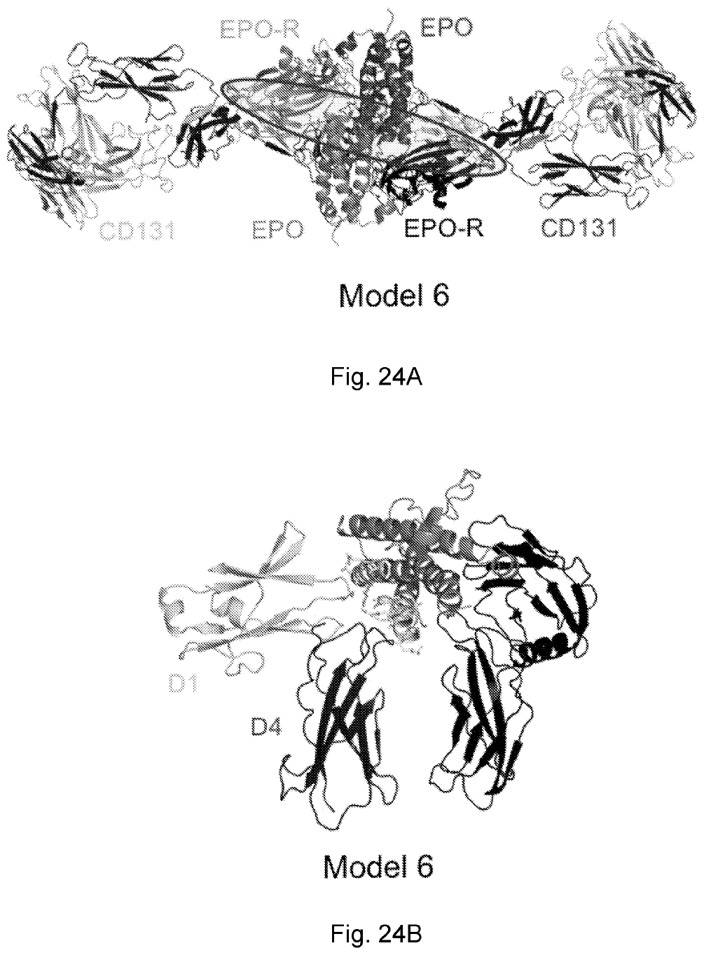

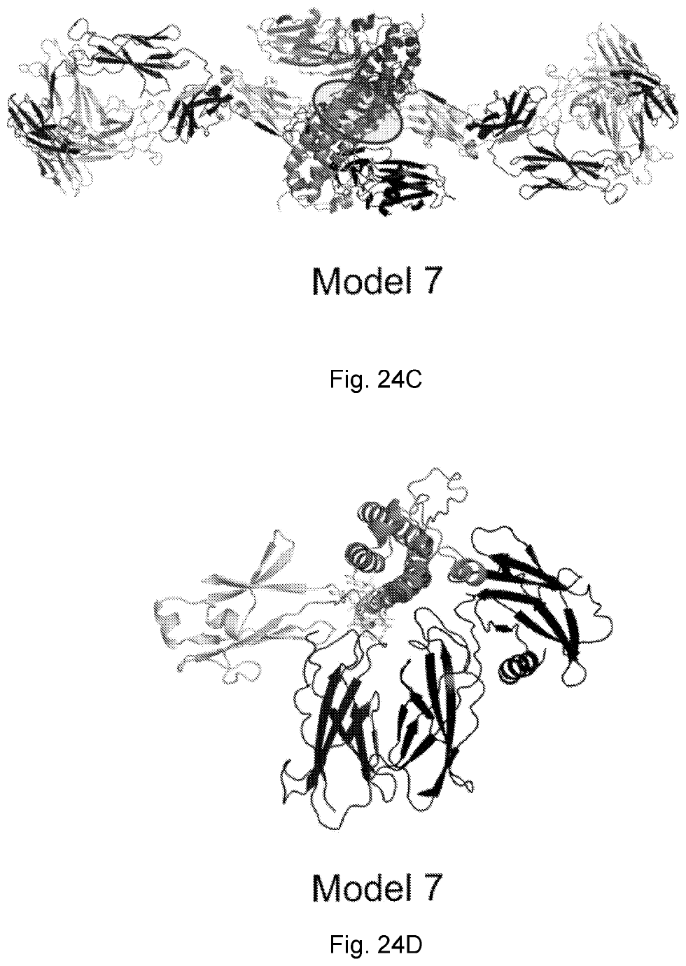

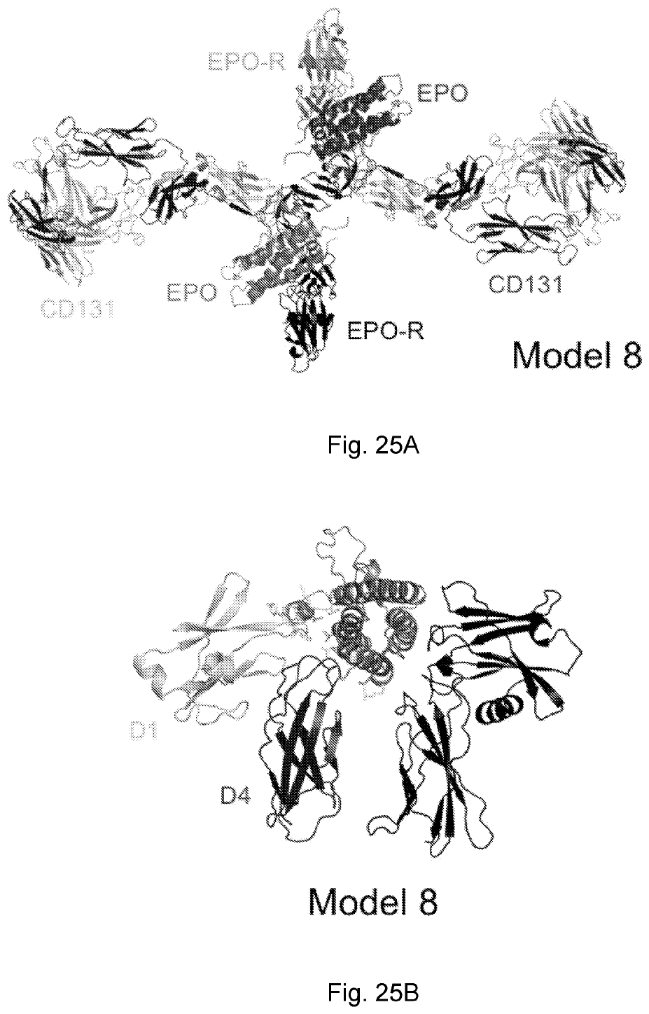

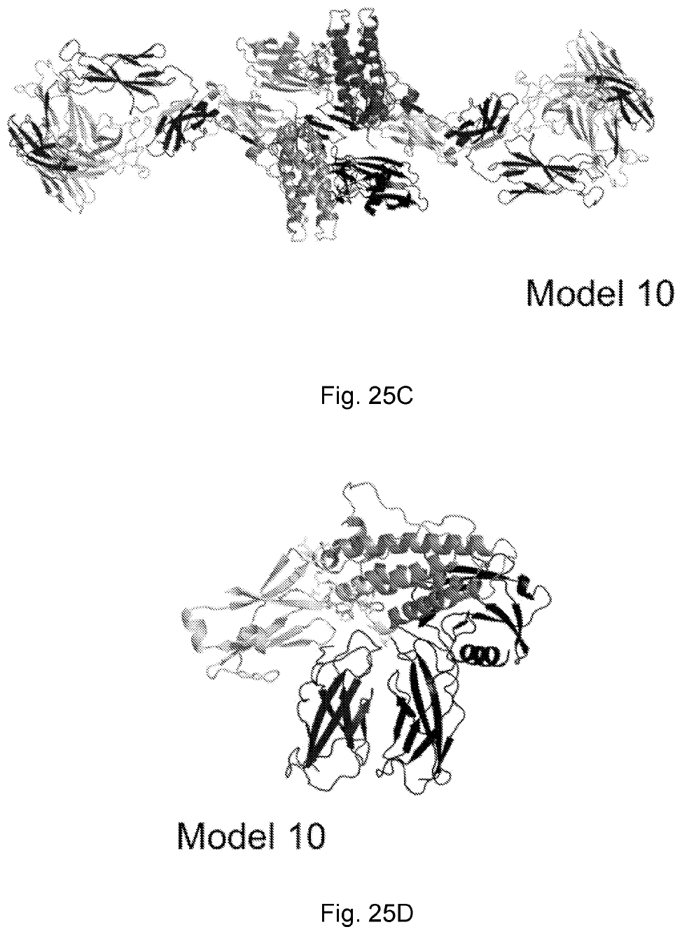

For the configuration in which EPO's weak side interacts with CD131, three additional structural models were generated (models 5-7) (see e.g., FIG. 17). For the other configuration in which EPO's strong side binds to CD131, three structural models were generated in a similar manner (models 8-10) (see e.g., FIG. 17). The alignment of EPO-R to GMRα (models 5 and 9) did not result in any meaningful contact between EPO and CD131. The alignment models that simulated the binding of CD131 on the weak side of EPO (models 6 and 7) resulted in steric clashes between two EPOs of the neighboring hexamers, disrupting the dodecamer formation (see e.g., FIG. 17 and FIG. 24A-24D). The formation of a dodecameric complex is essential for GM-CSF and IL-3 signaling but EPO might not necessarily rely on the dodecamer formation for successful signaling, as shown by the IL-5 complex. The other two models showing the binding of CD131 on the strong side of EPO (models 8 and 10) resulted in meaningful contacts between EPO and receptors without any significant steric clashes (see e.g., FIG. 17 and FIG. 25A-25D).

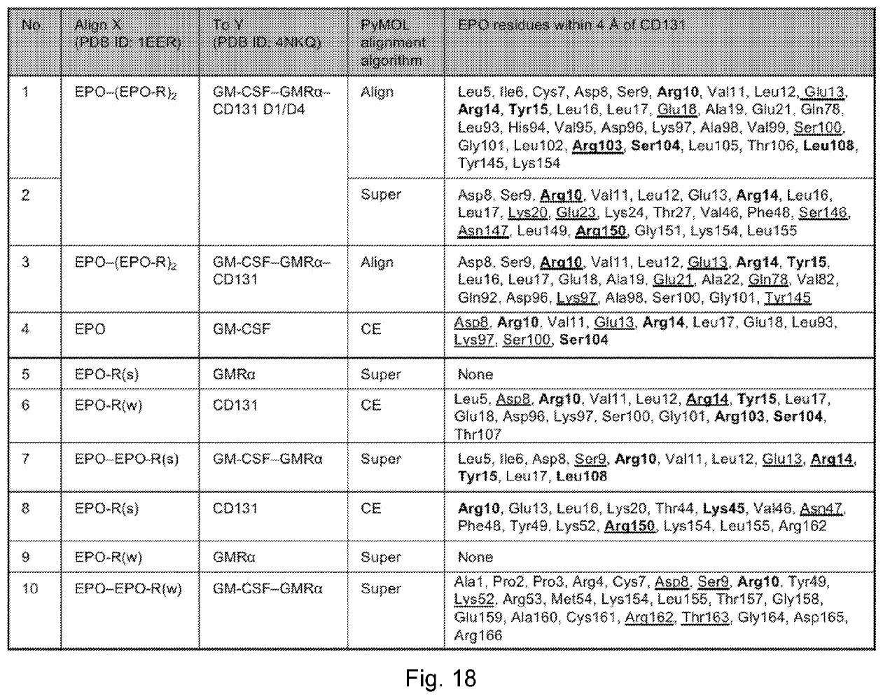

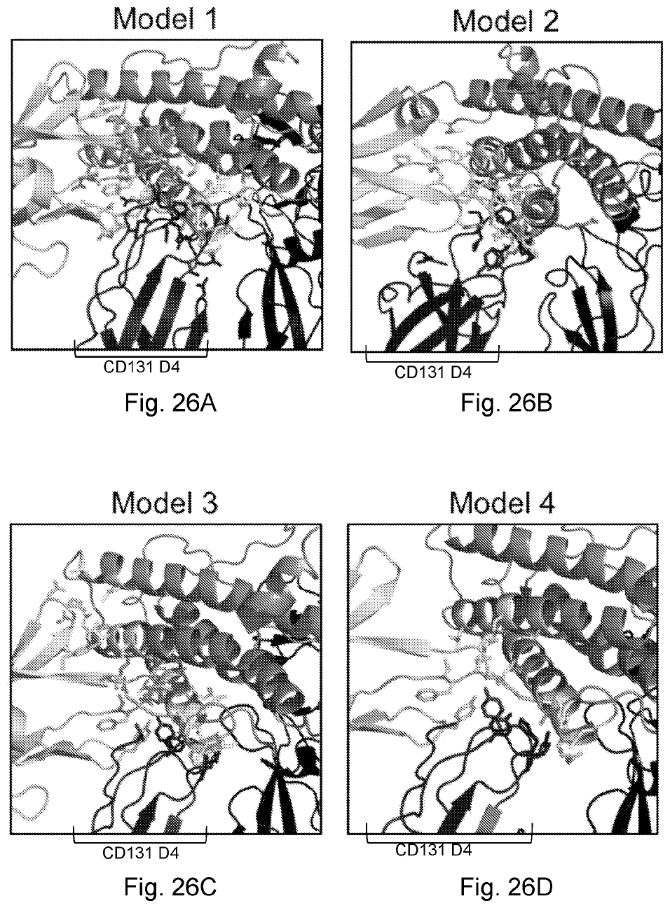

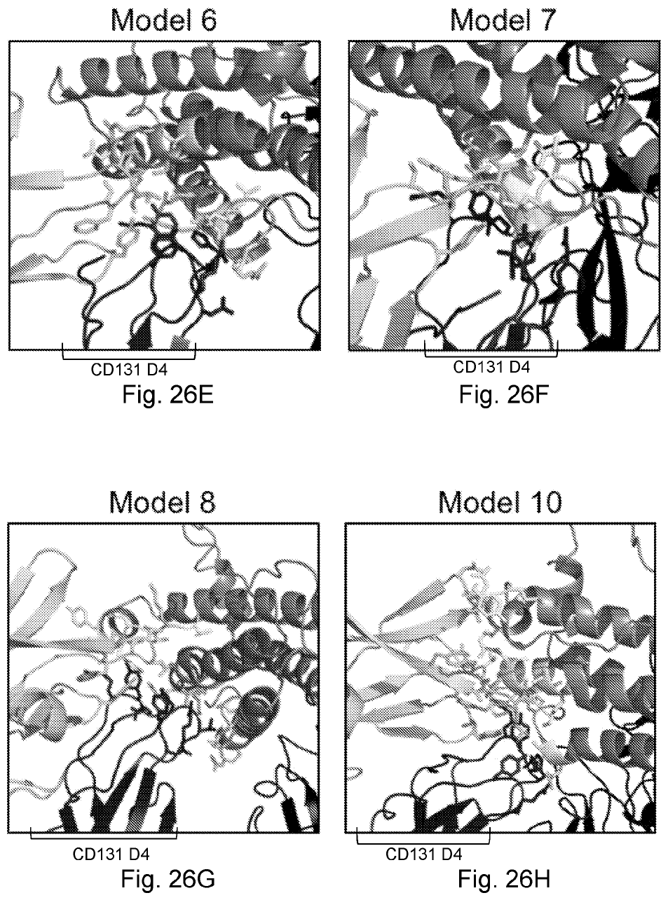

EPO residues that are important for receptor-ligand interaction and receptor activation were garnered from those within 4 Å of CD131 (see e.g., FIG. 18). Models 1, 3, 4, 6, and 7, in which CD131 binds at the weak side of EPO, showed that EPO residues in helices A and C could interact with the D1 and D4 domains of CD131. Arg10 and Arg14 of EPO were located in proximity to CD131 in all of these models. Tyr15, Arg103, Ser104, and Leu108 of EPO were found in some of these models (see e.g., FIG. 18 and FIG. 26A, FIG. 26C-26F). These results are consistent with other protein docking models generated. In these docking models, EPO interacts with CD131 via the weak side, and Arg10, Arg14, and Tyr15 of EPO are predicted to be crucial for EPO-CD131 interaction. On the other hand, models 2, 8, and 10, in which CD131 binds at the strong side of EPO, had contact residues in helices A and D as well as in the AB loop. Arg10 was found to be in close contacts with CD131 residues in all three models. More interestingly, Lys45 and Arg150 of EPO, which abolished the tissue-protective effect of EPO upon mutation, were within 4 Å of CD131 in models 2 and 8 (see e.g., FIG. 18, FIG. 26B, FIG. 26G, and FIG. 26H). In model 10, EPO residues at the N- and C-termini pointed at CD131 but those lining the major helices and loops were not directly in contact with CD131, indicating that parts other than the strong and weak sides of EPO may interact with CD131 (see e.g., FIG. 18 and FIG. 26H). Structural models based on the GM-CSF receptor complex showed that all four hypotheses for the formation of EPO-EPO—R-CD131 are plausible and that EPO residues that are close to CD131 are in a good agreement to those that were selected for mutagenesis herein as well as those resulting from other docking studies. See e.g., Shing et al., 2018, supra; the content of which is incorporated herein by reference in its entirety.

Mutagenesis studies on the GM-CSF signaling complex show that mutations that abolish bioactivity of the ligand are the ones that dramatically disrupt the binding of CD131, not the receptor alpha subunit. For instance, GMRα (K195D) completely lost GM-CSF binding but reduced GM-CSF activity only by about 10-fold in vitro. Similarly, when the affinity of GM-CSF(D112K) for GMRα was reduced by about 230-fold, the in vitro cell-proliferative activity of the same mutant was reduced only by about 8-fold. These results indicate that even total disruption of ligand binding to the receptor alpha subunit may not necessarily block bioactivity of the ligand, and highlight the importance of affinity conversion by CD131. This is consistent with previous observations that mutations that disrupt the GM-CSF-CD131 interaction completely eliminated cellular responses to GM-CSF. If the EPO-EPO—R-CD131 heterocomplex follows a similar assembly mechanism as the GM-CSF signaling complex, then one can deduce that residues that remove tissue-protective effects but not erythropoietic effects upon mutation are likely to be essential for CD131 interaction. None of the EPO mutants tested herein ablated tissue-protective activity while still maintaining erythropoietic activity independently of IH4 fusion. Thus, mutagenesis studies can be performed to identify residues that are important for CD131 interaction, if EPO and CD131 indeed form direct contacts. See e.g., Broughton et al., 2016, supra; Lopez et al, Immunology today 13(12), 495-500, 1992; Rozwarski et al., Proteins: Structure, Function, and Bioinformatics 26(3), 304-313, 1996; the contents of each of which are incorporated herein by reference in their entireties.

Next, experimental data were examined under the four hypothetical scenarios proposed above. In in vitro cell-based assays, mutating Lys45 or Arg150 resulted in the loss of both erythropoietic and tissue protective activity (see e.g., FIG. 12A-12B and FIG. 14D-14E). This behavior of the Lys45Asp (K45D) and Arg150Ala (R150A) mutants does not rule out any hypothesis. Erythroid cell proliferation assay data indicate that these two mutants interfere with EPO-R binding at the strong side. Hence, their loss of tissue-protective effects can also be caused by the disruption of EPO interaction with EPO-R. Hypotheses 1, 3, and 4, in which the strong side of EPO may interact with EPO-R, fit with this set of data. Hypothesis 2, in which the strong side of EPO may interact with CD131, cannot be rejected by these data either because tissue protective activity of R150A and K45D may be significantly reduced if these mutants hinder with the binding of CD131.

On the contrary, the activity of the Ser104Ile (S104I) and Leu108Ala (L108A) mutants ruled out the possibility of CD131 binding at the strong side of EPO (hypothesis 2). Erythroid cell proliferation assay data show that these mutants completely ablate EPO-R homodimer signaling by disrupting the receptor interaction at the weak side of EPO (see e.g., FIG. 12A-12B). This indicates that the binding of EPO to EPO-R and CD131 via the weak and strong sides, respectively, should also be disrupted by these mutants. However, these mutants were able to protect neuronal cells from cobalt-induced death in cell-based assays (see e.g., FIG. 14C, FIG. 14H, FIG. 14I), providing a counterevidence for hypothesis 2. These data do not reject the other three hypotheses, as these mutations may not reduce CD131 binding affinity strongly enough to affect signaling (hypothesis 1), or may not directly interact with CD131 at all (hypotheses 3 and 4).

Lastly, erythropoietic and tissue-protective activity of darbepoetin (see e.g., FIG. 12A-12B and FIG. 14B) rules out the possibility of EPO interaction with CD131 via the pointy end of the prolate-spheroid-shaped EPO that is opposite to the N- and C-termini (that is, regions near the AB and BC loops). Darbepoetin has two additional N-linked glycosylation sites (Asn30 and Asn88) engineered into wild-type EPO. The added sugar moieties are located at a region of EPO that does not directly bind to EPO-R but are bulky and negatively charged so that they cause steric and electrostatic hindrance with receptors, as evidenced by slightly reduced erythroid cell proliferative activity compared to wild-type EPO (see e.g., FIG. 12A). Despite these bulky changes, darbepoetin was able to promote tissue protection in vitro, suggesting that CD131 likely does not form a direct contact with EPO via the region with extra glycosylation. These data partially but not entirely rebut the hypothesis that CD131 may bind EPO via regions other than the strong and weak side (hypothesis 3).

Structural alignment models and cell-based experiments show that three out of four proposed hypotheses can explain the potential binding mode of the EPO-EPO—R-CD131 complex. The second hypothesis that EPO may interact with EPO-R and CD131 via the weak and strong sides, respectively, is kinetically less favorable than the other hypothesized configurations, and is eventually rejected by the weak-side EPO mutants that failed to stimulate erythroid cell proliferation but exhibited tissue-protective activity. These analyses help visualize potential three-dimensional structures of the EPO-EPO—R-CD131 heterocomplex and estimate the regions of EPO that might be involved in receptor activation for tissue protection. More structural and mutagenesis studies can be performed to validate or disprove the proposed hypotheses and structural alignment models, and to broaden understanding of mechanisms of the pleiotropic actions of EPO.

### Conclusions

Attachment of different EPO mutants to an antibody element permits the mixing and matching of different flavors of EPO action for various therapeutic applications. For instance, a form of EPO that stimulates both RBC production and tissue protection without pro-thrombotic side effects is useful for the treatment of high-altitude-related illnesses, as well as for general performance enhancement and prophylactic protection from chemical and physical damage in the military. A version of EPO that stimulates only tissue protection without RBC production and pro-thrombotic side effects is useful for the treatment of TBI and neurodegenerative diseases as well as surgery enhancement. In addition to a broader spectrum of therapeutic applications, incorporating a mutation that significantly reduces side effects and maintains the desired effect increases the maximum allowed dose of EPO in clinical studies, thereby allowing studies to be conducted at more clinically relevant doses. Currently, the maximum allowed dose of EPO is limited by its known effects on anemia in patients with chronic kidney diseases. The effective dose of EPO for protection of neuronal cells, however, is predicted to be much higher than that for the treatment of anemia because only <1% of administered EPO is able to cross the BBB, drastically reducing the local concentration of EPO available for target cells. If doses are limited by the value set for anemic patients, then EPO is likely to fail clinical trials for neuroprotection because doses are insufficient to reach an effective local concentration in the target tissue and not necessarily because the drug itself is not effective. Engineering strategies described herein permit one to harness different sets of beneficial effects of EPO according to different medical needs, and to improve the chances of successful clinical trials by allowing the administration of more clinically relevant doses.

### Materials and Methods

Materials and Methods for cell culture of 293-F, CHO—S and CHO DG44 cell lines, DNA construction of EPO variants, protein expression and his-tag purification, in vitro measurement of erythropoietic activity by cell proliferation assays, and measurement of mouse reticulocytes and reticulated platelets by flow cytometry are described in Example 1 or in International Patent Application WO 2020/132234.

Cell Culture. Human neuroblastoma cell line, SH-SY5Y, was obtained from ATCC. SH-SY5Y cells were cultured in 1:1 DMEM/F-12 with 10% FBS at 37° C. in 5% CO₂.

Neuroprotection Assay. SH-SY5Y cells were seeded in a 96-well plate at 4.0×10⁴ cells per well in 80 μL of 1:1 DMEM/F-12 with 10% FBS, and were let adhere overnight in the incubator (37° C., 5% CO₂). Cells were pre-treated with varying concentrations of purified proteins for 24 hr at 37° C. in 5% CO₂. Then 100 μM of cobalt chloride (CoCl₂), a hypoxia-mimicking agent, was added to cells. After 24 hr, cell viability was measured by CELLTITER 96® AQUEOUS ONE SOLUTION CELL PROLIFERATION ASSAY (PROMEGA). Absorbance at 490 nm was read on a BIOTEK SYNERGY NEO HTS microplate reader. Reported data represent mean±S.E.M of two or three replicates.

Structural Alignment. The crystal structures of EPO-(EPO-R)₂ (see e.g., PDB ID: lEER) and GM-CSF-GMRα-CD131 (see e.g., PDB ID: 4NKQ) were obtained from Research Collaboratory for Structural Bioinformatics Protein Data Bank™ (RCSB PDB). The structural alignments of these two receptor-ligand complexes were performed and root-mean-square deviation (RMSD) values were calculated using three algorithms, Align, Super, and CE, in PyMOL. Super and CE were preferred to Align, as the sequence identities between EPO and GM-CSF and between EPO-R and GMRα or CD131 were lower than 30%. Super and CE were excluded from analyses of some alignment models due to the following limitations. Super had a tendency to align EPO-R with CD131 homodimer interchain region, orienting domain 2 of EPO-R parallel to the membrane. CE aligned solely based on protein domains with most structural similarity, ignoring the rest of the complex being aligned. Residues that are within 4 Å of interacting receptor or ligand were selected using PyMOL.

1. A polypeptide comprising:
a) an engineered erythropoietin (EPO) comprising at least one affinity-decreasing mutation in the weak face of EPO relative to wild-type EPO;
b) an anti-glycophorin A (GYPA) antibody reagent that binds an epitope of SEQ ID NO: 89; and
c) a linker sequence separating the anti-GYPA antibody reagent and the engineered erythropoietin.

2. The polypeptide of claim 1, wherein the weak face of EPO binds with a dissociation constant (K_{d}) of at least 1 μM to the EPO receptor (EPOR).

3. The polypeptide of claim 1, wherein the at least one mutation is:
in Helix A (Ser9-Gly28 of SEQ ID NO: 1) and/or Helix C (Pro90-Leu112 of SEQ ID NO: 1) relative to wild-type EPO of SEQ ID NO: 1;
at an amino acid residue relative to SEO ID NO: 1 selected from S104, R14, Y15, R103, and L108;
S104I, R14E, R140, R14N, Y15I, R103I, R103Q, R103K, or L108A;
at an amino acid residue relative to SEO ID NO: 1 selected from R14, R103, and L108;
R14N, R103I, R103K, or L108A;
at an amino acid residue relative to SEQ ID NO: 1 selected from R103 and L108; or
R103K or L108A.

4-9. (canceled)

10. The polypeptide of claim 1, wherein the at least one mutation does not substantially affect binding to CD131.

11. The polypeptide of claim 1, wherein the engineered erythropoietin does not comprise a mutation:
in a region of wild-type EPO that binds to CD131;
in a strong face of EPO relative to wild-type EPO, wherein the strong face of EPO binds with a dissociation constant (K_{d}) of no more than 1 nM to EPOR;
in Helix D (F138-C161 of SEQ ID NO: 1) or the AB loop (C29-E55 of SEQ ID NO: 1) relative to wild-type EPO of SEQ ID NO: 1;
at an amino acid residue relative to SEQ ID NO: 1 selected from R150, K45, A30, H32, P87, W88, P90, R53, and E55; or
selected from R150A, K45D, A30N, H32T, P87V, W88N, P90T, R53N, or E55T.

12-16. (canceled)

17. The polypeptide of claim 1, wherein the anti-GYPA antibody reagent comprises;
one or more CDRs of IH4;
the three CDRs of IH4;
a VHH having the sequence of SEQ ID NO: 2 or SEQ ID NO: 50;
one or more CDRs of an antibody reagent selected from the group consisting of 10F7, 1C3, 2B-11, 2B-12, 2B-13, 2B-18, 2B-19, 2B-20, 2B-21, 2B-25, 2B-4, 2B-9, A63-B/C2, A88-A/F9, A88-D/C7, A88-E/H2, A96-D/A7, A96-E/F7, B14 (also known as BRIC 14), B89 (also known as BRIC 89), BRIC 116, BRIC 117, BRIC 119, BRIC 93, GPA 105, GPA 33, IH4, IH4v1, Mab 158, NaM10-2H12, NaM10-6G4, NaM16-IB10, NaM70-3C10, OSK4-1, R 10, R7, and R18;
one or more CDRs of an antibody reagent selected from R18, IH4, IH4v1, 10F7, and Table 13;
the CDRs of an antibody reagent selected from R18, IH4, IH4v1, 10F7, and Table 13; or
the VH and VL sequences of an antibody reagent selected from R18, IH4, IH4v1, 10F7, and Table 13.

18-23. (canceled)

24. The polypeptide of claim 1, wherein the anti-GYPA antibody reagent comprises an antibody reagent selected from;
R18, IH4, IH4v1, 10F7, and Table 13;
10F7, R18, IH4, IH4v1, 2B-21, 2B-25, 2B-9, 2B-20, 2B-19, NaM70-3C10, 2B-4, B14, B89, R7, and BRIC 93; or
10F7, IH4, IH4v1, 2B-9, 2B-20, 2B-19, NaM70-3C10, 2B-4, B14, B89, R7, and BRIC 93.

25-26. (canceled)

27. The polypeptide of claim 1, wherein the linker sequence is;
no more than 17 amino acids in length;
1, 2, 3, 4 or 5 amino acids in length;
at least 5 amino acids in length;
5-35 amino acids in length;
5-7 amino acids in length; or
7 or fewer amino acids in length.

28-32. (canceled)

33. The polypeptide of claim 1, wherein the polypeptide comprises the amino acid sequence of SEQ ID NO: 148.

34. (canceled)

35. The polypeptide of claim 1, wherein the polypeptide comprises the amino acid sequence of SEQ ID NO: 148 and a detectable tag.

36. (canceled)

37. The polypeptide of claim 1, wherein the polypeptide consists of the amino acid sequence of SEQ ID NO: 144.

38. A nucleic acid encoding the polypeptide of claim 1.

39. A vector comprising the nucleic acid of claim 38.

40. A cell comprising the nucleic acid of claim 38.

41. A pharmaceutical composition comprising the polypeptide of claim 1.

42. A method of increasing erythropoiesis comprising contacting a red blood cell with a polypeptide of claim 1.

43. A method of decreasing neurodegeneration comprising contacting a neuron with a polypeptide of claim 1.

44. (canceled)

45. A method of treating a neurodegenerative disease or disorder, hypoxic tissue damage, traumatic brain injury, or stroke in a subject in need thereof, the method comprising administering an effective amount of a polypeptide of claim 1 to the subject.

46. A method of treating altitude sickness or hypoxic tissue damage in a subject in need thereof, the method comprising administering an effective amount of a polypeptide of claim 1 to the subject.

47. A method of enhancing physical performance in a subject in need thereof, the method comprising administering an effective amount of a polypeptide of claim 1 to the subject.

48-51. (canceled)


SEQUENCE LISTING (149 sequences)

SEQ ID NO: 1 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 2 - protein, 123 aa, Artificial Sequence
  synthetic polypeptide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVS

SEQ ID NO: 3 - protein, 5 aa, Artificial Sequence
  synthetic polypeptide
SGGGS

SEQ ID NO: 4 - DNA, 369 bp, Artificial Sequence
  synthetic polynucleotide
caggtccaactgcaagagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagc

SEQ ID NO: 5 - DNA, 15 bp, Artificial Sequence
  synthetic polynucleotide
tctggtggtggttcc

SEQ ID NO: 6 - DNA, 498 bp, Homo sapiens
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 7 - protein, 11 aa, Artificial Sequence
  synthetic polypeptide
  misc_feature (1)..(1): Xaa indicates pyroglutamate
XEELERALNSS

SEQ ID NO: 8 - protein, 19 aa, Artificial Sequence
  synthetic polypeptide
GCAEHCSLNENITVPDTKV

SEQ ID NO: 9 - protein, 12 aa, Artificial Sequence
  synthetic polypeptide
SGLRSLTTLLRA

SEQ ID NO: 10 - protein, 5 aa, Artificial Sequence
  synthetic polypeptide
VTVSS

SEQ ID NO: 11 - protein, 294 aa, Artificial Sequence
  synthetic polypeptide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVSSGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 12 - DNA, 882 bp, Artificial Sequence
  synthetic polynucleotide
caggtccaactgcaagagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 13 - protein, 166 aa, Artificial Sequence
  synthetic polypeptide
APPRLICDSRVLERYLLEAKEAENITTGCAEHCSLNENITVPDTDVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 14 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLAGKLKLYTGEACRTGDR

SEQ ID NO: 15 - protein, 166 aa, Artificial Sequence
  synthetic polypeptide
APPRLICDSRVLEEYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 16 - protein, 166 aa, Artificial Sequence
  synthetic polypeptide
APPRLICDSRVLEQYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 17 - protein, 166 aa, Artificial Sequence
  synthetic polypeptide
APPRLICDSRVLENYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 18 - protein, 166 aa, Artificial Sequence
  synthetic polypeptide
APPRLICDSRVLERILLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 19 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLISLTTLLRALGAQKEAISPPDAASAAPLRTITADTFRKLFRVYSNFLRGKLKLYTGEACRTGDR

SEQ ID NO: 20 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLQSLTTLLRALGAQKEAISPPDAASAAPLRTITADTFRKLFRVYSNFLRGKLKLYTGEACRTGDR

SEQ ID NO: 21 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLKSLTTLLRALGAQKEAISPPDAASAAPLRTITADTFRKLFRVYSNFLRGKLKLYTGEACRTGDR

SEQ ID NO: 22 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRILTTLLRALGAQKEAISPPDAASAAPLRTITADTFRKLFRVYSNFLRGKLKLYTGEACRTGDR

SEQ ID NO: 23 - protein, 166 aa, Artificial Sequence
  synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDR

SEQ ID NO: 24 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactgatgttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 25 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgg

SEQ ID NO: 26 - DNA, 498 bp, Artificial Sequence
  synthetic polynucleotide
gccccacctagattgatttgtgattccagagttttggaagaatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 27 - DNA, 498 bp, Artificial Sequence
  synthetic polynucleotide
gccccacctagattgatttgtgattccagagttttggaacaatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 28 - DNA, 498 bp, Artificial Sequence
  synthetic polynucleotide
gccccacctagattgatttgtgattccagagttttggaaaactacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 29 - DNA, 498 bp, Artificial Sequence
  synthetic polynucleotide
gccccacctagattgatttgtgattccagagttttggaaagaatcttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 30 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgatctctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 31 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgcagtctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 32 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgaaatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 33 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagaatcttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 34 - DNA, 498 bp, Artificial Sequence
  synthetic polynucleotide
gccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 35 - protein, 5 aa, Artificial Sequence
  synthetic polypeptide
YAATP

SEQ ID NO: 36 - protein, 4 aa, Artificial Sequence
  synthetic polypeptide
RTVY

SEQ ID NO: 37 - protein, 4 aa, Artificial Sequence
  synthetic polypeptide
YPPE

SEQ ID NO: 38 - DNA, 369 bp, Artificial Sequence
  synthetic polynucleotide
caggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagc

SEQ ID NO: 39 - DNA, 366 bp, Artificial Sequence
  synthetic polynucleotide
caggtccaactgcaagagagcggaggagggtctgttcaagctggcggttccctccggctttcttgcgtggcgtcaggctatactgacagcacatactgcgtgggctggttcaggcaggcccctggaaaggagcgcgagggcgtagcccgcataaatactatatctggcagaccgtggtacgctgacagcgtgaagggacggtttacaatcagtcaagataactctaaaaacaccgtgtttcttcaaatgaattctttgaaacccgaagatactgccatctattattgcacacttacgaccgcgaactcacgcggtttttgtagcggaggatataactataaagggcaagggcaggtaactgtatcc

SEQ ID NO: 40 - DNA, 726 bp, Artificial Sequence
  synthetic polynucleotide
caagttaagttgcaacaatctggtgctgaattggttaagccaggtgcttctgttaagttgtcttgtaaggcttctggttacaccttcaactcttactttatgcattggatgaagcaaagaccagttcaaggtttggaatggattggtatgattagaccaaacggtggtactaccgattacaacgagaagtttaagaacaaggctactttgactgttgataagtcctctaacactgcttacatgcaattgaactctttgacttctggtgattctgctgtttactactgtgctagatgggaaggttcttactacgctttggattactggggtcaaggtaccactgttactgtttcttccggtggaggtggatctggtggtggaggatcttcaggaggtggtggatcttccgatattgagttgactcaatctccagctattatgtctgctaccttgggtgagaaggttactatgacttgtagagcttcatctaacgttaagtacatgtactggtaccaacagaagtctggtgcttctccaaagttgtggatttactacacttctaacttggcttctggtgttccaggtagattttctggttcaggttctggtacttcctactctttgactatttcctctgttgaagctgaagatgctgctacttactactgtcaacaattcacttcttccccatacacttttggaggaggtactaagttggaaatcaag

SEQ ID NO: 41 - DNA, 747 bp, Artificial Sequence
  synthetic polynucleotide
gaagtccgtctgctggaaagcgggggtggtcctgtgcagcctggtgggtccctgaaactgtcctgtgccgcaagcgggttcgatttttccagatactggatgaactgggtgaggagggctccaggcaagggcctggagtggatcggcgagatcaaccagcagtccagcaccatcaattactctccccctctgaaggacaagttcatcatcagccgcgataacgctaagtctacactgtatctgcagatgaataaggtgagaagcgaggacaccgccctgtactattgcgctcgcctgtctctgacagccgctggctttgcctattggggccagggcaccctggtgacagtgtctgctggaggaggctcttccggaggatccggcagctctggcggctccagctctggcggcgatatcgtgatgagccagtctccctccagcctggccgtgtccgtgggagagaaggtgtccatgagctgtaagtcttcccagtctctgttcaactccagaacccgcaagaattacctgacatggtatcagcagaagcctggccagagccccaagcctctgatctactgggccagcaccagagagtctggagtgccagaccgcttcaccggctctggatccggcacagacttcaccctgacaatcagctctgtgcaggccgaggacctggctgattactattgcaagcagtcctataatctgaggacctttggcggcggcacaaagctggagatcaag

SEQ ID NO: 42 - DNA, 741 bp, Artificial Sequence
  synthetic polynucleotide
caggttaaactccagcaaagtggtggcgggctcgtacaaccaggcggttccctcaagttgtcctgcgccgcatcagggtttacatttagctcttatggtatgtcttggtttcgccagacgcctgacaagcgactcgagctggtcgctatcatcaatagtaacggaggtactacatattatcccgacagtgtgaaggggcgatttaccattagccgggacaacgccaaaaatacactgtacctccagatgtcaagcttgaaatcagaagatacggccatgtactattgcgctagggggggtggaaggtggcttctggactattatggtcagggtacaacagtgacagtatcctccggtggaggtggatctggtggtggaggatcttcaggaggtggtggatcttccgacatagagcttacacaatctccgtcatcactggcagtctcagccggggaaaaagtgacaatgtcatgcaagtcaagccagagcgttctttattcatctaatcagaagaactacctggcatggtatcagcagaagccgggacagtcccctaagctcctcatctactgggcaagcaccagggaatccggagtgccggacaggtttactgggtccggttctgggacggattttacgcttacgatatcaagtgtccaagctgaggacctcgcagtatactactgtcaccagtacctgtcttcttctacttttgggggtggaacgaaactggaaataaaa

SEQ ID NO: 43 - DNA, 498 bp, Artificial Sequence
  synthetic polynucleotide
gctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 44 - DNA, 498 bp, Artificial Sequence
  synthetic polynucleotide
gctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttggccggtaagttgaagttgtacactggtgaagcttgtagaactggtgatcgg

SEQ ID NO: 45 - DNA, 498 bp, Artificial Sequence
  synthetic polynucleotide
gctccacctagattgatttgtgattccagagttttggaaagatacttgttggaagctaaggaggctgaaaatattactactggttgtgctgaacattgttctttgaacgagaatattactgttccagatactgatgttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccttgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 46 - DNA, 21 bp, Artificial Sequence
  synthetic polynucleotide
ggaggatctggtggtggttcc

SEQ ID NO: 47 - DNA, 51 bp, Artificial Sequence
  synthetic polynucleotide
ggaggatccggtggtggaggatcatctggtggaggatctggtggtggttcc

SEQ ID NO: 48 - DNA, 87 bp, Artificial Sequence
  synthetic polynucleotide
ggaggaagttccggtggtggatcttcttctggaggtggaggatccggtggtggaggatcatctggtggaggatctggtggtggttcc

SEQ ID NO: 49 - DNA, 105 bp, Artificial Sequence
  synthetic polynucleotide
ggtggaggtggttccggaggaggaagttccggtggtggatcttcttctggaggtggaggatccggtggtggaggatcatctggtggaggatctggtggtggttcc

SEQ ID NO: 50 - protein, 122 aa, Artificial Sequence
  synthetic polypeptide
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGQVTVS

SEQ ID NO: 51 - protein, 242 aa, Artificial Sequence
  synthetic polypeptide
QVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVSSGGGGSGGGGSSGGGGSSDIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIK

SEQ ID NO: 52 - protein, 249 aa, Artificial Sequence
  synthetic polypeptide
EVRLLESGGGPVQPGGSLKLSCAASGFDFSRYWMNWVRRAPGKGLEWIGEINQQSSTINYSPPLKDKFIISRDNAKSTLYLQMNKVRSEDTALYYCARLSLTAAGFAYWGQGTLVTVSAGGGSSGGSGSSGGSSSGGDIVMSQSPSSLAVSVGEKVSMSCKSSQSLFNSRTRKNYLTWYQQKPGQSPKPLIYWASTRESGVPDRFTGSGSGTDFTLTISSVQAEDLADYYCKQSYNLRTFGGGTKLEIK

SEQ ID NO: 53 - protein, 247 aa, Artificial Sequence
  synthetic polypeptide
QVKLQQSGGGLVQPGGSLKLSCAASGFTFSSYGMSWFRQTPDKRLELVAIINSNGGTTYYPDSVKGRFTISRDNAKNTLYLQMSSLKSEDTAMYYCARGGGRWLLDYYGQGTTVTVSSGGGGSGGGGSSGGGGSSDIELTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSSTFGGGTKLEIK

SEQ ID NO: 54 - protein, 7 aa, Artificial Sequence
  synthetic polypeptide
GGSGGGS

SEQ ID NO: 55 - protein, 17 aa, Artificial Sequence
  synthetic polypeptide
GGSGGGGSSGGGSGGGS

SEQ ID NO: 56 - protein, 29 aa, Artificial Sequence
  synthetic polypeptide
GGSSGGGSSSGGGGSGGGGSSGGGSGGGS

SEQ ID NO: 57 - protein, 35 aa, Artificial Sequence
  synthetic polypeptide
GGGGSGGGSSGGGSSSGGGGSGGGGSSGGGSGGGS

SEQ ID NO: 58 - protein, 126 aa, Artificial Sequence
  synthetic polypeptide
QVQLVESGGGSVQAGGSLRLSCAASGYTVSTYCMGWFRQAPGKEREGVATILGGSTYYGDSVKGRFTISQDNAKNTVYLQMNSLKPEDTAIYYCAGSTVASTGWCSRLRPYDYHYRGQGTQVTVSS

SEQ ID NO: 59 - protein, 125 aa, Artificial Sequence
  synthetic polypeptide
QVQLQESGGGSVQAGGSLRLSCTASGYTYRKYCMGWFRQAPGKEREGVACINSGGGTSYYADSVKGRFTISQDNAKDTVFLRMNSLKPEDTAIYYCALSSNSVCPPGHVAWYNDWGQGTQVTVSS

SEQ ID NO: 60 - protein, 128 aa, Artificial Sequence
  synthetic polypeptide
QVQLQESGGGLVQAGGSLRLSCAASGFTLDSYAIGWFRQAPGKEREGVSCISASGGSTNYADSVKGRFTISRDNAKNTVYLQMNSLKSEDTAVYYCAADHPGLCTSESGRRRYLEVWGQGTQVTVSSA

SEQ ID NO: 61 - protein, 115 aa, Artificial Sequence
  synthetic polypeptide
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 62 - protein, 126 aa, Artificial Sequence
  synthetic polypeptide
GQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSH

SEQ ID NO: 63 - protein, 121 aa, Artificial Sequence
  synthetic polypeptide
QVQLQESGGGSVQAGGSLRLSCSASGYTYISGWFRQAPGKEREGVAAIRSSDGTTYYADSVKGRFTISQDNAKNTVYLQMNSLKPEDTAMYYCAATEVAGWPLDIGIYDYWGQGTEVTVSS

SEQ ID NO: 64 - protein, 118 aa, Artificial Sequence
  synthetic polypeptide
QVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVS

SEQ ID NO: 65 - protein, 18 aa, Artificial Sequence
  synthetic polypeptide
SGGGGSGGGGSSGGGGSS

SEQ ID NO: 66 - protein, 106 aa, Artificial Sequence
  synthetic polypeptide
DIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIK

SEQ ID NO: 67 - protein, 118 aa, Artificial Sequence
  synthetic polypeptide
EVRLLESGGGPVQPGGSLKLSCAASGFDFSRYWMNWVRRAPGKGLEWIGEINQQSSTINYSPPLKDKFIISRDNAKSTLYLQMNKVRSEDTALYYCARLSLTAAGFAYWGQGTLVTVS

SEQ ID NO: 68 - protein, 112 aa, Artificial Sequence
  synthetic polypeptide
DIVMSQSPSSLAVSVGEKVSMSCKSSQSLFNSRTRKNYLTWYQQKPGQSPKPLIYWASTRESGVPDRFTGSGSGTDFTLTISSVQAEDLADYYCKQSYNLRTFGGGTKLEIK

SEQ ID NO: 69 - protein, 118 aa, Artificial Sequence
  synthetic polypeptide
QVKLQQSGGGLVQPGGSLKLSCAASGFTFSSYGMSWFRQTPDKRLELVAIINSNGGTTYYPDSVKGRFTISRDNAKNTLYLQMSSLKSEDTAMYYCARGGGRWLLDYWGQGTTVTVSS

SEQ ID NO: 70 - protein, 112 aa, Artificial Sequence
  synthetic polypeptide
DIELTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSSTFGGGTKLEIK

SEQ ID NO: 71 - protein, 150 aa, Homo sapiens
MYGKIIFVLLLSEIVSISALSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 72 - protein, 11 aa, Artificial Sequence
  synthetic polypeptide
SGYTDSTYCVG

SEQ ID NO: 73 - protein, 17 aa, Artificial Sequence
  synthetic polypeptide
RINTISGRPWYADSVKG

SEQ ID NO: 74 - protein, 15 aa, Artificial Sequence
  synthetic polypeptide
TTANSRGFCSGGYNY

SEQ ID NO: 75 - protein, 6 aa, Artificial Sequence
  synthetic polypeptide
AATPRA

SEQ ID NO: 76 - protein, 11 aa, Artificial Sequence
  synthetic polypeptide
AATPRAHEVSE

SEQ ID NO: 77 - protein, 10 aa, Artificial Sequence
  synthetic polypeptide
ATPRAHEVSE

SEQ ID NO: 78 - protein, 5 aa, Artificial Sequence
  synthetic polypeptide
PRAHE

SEQ ID NO: 79 - protein, 6 aa, Artificial Sequence
  synthetic polypeptide
PRAHEV

SEQ ID NO: 80 - protein, 7 aa, Artificial Sequence
  synthetic polypeptide
PRAHEVS

SEQ ID NO: 81 - protein, 8 aa, Artificial Sequence
  synthetic polypeptide
PRAHEVSE

SEQ ID NO: 82 - protein, 10 aa, Artificial Sequence
  synthetic polypeptide
ISVRTVYPPE

SEQ ID NO: 83 - protein, 8 aa, Artificial Sequence
  synthetic polypeptide
RTVYPPEE

SEQ ID NO: 84 - protein, 5 aa, Artificial Sequence
  synthetic polypeptide
YPPEE

SEQ ID NO: 85 - protein, 6 aa, Artificial Sequence
  synthetic polypeptide
YPPEEE

SEQ ID NO: 86 - protein, 7 aa, Artificial Sequence
  synthetic polypeptide
YPPEEET

SEQ ID NO: 87 - protein, 5 aa, Artificial Sequence
  synthetic polypeptide
PPEEE

SEQ ID NO: 88 - protein, 6 aa, Artificial Sequence
  synthetic polypeptide
PPEEET

SEQ ID NO: 89 - protein, 85 aa, Artificial Sequence
  synthetic polypeptide
LSTTEVAMHTSTSSSVTKSYISSQTNDTHKRDTYAATPRAHEVSEISVRTVYPPEEETGERVQLAHHFSEPEITLIIFGVMAGVI

SEQ ID NO: 90 - DNA, 117 bp, Artificial Sequence
  synthetic polynucleotide
ggtggaagtagtggtggaggtggttccggaggaggaagttccggtggtggatcttcttctggaggtggaggatccggtggtggaggatcatctggtggaggatctggtggtggttcc

SEQ ID NO: 91 - protein, 17 aa, Artificial Sequence
  synthetic polypeptide
GGGGSGGGGSSGGGGSS

SEQ ID NO: 92 - protein, 39 aa, Artificial Sequence
  synthetic polypeptide
GGSSGGGGSGGGSSGGGSSSGGGGSGGGGSSGGGSGGGS

SEQ ID NO: 93 - protein, 3 aa, Artificial Sequence
  synthetic polypeptide
GGS

SEQ ID NO: 94 - protein, 6 aa, Artificial Sequence
  synthetic polypeptide
GGSGGS

SEQ ID NO: 95 - protein, 9 aa, Artificial Sequence
  synthetic polypeptide
GGSGGSGGS

SEQ ID NO: 96 - protein, 12 aa, Artificial Sequence
  synthetic polypeptide
GGSGGSGGSGGS

SEQ ID NO: 97 - protein, 15 aa, Artificial Sequence
  synthetic polypeptide
GGSGGSGGSGGSGGS

SEQ ID NO: 98 - protein, 5 aa, Artificial Sequence
  synthetic polypeptide
KLEIK

SEQ ID NO: 99 - protein, 5 aa, Artificial Sequence
  synthetic polypeptide
QVTVS

SEQ ID NO: 100 - protein, 18 aa, Artificial Sequence
  synthetic polypeptide
GGSGGSGGSGGSGGSGGS

SEQ ID NO: 101 - protein, 21 aa, Artificial Sequence
  synthetic polypeptide
GGSGGSGGSGGSGGSGGSGGS

SEQ ID NO: 102 - protein, 24 aa, Artificial Sequence
  synthetic polypeptide
GGSGGSGGSGGSGGSGGSGGSGGS

SEQ ID NO: 103 - protein, 27 aa, Artificial Sequence
  synthetic polypeptide
GGSGGSGGSGGSGGSGGSGGSGGSGGS

SEQ ID NO: 104 - protein, 30 aa, Artificial Sequence
  synthetic polypeptide
GGSGGSGGSGGSGGSGGSGGSGGSGGSGGS

SEQ ID NO: 105 - protein, 4 aa, Artificial Sequence
  synthetic polypeptide
GGGS

SEQ ID NO: 106 - protein, 8 aa, Artificial Sequence
  synthetic polypeptide
GGGSGGGS

SEQ ID NO: 107 - protein, 12 aa, Artificial Sequence
  synthetic polypeptide
GGGSGGGSGGGS

SEQ ID NO: 108 - protein, 16 aa, Artificial Sequence
  synthetic polypeptide
GGGSGGGSGGGSGGGS

SEQ ID NO: 109 - protein, 20 aa, Artificial Sequence
  synthetic polypeptide
GGGSGGGSGGGSGGGSGGGS

SEQ ID NO: 110 - protein, 24 aa, Artificial Sequence
  synthetic polypeptide
GGGSGGGSGGGSGGGSGGGSGGGS

SEQ ID NO: 111 - protein, 28 aa, Artificial Sequence
  synthetic polypeptide
GGGSGGGSGGGSGGGSGGGSGGGSGGGS

SEQ ID NO: 112 - protein, 32 aa, Artificial Sequence
  synthetic polypeptide
GGGSGGGSGGGSGGGSGGGSGGGSGGGSGGGS

SEQ ID NO: 113 - protein, 36 aa, Artificial Sequence
  synthetic polypeptide
GGGSGGGSGGGSGGGSGGGSGGGSGGGSGGGSGGGS

SEQ ID NO: 114 - protein, 40 aa, Artificial Sequence
  synthetic polypeptide
GGGSGGGSGGGSGGGSGGGSGGGSGGGSGGGSGGGSGGGS

SEQ ID NO: 115 - protein, 5 aa, Artificial Sequence
  synthetic polypeptide
GGGGS

SEQ ID NO: 116 - protein, 10 aa, Artificial Sequence
  synthetic polypeptide
GGGGSGGGGS

SEQ ID NO: 117 - protein, 15 aa, Artificial Sequence
  synthetic polypeptide
GGGGSGGGGSGGGGS

SEQ ID NO: 118 - protein, 20 aa, Artificial Sequence
  synthetic polypeptide
GGGGSGGGGSGGGGSGGGGS

SEQ ID NO: 119 - protein, 25 aa, Artificial Sequence
  synthetic polypeptide
GGGGSGGGGSGGGGSGGGGSGGGGS

SEQ ID NO: 120 - protein, 30 aa, Artificial Sequence
  synthetic polypeptide
GGGGSGGGGSGGGGSGGGGSGGGGSGGGGS

SEQ ID NO: 121 - protein, 35 aa, Artificial Sequence
  synthetic polypeptide
GGGGSGGGGSGGGGSGGGGSGGGGSGGGGSGGGGS

SEQ ID NO: 122 - protein, 40 aa, Artificial Sequence
  synthetic polypeptide
GGGGSGGGGSGGGGSGGGGSGGGGSGGGGSGGGGSGGGGS

SEQ ID NO: 123 - protein, 45 aa, Artificial Sequence
  synthetic polypeptide
GGGGSGGGGSGGGGSGGGGSGGGGSGGGGSGGGGSGGGGSGGGGS

SEQ ID NO: 124 - protein, 50 aa, Artificial Sequence
  synthetic polypeptide
GGGGSGGGGSGGGGSGGGGSGGGGSGGGGSGGGGSGGGGSGGGGSGGGGS

SEQ ID NO: 125 - protein, 6 aa, Artificial Sequence
  synthetic polypeptide
GGGGGS

SEQ ID NO: 126 - protein, 12 aa, Artificial Sequence
  synthetic polypeptide
GGGGGSGGGGGS

SEQ ID NO: 127 - protein, 18 aa, Artificial Sequence
  synthetic polypeptide
GGGGGSGGGGGSGGGGGS

SEQ ID NO: 128 - protein, 24 aa, Artificial Sequence
  synthetic polypeptide
GGGGGSGGGGGSGGGGGSGGGGGS

SEQ ID NO: 129 - protein, 30 aa, Artificial Sequence
  synthetic polypeptide
GGGGGSGGGGGSGGGGGSGGGGGSGGGGGS

SEQ ID NO: 130 - protein, 36 aa, Artificial Sequence
  synthetic polypeptide
GGGGGSGGGGGSGGGGGSGGGGGSGGGGGSGGGGGS

SEQ ID NO: 131 - protein, 42 aa, Artificial Sequence
  synthetic polypeptide
GGGGGSGGGGGSGGGGGSGGGGGSGGGGGSGGGGGSGGGGGS

SEQ ID NO: 132 - protein, 48 aa, Artificial Sequence
  synthetic polypeptide
GGGGGSGGGGGSGGGGGSGGGGGSGGGGGSGGGGGSGGGGGSGGGGGS

SEQ ID NO: 133 - protein, 54 aa, Artificial Sequence
  synthetic polypeptide
GGGGGSGGGGGSGGGGGSGGGGGSGGGGGSGGGGGSGGGGGSGGGGGSGGGGGS

SEQ ID NO: 134 - protein, 60 aa, Artificial Sequence
  synthetic polypeptide
GGGGGSGGGGGSGGGGGSGGGGGSGGGGGSGGGGGSGGGGGSGGGGGSGGGGGSGGGGGS

SEQ ID NO: 135 - DNA, 1943 bp, Artificial Sequence
  synthetic polynucleotide
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgggggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 136 - DNA, 51 bp, Artificial Sequence
  synthetic polynucleotide
ggtggaggtggatctggtggtggaggatcttcaggaggtggtggatcttcc

SEQ ID NO: 137 - DNA, 10 bp, Artificial Sequence
  synthetic polynucleotide
gccrccatgg

SEQ ID NO: 138 - protein, 247 aa, Artificial Sequence
  synthetic polypeptide
QVKLQQSGGGLVQPGGSLKLSCAASGFTFSSYGMSWFRQTPDKRLELVAIINSNGGTTYYPDSVKGRFTISRDNAKNTLYLQMSSLKSEDTAMYYCARGGGRWLLDYWGQGTTVTVSSGGGGSGGGGSSGGGGSSDIELTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSSTFGGGTKLEIK

SEQ ID NO: 139 - DNA, 741 bp, Artificial Sequence
  synthetic polynucleotide
caggttaaactccagcaaagtggtggcgggctcgtacaaccaggcggttccctcaagttgtcctgcgccgcatcagggtttacatttagctcttatggtatgtcttggtttcgccagacgcctgacaagcgactcgagctggtcgctatcatcaatagtaacggaggtactacatattatcccgacagtgtgaaggggcgatttaccattagccgggacaacgccaaaaatacactgtacctccagatgtcaagcttgaaatcagaagatacggccatgtactattgcgctagggggggtggaaggtggcttctggactattggggtcagggtacaacagtgacagtatcctccggtggaggtggatctggtggtggaggatcttcaggaggtggtggatcttccgacatagagcttacacaatctccgtcatcactggcagtctcagccggggaaaaagtgacaatgtcatgcaagtcaagccagagcgttctttattcatctaatcagaagaactacctggcatggtatcagcagaagccgggacagtcccctaagctcctcatctactgggcaagcaccagggaatccggagtgccggacaggtttactgggtccggttctgggacggattttacgcttacgatatcaagtgtccaagctgaggacctcgcagtatactactgtcaccagtacctgtcttcttctacttttgggggtggaacgaaactggaaataaaa

SEQ ID NO: 140 - protein, 19 aa, Artificial Sequence
  synthetic polypeptide
MYGKIIFVLLLSEIVSISA

SEQ ID NO: 141 - protein, 5 aa, Artificial Sequence
  synthetic polypeptide
LSTTE

SEQ ID NO: 142 - DNA, 1011 bp, Artificial Sequence
  synthetic polynucleotide
atggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgggggcccgaacaaaaactcatctcagaagaggatctgaatagcgccgtcgaccatcatcatcatcatcat

SEQ ID NO: 143 - protein, 337 aa, Artificial Sequence
  synthetic polypeptide
METDTLLLWVLLLWVPGSTGQVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVSSGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDRGPEQKLISEEDLNSAVDHHHHHH

SEQ ID NO: 144 - protein, 317 aa, Artificial Sequence
  synthetic polypeptide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVSSGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDRGPEQKLISEEDLNSAVDHHHHHH

SEQ ID NO: 145 - DNA, 1874 bp, Artificial Sequence
  synthetic polynucleotide
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcggtgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 146 - DNA, 942 bp, Artificial Sequence
  synthetic polynucleotide
atggagacagacacactcctgctatgggtactgctgctctgggttccaggttccactggtcaggtccaactgcaggagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgctccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcgttgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg

SEQ ID NO: 147 - protein, 314 aa, Artificial Sequence
  synthetic polynucleotide
METDTLLLWVLLLWVPGSTGQVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVSSGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDR

SEQ ID NO: 148 - protein, 294 aa, Artificial Sequence
  synthetic polynucleotide
QVQLQESGGGSVQAGGSLRLSCVASGYTDSTYCVGWFRQAPGKEREGVARINTISGRPWYADSVKGRFTISQDNSKNTVYLQMNSLKPEDTAIYYCTLTTANSRGFCSGGYNYKGQGTQVTVSSGGGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTALRALGAQKEAISPPDAASAAPLRTITADTFRKLFRVYSNFLRGKLKLYTGEACRTGDR

SEQ ID NO: 149 - DNA, 882 bp, Artificial Sequence
  synthetic polynucleotide
caggtccaactgcaagagagcggcggggggtcagttcaggcgggggggagtctgcggttgagctgcgtagcttcaggctacactgacagcacctactgcgtgggatggtttcggcaggcacccggcaaggaacgagagggcgttgcacggatcaacactatctccggtcggccttggtacgcagatagtgttaagggacggtttactattagtcaggataactctaagaataccgtctaccttcagatgaatagcctgaaaccggaagacacggctatttactattgcacccttacaactgccaacagcagagggttttgttctgggggatataactacaaaggacaggggacccaagtcactgtcagctctggtggtggttccgccccacctagattgatttgtgattccagagttttggaaagatacttgttggaagctaaggaggctgaaaatattactactggttgtgctgaacattgttctttgaacgagaatattactgttccagatactaaggttaacttttacgcttggaagagaatggaagttggtcagcaagctgttgaagtttggcaaggtttggctttgttgtctgaagctgttttgagaggtcaagctttgttggttaattcttctcaaccatgggaaccattgcaattgcatgttgataaggctgtttctggtttgagatctttgactaccgcattgagagctttgggtgctcaaaaggaagctatttctcctccagatgctgcttctgccgctccattgagaactattactgctgatacttttagaaagttgtttagagtttactctaacttcttgagaggtaagttgaagttgtacactggtgaagcttgtagaactggtgatcgg